(19) United States
(12) Patent Application Publication
(10) Pub. No.: US 2026/0355212 A1
(43) Pub. Date: Nov. 12, 2026

(54) NOVEL PROTEASE INHIBITORS FOR TREATMENT OF CORONAVIRUS INFECTIONS

(71) Applicants: Takeda Pharmaceutical Company Limited, Osaka (JP); SCHRODINGER, INC., New York, NY (US)

(72) Inventors: Daniel CARNEY, San Diego, CA (US); Edcon CHANG, San Diego, CA (US); Mallareddy KOMANDLA, San Diego, CA (US); Leah L. FRYE, New York, NY (US); Abba LEFFLER, New York, NY (US)

(21) Appl. No.: 19/111,262

(22) PCT Filed: Sep. 12, 2023
(86) PCT No.: PCT/US2023/032564

§ 371 Date: Mar. 12, 2025

Related U.S. Application Data
(60) Provisional application No. 63/406,060, filed on Sep. 13, 2022.

(51) Int. Cl. A61K 31/55; A61K 31/404; A61K 31/407; A61K 31/428; A61K 31/437; A61K 31/454; A61P 31/14; C07D 401/12; C07D 401/14; C07D 403/12; C07D 403/14; C07D 405/14; C07D 417/14; C07D 471/04; C07D 491/048; C07D 491/056; C07D 495/04

(52) U.S. Cl. CPC A61K 31/55; A61K 31/404; A61K 31/407; A61K 31/428; A61K 31/437; A61K 31/454; A61P 31/14; C07D 401/12; C07D 401/14; C07D 403/12; C07D 403/14; C07D 405/14; C07D 417/14; C07D 471/04; C07D 491/048; C07D 491/056; C07D 495/04

(57) ABSTRACT

Disclosed are compounds represented by formulae (I) and (II): or a pharmaceutically acceptable salt thereof. Compounds of formula (I) and (II) are useful for treating coronavirus infection via inhibition of the protease Mpro.

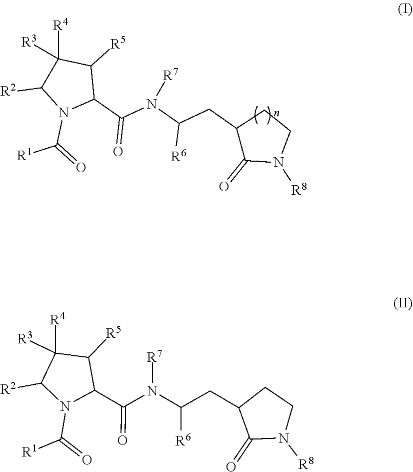

### CROSS-REFERENCE TO RELATED APPLICATIONS

This International PCT Application claims priority to and the benefit of U.S. Provisional Application No. 63/406,060 filed on Sep. 13, 2022, the contents of which are herein incorporated by reference in their entirety.

### TECHNICAL FIELD

The present disclosure relates to novel protease inhibitors useful for inhibiting coronavirus replication and methods for treating a subject infected by coronavirus including the SARS-CoV-2 virus.

### BACKGROUND

Coronaviruses are a family of enveloped, single-stranded, positive-sense, RNA viruses that are known to cause disease in a wide variety of animals. Zumla, A., et al., “Coronaviruses-drug discovery and therapeutic options,” Nature Reviews Drug Discovery 15(5):327-347 (2016). Seven coronaviruses are known to infect humans: SARS-CoV-1, SARS-CoV-2, MERS-CoV, HCoV-229E, HCoV—HKU1, HCoV-NL63 and HCoV-OC43. Shamsi, A., et al., “Potential drug targets of SARS-CoV-2: From genomics to therapeutics,” International Journal of Biological Macromolecules 177:1-9 (2021); and Ullrich, S. and Nitsche, C., “The SARS-CoV-2 main protease as drug target,” Bioorganic & Medicinal Chemistry Letters 30(17):127377 (2020). Most notably, SARS-CoV-2 is responsible for the COVID-19 respiratory illness and the ensuing pandemic, which as of March 2022, has caused more than 6 million deaths worldwide (WHO COVID-19 dashboard https://covid19.who.int/). Although safe and effective vaccines have been developed, global implementation has been hampered by limited access and recipient hesitancy. Furthermore, breakthrough infections and illness in vaccinated individuals have been widely documented. Most experts believe that COVID-19 will become an endemic illness that will persist in humans for many years to come, if not indefinitely. Therapeutic options for COVID-19 are currently limited. There is a clear unmet medical need for targeted antiviral therapeutics for the treatment COVID-19. Recent history of severe respiratory illnesses cause by coronaviruses (SARS-CoV-1, MERS-CoV, SARS-CoV-2) highlights the importance of pan-coronavirus therapeutics for future pandemic preparedness.

The SARS-CoV-2 genome contains 13-15 open reading frames that encode for a variety of structural and non-structural proteins (nsp's). Lu, R. et al., “Genomic characterisation and epidemiology of 2019 novel coronavirus: implications for virus origins and receptor binding,” The Lancet 395(10224):565-574 (2020). The largest open reading frame (ORF1ab) encodes for 2 overlapping polyproteins (pp1a, pp1b) that are cleaved at sequence specific sites into 16 distinct nsp's that are involved in viral replication, viral assembly, and host immune modulation. Shamsi, A. et al. (2016). Cleavage of pp1a and pp1b into individual nsp's is mediated by two viral proteases, the main protease (Mpro) and the papin-like protease (Plp). Shamsi, A. et al. (2016); Ullrich, S. and Nitsche, C. (2020). The function of both of these proteases are necessary for coronavirus replication and pathogenesis. Shamsi, A. et al. (2016); Ullrich, S. and Nitsche, C. (2020). The main protease cleaves peptides after a glutamine (P1) that is adjacent to a hydrophobic residue such as leucine, phenylalanine, or valine (P2). Ullrich, S. and Nitsche, C. (2020). This substrate recognition is distinct from all known human proteases. Id. Proteolysis is mediated by a cysteine—histidine catalytic diad along with a buried water molecule that is hydrogen bonded to the catalytic histidine. The main protease sequence is well conserved across human coronaviruses, particularly the active site and substrate binding pocket. Banerjee, R. et al., “Potential SARS-CoV-2 main protease inhibitors,” Drug Discovery Today 26(3):804-816 (2021). Therefore, the main protease constitutes an attractive drug target for COVID-19 with a high potential for enabling pan-coronavirus therapeutics. Inhibition of the main protease has been demonstrated to block SARS-CoV-2 infection in cells, and reduce lung viral titer in infected mice. Owen, D. R., et al., “An oral SARS-CoV-2 M^{pro} inhibitor clinical candidate for the treatment of COVID-19,” Science 374(6575):1586-1593 (2021). Recent clinical data indicate that administration of an Mpro inhibitor to COVID-19 patients shortly after symptom onset significantly reduces hospitalization and death (Pfizer press release: https://www.pfizer.com/news/press-release/press-release-detail/pfizers-novel-covid-19-oral-antiviral-treatment-candidate).

Therefore, there exists a need to develop new compounds for treating coronavirus infection via inhibition of the protease Mpro.

One aspect of the present disclosure provides a compound of formula I:

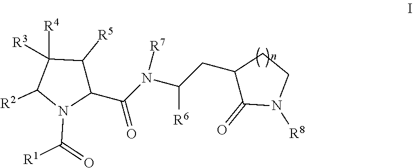

or a pharmaceutically acceptable salt thereof, wherein:
R¹ is
   (a) —C₁₋₆alkyl,
   (b) —C₁₋₆haloalkyl,
   (c) —C₁₋₆alkylene-NHCO—C₁₋₆haloalkyl,
   (d) —C₀₋₆alkylene-(saturated cycloalkyl),
   (e) —C₀₋₆alkylene-heterocycloalkyl,
   (f) —C₀₋₆alkylene-(tricyclic heteroaryl),
   (g) -heteroarylene-cycloalkyl,
   (h) -heteroarylene-aryl,
   (i)

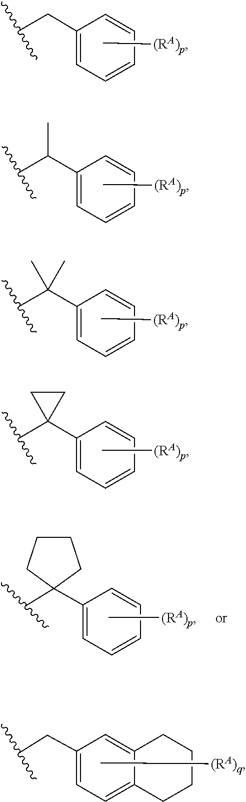

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl;
   p is 0, 1, 2, 3, 4, or 5; and
   q is 0, 1, 2, 3, 4, 5, 6, 7, 8, 9, 10, or 11;
(j)

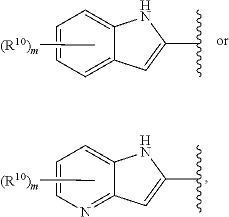

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; or
(k)

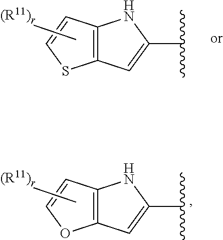

wherein R¹¹ is halo, CN, —C₁₋₆alkyl, —C₁₋₆haloalkyl, —C₁₋₆alkoxy, or —SO₂(C₁₋₆alkyl); and r is 0 or 1;
R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl;
R² is H; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
R⁶ is

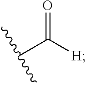

R⁷ and R⁹ are each independently H or C₁₋₆alkyl; and
n is 1, 2, or 3;
wherein at each occurrence, cycloalkyl, saturated cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl);
provided that where n is 1; R², R⁷, and R⁸ are H; R³ and R⁵ or R⁴ and R⁵ together with the carbon atom to which they are attached form a 3 membered or 5 membered-cycloalkyl; then R¹ is not

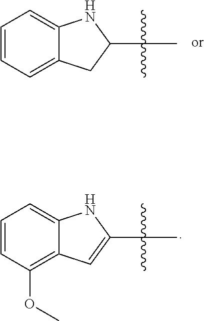 [SMILES: *C1Cc2ccccc2N1 ; *c1cc2c(OC)cccc2[nH]1]

Another aspect of the present disclosure provides a compound of formula II:

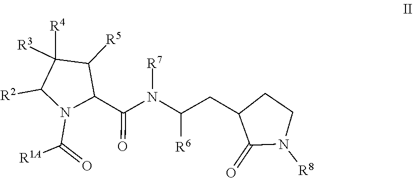

or a pharmaceutically acceptable salt thereof, wherein:
   R^{1A} is H,
   —C₁₋₆alkylene-NHCONH—C₁₋₆alkyl,
   —C₁₋₆alkylene-NHCO(heteroaryl),
   —C₁₋₆alkylene-cycloalkyl,

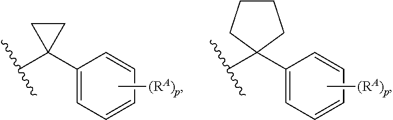

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5;

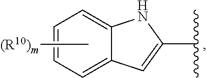

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2;
R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl;
R² is H; or
R² and R³ together with the carbon atoms to which they are attached, form a C₃-C₆cycloalkyl; or
R⁶ is

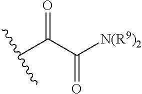

or —COR¹²,
R⁷ and R⁸ are each independently H or C₁₋₆alkyl;
each R⁹ is independently H, C₁₋₆alkyl, cycloalkyl, or heteroaryl; and R¹² is heterocyclyl or heteroaryl;
wherein at each occurrence, cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

Another aspect of the present disclosure provides compounds selected from any of the compounds prepared in the examples, or a pharmaceutically acceptable salt thereof.

A further aspect of the present disclosure provides a pharmaceutical composition comprising the compound of the disclosure, or a pharmaceutically acceptable salt thereof, and a pharmaceutically acceptable carrier.

An additional aspect of the present disclosure provides a method of treating a viral infection in a patient, comprising administering to the patient a therapeutically effective amount of a compound of the disclosure, or a pharmaceutically acceptable salt thereof, or a pharmaceutical composition comprising the compound of the disclosure.

Another aspect of the present disclosure provides a compound of the disclosure or a pharmaceutically acceptable salt or ester thereof, for use in treating a coronavirus infection in a human.

A further aspect of the present disclosure provides a compound of the disclosure or a pharmaceutically acceptable salt or ester thereof, for use in treating SARS-CoV-2 infection in a human.

An additional aspect of the present disclosure relates to a medical use of the compounds or a pharmaceutically acceptable salt thereof in the manufacture of a medicament for treating a coronavirus infection.

### DETAILED DESCRIPTION

As used herein, all technical and scientific terms have the same meaning as commonly understood by one of ordinary skill in the art to which this disclosure belongs. All publications, patent applications, patents, and other references mentioned herein are incorporated by reference in their entirety.

### Definitions

The term “disease” or “condition” refers to disturbances and/or anomalies that are regarded as being pathological conditions or functions, and that can manifest themselves in the form of particular signs, symptoms, and/or malfunctions. As demonstrated below, compounds of the present disclosure are inhibitors of Mpro and can be used in treating or preventing diseases and conditions wherein inhibition of Mpro provides a benefit.

As used herein, the terms “treatment,” “treat,” and “treating” are interchangeable and refer to reversing, alleviating, delaying the onset of, or inhibiting the progress of a disease or disorder, or one or more symptoms thereof, including but not limited to therapeutic benefit.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life).

Therapeutic benefit includes eradication and/or amelioration of the underlying disorder being treated. It also includes the eradication and/or amelioration of one or more of the symptoms associated with the underlying disorder such that an improvement is observed in the subject, notwithstanding that the subject may still be afflicted with the underlying disorder.

The terms “prevent,” “preventing,” and “prevention” refer to a method of preventing or delaying the onset of a disease or condition and/or its attendant symptoms or barring a subject from acquiring a disease. These terms may include “prophylactic treatment”, which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

As used herein, “administering” or “administration” of the compound of formula (I) or formula (I-A) or a pharmaceutically acceptable salt thereof encompasses the delivery to a patient a compound or a pharmaceutically acceptable salt thereof, or a prodrug or other pharmaceutically acceptable derivative thereof, using any suitable formulation or route of administration, e.g., as described herein.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, “patient” or “subject” refers to human subject of any age groups and/or other primates (e.g., cynomolgus monkeys, rhesus monkeys).

As used herein, “pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The term “orally” refers to administering a composition that is intended to be ingested. Examples of oral forms include, but are not limited to, tablets, pills, capsules, powders, granules, solutions or suspensions, and drops. Such forms may be swallowed whole or may be in chewable form.

The terms “a,” “an,” and “the” are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “halo” refers to —Cl, —F, —Br, or —I.

The term “alkyl” refers to a straight- or branched-chain saturated hydrocarbon. The chain may contain an indicated number of carbon atoms. For example, C₁-C₁₀ or C₁₋₁₀ indicates that the group may have from 1 to 10 (inclusive) carbon atoms in it; C₁-C₆ or C₁₋₆ indicates that the group may have from 1 to 6 (inclusive) carbon atoms in it. If not otherwise indicated, an alkyl group contains from 1 to about 20 carbon atoms. In some aspects, alkyl groups have 1 to about 10 carbon atoms. In some aspects, alkyl groups (“lower alkyl”) have 1 to 8, 1 to 6, or 1 to 3 carbon atoms in the chain. Examples may include, but are not limited to methyl, ethyl, propyl, isopropyl, butyl, sec-butyl, iso-butyl, tert-butyl, pentyl, neopentyl, 2-methylbutyl, 1,1-dimethylpropyl, hexyl, heptyl, octyl, nonyl, decyl, and docecyl.

The term “alkylene” as used herein includes an alkyl group that is substituted at two points. An example is methylene (—CH₂—), propylene (—CH₂CH₂CH₂—), and the like.

The term “substituted with 0, 1, 2, 3, or 4 substituents” refers to that a group may be unsubstituted when the group is substituted with 0 substituent, or that the group is substituted with 1, 2, 3, or 4 substituents.

The term “optionally substituted” refers to that a group may be unsubstituted, or substituted with one or more of the indicated substituents. The term “substituted with 0, 1, 2, 3, or 4 substituents” has the same meaning and is equivalent to the term “optionally substituted with 1, 2, 3, or 4 substituents”.

The term “alkoxy” refers to an alkyl group attached to a terminal oxygen atom. Non-limiting exemplary alkoxy groups include methoxy, ethoxy, and tert-butoxy.

The term “amino protecting group” refers to a protecting group that is suitable for preventing undesired reactions at an amino nitrogen. Representative amino-protecting groups include, but are not limited to, formyl; acyl groups, such as acetyl and trifluoroacetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; and the like.

The term “cycloalkyl” refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. A cycloalkyl group may be unsubstituted or substituted with 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl. In some embodiments, the benzyloxy, cycloalkyl, aryl, and heteroaryl may be further substituted with 0, 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl.

The term “cycloalkylene” as used herein includes a cycloalkyl group that is substituted at two points.

The term “aryl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, tetrahydroanthracenyl, or anthracenyl) ring systems having six to fourteen carbon atoms in which the monocyclic ring system is aromatic or at least one of the rings in a bicyclic or tricyclic ring system is aromatic. The other ring(s) in the bicyclic or tricyclic ring system may be saturated, partially unsaturated, or fully unsaturated. The bicyclic and tricyclic groups include benzofused carbocyclic rings. For example, a benzofused group includes a phenyl group fused with one or two 4 to 8 membered carbocyclic moieties. An aryl group may be unsubstituted or substituted with 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl. In some embodiments, the benzyloxy, cycloalkyl, aryl, and heteroaryl may be further substituted with 0, 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl.

The term “heteroaryl” refers to monocyclic, bicyclic, or tricyclic ring systems containing five to fourteen ring atoms including carbon, zero, one, two, three, or four heteroatoms. Each heteroatom is independently oxygen, sulfur, or nitrogen. The monocyclic ring system is aromatic and at least one of the rings in the bicyclic or tricyclic ring systems is aromatic. The other ring(s) in the bicyclic or tricyclic ring system may be saturated, partially unsaturated, or fully unsaturated. In one embodiment, the heteroaryl has three heteroatoms. In another embodiment, the heteroaryl has two heteroatoms. In another embodiment, the heteroaryl has one heteroatom. In some embodiments, the heteroaryl has five to twelve ring atoms. In some embodiments, the heteroaryl has eight to twelve ring atoms. In some embodiments, the heteroaryl has eight or nine ring atoms. In some embodiments, the heteroaryl has nine ring atoms. Non-limiting exemplary heteroaryl groups include thienyl (e.g., thien-2-yl and thien-3-yl), benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrazinyl, pyrimidinyl (e.g., pyrimidin-2-yl, pyrimidin-4-yl, and pyrimidin-5-yl), pyridazinyl, isoindolyl, 3H-indolyl, 1H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl (e.g., thiazol-2-yl, thiazol-4-yl, and thiazol-5-yl), isothiazolyl (e.g., isothiazol-3-yl, isothiazol-4-yl, and isothiazol-5-yl), phenothiazolyl, oxazolyl (e.g., oxazol-2-yl, oxazol-4-yl, and oxazol-5-yl), isoxazolyl (e.g., isoxazol-3-yl, isoxazol-4-yl, and isoxazol-5-yl), furazanyl, phenoxazinyl, triazolyl, triazinyl, and tetrazolyl. In some embodiments, the heteroaryl group is indolyl. In some embodiments, the heteroaryl group is 1H-indolyl. In some embodiments, the heteroaryl group is 5H-[1,3]dioxolo[4,5-f]indole. The term heteroaryl also includes N-oxides. A non-limiting exemplary N-oxide is pyridyl N-oxide.

A heteroaryl group may be unsubstituted or substituted with 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl. In some embodiments, the benzyloxy, cycloalkyl, aryl, and heteroaryl may be further substituted with 0, 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl.

The term “heteroarylene” as used herein includes a heteroaryl group that is substituted at two points.

The term “heterocycloalkyl” refers to saturated and partially unsaturated, e.g., containing one or two double bonds, monocyclic or bicyclic ring structure containing three to fourteen ring members, in which zero, one, two, or three of the ring atoms is a heteroatom. Each heteroatom is independently oxygen, sulfur, or nitrogen. In one embodiment, the heterocycloalkyl has three heteroatoms. In another embodiment, the heterocycloalkyl has two heteroatoms. In another embodiment, the heterocycloalkyl has one heteroatom. Non-limiting exemplary heterocycloalkyl groups include thiacyclohexanyl, oxiranyl, aziridinyl, azetidinyl, oxetanyl and thietanyl, oxazinyl, oxetanyl, azetidinyl, thietanyl, dihydrofuranyl, tetrahydrofuranyl, tetrahydropyranyl, tetrahydrothiophenyl, dihydrothiophenyl, tetrahydro-2H-pyran, dihydropyranyl, dioxanyl, 1,3-dioxolanyl, 1,4-dithianyl, hexahydropyrimidine, pyrrolidinyl, dihydropyrrolyl, morpholinyl, piperazinyl, piperidinyl, 2H-pyranyl, 4H-pyranyl, pyrazolidinyl, pyrazolinyl, tetrahydropyridinyl, tetrahydrothiopyranyl, thiomorpholinyl, thioxanyl, trithianyl, bicyclo[1.1.1]pentanyl, imidazolidinyl, dioxolanyl, oxathiolanyl and dithiolanyl, triazolinyl, oxadiazolinyl, thiadiazolinyl, dihydropyridinyl, thianyl, dioxanyl, triazinanyl, azepanyl, oxepanyl, thiepanyl, azocanyl, oxecanyl, and thiocanyl.

A heterocycloalkyl group may be unsubstituted or substituted with 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl. In some embodiments, the benzyloxy, cycloalkyl, aryl, and heteroaryl may be further substituted with 0, 1, 2, 3, or 4 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), —CO(C₁₋₆alkyl), benzyloxy, cycloalkyl, aryl, and heteroaryl.

The term “haloalkyl” refers to an alkyl group where at least one hydrogen has been replaced with a halo group. In certain embodiments, the haloalkyl group has one halo group. In certain embodiments, the haloalkyl group has two or three halo groups. In certain embodiments, the haloalkyl group is a perfluorinated alkyl group. Examples may include, but are not limited to, chloromethyl, trifluoromethyl, and the like.

The term “hydroxyalkyl” refers to an alkyl group where at least one hydrogen has been replaced with an alcohol (—OH) group. In certain embodiments, the hydroxyalkyl group has one alcohol group. In certain embodiments, the hydroxyalkyl group has two alcohol groups, each on a different carbon atom. Examples may include, but are not limited to, hydroxymethyl, 2-hydroxyethyl, and 1-hydroxyethyl.

When any two substituent groups or any two instances of the same substituent group are “independently” selected from a list of alternatives, the groups may be the same or different. For example, if R^{a} and R^{b} are each independently alkyl, —F, —NH₂, or —OH, then a molecule with two R^{a} groups and two R^{b} groups could have all groups be an alkyl group (e.g., four different alkyl groups). Alternatively, the first R^{a} could be alkyl, the second R^{a} could be —F, the first R^{b} could be —OH, and the second R^{b} could be —NH₂ (or any other substituents taken from the group). Alternatively, both R^{a} and the first R^{b} could be —F, while the second R^{b} could be alkyl (i.e., some pairs of substituent groups may be the same, while other pairs may be different).

Compounds of the present disclosure may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

The stereochemical terms and conventions used in the specification are meant to be consistent with those described in Pure & Appl. Chem 68:2193 (1996), unless otherwise indicated.

### Compounds of the Disclosure

In some embodiments, the present disclosure provides a compound of formula I:

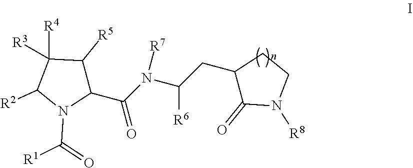

or a pharmaceutically acceptable salt thereof, wherein:
   R¹ is
   (a) —C₁₋₆alkyl,
   (b) —C₁₋₆haloalkyl,
   (c) —C₁₋₆alkylene-NHCO—C₁₋₆haloalkyl,
   (d) —C₀₋₆alkylene-(saturated cycloalkyl),
   (e) —C₀₋₆alkylene-heterocycloalkyl,
   (f) —C₀₋₆alkylene-(tricyclic heteroaryl),
   (g) -heteroarylene-cycloalkyl,
   (h) -heteroarylene-aryl,
   (i)

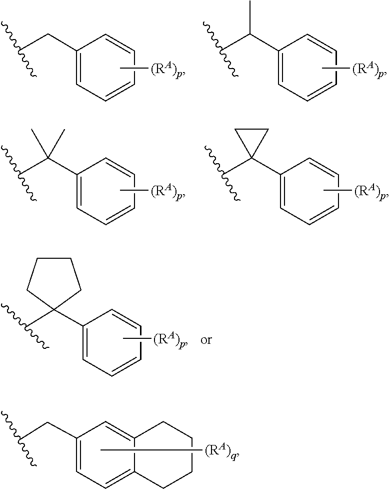

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl;
   p is 0, 1, 2, 3, 4, or 5; and
   q is 0, 1, 2, 3, 4, 5, 6, 7, 8, 9, 10, or 11;
(j)

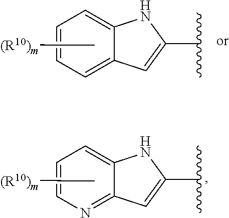

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; or
(k)

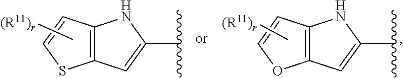

wherein R¹¹ is halo, CN, —C₁₋₆alkyl, —C₁₋₆haloalkyl, —C₁₋₆alkoxy, or —SO₂(C₁₋₆alkyl); and r is 0 or 1;
R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; R² is H; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
R⁶ is

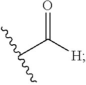

R⁷ and R⁸ are each independently H or C₁₋₆alkyl; and
n is 1, 2, or 3;
wherein at each occurrence, cycloalkyl, saturated cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl);
provided that where n is 1; R², R⁷, and R⁸ are H; R³ and R⁵ or R⁴ and R⁵ together with the carbon atom to which they are attached form a 3 membered or 5 membered-cycloalkyl; then
R¹ is not

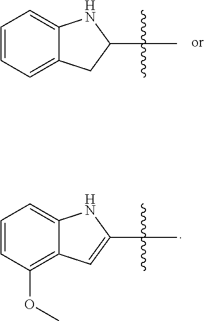 [SMILES: *C1Cc2ccccc2N1 ; *c1cc2c(OC)cccc2[nH]1]

In some embodiments of formula I, the compound is not of the formula:

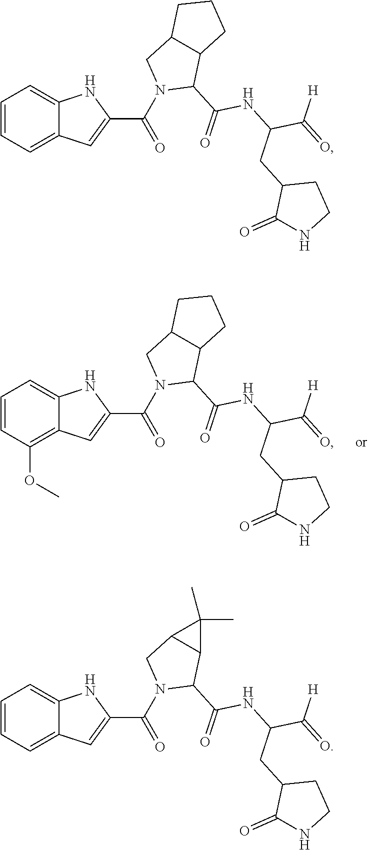 [SMILES: O=CC(CC1CCNC1=O)NC(=O)C1C2CCCC2CN1C(=O)c1cc2ccccc2[nH]1 ; COc1cccc2[nH]c(C(=O)N3CC4CCCC4C3C(=O)NC(C=O)CC3CCNC3=O)cc12 ; CC1(C)C2CN(C(=O)c3cc4ccccc4[nH]3)C(C(=O)NC(C=O)CC3CCNC3=O)C21]

In some embodiments of formula I,
R¹ is
(a) —C₁₋₆alkyl,
(b) —C₁₋₆alkylene-NHCO—C₁₋₆haloalkyl,
(c) —C₀₋₆alkylene-(saturated cycloalkyl),
(d) —C₀₋₆alkylene-heterocycloalkyl,
(e) —C₀₋₆alkylene-(tricyclic heteroaryl),
(f) -heteroarylene-cycloalkyl,
(g)

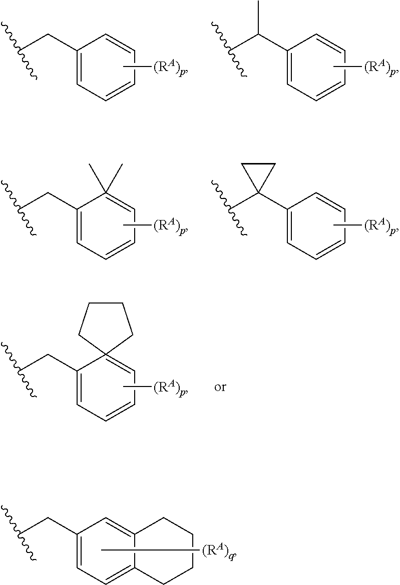

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl;
   p is 0, 1, 2, 3, 4, or 5, and
   q is 0, 1, 2, 3, 4, 5, 6, 7, 8, 9, 10, or 11;
(h)

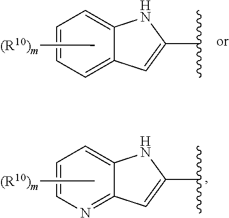

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(i)

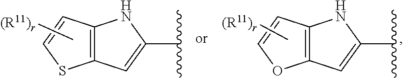

wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, cycloalkyl, heterocycloalkyl, aryl, phenyl, and heteroaryl are each independently substituted with 0, 1, or 2 substituents independently selected from the group consisting of halo, —OH, —CN, —C₁₋₆alkyl, —C₁₋₆haloalkyl, —C₁₋₆alkoxy, —SO₂(C₁₋₆alkyl), and —CO(C₁₋₆alkyl).

In some embodiments of formula I, R¹ is
(a) —C₁₋₆alkyl,
(b) —C₁₋₆alkylene-NHCO—C₁₋₆haloalkyl,
(c) —C₀₋₆alkylene-(saturated cycloalkyl), wherein cycloalkyl is substituted with 0 or 1 phenyl,
(d) —C₀₋₆alkylene-(3-7 membered heterocycloalkyl),
(e) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl,
(f) -(5-12 membered heteroarylene)-C₃₋₆cycloalkyl,
(g) -(5-12 membered heteroarylene)-phenyl,
(h)

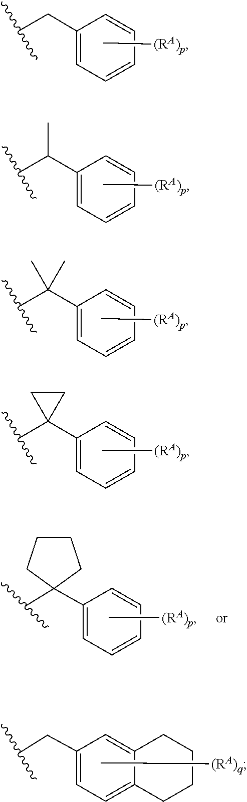

(i)

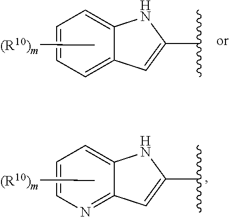

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(j)

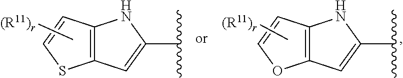

wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, heterocycloalkyl, aryl, phenyl, and heteroaryl are each independently substituted with 0, 1, or 2 substituents independently selected from the group consisting of halo, —OH, —CN, —C₁₋₆alkyl, —C₁₋₆haloalkyl, —C₁₋₆alkoxy, —SO₂(C₁₋₆alkyl), and —CO(C₁₋₆alkyl).

In some embodiments of formula I, R¹ is —C₀₋₆alkylene-(C₃₋₆ saturated cycloalkyl), wherein the cycloalkyl is substituted with 0, 1, or 2 substituents independently selected from the group consisting of halo, —OH, —CN, —C₁₋₆alkyl, —C₁₋₆haloalkyl, and —C₁₋₆alkoxy.

In some embodiments, R¹ in embodiment one is selected from

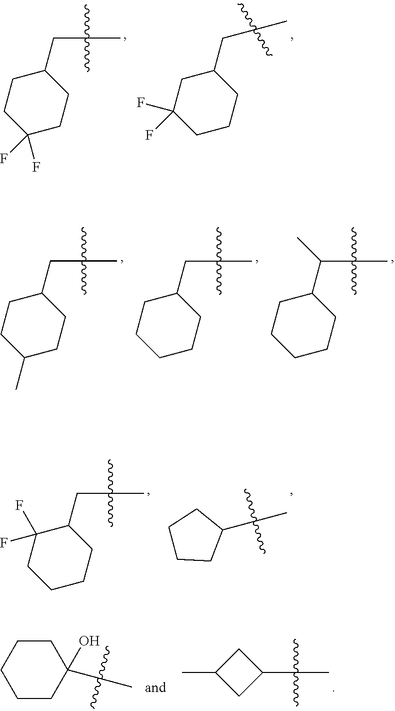

In some embodiments of formula I, R¹ is

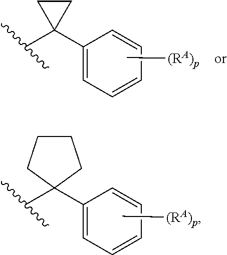

wherein R¹ is substituted with 0, 1, or 2 R^{A} substituents independently selected from the group consisting of halo, —OH, —CN, —C₁₋₆alkyl, —C₁₋₆haloalkyl, and —C₁₋₆alkoxy; and p is 0, 1, or 2.

In some embodiments of formula I, R¹ is halo or —C₁₋₆alkyl; and P is 0 or 1.

In some embodiments of formula I, R¹ is selected from

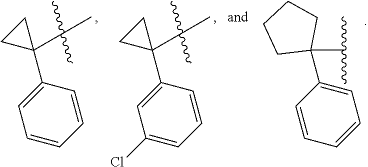

In some embodiments of formula I, R¹ is —C₀₋₆alkylene-heterocycloalkyl, wherein the heterocycloalkyl is substituted with 0, 1, or 2 substituents selected from the group consisting of halo, —OH, —CN, —C₁₋₆alkyl, —C₁₋₆haloalkyl, and —C₁₋₆alkoxy.

In some embodiments of formula I, R¹ is —C₀₋₃alkylene-(5-7 membered heterocycloalkyl.

In some embodiments of formula I, R¹ is selected from

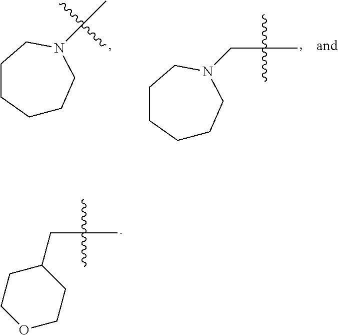 [SMILES: *N1CCCCCC1 ; *CN1CCCCCC1 ; *CC1CCOCC1]

In some embodiment of formula I, R¹ is selected from

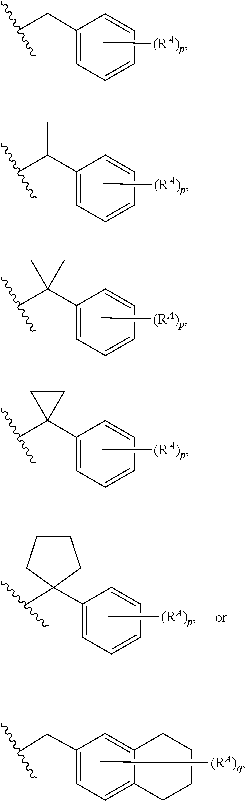

wherein R^{A} is halo, —OH, —CN, —C₁₋₆alkyl, —C₁₋₆haloalkyl, or —C₁₋₆alkoxy.

In some embodiments of formula I, R¹ is selected from

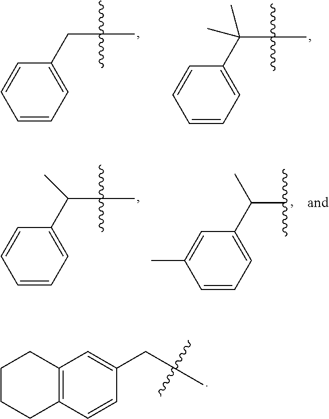

In some embodiments of formula I, R¹ is
(a) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl,
(b) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl, or
(c) -(5-12 membered heteroarylene)-phenyl.

In some embodiments, R¹ in formula I is

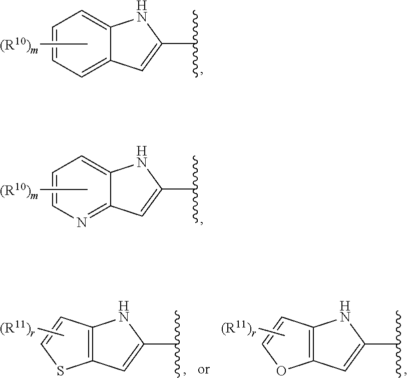

wherein R¹⁰ or R¹¹ is independently halo, —CN, —C₁₋₆alkyl, —C₁₋₆haloalkyl, —C₁₋₆alkoxy, or —SO₂(C₁₋₆alkyl).

In some embodiments of formula I, R¹ is

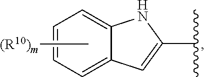

wherein R¹⁰ is halo, CN, —C₁₋₆alkyl, —C₁₋₆haloalkyl, —C₁₋₆alkoxy, or —SO₂(C₁₋₆alkyl); and m is 0, 1, or 2.

In some embodiments of formula I, R¹ is a 5 to 12 membered heteroaryl, (5-12 membered heteroarylene)-C₃₋₆cycloalkyl, or -(5-12 membered heteroarylene)-phenyl, wherein the heteroaryl is substituted with 0, 1, or 2 substituents selected from the group consisting of halo, —CN, —C₁₋₆alkyl, —C₁₋₆haloalkyl, —C₁₋₆alkoxy, and —SO₂(C₁₋₆alkyl).

In some embodiments of formula I, R¹ is a 8 to 12 membered heteroaryl, (8-12 membered heteroarylene)-C₃₋₆cycloalkyl, or -(8-12 membered heteroarylene)-phenyl, wherein the heteroaryl is substituted with 0, 1, or 2 substituents selected from the group consisting of halo, —CN, —C₁₋₆alkyl, —C₁₋₆haloalkyl, —C₁₋₆alkoxy, and —SO₂(C₁₋₆alkyl).

In some embodiments of formula I, R¹ is a 8 to 12 membered heteroaryl substituted with 0, 1, or 2 substituents selected from the group consisting of halo, —CN, —C₁₋₆alkyl, —C₁₋₆haloalkyl, —C₁₋₆alkoxy, and —SO₂(C₁₋₆alkyl).

In some embodiments of formula I, R¹ is selected from any of the moieties in Table 1.

TABLE 1

| | | |
|---|---|---|
| 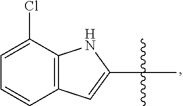 [SMILES: *c1cc2cccc(Cl)c2[nH]1] | 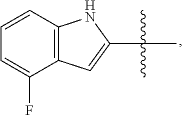 [SMILES: *c1cc2c(F)cccc2[nH]1] | 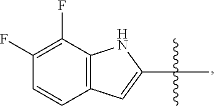 [SMILES: *c1cc2ccc(F)c(F)c2[nH]1] |
| 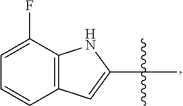 [SMILES: *c1cc2cccc(F)c2[nH]1] | 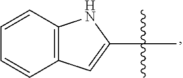 [SMILES: *c1cc2ccccc2[nH]1] | 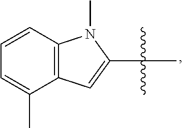 [SMILES: *c1cc2c(C)cccc2n1C] |
| 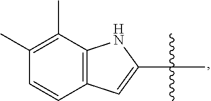 [SMILES: *c1cc2ccc(C)c(C)c2[nH]1] | 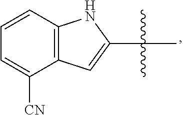 [SMILES: *c1cc2c(C#N)cccc2[nH]1] | 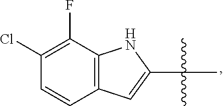 [SMILES: *c1cc2ccc(Cl)c(F)c2[nH]1] |
| 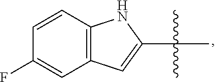 [SMILES: *c1cc2cc(F)ccc2[nH]1] | 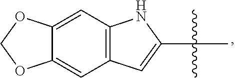 [SMILES: *c1cc2cc3c(cc2[nH]1)OCO3] | 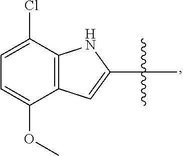 [SMILES: *c1cc2c(OC)ccc(Cl)c2[nH]1] |
| 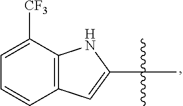 [SMILES: *c1cc2cccc(C(F)(F)F)c2[nH]1] | 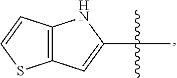 [SMILES: *c1cc2sccc2[nH]1] | 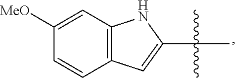 [SMILES: *c1cc2ccc(OC)cc2[nH]1] |
| 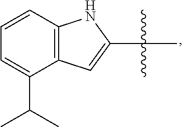 [SMILES: *c1cc2c(C(C)C)cccc2[nH]1] | 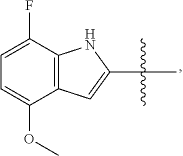 [SMILES: *c1cc2c(OC)ccc(F)c2[nH]1] | 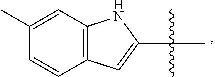 [SMILES: *c1cc2ccc(C)cc2[nH]1] |
| 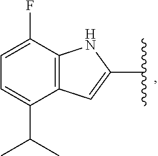 [SMILES: *c1cc2c(C(C)C)ccc(F)c2[nH]1] | 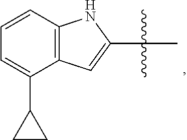 [SMILES: *c1cc2c(C3CC3)cccc2[nH]1] | 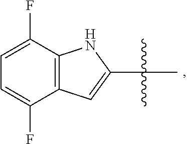 [SMILES: *c1cc2c(F)ccc(F)c2[nH]1] |
| 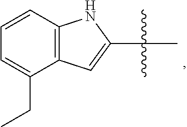 [SMILES: *c1cc2c(CC)cccc2[nH]1] | 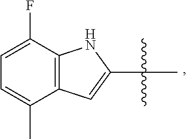 [SMILES: *c1cc2c(C)ccc(F)c2[nH]1] | 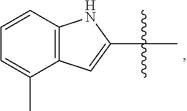 [SMILES: *c1cc2c(C)cccc2[nH]1] |
| 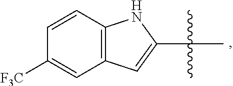 [SMILES: *c1cc2cc(C(F)(F)F)ccc2[nH]1] | 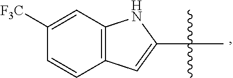 [SMILES: *c1cc2ccc(C(F)(F)F)cc2[nH]1] | 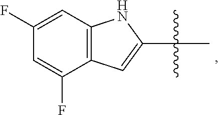 [SMILES: *c1cc2c(F)cc(F)cc2[nH]1] |
| 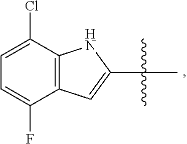 [SMILES: *c1cc2c(F)ccc(Cl)c2[nH]1] | 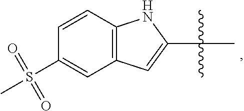 [SMILES: *c1cc2cc(S(C)(=O)=O)ccc2[nH]1] | 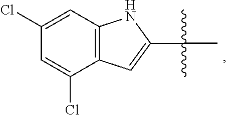 [SMILES: *c1cc2c(Cl)cc(Cl)cc2[nH]1] |
| 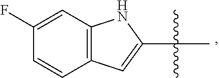 [SMILES: *c1cc2ccc(F)cc2[nH]1] | 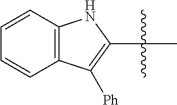 [SMILES: *c1[nH]c2ccccc2c1-c1ccccc1] | 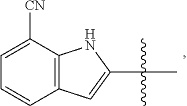 [SMILES: *c1cc2cccc(C#N)c2[nH]1] |
| 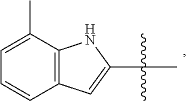 [SMILES: *c1cc2cccc(C)c2[nH]1] | 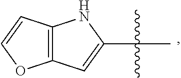 [SMILES: *c1cc2occc2[nH]1] | 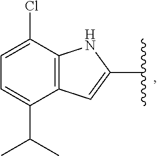 [SMILES: *c1cc2c(C(C)C)ccc(Cl)c2[nH]1] |
| 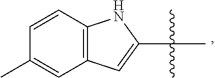 [SMILES: *c1cc2cc(C)ccc2[nH]1] | 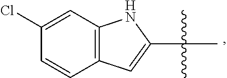 [SMILES: *c1cc2ccc(Cl)cc2[nH]1] | 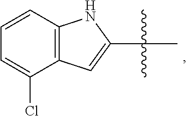 [SMILES: *c1cc2c(Cl)cccc2[nH]1] |
| 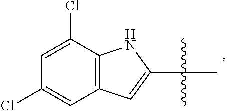 [SMILES: *c1cc2cc(Cl)cc(Cl)c2[nH]1] | 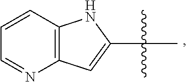 [SMILES: *c1cc2ncccc2[nH]1] | 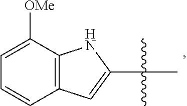 [SMILES: *c1cc2cccc(OC)c2[nH]1] |
| 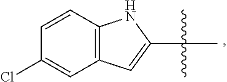 [SMILES: *c1cc2cc(Cl)ccc2[nH]1] | 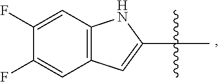 [SMILES: *c1cc2cc(F)c(F)cc2[nH]1] | 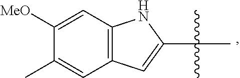 [SMILES: *c1cc2cc(C)c(OC)cc2[nH]1] |
| 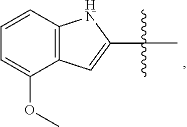 [SMILES: *c1cc2c(OC)cccc2[nH]1] | 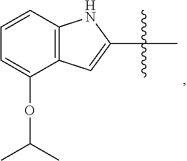 [SMILES: *c1cc2c(OC(C)C)cccc2[nH]1] | 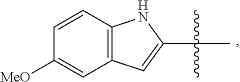 [SMILES: *c1cc2cc(OC)ccc2[nH]1] |
| 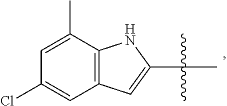 [SMILES: *c1cc2cc(Cl)cc(C)c2[nH]1] | 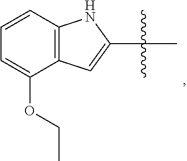 [SMILES: *c1cc2c(OCC)cccc2[nH]1] | 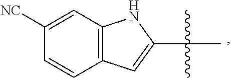 [SMILES: *c1cc2ccc(C#N)cc2[nH]1] |
| 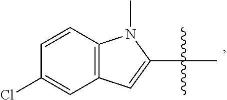 [SMILES: *c1cc2cc(Cl)ccc2n1C] | 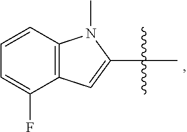 [SMILES: *c1cc2c(F)cccc2n1C] | 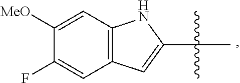 [SMILES: *c1cc2cc(F)c(OC)cc2[nH]1] |
| 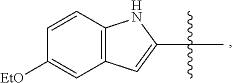 [SMILES: *c1cc2cc(OCC)ccc2[nH]1] | 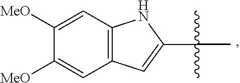 [SMILES: *c1cc2cc(OC)c(OC)cc2[nH]1] | and |
| 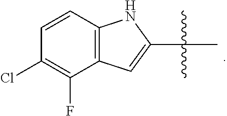 [SMILES: *c1cc2c(F)c(Cl)ccc2[nH]1] | | |

In some embodiments of formula I, R¹ is selected from any of the moieties in Table 2.

TABLE 2

| | | |
|---|---|---|
| 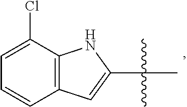 [SMILES: *c1cc2cccc(Cl)c2[nH]1] | 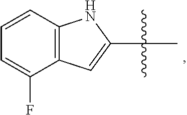 [SMILES: *c1cc2c(F)cccc2[nH]1] | 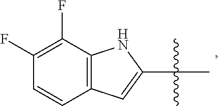 [SMILES: *c1cc2ccc(F)c(F)c2[nH]1] |
| 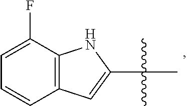 [SMILES: *c1cc2cccc(F)c2[nH]1] | 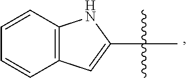 [SMILES: *c1cc2ccccc2[nH]1] | 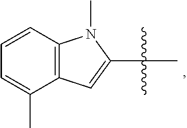 [SMILES: *c1cc2c(C)cccc2n1C] |
| 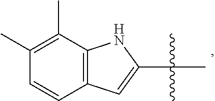 [SMILES: *c1cc2ccc(C)c(C)c2[nH]1] | 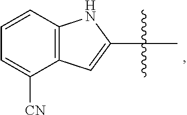 [SMILES: *c1cc2c(C#N)cccc2[nH]1] | 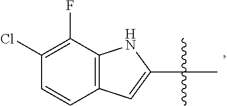 [SMILES: *c1cc2ccc(Cl)c(F)c2[nH]1] |
| 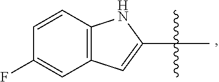 [SMILES: *c1cc2cc(F)ccc2[nH]1] | 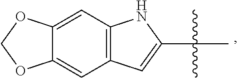 [SMILES: *c1cc2cc3c(cc2[nH]1)OCO3] | 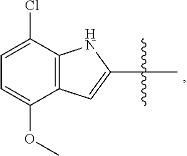 [SMILES: *c1cc2c(OC)ccc(Cl)c2[nH]1] |
| 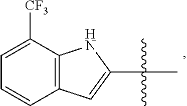 [SMILES: *c1cc2cccc(C(F)(F)F)c2[nH]1] | 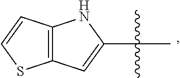 [SMILES: *c1cc2sccc2[nH]1] | 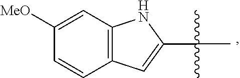 [SMILES: *c1cc2ccc(OC)cc2[nH]1] |
| 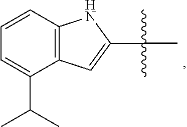 [SMILES: *c1cc2c(C(C)C)cccc2[nH]1] | 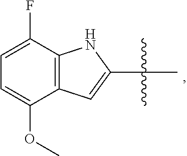 [SMILES: *c1cc2c(OC)ccc(F)c2[nH]1] | 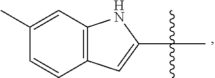 [SMILES: *c1cc2ccc(C)cc2[nH]1] |
| 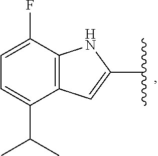 [SMILES: *c1cc2c(C(C)C)ccc(F)c2[nH]1] | 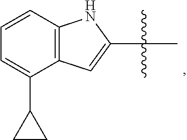 [SMILES: *c1cc2c(C3CC3)cccc2[nH]1] | 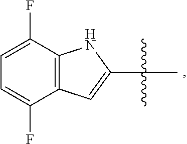 [SMILES: *c1cc2c(F)ccc(F)c2[nH]1] |
| 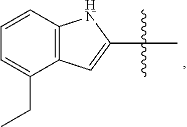 [SMILES: *c1cc2c(CC)cccc2[nH]1] | 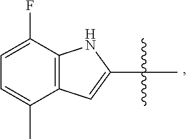 [SMILES: *c1cc2c(C)ccc(F)c2[nH]1] | 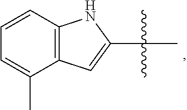 [SMILES: *c1cc2c(C)cccc2[nH]1] |
| 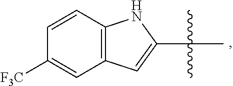 [SMILES: *c1cc2cc(C(F)(F)F)ccc2[nH]1] | 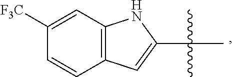 [SMILES: *c1cc2ccc(C(F)(F)F)cc2[nH]1] | 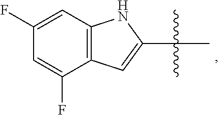 [SMILES: *c1cc2c(F)cc(F)cc2[nH]1] |
| 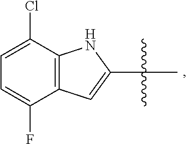 [SMILES: *c1cc2c(F)ccc(Cl)c2[nH]1] | 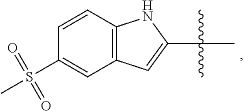 [SMILES: *c1cc2cc(S(C)(=O)=O)ccc2[nH]1] | 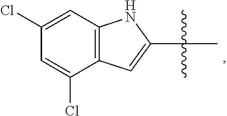 [SMILES: *c1cc2c(Cl)cc(Cl)cc2[nH]1] |
| 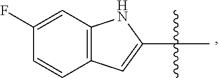 [SMILES: *c1cc2ccc(F)cc2[nH]1] | 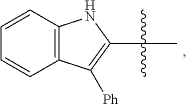 [SMILES: *c1[nH]c2ccccc2c1-c1ccccc1] | 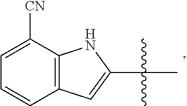 [SMILES: *c1cc2cccc(C#N)c2[nH]1] |
| 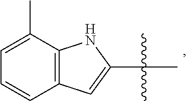 [SMILES: *c1cc2cccc(C)c2[nH]1] | 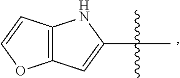 [SMILES: *c1cc2occc2[nH]1] | 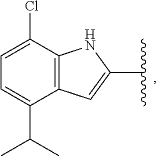 [SMILES: *c1cc2c(C(C)C)ccc(Cl)c2[nH]1] |
| 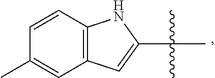 [SMILES: *c1cc2cc(C)ccc2[nH]1] | 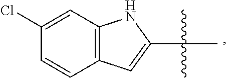 [SMILES: *c1cc2ccc(Cl)cc2[nH]1] | 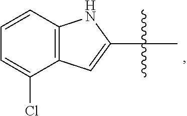 [SMILES: *c1cc2c(Cl)cccc2[nH]1] |
| 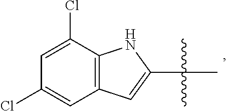 [SMILES: *c1cc2cc(Cl)cc(Cl)c2[nH]1] | 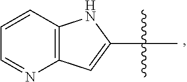 [SMILES: *c1cc2ncccc2[nH]1] | 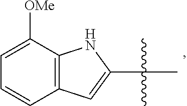 [SMILES: *c1cc2cccc(OC)c2[nH]1] |
| 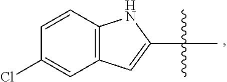 [SMILES: *c1cc2cc(Cl)ccc2[nH]1] | 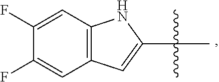 [SMILES: *c1cc2cc(F)c(F)cc2[nH]1] | 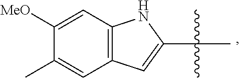 [SMILES: *c1cc2cc(C)c(OC)cc2[nH]1] |
| 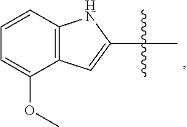 [SMILES: *c1cc2c(OC)cccc2[nH]1] | 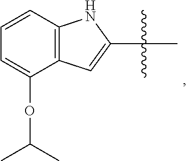 [SMILES: *c1cc2c(OC(C)C)cccc2[nH]1] | 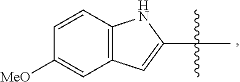 [SMILES: *c1cc2cc(OC)ccc2[nH]1] |
| 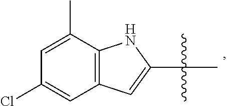 [SMILES: *c1cc2cc(Cl)cc(C)c2[nH]1] | 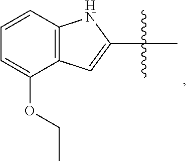 [SMILES: *c1cc2c(OCC)cccc2[nH]1] | 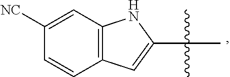 [SMILES: *c1cc2ccc(C#N)cc2[nH]1] |
| 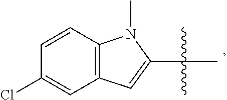 [SMILES: *c1cc2cc(Cl)ccc2n1C] | 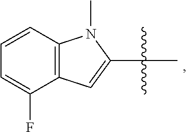 [SMILES: *c1cc2c(F)cccc2n1C] | 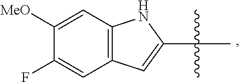 [SMILES: *c1cc2cc(F)c(OC)cc2[nH]1] |
| 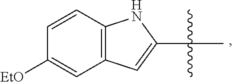 [SMILES: *c1cc2cc(OCC)ccc2[nH]1] | 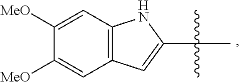 [SMILES: *c1cc2cc(OC)c(OC)cc2[nH]1] | 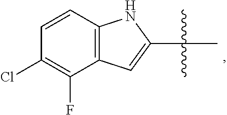 [SMILES: *c1cc2c(F)c(Cl)ccc2[nH]1] |
| 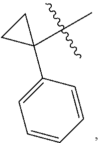 [SMILES: *C1(c2ccccc2)CC1] | 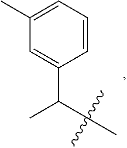 [SMILES: *C(C)c1cccc(C)c1] | 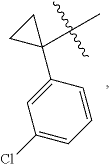 [SMILES: *C1(c2cccc(Cl)c2)CC1] |
| 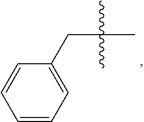 [SMILES: *Cc1ccccc1] | 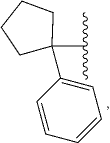 [SMILES: *C1(c2ccccc2)CCCC1] | 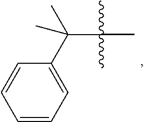 [SMILES: *C(C)(C)c1ccccc1] |
| 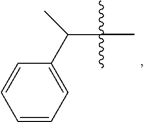 [SMILES: *C(C)c1ccccc1] | 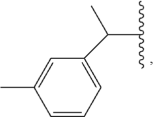 [SMILES: *C(C)c1cccc(C)c1] | 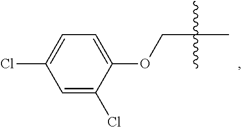 [SMILES: *COc1ccc(Cl)cc1Cl] |
| 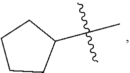 [SMILES: *C1CCCC1] | 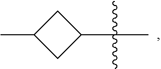 [SMILES: *C1CC(C)C1] | 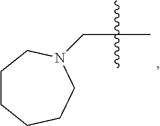 [SMILES: *CN1CCCCCC1] |
| 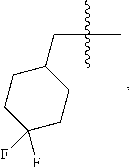 [SMILES: *CC1CCC(F)(F)CC1] | 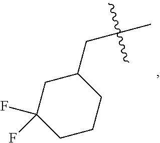 [SMILES: *CC1CCCC(F)(F)C1] | 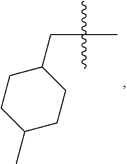 [SMILES: *CC1CCC(C)CC1] |
| 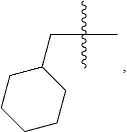 [SMILES: *CC1CCCCC1] | 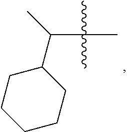 [SMILES: *C(C)C1CCCCC1] | 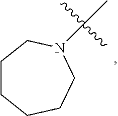 [SMILES: *N1CCCCCC1] |
| 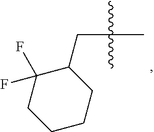 [SMILES: *CC1CCCCC1(F)F] | 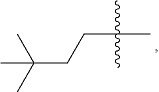 [SMILES: *CCC(C)(C)C] | 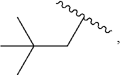 [SMILES: *CC(C)(C)C] |
| 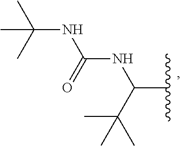 [SMILES: *C(NC(=O)NC(C)(C)C)C(C)(C)C] | 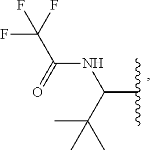 [SMILES: *C(NC(=O)C(F)(F)F)C(C)(C)C] | 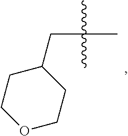 [SMILES: *CC1CCOCC1] |
| 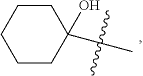 [SMILES: *C1(O)CCCCC1] | and | 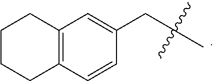 [SMILES: *Cc1ccc2c(c1)CCCC2] |

In some embodiments of formula I,
R² is H;
R³ and R⁴ are each independently —H, halo, —OH, —CN, —NH₂, —NO₂, methylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl;
R⁵ is —H, halo, —C₁₋₆alkyl, or —C₁₋₆haloalkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
wherein at each occurrence, the cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

In some embodiments of formula I,
R² is H;
R³ and R⁴ are each independently —H, halo, —OH, —CN, methylenyl, —C₁₋₆alkyl, —C₁₋₆alkoxy, phenyl, phenoxy, benzyl, C₃-C₆cycloalkyl, 5-7 membered heterocycloalkyl, or 5-9 membered heteroaryl;
R⁵ is —H, halo, —C₁₋₆alkyl, or —C₁₋₆haloalkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
wherein at each occurrence, the cycloalkyl, heterocycloalkyl, phenyl, phenoxy, benzyl, and heteroaryl are each independently substituted with 0, 1, or 2 substituents selected from the group consisting of halo, —OH, —CN, —C₁₋₆alkyl, —C₁₋₆haloalkyl, and —C₁₋₆alkoxy.

In some embodiments of formula I,
R² is H;
R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl;
R⁵ is —H, halo, or —C₁₋₆alkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
   wherein at each occurrence, the cycloalkyl, heterocycloalkyl, phenyl, phenoxy, and benzyl are each independently substituted with 0, 1, or 2 substituents selected from the group consisting of halo and —C₁₋₆alkyl.

In some embodiments of formula I, R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0, 1, or 2 substituents selected from the group consisting of halo and —C₁₋₆alkyl.

In some embodiments of formula I, R³ is —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl; and R⁴ is —H, halo, or —C₁₋₆alkyl.

In some embodiments of formula I, R³ is —H, —F, -Me, -Et, —OH, —CN, methylenyl, —OMe, phenyl, phenoxy,

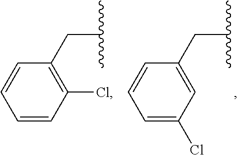

or cyclohexyl; and R⁴ is —H, —F, -Me, or -Et.

In some embodiments of formula I, R² is H and R⁵ is —H or —C₁₋₆alkyl.

In some embodiments of formula I, R² and R⁵ are H.

In some embodiments of formula I, R³ is —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl; R⁴ is —H, halo, or —C₁₋₆alkyl; R² is —H; and R⁵ is —H or —C₁₋₆alkyl.

In some embodiments of formula I,
R³ in embodiment one is —H, —F, -Me, -Et, —OH, —CN, methylenyl, —OMe, phenyl, phenoxy,

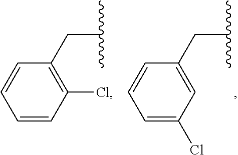

or cyclohexyl;
R⁴ is —H, —F, -Me, or -Et; and R² and R⁵ are —H.

In some embodiments of formula I, R³ and R⁴ together with the carbon atom to which they are attached, form a C₃-C₆cycloalkyl, wherein the cycloalkyl is substituted with 0, 1, or 2 substituents selected from the group consisting of halo and —C₁₋₆alkyl.

In some embodiments of formula I, R³ and R⁴ together with the carbon atom to which they are attached, form a C₃-C₆cycloalkyl; R² is H; and R⁵ is —H or —C₁₋₆alkyl.

In some embodiments of formula I, R³ and R⁴ together with the carbon atom to which they are attached, form a C₃-C₄cycloalkyl; and R² and R⁵ are —H.

In some embodiments of formula I, R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl.

In some embodiments of formula I, R³ and R⁵ together with the carbon atoms to which they are attached, form a C₃-C₆cycloalkyl, wherein the cycloalkyl is substituted with 0, 1, or 2 substituents which is —C₁₋₆alkyl; R² is H; and R⁴ is —H or —C₁₋₆alkyl.

In some embodiments of formula I, R³ and R⁵ together with the carbon atoms to which they are attached, form a C₃-C₅cycloalkyl, wherein the cycloalkyl is substituted with 0, 1, or 2 substituents which is -Me; and R² and R⁴ are —H.

In some embodiments of formula I, R⁴ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl.

In some embodiments of formula I, R⁴ and R⁵ together with the carbon atoms to which they are attached, form a C₃-C₆cycloalkyl, wherein the cycloalkyl is substituted with 0, 1, or 2 substituents which is —C₁₋₆alkyl; R² is H; and R³ is —H or —C₁₋₆alkyl.

In some embodiments of formula I, R⁴ and R⁵ together with the carbon atoms to which they are attached, form a C₃-C₅cycloalkyl, wherein the cycloalkyl is substituted with 0, 1, or 2 substituents which is -Me; and R² and R³ are —H.

In some embodiments of formula I, R² and R³ together with the carbon atoms to which they are attached, form a C₃-C₆cycloalkyl, wherein the cycloalkyl is substituted with 0, 1, or 2 substituents selected from the group consisting of halo and —C₁₋₆alkyl.

In some embodiments of formula I, R² and R³ together with the carbon atoms to which they are attached, form a C₃-C₆cycloalkyl; and R⁴ and R⁵ are each independently —H or —C₁₋₆alkyl.

In some embodiments of formula I, R² and R³ together with the carbon atoms to which they are attached, form a C₃-C₆cycloalkyl; and R⁴ and R⁵ are —H.

In some embodiments of formula I, R² and R⁵ together with the pyrrolidine ring to which they are attached form a bridged 6 to 8 membered heterocycloalkyl, wherein the heterocycloalkyl is substituted with 0, 1, or 2 substituents selected from the group consisting of halo and —C₁₋₆alkyl.

In some embodiments of formula I, R² and R⁵ together with the pyrrolidine ring to which they are attached form a bridged 6 to 7 membered heterocycloalkyl, wherein the heterocycloalkyl is substituted with 0 or 1 substituent selected from the group consisting of halo and —C₁₋₆alkyl; and R³ and R⁴ are each independently —H or —C₁₋₆alkyl.

In some embodiments of formula I, R² and R⁵ together with the pyrrolidine ring to which they are attached form a bridged 7 membered heterocycloalkyl, wherein the heterocycloalkyl is substituted with —F; and R³ and R⁴ are —H.

In some embodiments of formula I, R⁷ and R⁸ are H.

In some embodiments of formula I, n is 1 or 2.

In some embodiments of formula I, n is 1.

In some embodiments, the compound of formula I is a compound of formula A

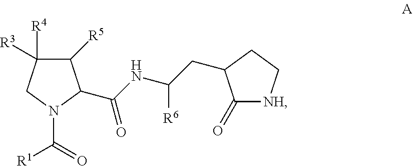

wherein the variables are as defined herein.

In some embodiments of formula I or formula A, R¹ is
(a) —C₁₋₆alkyl,
(b) —C₁₋₆alkylene-NHCO—C₁₋₆haloalkyl,
(c) —C₀₋₆alkylene-(saturated cycloalkyl),
(d) —C₀₋₆alkylene-heterocycloalkyl,
(e) -heteroarylene-cycloalkyl, or
(f) -heteroarylene-aryl;
(g)

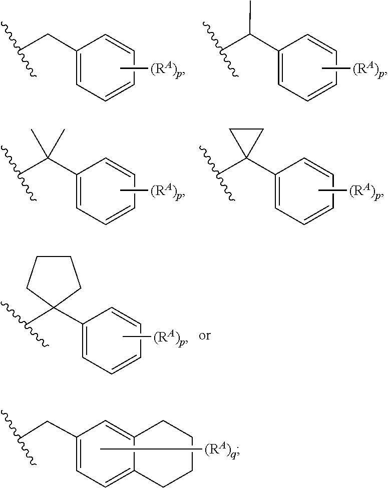

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; and q is 0, 1, 2, 3, 4, 5, 6, 7, 8, 9, 10, or 11;
(h)

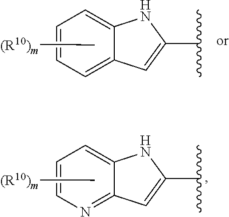

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2;
(i)

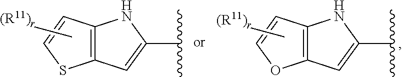

wherein R¹¹ is halo, CN, —C₁₋₆alkyl, —C₁₋₆haloalkyl, —C₁₋₆alkoxy, or —SO₂(C₁₋₆alkyl); and r is 0 or 1;
R³ and R⁴ are each independently —H, halo, —OH, —CN, —NH₂, —NO₂, methylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl;
R⁵ is —H, halo, —C₁₋₆alkyl, or —C₁₋₆haloalkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; and
R⁶ is

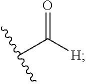

wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

In some embodiments of compounds of formula I or formula A, R¹ is
(a) —C₁₋₆alkylene-NHCO—C₁₋₆haloalkyl,
(b) —C₀₋₆alkylene-(saturated cycloalkyl),
(c) —C₀₋₆alkylene-(tricyclic heteroaryl),
(d) —C₀₋₆alkylene-heterocycloalkyl,
(e)

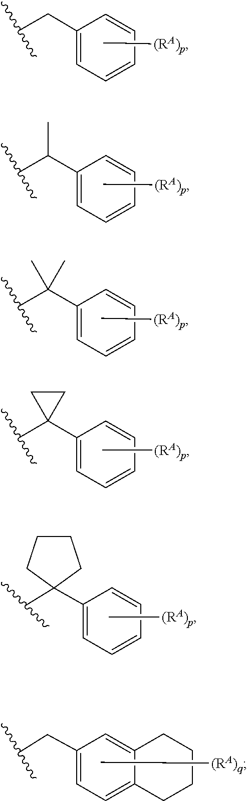

wherein R is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(f)

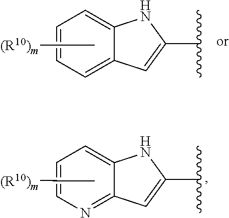

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(g)

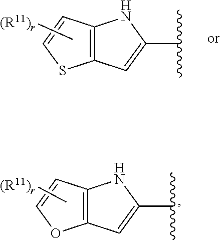

wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

In some embodiments of formula I or formula A, R¹ is selected from any of the moieties in Table 3.

TABLE 3

| | | |
|---|---|---|
| 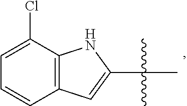 [SMILES: *c1cc2cccc(Cl)c2[nH]1] | 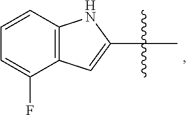 [SMILES: *c1cc2c(F)cccc2[nH]1] | 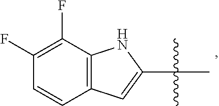 [SMILES: *c1cc2ccc(F)c(F)c2[nH]1] |
| 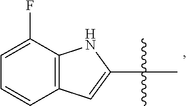 [SMILES: *c1cc2cccc(F)c2[nH]1] | 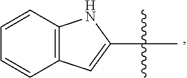 [SMILES: *c1cc2ccccc2[nH]1] | 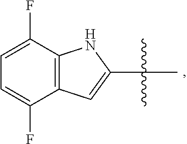 [SMILES: *c1cc2c(F)ccc(F)c2[nH]1] |
| 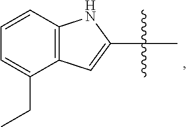 [SMILES: *c1cc2c(CC)cccc2[nH]1] | 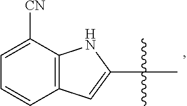 [SMILES: *c1cc2cccc(C#N)c2[nH]1] | 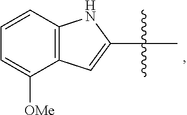 [SMILES: *c1cc2c(OC)cccc2[nH]1] |
| 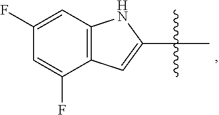 [SMILES: *c1cc2c(F)cc(F)cc2[nH]1] | 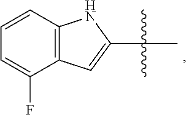 [SMILES: *c1cc2c(F)cccc2[nH]1] | 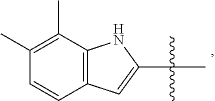 [SMILES: *c1cc2ccc(C)c(C)c2[nH]1] |
| 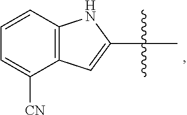 [SMILES: *c1cc2c(C#N)cccc2[nH]1] | 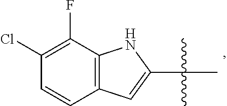 [SMILES: *c1cc2ccc(Cl)c(F)c2[nH]1] | 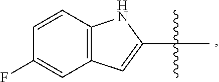 [SMILES: *c1cc2cc(F)ccc2[nH]1] |
| 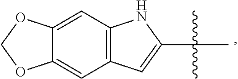 [SMILES: *c1cc2cc3c(cc2[nH]1)OCO3] | 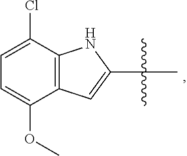 [SMILES: *c1cc2c(OC)ccc(Cl)c2[nH]1] | 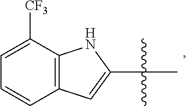 [SMILES: *c1cc2cccc(C(F)(F)F)c2[nH]1] |
| 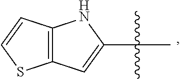 [SMILES: *c1cc2sccc2[nH]1] | 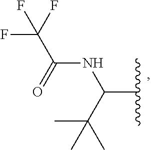 [SMILES: *C(NC(=O)C(F)(F)F)C(C)(C)C] | 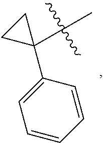 [SMILES: *C1(c2ccccc2)CC1] |
| 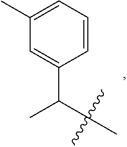 [SMILES: *C(C)c1cccc(C)c1] | 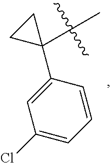 [SMILES: *C1(c2cccc(Cl)c2)CC1] | 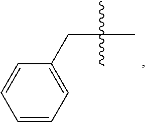 [SMILES: *Cc1ccccc1] |
| 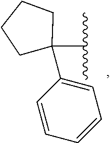 [SMILES: *C1(c2ccccc2)CCCC1] | 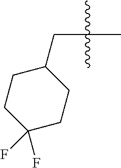 [SMILES: *CC1CCC(F)(F)CC1] | and |
| 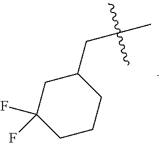 [SMILES: *CC1CCCC(F)(F)C1] | | |

In some embodiments of formula I or formula A, R³ is —H, —F, -Me, -Et, —OH, —CN, methylenyl, —OMe, phenyl, phenoxy,

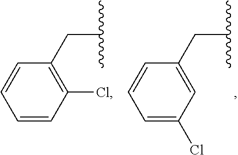

or cyclohexyl; R⁴ is —H, —F, -Me, or -Et; and R² and R⁵ are —H.

In some embodiments of formula I or formula A, R³ and R⁴ together with the carbon atom to which they are attached form a C₃-C₆cycloalkyl; and R⁵ is H

In some embodiments of formula I or formula A, R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl; and R⁴ is H.

In some embodiments of compounds of formula I or formula A, R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl; and R⁵ is —H or —C₁₋₆alkyl.

In some embodiments of formula I or formula A, R³ and R⁴ together with the carbon atom to which they are attached, form a C₃-C₆cycloalkyl; and R⁵ is H.

In some embodiments of formula I or formula A, which is a compound of formula B

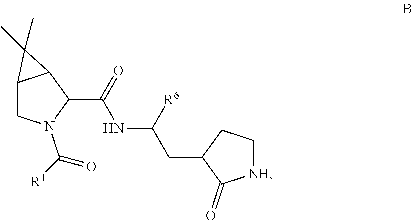

wherein R¹ and R⁶ are as defined herein.

In some embodiments of formula B, which is a compound of formula B-1

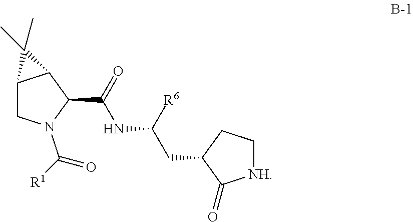

In some embodiments of formula I or formula A, which is a compound of formula C

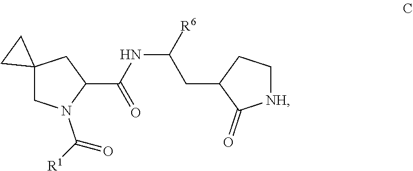

wherein R¹ and R⁶ are as defined herein.

In some embodiments of formula C, which is a compound of formula C-1

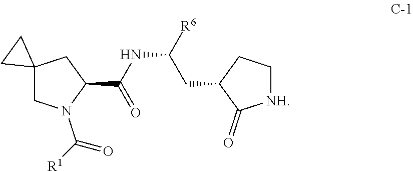

In some embodiments of formula I, formula A, formula B, formula B-1, formula C, or formula C-1, wherein
R¹ is
   (a) —C₁₋₆alkylene-NHCO—C₁₋₆haloalkyl,
   (b) —C₀₋₆alkylene-(saturated cycloalkyl),
   (c) —C₀₋₆alkylene-heterocycloalkyl,
   (d) —C₀₋₆alkylene-(tricyclic heteroaryl),
   (e)

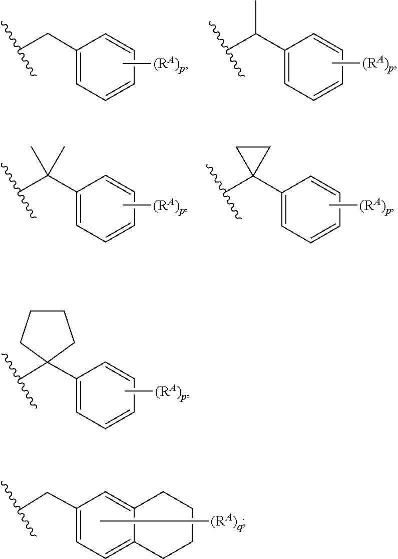

R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(f)

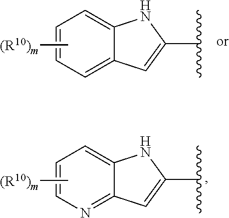

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; or
(g)

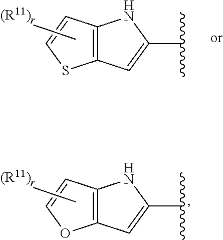

wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl); and
R⁶ is H

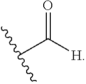

In some embodiments of formula I, formula A, formula B, formula B-1, formula C, or formula C-1, wherein
R¹ is
(a) —C₁₋₆alkylene-NHCO—C₁₋₆haloalkyl,
(b) —C₀₋₆alkylene-(saturated cycloalkyl),
(c) —C₀₋₆alkylene-(tricyclic heteroaryl),
(d)

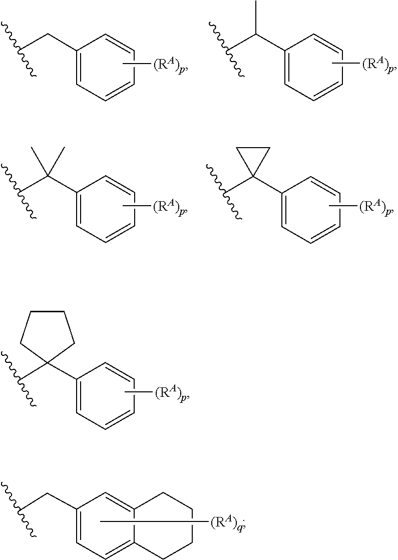

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(e)

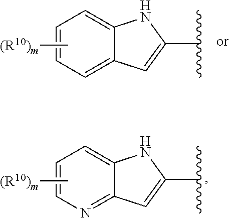

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; or
(f)

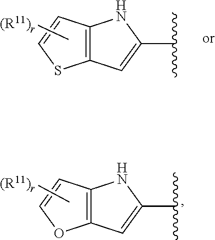

wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, phenyl, and heteroaryl are each independently substituted with 0, 1, or 2 substituents selected from the group consisting of halo, —OH, —CN, —NH₂, —NH(C₁₋₆alkyl), —N(C₁₋₆alkyl)₂, —C₁₋₆alkyl, —C₁₋₆haloalkyl, —C₁₋₆alkoxy, —SO₂(C₁₋₆alkyl), and —CO(C₁₋₆alkyl); and
R⁶ is N H.

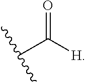

In some embodiments of formula I, formula A, formula B, formula B-1, formula C, or formula C-1, wherein
R¹ is selected from the group consisting of

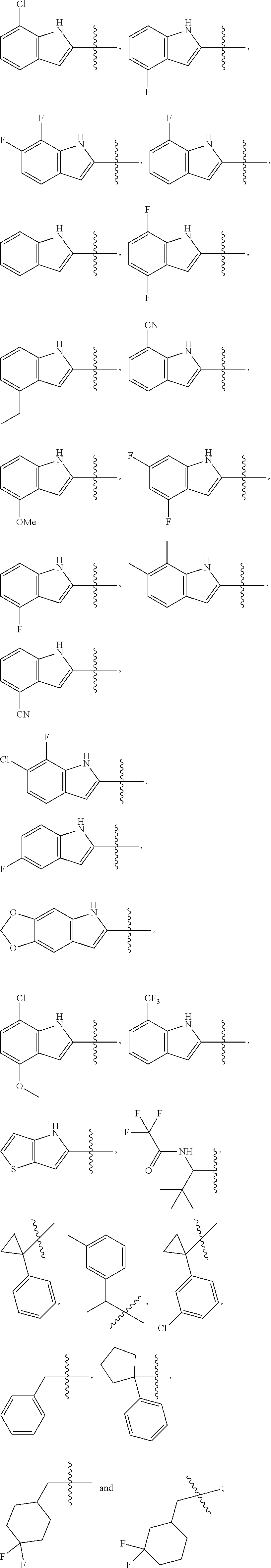

and R⁶

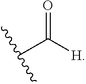

In some embodiments, the present disclosure provides a compound of formula IL

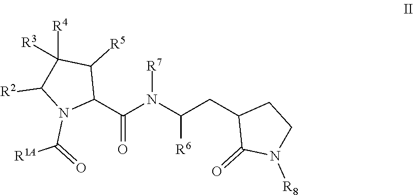

or a pharmaceutically acceptable salt thereof, wherein:
R^{1A} is
H,
—C₁₋₆alkylene-NHCONH—C₁₋₆alkyl,
—C₁₋₆alkylene-NHCO(heteroaryl),
—C₁₋₆alkylene-cycloalkyl,

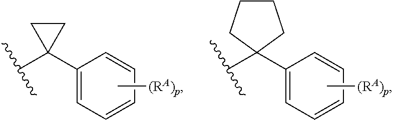

wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5;

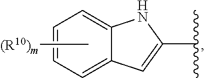

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl;
R² is H; or
R² and R³ together with the carbon atoms to which they are attached, form a C₃-C₆cycloalkyl; or
R⁶ is

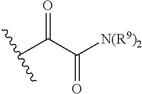

or —COR¹²;
R⁷ and R⁸ are each independently H or C₁₋₆alkyl;
each R⁹ is independently H, C₁₋₆alkyl, cycloalkyl, or heteroaryl;
R¹² is heterocyclyl or heteroaryl; and
n is 1, 2, or 3;
wherein at each occurrence, cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

In some embodiments of formula II, R^{1A} is —C₁₋₆alkylene-NHCONH—C₁₋₆alkyl.

In some embodiments of formula II, R^{1A} is

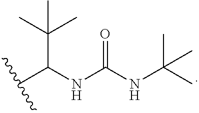 [SMILES: *C(NC(=O)NC(C)(C)C)C(C)(C)C]

In some embodiments of formula II, R^{1A} is —C₁₋₆alkylene-NHCO(heteroaryl).

In some embodiments of formula II, R¹ is

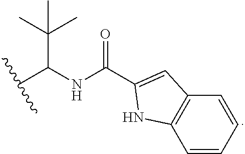 [SMILES: *C(NC(=O)c1cc2ccccc2[nH]1)C(C)(C)C]

In some embodiments of formula II, R^{1A} is —C₁₋₆alkylene-cycloalkyl, wherein the cycloalkyl is optionally substituted by halo, —C₁₋₆alkyl, —OH, —CN, —NH₂, or —NO₂.

In some embodiments of formula II R^{1A} is

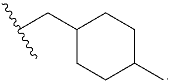 [SMILES: *CC1CCC(C)CC1]

In some embodiments of formula II, R^{1A} is

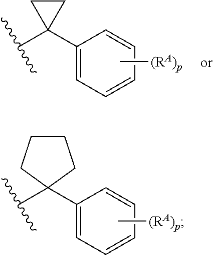

R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; and p is 0, 1, 2, or 3.

In some embodiments of formula II, R¹ is

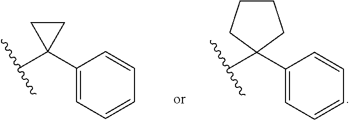 [SMILES: *C1(c2ccccc2)CC1 ; *C1(c2ccccc2)CCCC1]

In some embodiments of formula II, R^{1A} is

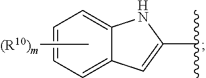

wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2.

In some embodiments of formula II, R¹ is

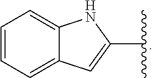 [SMILES: *c1cc2ccccc2[nH]1]

In some embodiments of formula II, R¹ is R² is H.

In some embodiments of formula II, R³ and R⁴ together with the carbon atom to which they are attached, form a C₃-C₆cycloalkyl; and R⁵ is H.

In some embodiments of formula II, R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl; and R⁴ is H.

In some embodiments of formula II, R⁶ is

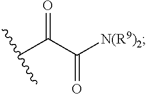

and each R⁹ is independently H, C₁₋₆alkyl, or cycloalkyl

In some embodiments of formula II, R⁶ is

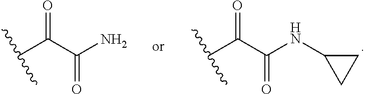 [SMILES: *C(=O)C(N)=O ; *C(=O)C(=O)NC1CC1]

In some embodiments of formula II, R⁶ is

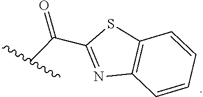 [SMILES: *C(=O)c1nc2ccccc2s1]

In some embodiments of formula II is a compound of formula II-A or II-B:

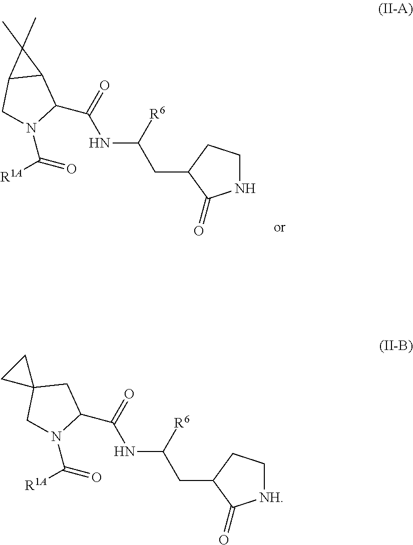

In some embodiments of formula II is a compound of formula II-A or II-B, wherein the compound is of the formula:

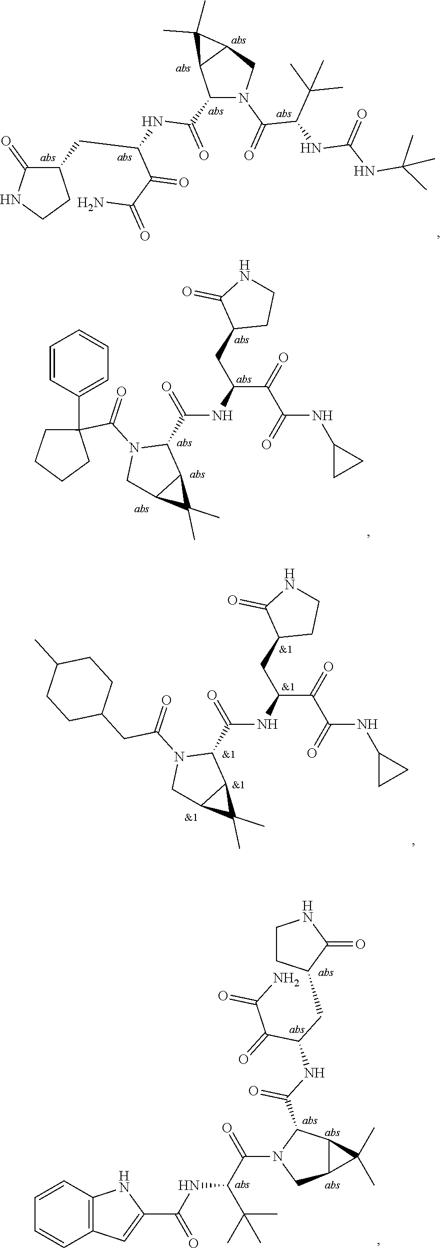

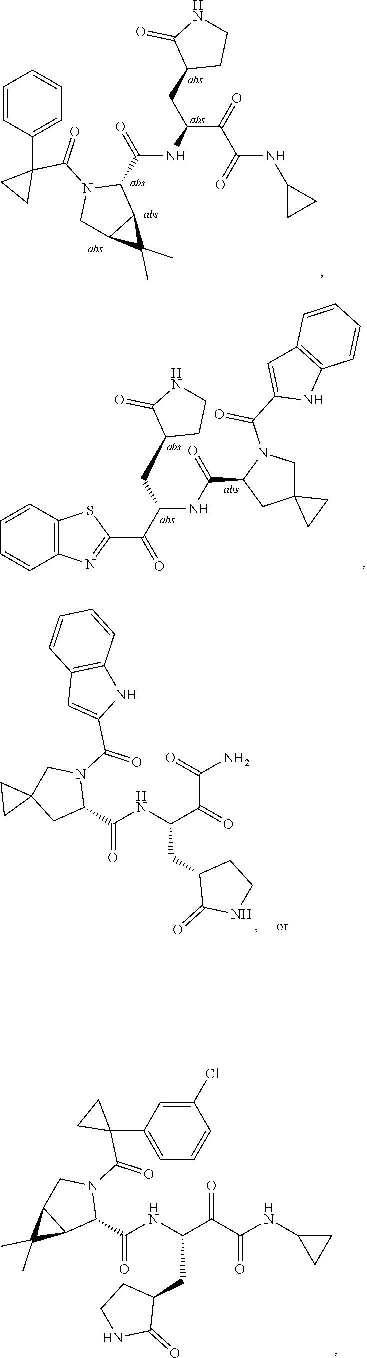

or a pharmaceutically acceptable salt thereof.

In some embodiments, provided herein in is a pharmaceutical composition for treating a coronavirus infection in a human in need thereof, comprising a compound of formula (I) and formula (II), or a pharmaceutically acceptable salt thereof.

In some embodiments, provided herein in is a method of treating a viral infection in a patient, comprising administering to the patient a therapeutically effective amount of a compound of formula (I) or formula (II), or a pharmaceutically acceptable salt thereof.

In some embodiments, wherein the viral infection is caused by a coronavirus.

In some embodiments, wherein the viral infection is a coronavirus infection caused by SARS-CoV-2, SARS-CoV, MERS-CoV, 229E, NL63, OC43, or HKUl.

In some embodiments, wherein the coronavirus infection is caused by SARS-CoV-2.

In some embodiments, wherein the patient is suffering from COVID-19.

In some embodiments, wherein the coronavirus infection is caused by SARS-CoV.

In some embodiments, wherein the coronavirus infection is caused by MERS-CoV.

In some embodiments, wherein the treatment method inhibits Mpro.

In some embodiments, wherein the treatment method is administered orally.

In some embodiments, wherein the treatment is administered once a day, twice a day, or three times a day.

In some embodiments, wherein the therapeutically effective amount of the treatment is from 0.5 mg to 100 mg, 1 mg to 80 mg, or from 1 mg to 60 mg.

In some embodiments, wherein in the treatment, the compound of formula (I) or (II) is formulated in a solid dosage form.

In some embodiments, the present disclosure provides a compound selected from any of the compounds 1-138 described in the examples, or a pharmaceutically acceptable salt thereof.

### Pharmaceutically Acceptable Salts

The present disclosure encompasses the preparation and use of salts of compounds of the present disclosure. As used herein, the pharmaceutical “pharmaceutically acceptable salt” refers to salts or zwitterionic forms of compounds of the present disclosure. Salts of compounds of the present disclosure can be prepared during the final isolation and purification of the compounds or separately by reacting the compound with a suitable acid. The pharmaceutically acceptable salts can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the present disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the present disclosure can be quaternized with methyl, ethyl, propyl, and butyl chlorides, bromides, and iodides; dimethyl, diethyl, dibutyl, and diamyl sulfates; decyl, lauryl, myristyl, steryl chlorides, bromides, and iodides; and benzyl and phenethyl bromides. In light of the foregoing, any reference compounds of the present disclosure appearing herein is intended to include compounds of the present disclosure as well as pharmaceutically acceptable salts, hydrates, or solvates thereof.

The present disclosure encompasses the preparation and use of solvates of compounds of the present disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the present disclosure can be present as solvated forms with a pharmaceutically acceptable solvent, such as water, methanol, and ethanol, and it is intended that the disclosure includes both solvated and unsolvated forms of compounds of the present disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a compound of the present disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### Medical Treatments or Uses

Compounds of the present disclosure inhibit protease Mpro and are thus useful in the treatment or prevention of a variety of diseases and conditions. In particular, compounds of the present disclosure are useful in methods of treating or preventing a disease or condition wherein inhibition of Mpro provides a benefit. These diseases and conditions include viral infections.

In certain embodiments, the viral infection is caused by a coronavirus.

In certain embodiments, the coronavirus infection is caused by a coronavirus including SARS-CoV-1, SARS-CoV-2, MERS-CoV, HCoV-229E, HCoV—HKU1, HCoV-NL63 and HCoV-OC43.

In certain embodiments, the coronavirus infection is caused by a coronavirus including SARS-CoV and MERS-CoV.

In certain embodiments, the coronavirus infection is caused by SARS-CoV-2.

In certain embodiments, the patient is suffering from COVID-19.

In certain embodiments, the patient is suffering from upper respiratory illness, severe interstitial pneumonia, and/or acute respiratory distress syndrome, aggravated by thrombosis in the pulmonary microcirculation. In certain embodiments, the patient is suffering from multi-organ failure.

In certain embodiments, the patient is suffering from fever, cough, tiredness, loss of taste or smell, shortness of breath or difficulty breathing, muscle aches, chills, sore throat, runny nose, headache, chest pain, and/or pink eye.

In certain embodiments, the coronavirus replication is inhibited.

In certain embodiments, the coronavirus polymerase is inhibited.

In certain embodiments, the coronaviral membrane fusion is inhibited.

In certain embodiments, the inhibition is in vitro or in vivo.

In some embodiments, the present disclosure relates to a method of treating an individual suffering from a disease or condition wherein inhibition of Mpro provides a benefit comprising administering a therapeutically effective amount of a compound of the present disclosure to an individual in need thereof.

Compounds of the present disclosure are inhibitors of Mpro protein. Accordingly, diseases and conditions mediated by Mpro can be treated by employing these compounds. The present disclosure is thus directed generally to a method for treating a condition or disorder responsive to Mpro inhibition in a subject, e.g., a human subject, suffering from, or at risk of suffering from, the condition or disorder, the method comprising administering to the subject an effective amount of one or more compounds of the present disclosure.

In some embodiments, the present disclosure is directed to a method of inhibiting Mpro in a subject in need thereof, said method comprising administering to the subject an effective amount of at least one compound of the present disclosure.

In certain embodiments, the Mpro is SARS-CoV-2 Mpro.

The methods of the present disclosure can be accomplished by administering a compound of the present disclosure as the neat compound or as a pharmaceutical composition.

In some embodiments, a human subject is treated with a compound of the present disclosure, or a pharmaceutical composition comprising a compound of the present disclosure, wherein the compound is administered in an amount sufficient to inhibit Mpro in the subject.

### Combination Therapy

In some embodiments, a compound of the present disclosure is administered in conjunction with a second therapeutic agent useful in the treatment of a disease or condition wherein inhibition of Mpro provides a benefit. The second therapeutic agent is different from the compound of the present disclosure.

The second therapeutic agent can be an antiviral agent or an anti-inflamatory/host immune modulator. The antiviral agent may include anti-SARS-CoV-2 monoclonal antibodies such as bamlanivimab/etesevimab, casirivimab/imdevimab, and Sotrovimab; RNA-polymerase inhibitors such as molnupiravir and remdesivir; papin-like protease inhibitors; and methyl transferase inhibitors. The anti-inflamatory/host immune modulator may include steroidal such as dexamethasone; JAK inhibitors such as baricitinib and tofacitinib; and IL-6R blocking monoclonal antibodies such as tocilizumab and sarilumab.

A compound of the present disclosure and the second therapeutic agent can be administered simultaneously or sequentially to achieve the desired effect. In addition, the compound of the present disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A compound of the present disclosure and the second therapeutic agent can be administered together as a single-unit dose or separately as multi-unit doses, wherein the compound of the present disclosure is administered before the second therapeutic agent or vice versa. One or more doses of the compound of the present disclosure and/or one or more dose of the second therapeutic agent can be administered. The compound of the present disclosure therefore can be used in conjunction with one or more second therapeutic agents, for example, but not limited to, anticancer agents.

### Administrations and Dosage

A compound of the present disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique. In some embodiments, the compound or a pharmaceutically acceptable salt thereof is administered orally.

Pharmaceutical compositions include those wherein a compound of the present disclosure is administered in an effective amount to achieve its intended purpose. The exact formulation, route of administration, and dosage is determined by an individual physician in view of the diagnosed condition or disease. Dosage amount and interval can be adjusted individually to provide levels of a compound of the present disclosure that is sufficient to maintain therapeutic effects.

Toxicity and therapeutic efficacy of the compounds of the present disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a compound of the present disclosure required for use in therapy varies with the nature of the condition being treated, the length of time that activity is desired, and the age and the condition of the subject, and is ultimately determined by the attendant physician. Dosage amounts and intervals can be adjusted individually to provide plasma levels of the compound of the present disclosure that are sufficient to maintain the desired therapeutic effects. The desired dose can be administered in a single dose, or as multiple doses administered at appropriate intervals, for example as one, two, three, four or more subdoses per day. Multiple doses sometimes are desired, or required. For example, a compound of the present disclosure can be administered at a frequency of four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two-day rest, and another five daily doses (5/2/5); or, any dose regimen determined to be appropriate for the circumstance. In some embodiments, the compound or a pharmaceutically acceptable salt thereof is administered once a day, twice a day, or three times a day.

A compound of the present disclosure used in a method of the present disclosure can be administered in an amount of about 0.005 to about 500 milligrams per dose, about 0.05 to about 250 milligrams per dose, or about 0.5 to about 100 milligrams per dose. For example, a compound of the present disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams. In some embodiments, the therapeutically effective amount is from 0.5 mg to 100 mg, 1 mg to 80 mg, or from 1 mg to 60 mg.

The dosage of a composition containing a compound of the present disclosure, or a composition containing the same, can be from about 1 μg/kg to about 200 mg/kg, about 1 g/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more.

The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

### Pharmaceutical Compositions

In some embodiments, compounds of the present disclosure typically are administered in admixture with a pharmaceutical carrier to give a pharmaceutical composition selected with regard to the intended route of administration and standard pharmaceutical practice.

Pharmaceutical compositions for use in accordance with the present disclosure are formulated in a conventional manner using one or more physiologically acceptable carriers comprising excipients and/or auxiliaries that facilitate processing of compound of the present disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the compound of the present disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a compound of the present disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound of the present disclosure.

When a therapeutically effective amount of a compound of the present disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the present disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated.

Pharmaceutical preparations for oral use can be obtained by adding the compound of the present disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the present disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a compound of the present disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the present disclosure also can be formulated in rectal compositions, such as suppositories or retention enemas, e.g., containing conventional suppository bases. In addition to the formulations described previously, the compound of the present disclosure also can be formulated as a depot preparation. Such long-acting formulations can be administered by implantation (for example, subcutaneously or intramuscularly) or by intramuscular injection. Thus, for example, the compound of the present disclosure can be formulated with suitable polymeric or hydrophobic materials (for example, as an emulsion in an acceptable oil) or ion exchange resins.

In particular, the compounds of the present disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the present disclosure also can be injected parenterally, for example, intravenously, intramuscularly, subcutaneously, or intracoronarily.

For parenteral administration, the compound of the present disclosure are typically used in the form of a sterile aqueous solution which can contain other substances, for example, salts or monosaccharides, such as mannitol or glucose, to make the solution isotonic with blood.

### Medical Kits

In some embodiments, the present disclosure provides kits which comprise a compound of the present disclosure (or a composition comprising a compound of the present disclosure) packaged in a manner that facilitates their use to practice methods of the present disclosure. In one embodiment, the kit includes a compound of the present disclosure (or a composition comprising a compound of the present disclosure) packaged in a container, such as a sealed bottle or vessel, with a label affixed to the container or included in the kit that describes use of the compound or composition to practice the method of the present disclosure.

In some embodiments, the kit further can include a device suitable for administering the composition according to the intended route of administration.

### General Synthetic Methods

In some embodiments, the compounds of the present disclosure and intermediates can be prepared according to Scheme 1 below. In the general scheme, variables such as R¹, R², R³, R⁴, R⁵, R⁶, R⁷, R⁸ and n are the same as defined herein. R¹⁰ is alkyl such as C₁₋₆alkyl. The expression “P” refers to H or an amino protecting group such as Boc. In some embodiments, R¹ is selected from any of the groups in Table 1, Table 2, or Table 3.

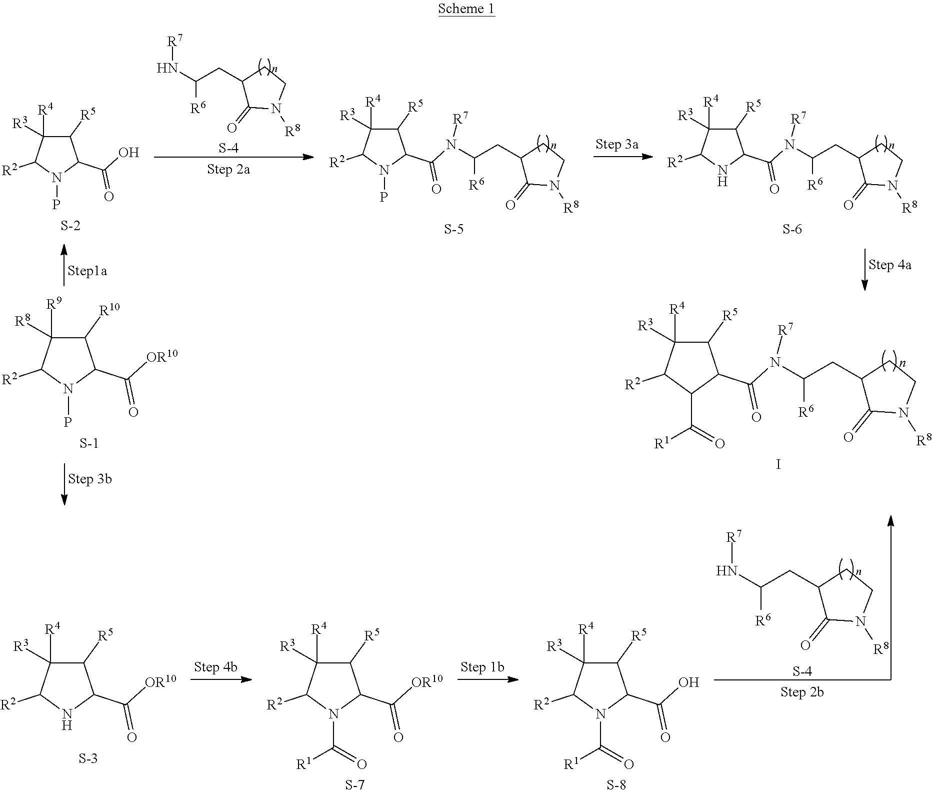

Steps 1a & 1b: Steps 1a and 1b can be carried out in the presence of a suitable base and solvent. The base may be an inorganic base or an organic base. Non-limiting examples of the inorganic base may include hydroxides such as sodium hydroxide, lithium hydroxide, or potassium hydroxide; bicarbonates; carbonates; phosphates; and acetates. The organic base may include but is not limited to amines, e.g., tertiary amines; pyridine; and piperidine. The suitable solvent includes but is not limited to protic and aprotic solvents such as water, methanol, ethanol, DMF, DCM, EA, THF, DMSO, ether, ketone, 1,4-dioxane, and the like. The suitable solvent may also be a combination of two or three solvents. The hydrolysis product may be acidified to afford the free acid.

Steps 2a & 2b: Steps 2a & 2b can be performed in the presence of a suitable base, a coupling reagent, and a solvent. Non-limiting examples of the inorganic base may include bicarbonates, carbonates, phosphates, and acetates. The organic base may include but is not limited to amines, e.g., tertiary amines such as DIEA. The coupling reagent can be a suitable peptide coupling reagent including, without limitation, T₃P, HOAt, HOBt, HUTA, DCC, EDC, DIC, BOP, PyBOP, HATU, HBTU, TOTU, COMU, and TBTU. The suitable solvent includes but is not limited to DCM, DMF, THF, DMSO, acetonitrile, ethers, ethyl acetate, 1,4-dioxane, and the like.

Steps 3a & 3b: Steps 3a & 3b can be performed in the presence of a suitable acid and a solvent. The acid may be an inorganic acid or an organic acid. Non-limiting examples of the inorganic base may include hydrochloric acid, phosphoric acid, and sulfuric acid. The organic acid may include but is not limited to TFA and p-toluenesulfonic acid. The suitable solvent includes but is not limited to protic and aprotic solvents such as water, methanol, ethanol, DMF, DCM, EA, THF, DMSO, ether, ketone, 1,4-dioxane, and the like. The suitable solvent may also be a combination of two or three solvents.

Steps 4a & 4b: Steps 4a & 4b can be performed in the presence of a suitable base, a coupling reagent, and a solvent. Non-limiting examples of the inorganic base may include bicarbonates, carbonates, phosphates, and acetates. The organic base may include but is not limited to amines, e.g., tertiary amines such as DIEA. The coupling reagent can be a suitable peptide coupling reagent including, without limitation, T₃P, HOAt, HOBt, HUTA, DCC, EDC, DIC, BOP, PyBOP, HATU, HBTU, TOTU, COMU, and TBTU. The suitable solvent includes but is not limited to DCM, DMF, THF, DMSO, acetonitrile, ethers, ethyl acetate, 1,4-dioxane, and the like.

In some embodiments, R⁶ is —COOR⁹, and the process further comprises the following steps:

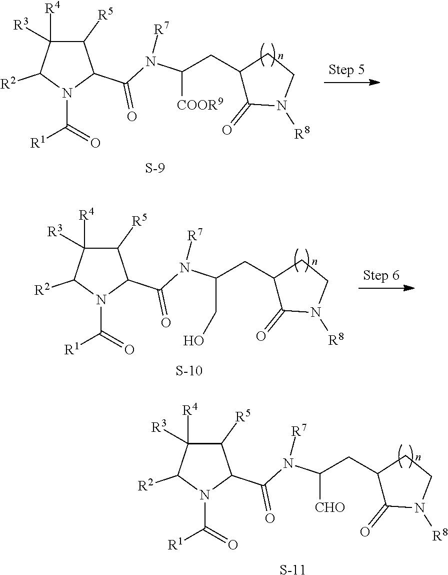

Step 5: Step 5 can be performed in the presence of a reducing reagent and a suitable solvent. A suitable solvent includes protic solvents or aprotic solvents. Protic solvents include but is not limited to water and alcohols such as methanol, ethanol, propanol, and the like. Aprotic solvents include but is not limited to solvents such as DCM, THF, DMF, acetonitrile, and the like. The suitable solvent may also be a combination of two or three solvents. The reducing reagent includes but is not limited to a borohydride reagent or a metal hydride reagent. Non-limiting examples are lithium borohydride, sodium borohydride, sodium cyanoborohydride, sodium triacetoxyborohydride, and lithium aluminium hydride.

Step 6: Step 6 can be performed in the presence of an oxidizing reagent and a suitable solvent. A suitable solvent includes protic solvents or aprotic solvents. Protic solvents include but is not limited to water and alcohols such as methanol, ethanol, propanol, and the like. Aprotic solvents include but is not limited to solvents such as DCM, THF, DMF, acetonitrile, and the like. The suitable solvent may also be a combination of two or three solvents. The oxidizing reagent includes but is not limited to dichromate, Collins reagent, Dess-Martin periodinane, PCC, PDC, and DMSO/oxalyl chloride.

### EXAMPLES

The following abbreviations may be used throughout the specification, including in the description of the synthesis of each of the example compounds.

| Abbreviations/Acronyms | Full Name/Description |
|---|---|
| AcOH | Acetic acid |
| AcOK | Potassium acetate |
| AcCl | Acetyl chloride |
| BOP | Benzotriazole-1-yl-oxytripyrrolidinophosphonium |
| | hexafluorophosphate |
| PyBOP | Benzotriazol-1-yl-oxytripyrrolidinophosphonium |
| | hexafluorophosphate |
| t-BuOK | Potassium tertbutoxide/potassium2-methylpropan-2-olate |
| CDI | 1,1′-Carbonyldiimidazole |
| COMU | (1-Cyano-2-ethoxy-2-oxoethylidenaminooxy)dimethylamino- |
| | morpholino-carbenium hexafluorophosphate |
| DCC | N,N′-dicyclohexylcarbodiimide |
| DCM | Dichloromethane |
| DIC | N,N′-Diisopropylcarbodiimide |
| DIEA/DIPEA | N,N-Diisopropylethylamine |
| DME | Dimethyl ether |
| DMF | Dimethylformamide |
| DMP | Dess-Martin periodinane |
| DMSO | Dimethylsulfoxide |
| EA | Ethyl acetate |
| EDC | 1-(3-dirnethylarninopropyl)-3-ethylcarbodiirnide |
| EtOH | Ethanol |
| HATU | 1-[Bis(dimethylamino)methylene]-1H-1,2,3-triazolo[4,5- |
| | b]pyridinium 3-oxid hexafluorophosphate |
| HBTU | N,N,N′,N′-Tetramethyl-O-(1H-benzotriazol-1-yl)uranium |
| | hexafluorophosphate |
| HOAt | 1-Hydroxy-7-azabenzotriazole |
| HOBt | Hydroxybenzotriazole |
| MeOH | Methanol |
| MsCl | Methanesulfonyl chloride |
| NMO | 4-Methylmorpholine N-oxide |
| NBS | N-Bromosuccinimide |
| PCC | Pyridinium chlorochromate |
| PDC | Pyridinium dichromate |
| PdCl₂(dppf)- | [1,1′-Bis(diphenylphosphino)ferrocene]dichloropalladium(II), |
| CH₂Cl₂adduct | complex with dichloromethane |
| TBTU | O-(benzotriazol-1-yl)-N,N,N′N′-tetramethyluronium |
| | tetrafluoroborate |
| TEA | Triethylamine |
| TFA | Trifluoroacetic acid |
| TFE | 2,2,2-Trifluoroethanol |
| THF | Tetrahydrofuran |
| TOTU | O-[(ethoxycarbonyl)cyanomethylenamino]-N,N,N′,N′- |
| | tetramethyluronium tetrafluoroborate |
| T₃P | Propylphosphonic anhydride |

### Example 1: (1R,2S,5S)-6,6-dimethyl-N—((S)-1-oxo-3-((S)-2-oxopyrrolidin-3-yl)propan-2-yl)-3-(1-phenylcyclopropanecarbonyl)-3-azabicyclo[3.1.0]hexane-2-carboxamide (Compound 1)

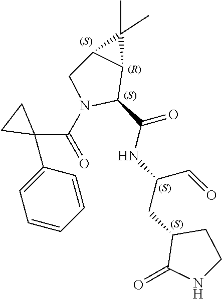

### Step 1: (1R,2S,5S)-3-tert-butyl 2-methyl 6,6-dimethyl-3-azabicyclo[3.1.0]hexane-2,3-dicarboxylate

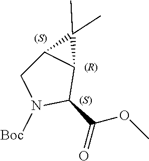

To a solution of (1R,2S,5S)-methyl 6,6-dimethyl-3-azabicyclo[3.1.0]hexane-2-carboxylate hydrochloride (2.70 g, 13.13 mmol, HCl) in DCM (10 mL) was added (Boc)₂O (4.30 g, 19.69 mmol) and TEA (3.98 g, 39.38 mmol) dropwise at 25° C. The mixture was stirred at 25° C. for 10 hours. The reaction mixture was poured into water (50 mL). The aqueous phase was extracted with DCM (20 mL×3). The combined organic phase was washed with brine (20 mL×2), dried with anhydrous Na₂SO₄, filtered and concentrated in vacuo. The title compound was obtained as yellow oil (3.3 g, 87.74% yield) which was used in the next step without further purification. ¹H NMR (400 MHz, CDCl₃) δ ppm 4.23-4.05 (m, 1H), 3.78-3.75 (m, 3H), 3.70-3.60 (m, 1H), 3.49-3.35 (m, 1H), 1.54-1.45 (m, 2H), 1.49-1.35 (m, 9H), 0.99-0.96 (m, 3H).

### Step 2: (1R,2S,5S)-3-(tert-butoxycarbonyl)-6,6-dimethyl-3-azabicyclo[3.1.0]hexane-2-carboxylic acid

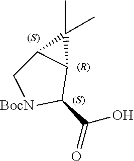

To a solution of (1R,2S,5S)-3-tert-butyl 2-methyl 6,6-dimethyl-3-azabicyclo[3.1.0]hexane-2,3-dicarboxylate (1.50 g, 5.57 mmol) in H₂O (4 mL) and THE (12 mL) was added LiOH·H₂O (467 mg, 11.14 mmol) in one portion at 25° C. The mixture was stirred at 25° C. for 10 hours. The mixture was poured into water (15 mL) and then acidified with 2M HCl (5 mL) to pH ~6. The aqueous phase was extracted with DCM (20 mL×4). The combined organic phase was dried with anhydrous Na₂SO₄, filtered and concentrated in vacuo. The title compound was obtained as yellow oil (1.2 g, 70.05% yield) which was used in the next step without further purification. ¹H NMR (400 MHz, CDCl₃) δ ppm 4.22-4.09 (m, 1H), 3.70-3.54 (m, 1H), 3.49-3.40 (m, 1H), 1.75 (d, J=7.6 Hz, 1H), 1.55-1.35 (m, 10H), 1.10-1.05 (m, 3H), 1.02-0.93 (m, 3H).

### Step 3: (S)-methyl 2-amino-3-((S)-2-oxopyrrolidin-3-yl)propanoate

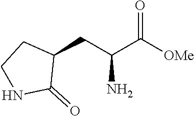 [SMILES: COC(=O)[C@@H](N)C[C@@H]1CCNC1=O]

To a solution of (S)-methyl 2-((tert-butoxycarbonyl)amino)-3-((S)-2-oxopyrrolidin-3-yl)propanoate (1.35 g, 4.72 mmol) in dioxane (10 mL) was added HCl/dioxane (4 M, 14 mL) in one portion at 25° C. The mixture was stirred at 25° C. for 2 hours. The mixture was concentrated under reduced pressure. The title compound was obtained as a white solid (1.05 g, crude, HCl) which was used to the next step without further purification.

### Step 4: (1R,2S,5S)-tert-butyl 2-(((S)-1-methoxy-1-oxo-3-((S)-2-oxopyrrolidin-3-yl)propan-2-yl)carbamoyl)-6,6-dimethyl-3-azabicyclo[3.1.0]hexane-3-carboxylate

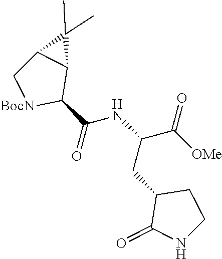 [SMILES: COC(=O)[C@H](C[C@@H]1CCNC1=O)NC(=O)[C@@H]1[C@@H]2[C@H](CN1C(=O)OC(C)(C)C)C2(C)C]

To a solution of (1R,2S,5S)-3-(tert-butoxycarbonyl)-6,6-dimethyl-3-azabicyclo[3.1.0]hexane-2-carboxylic acid (1.20 g, 4.70 mmol) and DIEA (2.43 g, 18.80 mmol) in DCM (15 mL) was added T₃P (4.49 g, 7.05 mmol, 50% purity in EtOAc) dropwise at 0° C. After addition, the reaction mixture was stirred at the same temperature for 30 mins. Then (S)-methyl 2-amino-3-((S)-2-oxopyrrolidin-3-yl)propanoate (1.05 g, 4.72 mmol, HCl) in DCM (10 mL) was added in one portion at 0° C. The mixture was stirred at 25° C. for 10 hours. The mixture was poured in to 5% H₃PO₄ (20 mL). The aqueous phase was extracted with DCM (20 mL×3). The combined organic phase was washed with sat.aq. NaHCO₃ (20 mL) and brine (10 mL), dried with anhydrous Na₂SO₄, filtered and concentrated under vacuum. The title compound was obtained as a white solid (1.00 g, 20.10% yield) which was used in the next step without further purification. ¹H NMR (400 MHz, CDCl₃) δ ppm 7.75-7.20 (m, 1H), 6.32-5.87 (m, 1H), 4.71-4.44 (m, 1H), 4.15-4.03 (m, 1H), 3.74 (s, 3H), 3.67-3.41 (m, 2H), 3.40-3.28 (m, 2H), 2.51-2.34 (m, 2H), 2.26-2.12 (m, 1H), 1.95-1.77 (m, 4H), 1.47-1.39 (m, 9H), 1.07-0.86 (m, 6H).

### Step 5: (S)-methyl 2-((1R,2S,5S)-6,6-dimethyl-3-azabicyclo[3.1.0]hexane-2-carboxamido)-3-((S)-2-oxopyrrolidin-3-yl)propanoate

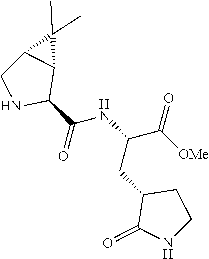 [SMILES: COC(=O)[C@H](C[C@@H]1CCNC1=O)NC(=O)[C@H]1NC[C@H]2[C@@H]1C2(C)C]

To a solution of (1R,2S,5S)-tert-butyl 2-(((S)-1-methoxy-1-oxo-3-((S)-2-oxopyrrolidin-3-yl)propan-2-yl)carbamoyl)-6,6-dimethyl-3-azabicyclo[3.1.0]hexane-3-carboxylate (300 mg, 708.38 mol) in dioxane (3 mL) was added HCl/dioxane (4 M, 4 mL) in one portion at 25° C. The mixture was stirred at 25° C. for 2 hours. The mixture was concentrated under reduce pressure. The title compound was obtained as a white solid (250 mg, crude, HCl) which was used in the next step without further purification. ¹H NMR (400 MHz, DMSO-d₆) δ ppm 10.47 (br s, 1H), 9.30 (d, J=7.2 Hz, 1H), 8.84 (brs, 1H), 7.72 (s, 1H), 4.42-4.29 (m, 1H), 4.05 (s, 1H), 3.66 (s, 3H), 3.63-3.58 (m, 1H), 3.23-3.00 (m, 3H), 2.44-2.29 (m, 1H), 2.22-2.01 (m, 2H), 1.77-1.59 (m, 4H), 1.13-1.02 (m, 6H).

### Step 6: (S)-methyl 2-((1R,2S,5S)-6,6-dimethyl-3-(1-phenylcyclopropanecarbonyl)-3-azabicyclo[3.1.0]hexane-2-carboxamido)-3-((S)-2-oxopyrrolidin-3-yl)propanoate

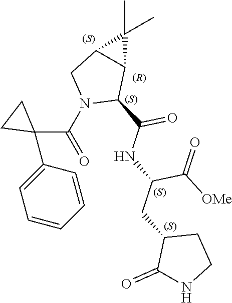

To a solution of 1-phenylcyclopropanecarboxylic acid (114 mg, 708.63 mol) and DIEA (366 mg, 2.83 mmol) in DCM (5 mL) was added T₃P (676 mg, 1.06 mmol, 50% purity in EtOAc) dropwise at 0° C. After addition, the mixture was stirred at the same temperature for 30 min, and then (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 708.63 μmol, HCl) was added at 25° C. The resulting mixture was stirred at 25° C. for 11.5 hrs. The mixture was diluted with sat.aq. NH₄Cl (8 mL), extracted with DCM (10 mL×3). The organic layer was dried over Na₂SO₄, filtered and concentrated to crude product. The residue was purified by prep-HPLC (column: Welch Ultimate XB—CN 250×50×10 μm; mobile phase: [Heptane-EtOH (0.1% NH₃H₂O)]; B %: 10%-%, 8 min). The title compound (135 mg, 37.89% yield) was obtained as a white solid. ESI-MS [M+H]⁺ calc'd for C₂₆H₃₃N₃O₅, 468.24; found, 468.3.

### Step 7: (1R,2S,5S)—N—((S)-1-hydroxy-3-((S)-2-oxopyrrolidin-3-yl)propan-2-yl)-6,6-dimethyl-3-(1-phenylcyclopropanecarbonyl)-3-azabicyclo[3.1.0]hexane-2-carboxamide

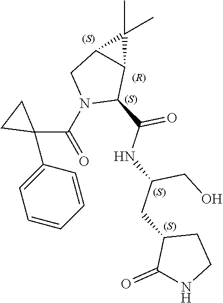

To a solution of (S)-methyl 2-((1R,2S,5S)-6,6-dimethyl-3-(1-phenylcyclopropanecarbonyl)-3-azabicyclo[3.1.0]hexane-2-carboxamido)-3-((S)-2-oxopyrrolidin-3-yl)propanoate (130 mg, 278.04 mol) in THF (4 mL) was added LiBH₄ (18 mg, 834.12 mol) at 0° C. The mixture was stirred at 0° C. for 1 hr. The mixture was quenched with H₂O (10 mL), and extracted with DCM (5 mL×3). The combined organic layer was dried over anhydrous Na₂SO₄, filtered and concentrated to dryness. The title compound (77 mg, 37.80% yield) was obtained as a white solid. ESI-MS [M+H]⁺ calc'd for C₂₅H₃₃N₃O₄, 440.25; found, 440.3.

### Step 8: (1R,2S,5S)-6,6-dimethyl-N—((S)-1-oxo-3-((S)-2-oxopyrrolidin-3-yl)propan-2-yl)-3-(1-phenylcyclopropanecarbonyl)-3-azabicyclo[3.1.0]hexane-2-carboxamide

A mixture of (1R,2S,5S)—N—((S)-1-hydroxy-3-((S)-2-oxopyrrolidin-3-yl)propan-2-yl)-6,6-dimethyl-3-(1-phenylcyclopropanecarbonyl)-3-azabicyclo[3.1.0]hexane-2-carboxamide (77 mg, 175.18 mol) and DMP (222 mg, 525.54 mol) in DCM (2 mL) was degassed and purged with N₂ for 3 times, and then the mixture was stirred at 25° C. for 0.5 hr under N₂ atmosphere. The mixture was filtered and the filtrate was concentrated to give crude product. The crude product was purified by prep-HPLC (column: Phenomenex C18 75×30 mm×3 μm; mobile phase: [water (NH₄HCO₃)-ACN]; B %: 22%-45%, 10 min). Compound 1 was obtained as a white solid (5.7 mg, 7.14% yield, 96% purity). ¹H NMR (400 MHz, CD₃OD) δ ppm 7.25-7.08 (m, 5H), 4.48 (s, 1H), 4.40-4.18 (m, 1H), 3.94-3.81 (m, 1H), 3.54-3.43 (m, 1H), 3.29-3.22 (m, 2H), 2.61-2.18 (m, 2H), 2.00-1.86 (m, 1H), 1.73-1.41 (m, 2H), 1.38-1.11 (m, 5H), 1.06-0.97 (m, 1H), 0.92-0.78 (m, 3H), 0.67-0.33 (m, 3H). ESI-MS [M+H]⁺ calc'd for C₂₅H₃₁N₃O₄, 438.23; found, 438.4.

### Example 2: (S)-5-(7-chloro-1H-indole-2-carbonyl)-N—((S)-1-oxo-3-((S)-2-oxopyrrolidin-3-yl)propan-2-yl)-5-azaspiro[2.4]heptane-6-carboxamide (Compound 2)

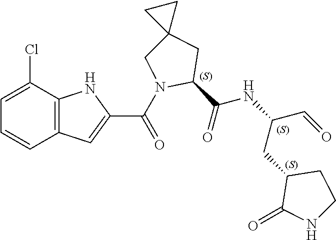

### Step 1: (S)-tert-butyl 6-(((S)-1-methoxy-1-oxo-3-((S)-2-oxopyrrolidin-3-yl)propan-2-yl)carbamoyl)-5-azaspiro[2.4]heptane-5-carboxylate

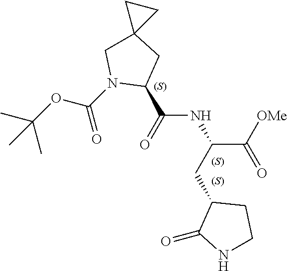

To a solution of (S)-5-(tert-butoxycarbonyl)-5-azaspiro[2.4]heptane-6-carboxylic acid (500 mg, 2.07 mmol) and DIEA (1.07 g, 8.29 mmol) in DCM (5 mL) was added dropwise T₃P (1.98 g, 3.11 mmol, 1.85 mL, 50% purity in EtOAc) at 0° C. After addition, the mixture was stirred at the same temperature for 30 min, and then (S)-methyl 2-amino-3-((S)-2-oxopyrrolidin-3-yl)propanoate hydrochloride (461 mg, 2.07 mmol, HCl) was added at 25° C. The resulting mixture was stirred at 25° C. for 10 hr. The reaction mixture was diluted with EtOAc (30 mL) and 5% H₃PO₄ (30 mL). The layers were separated. The aqueous phase was extracted with EtOAc (50 mL). The combined organic layers were washed with 5% H₃PO₄ (30 mL×3), sat.aq.NaHCO₃ (30 mL×4), water (30 mL), and brine (30 mL), and dried over anhydrous Na₂SO₄, filtered and concentrated in vacuo. The title compound (680 mg, 57.83% yield) was obtained as colorless oil. ¹H NMR (400 MHz, DMSO-d₆) δ ppm 8.52-8.29 (m, 1H), 7.72-7.58 (m, 1H), 4.37-4.26 (m, 1H), 4.25-4.19 (m, 1H), 3.61 (s, 3H), 3.30-3.19 (m, 2H), 3.17-3.04 (m, 2H), 2.41-2.18 (m, 2H), 2.16-2.00 (m, 2H), 1.74-1.51 (m, 3H), 1.42-1.29 (m, 9H), 0.59-0.36 (m, 4H).

### Step 2: (S)-methyl 3-((S)-2-oxopyrrolidin-3-yl)-2-((S)-5-azaspiro[2.4]heptane-6-carboxamido)propanoate

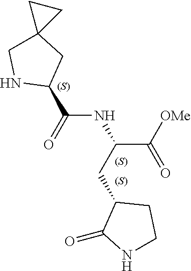

A mixture of (S)-tert-butyl 6-(((S)-1-methoxy-1-oxo-3-((S)-2-oxopyrrolidin-3-yl)propan-2-yl)carbamoyl)-5-azaspiro[2.4]heptane-5-carboxylate (650 mg, 1.59 mmol) in HCl/dioxane (7 mL, 4M) was degassed and purged with N₂ for 3 times, and then the mixture was stirred at 25° C. for 1 hr. The reaction mixture was concentrated to dryness. The title compound (548 mg, 99.83% yield, HCl) was obtained as a white solid which was used in the next step without further purification.

### Step 3: (S)-methyl 2-((S)-5-(7-chloro-1H-indole-2-carbonyl)-5-azaspiro[2.4]heptane-6-carboxamido)-3-((S)-2-oxopyrrolidin-3-yl)propanoate

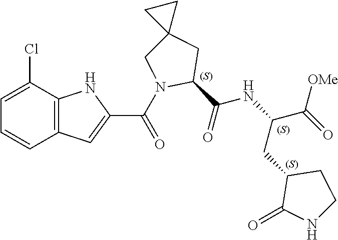

To a solution of compound (S)-methyl 3-((S)-2-oxopyrrolidin-3-yl)-2-((S)-5-azaspiro[2.4]heptane-6-carboxamido)propanoate (165 mg, 477.13 μmol, 1 eq, HCl), 7-chloro-1H-indole-2-carboxylic acid (93 mg, 477.13 mol) and DIEA (185 mg, 1.43 mmol, 249.31 μL) in DCM (3 mL) was added HATU (363 mg, 954.25 mol) slowly at 0° C. The mixture was stirred at 25° C. for 10 hr. Sat.aq.NH₄Cl (20 mL) was added to the residue. The resulting mixture was extracted with DCM (30 mL×3). The combined organic phase was washed with water (20 mL), dried over anhydrous Na₂SO₄, filtered and concentrated in vacuo. The residue was purified by prep-HPLC (column: Xtimate C18 150×40 mm×10 μm; mobile phase: [water (10 mM NH₄HCO₃)-ACN]; B %: 25%-55%, 10 min). The title compound (105 mg, 43.74% yield) was obtained as a yellow solid. ¹H NMR (400 MHz, CDCl₃) δ ppm 9.84-9.37 (m, 1H), 9.17-8.07 (m, 1H), 7.55 (d, J=8.0 Hz, 1H), 7.31-7.26 (m, 1H), 7.12-6.85 (m, 2H), 6.40-5.69 (m, 1H), 5.06-4.89 (m, 1H), 4.61-4.31 (m, 1H), 4.08-3.74 (m, 2H), 3.74-3.44 (m, 3H), 3.44-3.26 (m, 2H), 2.61-2.30 (m, 2H), 2.27-2.04 (m, 2H), 1.99-1.79 (m, 2H), 1.77-1.67 (m, 1H), 0.81-0.42 (m, 4H).

### Step 4: (S)-5-(7-chloro-1H-indole-2-carbonyl)-N—((S)-1-hydroxy-3-((S)-2-oxopyrrolidin-3-yl)propan-2-yl)-5-azaspiro[2.4]heptane-6-carboxamide

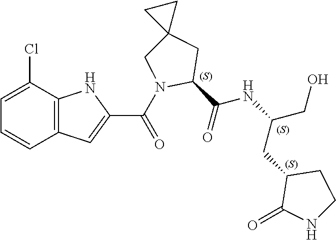

To a solution of (S)-methyl 2-((S)-5-(7-chloro-1H-indole-2-carbonyl)-5-azaspiro[2.4]heptane-6-carboxamido)-3-((S)-2-oxopyrrolidin-3-yl)propanoate (105 mg, 215.63 mol) in THE (2 mL) was added LiBH₄ (19 mg, 862.52 mol) at 0° C. The mixture was stirred at 25° C. for 1 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated in vacuo. The title compound (85 mg, crude) was obtained as a white solid. ¹H NMR (400 MHz, CDCl₃) δ ppm 9.99-9.53 (m, 1H), 8.84-8.11 (m, 1H), 7.60-7.41 (m, 1H), 7.30-7.26 (m, 1H), 7.09-6.99 (m, 1H), 6.98-6.79 (m, 1H), 6.03-5.61 (m, 1H), 5.04-4.79 (m, 2H), 4.08-3.80 (m, 2H), 3.67-3.45 (m, 2H), 3.40-3.16 (m, 2H), 2.64-2.46 (m, 1H), 2.46-2.08 (m, 3H), 2.07-1.95 (m, 1H), 1.82-1.56 (m, 3H), 0.77-0.47 (m, 4H).

### Step 5: (S)-5-(7-chloro-1H-indole-2-carbonyl)-N—((S)-1-oxo-3-((S)-2-oxopyrrolidin-3-yl)propan-2-yl)-5-azaspiro[2.4]heptane-6-carboxamide

To a solution of (S)-5-(7-chloro-1H-indole-2-carbonyl)-N—((S)-1-hydroxy-3-((S)-2-oxopyrrolidin-3-yl)propan-2-yl)-5-azaspiro[2.4]heptane-6-carboxamide (70 mg, 152.53 mol) in DCM (10 mL) was added DMP (129 mg, 305.05 mol). The mixture was stirred at 25° C. for 10 min. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 19%-54%, 10 min). Compound 2 (7 mg, 9.79% yield, 97.45% purity) was obtained as a white solid. ¹H NMR (400 MHz, CD₃OD) δ ppm 7.65-7.44 (m, 1H), 7.32-7.21 (m, 1H), 7.13-6.89 (m, 2H), 5.22-4.87 (m, 1H), 4.60-4.33 (m, 1H), 4.11-3.65 (m, 3H), 3.08-2.60 (m, 1H), 2.57-2.15 (m, 2H), 2.15-1.72 (m, 3H), 1.70-1.53 (m, 1H), 1.52-1.25 (m, 1H), 0.84-0.54 (m, 4H). ESI-MS [M+H]+ calc'd for C₂₃H₂₅ClN₄O₄, 457.16; found, 457.3.

### Example 3: (S)-5-(4-fluoro-1H-indole-2-carbonyl)-N—((S)-1-oxo-3-((S)-2-oxopyrrolidin-3-yl)propan-2-yl)-5-azaspiro[2.4]heptane-6-carboxamide (Compound 3)

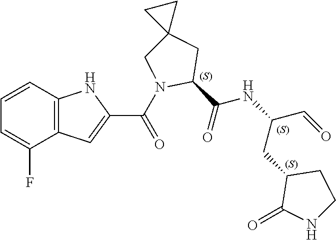

### Step 1: (S)-methyl 2-((S)-5-(4-fluoro-1H-indole-2-carbonyl)-5-azaspiro[2.4]heptane-6-carboxamido)-3-((S)-2-oxopyrrolidin-3-yl)propanoate

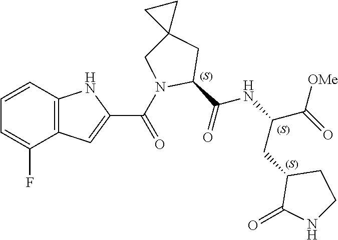

To a solution of (S)-methyl 3-((S)-2-oxopyrrolidin-3-yl)-2-((S)-5-azaspiro[2.4]heptane-6-carboxamido)propanoate (169 mg, 488.69 μmol, HCl) in DCM (2 mL) was added DIEA (189 mg, 1.47 mmol) and 4-fluoro-1H-indole-2-carboxylic acid (88 mg, 488.69 mol) dropwise at 0° C. The mixture was stirred at 0° C. for 20 mins. Then HATU (372 mg, 977.38 mol) in DCM (2 mL) was added in one portion at 0° C. The mixture was stirred at 25° C. 10 hr. H₂O (10 mL) was added and the resulting mixture was extracted with DCM (10 mL×3). The combined organic phase was washed with sat.aq.NaHCO₃ (10 mL×2), brine (10 mL) and water (10 mL), dried over anhydrous Na₂SO₄, filtered and concentrated. The residue was purified by prep-HPLC (column: Phenomenex Gemini-NX C18 75×30 mm×3 μm; mobile phase: [water (10 mM NH₄HCO₃)-ACN]; B %: 17%-57%, 11 min). The title compound (90 mg, 38.71% yield) was obtained as a white solid. ¹H NMR (400 MHz, CDCl₃) δ ppm 10.02-9.77 (m, 1H), 9.24-7.90 (m, 1H), 7.24-7.07 (m, 2H), 7.00-6.87 (m, 1H), 6.82-6.68 (m, 1H), 6.29-5.82 (m, 1H), 5.02-4.88 (m, 1H), 4.68-4.35 (m, 1H), 4.15-3.79 (m, 2H), 3.71 (s, 3H), 3.46-3.25 (m, 2H), 2.53-2.35 (m, 2H), 2.24-2.13 (m, 2H), 2.03-1.77 (m, 3H), 0.76-0.50 (m, 4H).

### Step 2: (S)-5-(4-fluoro-1H-indole-2-carbonyl)-N—((S)-1-hydroxy-3-((S)-2-oxopyrrolidin-3-yl)propan-2-yl)-5-azaspiro[2.4]heptane-6-carboxamide

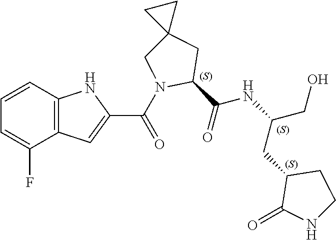

To a solution of (S)-methyl 2-((S)-5-(4-fluoro-1H-indole-2-carbonyl)-5-azaspiro[2.4]heptane-6-carboxamido)-3-((S)-2-oxopyrrolidin-3-yl)propanoate (90 mg, 191.29 mol) in THE (6 mL) was added LiBH₄ (12.5 mg, 573.87 mol) in one portion at 0° C. The mixture was stirred at 0° C. for 1 hr. H₂O (10 mL) was added to the reaction mixture. The resulting mixture was extracted with DCM (15 mL×3). The combined organic phase was washed with brine (25 mL), water (25 mL), dried over anhydrous Na₂SO₄, filtered and concentrated to dryness. The title compound (90 mg, crude) was obtained as a white solid. ¹H NMR (400 MHz, CDCl₃) δ ppm=10.35-9.85 (m, 1H), 8.51-7.88 (m, 1H), 7.24-7.12 (m, 2H), 6.95-6.84 (m, 1H), 6.75 (t, J=8.4 Hz, 1H), 6.10-5.85 (m, 1H), 5.03-4.78 (m, 1H), 4.07-3.92 (m, 2H), 3.91-3.82 (m, 1H), 3.72-3.43 (m, 2H), 3.31-3.20 (m, 2H), 2.60-2.44 (m, 1H), 2.42-2.27 (m, 1H), 2.25-2.17 (m, 1H), 2.13-2.00 (m, 2H), 1.81-1.69 (m, 1H), 1.57 (m, 1H), 1.35-1.18 (m, 1H), 0.76-0.55 (m, 4H).

### Step 3: (S)-5-(4-fluoro-1H-indole-2-carbonyl)-N—((S)-1-oxo-3-((S)-2-oxopyrrolidin-3-yl)propan-2-yl)-5-azaspiro[2.4]heptane-6-carboxamide

To a solution of (S)-5-(4-fluoro-1H-indole-2-carbonyl)-N—((S)-1-hydroxy-3-((S)-2-oxopyrrolidin-3-yl)propan-2-yl)-5-azaspiro[2.4]heptane-6-carboxamide (70 mg, 158.20 mol) in DCM (3 mL) was added DMP (134 mg, 316.40 mol) at 25° C., and the reaction mixture was stirred at 25° C. for 0.5 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 13%-53%, 11 min). Compound 3 (15.6 mg, 21.84% yield, 97.569% purity) was obtained as a white solid. ¹H NMR (400 MHz, CD₃OD) δ ppm=7.29-7.22 (m, 1H), 7.22-7.12 (m, 1H), 7.08-6.85 (m, 1H), 6.78-6.67 (m, 1H), 4.63-4.35 (m, 1H), 4.10-3.84 (m, 2H), 3.81-3.45 (m, 1H), 3.09-2.49 (m, 2H), 2.46-1.92 (m, 4H), 1.89-1.17 (m, 3H), 0.82-0.56 (m, 4H). ESI-MS [M+H]⁺ calc'd. for C₂₃H₂₅FN₄O₄, 441.19, found 441.4.

### Example 4: (1R,2S,5S)-3-(6,7-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 4)

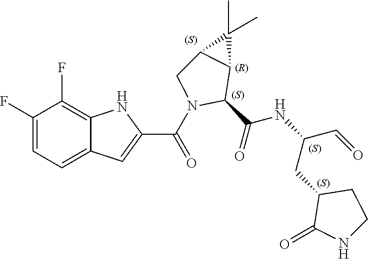

### Step 1: ethyl 6,7-difluoro-1H-indole-2-carboxylate

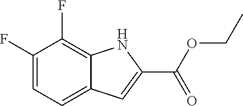 [SMILES: CCOC(=O)c1cc2ccc(F)c(F)c2[nH]1]

To a solution of (2,3-difluorophenyl)hydrazine (500 mg, 2.77 mmol, HCl) and ethyl 2-oxopropanoate (321 mg, 2.77 mmol, 0.31 mL) in toluene (10 mL) was added TsOH·H₂O (50 mg, 0.27 mmol) at 15° C. The mixture was stirred at 120° C. for 2 hr. TsOH·H₂O (1.58 g, 8.31 mmol) was added. The mixture was stirred at 120° C. for 10 hr. The reaction mixture was concentrated, sat.aq NaHCO₃ (30 mL) was added to the residue. The resulting mixture was extracted with EtOAc (30 mL×3). The combined organic phase was washed with brine (20 mL), dried over anhydrous Na₂SO₄, filtered and concentrated to dryness. The residue was purified by flash silica gel chromatography (eluent of 7% EtOAc/PE). The title compound (170 mg, 27.26% yield) was obtained as a yellow solid. ¹H NMR (400 MHz, CDCl₃) δ ppm 9.04 (brs 1H), 7.41-7.33 (m, 1H), 7.24-7.20 (m, 1H), 7.06-7.92 (m, 1H), 4.43 (q, J=7.2 Hz, 2H), 1.43 (t, J=7.2 Hz, 3H).

### Step 2: 6,7-difluoro-1H-indole-2-carboxylic acid

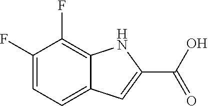 [SMILES: O=C(O)c1cc2ccc(F)c(F)c2[nH]1]

To a mixture of ethyl 6,7-difluoro-1H-indole-2-carboxylate (170 mg, 0.75 mmol,) in H₂O (1 mL) and MeOH (1 mL) was added LiOH·H₂O (63 mg, 1.51 mmol). The mixture was stirred at 50° C. for 3 hr. The reaction mixture was poured into water (20 mL) and then extracted with PE (50 mL×2). The aqueous phase was then adjusted to pH~5 by aqueous HCl (1M). The resulting mixture was extracted with EtOAc (50 mL×2). The combined organic phase was dried over anhydrous Na₂SO₄, filtered and concentrated to dryness. The title compound (114 mg, crude) was obtained as a yellow solid. ¹H NMR (400 MHz, CD₃OD) δ ppm 7.45-7.37 (m, 1H), 7.21-7.16 (m, 1H), 7.03-6.92 (m, 1H).

### Step 3: (S)-methyl-2-((1R,2S,5S)-3-(6,7-difluoro-1H-indole-2-carbonyl)-6,6-dimethyl-3-azabicyclo[3.1.0]hexane-2-carboxamido)-3-((S)-2-oxopyrrolidin-3-yl)propanoate

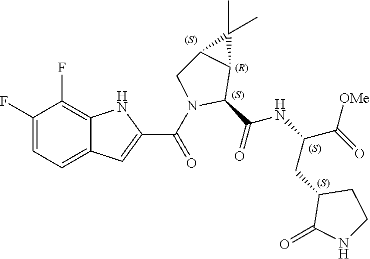

To a mixture of (S)-methyl 2-((1R,2S,5S)-6,6-dimethyl-3-azabicyclo[3.1.0]hexane-2-carboxamido)-3-((S)-2-oxopyrrolidin-3-yl)propanoate (182 mg, 0.51 mmol,) and 6,7-difluoro-1H-indole-2-carboxylic acid (100 mg, 0.51 mmol) in DCM (3 mL) was added DIEA (197 mg, 1.52 mmol, 0.26 mL). The mixture was stirred at 0° C. for 20 mins. HATU (386 mg, 1.01 mmol) was added to the above mixture. The mixture was stirred at 25° C. for 3 hr. The reaction was quenched with aqueous NaHCO₃ (10 mL) drop-wise. The resulting mixture was extracted with DCM (30 mL×2). The combined organic phase was washed with water (20 mL×2), dried over anhydrous Na₂SO₄, filtered and concentrated to dryness. The residue was purified by flash silica gel chromatography (eluent of 0-100% EtOAc/PE). The title compound was obtained as a white solid (207 mg, 48.07% yield). ¹H NMR (400 MHz, CDCl₃) δ ppm 9.91-9.50 (m, 1H), 8.54-7.53 (m, 1H), 7.40-7.30 (m, 1H), 7.02-6.96 (m, 1H), 6.96-6.85 (m, 1H), 4.72-4.56 (m, 1H), 4.49-4.17 (m, 1H), 4.01-3.90 (m, 1H), 3.81-3.56 (m, 4H), 3.54-3.34 (m, 2H), 2.78-2.63 (m, 1H), 2.52-2.34 (m, 1H), 2.22-2.12 (m, 1H), 2.03-1.80 (m, 2H), 1.70-1.52 (m, 1H), 1.46-1.42 (m, 1H), 1.15-0.83 (m, 6H).

### Step 4: (1R,2S,5S)-3-(6,7-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

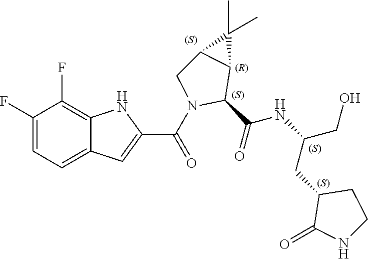

To a mixture of (S)-methyl 2-((1R,2S,5S)-3-(6,7-difluoro-1H-indole-2-carbonyl)-6,6-dimethyl-3-azabicyclo[3.1.0]hexane-2-carboxamido)-3-((S)-2-oxopyrrolidin-3-yl)propanoate (200 mg, 0.40 mmol) in THF (3 mL) was added LiBH₄ (20 mg, 0.92 mmol) at 0° C. The mixture was stirred at 25° C. for 1 hr. The reaction was quenched with water (10 mL) drop-wise. The resulting mixture was extracted with DCM (20 mL×3). The combined organic phase was washed with water (10 mL), dried over anhydrous Na₂SO₄, filtered and concentrated. The residue was purified by prep-HPLC (column: Phenomenex Gemini-NX C18 75×30 mm×3 μm; mobile phase: [water (10 mM NH₄HCO₃)-ACN]; B %: 16% -56%, 11 min). The title compound (37 mg, 18.79% yield) was obtained as a white solid. ¹H NMR (400 MHz, CDCl₃) δ ppm 10.20-9.76 (m, 1H), 8.17 (d, J=6.4 Hz, 1H), 7.38-7.31 (m, 1H), 7.04-6.83 (m, 2H), 5.72 (brs, 1H), 4.70-4.63 (m, 1H), 4.30-4.22 (m, 1H), 4.08-3.95 (m, 1H), 3.88-3.82 (m, 1H), 3.81-3.76 (m, 1H), 3.64-3.56 (m, 1H), 3.40-3.26 (m, 2H), 2.68-2.29 (m, 2H), 2.11-1.91 (m, 2H), 1.90-1.86 (m, 1H), 1.72-1.67 (m, 1H), 1.67-1.61 (m, 2H), 1.11-0.88 (m, 6H).

### Step 5: (1R,2S,5S)-3-(6,7-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(6,7-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (30 mg, 63.22 mol) in DCM (5 mL) was added DMP (81 mg, 189.67 mol) at 25° C. The reaction mixture was stirred at 25° C. for 0.5 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 22%-52%, 10 min). Compound 4 (10 mg, 33.16% yield, 99.065% purity) was obtained as a white solid. ¹H NMR (400 MHz, CD₃OD) δ ppm 7.47-7.23 (m, 1H), 7.15-6.85 (m, 2H), 4.65-4.62 (m, 1H), 4.53-3.88 (m, 3H), 3.83-3.68 (m, 1H), 3.26-2.97 (m, 1H), 2.68-2.41 (m, 1H), 2.40-1.77 (m, 2H), 1.74-1.67 (m, 1H), 1.62-1.30 (m, 3H), 1.16-0.92 (m, 6H). ESI-MS [M+H]⁺ calc'd for C₂₄H₂₆F₂N₄O₄, 473.19, found 473.4.

### Example 5: (1R,2S,5S)-3-(7-chloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 5)

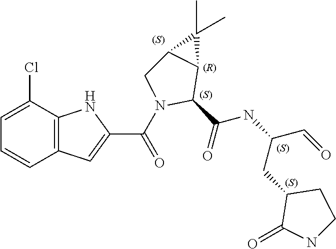

### Step 1: (S)-methyl 2-((1R,2S,5S)-3-(7-chloro-1H-indole-2-carbonyl)-6,6-dimethyl-3-azabicyclo[3.1.0]hexane-2-carboxamido)-3-((S)-2-oxopyrrolidin-3-yl)propanoate

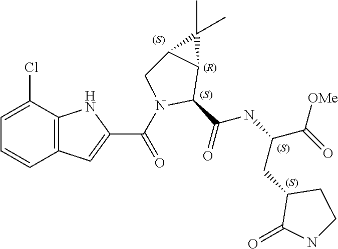

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (181 mg, 927.68 mol) and DIEA (360 mg, 2.78 mmol) in DCM (5 mL) was added 7-chloro-1H-indole-2-carboxylic acid (300 mg, 927.68 μmol, HCl). After addition, HATU (705 mg, 1.86 mmol) was added at 0° C. The mixture was stirred at 25° C. for 12 hr. The reaction mixture was poured into sat.aq NH₄Cl (20 mL). The resulting mixture was extracted with EtOAc (20 mL×2). The combined organic phase was washed with brine (30 mL), dried over anhydrous Na₂SO₄, filtered and concentrated to dryness. The residue was purified by prep-HPLC (column: Xtimate C18 150×40 mm×10 μm; mobile phase: [water (10 mM NH₄HCO₃)-ACN]; B %: 25%-55%, 10 min). The title compound (216 mg, 45.52% yield) was obtained as a white solid. ¹H NMR (400 MHz, CDCl₃) δ ppm 9.65-9.40 (m, 1H), 8.12-8.04 (m, 1H), 7.62-7.49 (m, 1H), 7.30-7.27 (m, 1H), 7.10-6.96 (m, 2H), 5.80-5.61 (m, 1H), 4.76-4.65 (m, 1H), 4.59-4.49 (m, 1H), 4.27-4.16 (m, 1H), 4.03-3.89 (m, 1H), 3.78-3.66 (m, 3H), 3.39-3.28 (m, 2H), 2.66-2.51 (m, 1H), 2.49-2.39 (m, 1H), 2.35-2.25 (m, 1H), 2.24-2.11 (m, 1H), 2.01-1.73 (m, 3H), 1.11-1.05 (m, 3H), 0.95-0.91 (m, 3H).

### Step 2: (1R,2S,5S)-3-(7-chloro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

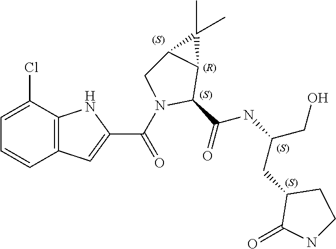

To a solution of (S)-methyl 2-((1R,2S,5S)-3-(7-chloro-1H-indole-2-carbonyl)-6,6-dimethyl-3-azabicyclo[3.1.0]hexane-2-carboxamido)-3-((S)-2-oxopyrrolidin-3-yl)propanoate (210 mg, 419.18 mol) in THE (8 mL) was added LiBH₄ (27 mg, 1.26 mmol) at 0° C. Then the mixture was stirred at 25° C. for 1 hr. Water (20 mL) was added to the mixture. The resulting mixture was extracted with ethyl acetate (20 mL×2). The combined organic layers were washed with brine (20 mL), dried over anhydrous Na₂SO₄, filtered and concentrated to dryness. The title compound (170 mg, crude) was obtained as a white solid. ¹H NMR (400 MHz, CDCl₃) δ ppm 10.00 (brs, 1H), 8.30-8.14 (m, 1H), 7.60-7.49 (m, 1H), 7.25-7.23 (m, 1H), 7.09-6.96 (m, 3H), 6.27-6.02 (m, 1H), 4.67 (s, 1H), 4.34-4.25 (m, 1H), 4.07-3.90 (m, 2H), 3.77-3.72 (m, 2H), 3.33-3.24 (m, 2H), 2.60-2.31 (m, 2H), 2.16-2.03 (m, 2H), 1.88-1.82 (m, 2H), 1.81-1.70 (m, 1H), 1.04 (s, 3H), 0.89 (s, 3H).

### Step 3: (1R,2S,5S)-3-(7-chloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-chloro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (80 mg, 169.15 mol) in DCM (3 mL) was added DMP (215 mg, 507.44 mol) at 25° C. The mixture was stirred at 25° C. for 1 hr. The residue was purified by prep-HPLC (column: Phenomenex Gemini-NX C18 75×30 mm×3 μm; mobile phase: [water (10 mM NH₄HCO₃)-ACN]; B %: 15%-55%, 11 min). Compound 5 (17 mg, 21.13% yield, 99% purity) was obtained as a white solid. ¹H NMR (400 MHz, CD₃OD) δ ppm 7.67-7.45 (m, 1H), 7.32-7.23 (m, 1H), 7.16-6.94 (m, 2H), 6.87-6.87 (m, 1H), 5.02-4.60 (m, 1H), 4.54-4.24 (m, 1H), 4.09-3.94 (m, 1H), 3.93-3.72 (m, 1H), 3.30-2.88 (m, 2H), 2.67-1.99 (m, 2H), 1.89-1.27 (m, 5H), 1.19-0.94 (m, 6H) ESI-MS [M+H]⁺ calc'd for C₂₄H₂₇ClN₄O₄, 471.17; found, 471.4.

### Example 6: (1R,2S,5S)-3-(7-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 6)

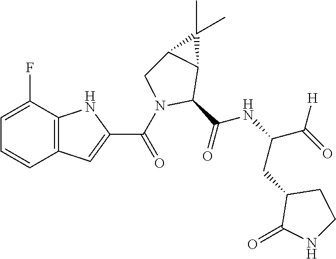 [SMILES: CC1(C)[C@@H]2[C@@H](C(=O)N[C@H](C=O)C[C@@H]3CCNC3=O)N(C(=O)c3cc4cccc(F)c4[nH]3)C[C@@H]21]

### Step 1: (S)-methyl 2-((1R,2S,5S)-3-(7-fluoro-1H-indole-2-carbonyl)-6,6-dimethyl-3-azabicyclo[3.1.0]hexane-2-carboxamido)-3-((S)-2-oxopyrrolidin-3-yl)propanoate

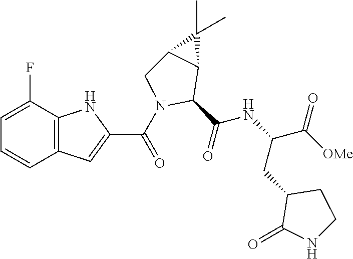 [SMILES: COC(=O)[C@H](C[C@@H]1CCNC1=O)NC(=O)[C@@H]1[C@@H]2[C@H](CN1C(=O)c1cc3cccc(F)c3[nH]1)C2(C)C]

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (250 mg, 694.74 μmol, HCl), 7-fluoro-1H-indole-2-carboxylic acid (124 mg, 692.17 mol) and DIEA (269.37 mg, 2.08 mmol) in DCM (5 mL) was added HATU (528.32 mg, 1.39 mmol) in one portion at 0° C. The mixture was stirred at 25° C. for 10 hours. The reaction mixture was poured into sat.aq.NH₄Cl (20 mL). The mixture was extracted with DCM (20 mL×2). The combined organic phase was washed with brine (10 mL×3), dried with anhydrous Na₂SO₄, filtered and concentrated in vacuum. The residue was purified by prep-HPLC (column: Xtimate C18 150×40 mm×10 m; mobile phase: [water (0.05% HCl)-ACN]; B %: 25%-55%, 10 min). The title compound was obtained as a white solid (160 mg, 47.44% yield). ¹H NMR (400 MHz, CD₃OD) δ ppm 7.49-7.33 (m, 1H), 7.15-6.90 (m, 3H), 5.00 (s, 1H), 4.57 (dd, J=4.0, 11.6 Hz, 1H), 4.39-4.21 (m, 1H), 4.06-3.89 (m, 1H), 3.80-3.64 (m, 3H), 3.35-3.32 (m, 1H), 3.06-2.64 (m, 1H), 2.61-2.29 (m, 1H), 2.25-1.94 (m, 1H), 1.92-1.75 (m, 2H), 1.74-1.36 (m, 3H), 1.16-0.94 (m, 6H).

### Step 2: (1R,2S,5S)-3-(7-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

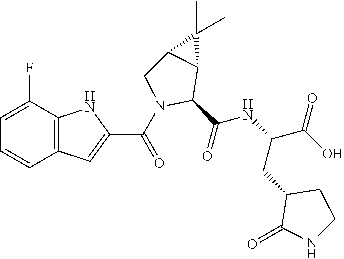 [SMILES: CC1(C)[C@@H]2[C@@H](C(=O)N[C@@H](C[C@@H]3CCNC3=O)C(=O)O)N(C(=O)c3cc4cccc(F)c4[nH]3)C[C@@H]21]

To a solution of (S)-methyl 2-((1R,2S,5S)-3-(7-fluoro-1H-indole-2-carbonyl)-6,6-dimethyl-3-azabicyclo[3.1.0]hexane-2-carboxamido)-3-((S)-2-oxopyrrolidin-3-yl)propanoate (160 mg, 330.22 mol) in THE (3 mL) was added LiBH₄ (21.6 mg, 990.67 mol) in one portion at 0° C. The mixture was stirred at 25° C. for 2 hr. The residue was poured into water (10 mL). The aqueous phase was extracted with DCM (5 mL×3). The combined organic phase was washed with brine (10 mL), dried with anhydrous Na₂SO₄, filtered and concentrated under vacuum. The residue was purified by prep-HPLC (column: Phenomenex Gemini-NX C18 75×30 mm×3 μm; mobile phase: [water (10 mM NH₄HCO₃)-ACN]; B %: 10%-50%, 11 min). The title compound was obtained as a white solid (60 mg, 37.41% yield).

¹H NMR (400 MHz, CD₃OD) δ ppm 7.49-7.32 (m, 1H), 7.14-6.91 (m, 3H), 4.95-4.90 (m, 1H), 4.35-4.27 (m, 1H), 4.08-3.99 (m, 1H), 3.95 (d, J=10.8 Hz, 1H), 3.83-3.72 (m, 1H), 3.56 (d, J=5.6 Hz, 1H), 3.49-3.41 (m, 1H), 3.30-3.25 (m, 1H), 3.00-2.59 (m, 1H), 2.46-2.31 (m, 1H), 2.03-1.93 (m, 1H), 1.86-1.66 (m, 3H), 1.60-1.44 (m, 2H), 1.15-0.97 (m, 6H).

### Step 3: (1R,2S,5S)-3-(7-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (55 mg, 120.48 mol) in DCM (3 mL) was added DMP (153.0 mg, 361.44 mol) in one portion at 25° C. The mixture was stirred at 25° C. for 1 hr. The reaction mixture was filtered. The cake was washed with DCM (2 mL). The combined organic phase was washed with sat.aq.NaHCO₃ (1 mL×2) and sat.aq.Na₂SO₃ (1 mL×2), dried with anhydrous Na₂SO₄, filtered and concentrated in vacuo. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 14%-54%, 11 min). Compound 6 was obtained as a white solid (4.2 mg, 7.62% yield, 99.38% purity). ¹H NMR (400 MHz, CD₃OD) δ ppm 7.50-7.31 (m, 1H), 7.15-6.88 (m, 3H), 5.00-4.91 (m, 1H), 4.54-4.37 (m, 1H), 4.34-3.89 (m, 2H), 3.81-3.66 (m, 1H), 3.29-3.19 (m, 1H), 2.98-2.56 (m, 1H), 2.41-2.28 (m, 1H), 2.10-1.80 (m, 1H), 1.74-1.52 (m, 3H), 1.45-1.32 (m, 1H), 1.17-0.95 (m, 6H). ESI-MS [M+H]⁺ calc'd for C₂₄H₂₇FN₄O₄, 455.20; found, 455.4.

### Example 7: (1R,2S,5S)-6,6-dimethyl-N—((S)-1-oxo-3-((S)-2-oxopyrrolidin-3-yl)propan-2-yl)-3-((R)-2-(m-tolyl)propanoyl)-3-azabicyclo[3.1.0]hexane-2-carboxamide (Compound 7)

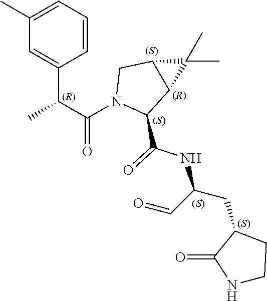

### Step 1: (S)-methyl 2-((1R,2S,5S)-6,6-dimethyl-3-((R)-2-(m-tolyl)propanoyl)-3-azabicyclo[3.1.0]hexane-2-carboxamido)-3-((S)-2-oxopyrrolidin-3-yl)propanoate

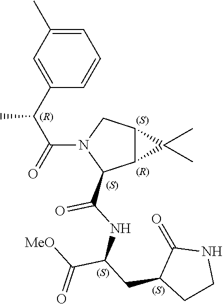

To a mixture of (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 788.53 mol) and (R)-2-(m-tolyl)propanoic acid (129 mg, 788.53 mol) in DCM (5 mL) was added DIEA (305 mg, 2.37 mmol) and HATU (599 mg, 1.58 mmol) at 0° C. The mixture was stirred at 25° C. for 12 hr. The mixture was diluted with sat.aq. NH₄Cl (8 mL), extracted with DCM (10 mL×3). The combined organic layer was dried over Na₂SO₄, filtered and concentrated to crude product. The crude product was purified by prep-HPLC (column: Welch Ultimate XB—CN 250×50×10 m; mobile phase: [Heptane-EtOH (0.1% NH₃H₂O)]; B %: 10%-30%, 8 min). The title compound (80 mg, 17.50% yield) was obtained as a white solid. ESI-MS [M+H]⁺ calc'd for C₂₆H₃₅N₃O₅, 470.26; found, 470.3.

### Step 2: (1R,2S,5S)—N—((S)-1-hydroxy-3-((S)-2-oxopyrrolidin-3-yl)propan-2-yl)-6,6-dimethyl-3-((R)-2-(m-tolyl)propanoyl)-3-azabicyclo[3.1.0]hexane-2-carboxamide

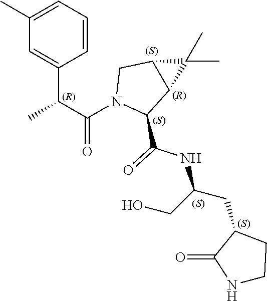

To a solution of (S)-methyl 2-((1R,2S,5S)-6,6-dimethyl-3-((R)-2-(m-tolyl)propanoyl)-3-azabicyclo[3.1.0]hexane-2-carboxamido)-3-((S)-2-oxopyrrolidin-3-yl)propanoate (80 mg, 170.37 mol) in THE (5 mL) was added LiBH₄ (11 mg, 511.10 mol) at 0° C. The mixture was stirred at 0° C. for 1 hr. The mixture was quenched with H₂O (10 mL), and extracted with DCM (5 mL×3). The combined organic layer was dried over anhydrous Na₂SO₄, filtered and concentrated to dryness. The crude product was used to next step directly. The title compound (80 mg, 63.81% yield) was obtained as a white solid. ESI-MS [M+H]⁺ calc'd for C₂₅H₃₅N₃O₄, 442.26; found, 442.3.

### Step 3: (1R,2S,5S)-6,6-dimethyl-N—((S)-1-oxo-3-((S)-2-oxopyrrolidin-3-yl)propan-2-yl)-3-((R)-2-(m-tolyl)propanoyl)-3-azabicyclo[3.1.0]hexane-2-carboxamide

A mixture of (1R,2S,5S)—N—((S)-1-hydroxy-3-((S)-2-oxopyrrolidin-3-yl)propan-2-yl)-6,6-dimethyl-3-((R)-2-(m-tolyl)propanoyl)-3-azabicyclo[3.1.0]hexane-2-carboxamide (80 mg, 181.18 mol) and DMP (230 mg, 543.53 mol) in DCM (2 mL) was degassed and purged with N₂ for 3 times, and then the mixture was stirred at 25° C. for 0.5 hr under N₂ atmosphere. The mixture was filtered and the filtrate was concentrated to give crude product. The residue was purified by prep-HPLC (column: Phenomenex C18 75×30 mm×3 m; mobile phase: [water (NH₄HCO₃)-ACN]; B %: 22%-50%, 8 min). Compound 7 (10.7 mg, 12.76% yield, 95% purity) was obtained as a white solid. ¹H NMR (400 MHz, CD₃OD) δ ppm 7.26-7.15 (m, 1H), 7.12-6.97 (m, 3H), 4.54-4.23 (m, 2H), 4.09-3.84 (m, 2H), 3.77 (q, J=6.6 Hz, 1H), 2.75-2.61 (m, 1H), 2.46-2.35 (m, 1H), 2.31 (s, 3H), 2.12-2.00 (m, 1H), 1.85-1.37 (m, 5H), 1.33 (dd, J=1.3, 6.9 Hz, 3H), 0.96 (s, 3H), 0.46 (s, 3H). ESI-MS [M+H]⁺ calc'd for C₂₅H₃₃N₃O₄, 440.25; found, 440.3.

### Example 8: (1R,2S,5S)-3-(1-(3-chlorophenyl)cyclopropanecarbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 8)

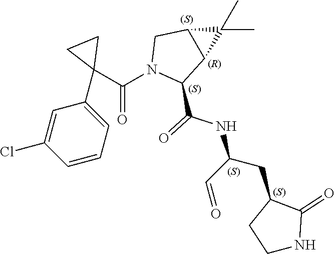

### Step 1: (S)-methyl 2-((1R,2S,5S)-3-(1-(3-chlorophenyl)cyclopropanecarbonyl)-6,6-dimethyl-3-azabicyclo[3.1.0]hexane-2-carboxamido)-3-((S)-2-oxopyrrolidin-3-yl)propanoate

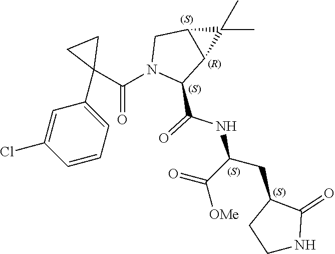

To a solution of 1-(3-chlorophenyl)cyclopropanecarboxylic acid (139 mg, 708.13 mol) in DCM (3 mL) was added (S)-methyl 2-((1R,2S,5S)-6,6-dimethyl-3-azabicyclo[3.1.0]hexane-2-carboxamido)-3-((S)-2-oxopyrrolidin-3-yl)propanoate (229 mg, 708.13 mol), DIEA (389 mg, 3.02 mmol, 525 μL) and HATU (269 mg, 708.13 mol). The mixture was stirred at 20° C. for 5 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: C18-6 100×30 mm×5 μm; mobile phase: [water (FA)-ACN]; B %: 42%-72%, 8 min). The title compound (71 mg, 19.97% yield) was obtained as yellow oil. ¹H NMR (400 MHz, CDCl₃) δ ppm 7.43-7.34 (m, 1H), 7.24-7.21 (m, 1H), 7.19-7.02 (m, 3H), 5.98-5.76 (m, 1H), 4.71-4.55 (m, 1H), 4.34 (s, 1H), 3.76 (s, 3H), 3.46-3.30 (m, 4H), 2.62-2.39 (m, 2H), 2.21-2.13 (m, 1H), 1.61-1.52 (m, 1H), 1.49-1.44 (m, 1H), 1.38-1.31 (m, 1H), 1.25-1.18 (m, 2H), 1.11-1.04 (m, 1H), 0.99 (s, 3H), 0.78 (s, 3H).

### Step 2: (1R,2S,5S)-3-(1-(3-chlorophenyl) cyclopropanecarbonyl)-N—((S)-1-hydroxy-3-((S)-2-oxopyrrolidin-3-yl)propan-2-yl)-6,6-dimethyl-3-azabicyclo[3.1.0]hexane-2-carboxamide

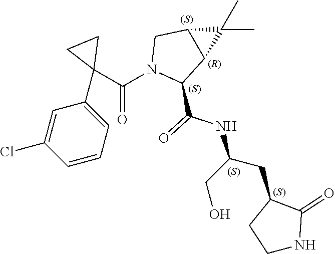

To a solution of (S)-methyl 2-((1R,2S,5S)-3-(1-(3-chlorophenyl)cyclopropanecarbonyl)-6,6-dimethyl-3-azabicyclo[3.1.0]hexane-2-carboxamido)-3-((S)-2-oxopyrrolidin-3-yl)propanoate (60 mg, 119.52 mol) in THE (4 mL) was added LiBH₄ (7 mg, 321.40 mol) dropwise at 0° C. The mixture was stirred at 0° C. for 1 hr. H₂O (10 mL) was added to the reaction mixture. The resulting mixture was extracted with DCM (5 mL×3). The combined organic phase was washed with brine (5 mL) and water (5 mL), dried over anhydrous Na₂SO₄, filtered and concentrated to dryness. The title compound (60 mg, 83.47% yield) was obtained as a white solid. ¹H NMR (400 MHz, CDCl₃) δ ppm 7.68-7.35 (m, 1H), 7.26-7.10 (m, 4H), 5.91 (brs, 1H), 4.40-4.28 (m, 1H), 4.10-4.00 (m, 1H), 3.83-3.67 (m, 2H), 3.62-3.50 (m, 2H), 3.40-3.28 (m, 2H), 2.62-2.40 (m, 2H), 2.06 (m, 1H), 1.93-1.82 (m, 2H), 1.71-1.53 (m, 2H), 1.38-1.31 (m, 1H), 1.30-1.25 (m, 1H), 1.22-1.09 (m, 2H), 1.05-0.67 (m, 6H).

### Step 3: (1R,2S,5S)-3-(1-(3-chlorophenyl)cyclopropanecarbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(1-(3-chlorophenyl)cyclopropanecarbonyl)-N—((S)-1-hydroxy-3-((S)-2-oxopyrrolidin-3-yl)propan-2-yl)-6,6-dimethyl-3-azabicyclo[3.1.0]hexane-2-carboxamide (55 mg, 116.04 mol) in DCM (6 mL) was added DMP (148 mg, 348.11 mol) at 25° C. The reaction mixture was stirred at 25° C. for 1 hr. The reaction mixture was filtered. The filtrate was concentrated. The residue was purified by prep-HPLC (column: Phenomenex C18 75×30 mm×3 μm; mobile phase: [water (NH₄HCO₃)-ACN]; B %: 25%-55%, 10 min). Compound 8 was obtained as a white solid (3.2 mg, 5.54% yield, 94.796% purity). ¹H NMR (400 MHz, CD₃OD) δ ppm 7.37-7.13 (m, 4H), 4.52-4.29 (m, 2H), 4.14-3.92 (m, 1H), 3.67-3.33 (m, 3H), 2.72-2.57 (m, 1H), 2.48-2.32 (m, 1H), 2.11-1.94 (m, 1H), 1.83-1.69 (m, 1H), 1.65-1.53 (m, 1H), 1.50-1.36 (m, 3H), 1.32-1.21 (m, 2H), 1.20-1.13 (m, 1H), 1.04-0.73 (m, 6H). ESI-MS [M+H]⁺ calc'd for C₂₅H₃₀C₁N₃O₄, 472.19; found, 472.4.

### Example 9: (S)-1-(7-chloro-1H-indole-2-carbonyl)-4,4-dimethyl-N—((S)-1-oxo-3—((S)-2-oxopyrrolidin-3-yl)propan-2-yl)pyrrolidine-2-carboxamide (Compound 9)

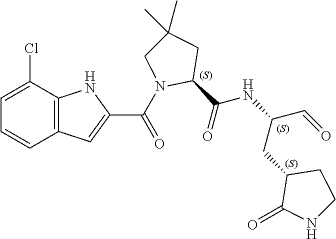

### Step 4: (S)-tert-butyl 2-(((S)-1-methoxy-1-oxo-3-((S)-2-oxopyrrolidin-3-yl)propan-2-yl)carbamoyl)-4,4-dimethylpyrrolidine-1-carboxylate

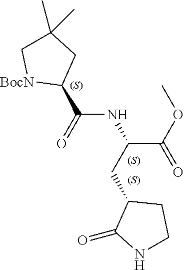

To a solution of (S)-1-(tert-butoxycarbonyl)-4,4-dimethylpyrrolidine-2-carboxylic acid (500 mg, 2.06 mmol) and DIEA (1.06 g, 8.22 mmol) in DCM (10 mL) was added dropwise T₃P (1.96 g, 3.08 mmol, 50% purity in EtOAc) at 0° C. After addition, the mixture was stirred at the same temperature for 30 min, and then (S)-methyl 2-amino-3-((S)-2-oxopyrrolidin-3-yl)propanoate hydrochloride (458 mg, 2.06 mmol, HCl) was added at 25° C. The resulting mixture was stirred at 25° C. for 10 hr. The reaction mixture was diluted with DCM (30 mL) and 5% H₃PO₄ (30 mL). The layers were separated and the aqueous phase was extracted with DCM (50 mL). The combined organic layer was washed with 5% H₃PO₄ (30 mL), sat.aq.NaHCO₃ (30 mL×2) and water (30 mL), dried over anhydrous Na₂SO₄, filtered and concentrated. The title compound (760 mg, crude) was obtained as a yellow solid. ¹H NMR (400 MHz, CDCl₃) δ ppm 7.73-7.29 (m, 1H), 6.23-5.45 (m, 1H), 4.70-4.41 (m, 1H), 4.26 (t, J=8.0 Hz, 1H), 3.80-3.63 (m, 3H), 3.53-3.22 (m, 3H), 3.18-3.02 (m, 1H), 2.59-2.27 (m, 2H), 2.25-2.12 (m, 1H), 2.01-1.65 (m, 4H), 1.44 (s, 9H), 1.17-0.91 (m, 6H).

### Step 5: (S)-methyl 2-((S)-4,4-dimethylpyrrolidine-2-carboxamido)-3-((S)-2-oxopyrrolidin-3-yl)propanoate

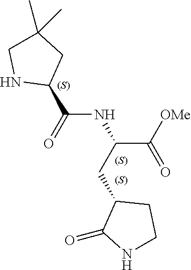

To a mixture of (S)-tert-butyl 2-(((S)-1-methoxy-1-oxo-3-((S)-2-oxopyrrolidin-3-yl)propan-2-yl)carbamoyl)-4,4-dimethylpyrrolidine-1-carboxylate (200 mg, 486.04 mol) in dioxane (2 mL) was added HCl/dioxane (4 M, 2 mL). The mixture was stirred at 25° C. for 1 hr. The reaction mixture was concentrated to dryness directly. The title compound (168 mg, crude, HCl) was obtained as a white solid, which was used to next step without further purification.

### Step 6: (S)-methyl 2-((S)-1-(7-chloro-1H-indole-2-carbonyl)-4,4-dimethylpyrrolidine-2-carboxamido)-3-((S)-2-oxopyrrolidin-3-yl)propanoate

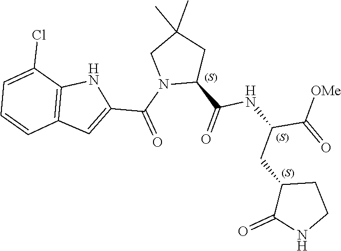

To a solution of (S)-methyl 2-((S)-4,4-dimethylpyrrolidine-2-carboxamido)-3-((S)-2-oxopyrrolidin-3-yl)propanoate (165 mg, 474.36 mol HCl), 7-chloro-1H-indole-2-carboxylic acid (93 mg, 474.36 mol) and DIEA (184 mg, 1.42 mmol) in DCM (3 mL) was added HATU (361 mg, 948.72 mol) slowly at 0° C. The mixture was stirred at 25° C. for 10 hr. Sat.aq.NH₄Cl (20 mL) was added. The resulting mixture was extracted with DCM (30 mL×3). The combined organic phase was washed with water (30 mL), dried over anhydrous Na₂SO₄, filtered and concentrated to dryness. The residue was purified by prep-HPLC (column: Xtimate C18 150×40 mm×10 μm; mobile phase: [water (10 mM NH₄HCO₃)-ACN]; B %: 25%-55%, 10 min). The title compound (80 mg, 33.74% yield) was obtained as a white solid. ¹H NMR (400 MHz, CDCl₃) δ ppm 9.65-9.39 (m, 1H), 8.69-8.00 (m, 1H), 7.62-7.42 (m, 1H), 7.31-7.26 (m, 1H), 7.06 (t, J=8.0 Hz, 1H), 6.97-6.89 (m, 1H), 6.14-5.67 (m, 1H), 4.98-4.69 (m, 1H), 4.61-4.14 (m, 1H), 3.82-3.75 (m, 1H), 3.73-3.65 (m, 4H), 3.55-3.27 (m, 2H), 2.67-2.54 (m, 1H), 2.49-2.37 (m, 1H), 2.23-1.99 (m, 3H), 1.98-1.71 (m, 2H), 1.30-0.96 (m, 6H).

### Step 7: (S)-1-(7-chloro-1H-indole-2-carbonyl)-N—((S)-1-hydroxy-3-((S)-2-oxopyrrolidin-3-yl)propan-2-yl)-4,4-dimethylpyrrolidine-2-carboxamide

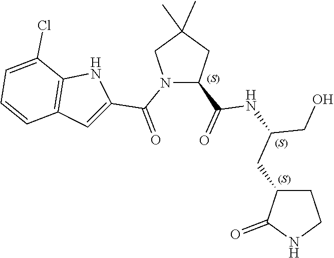

To a solution of (S)-methyl 2-((S)-1-(7-chloro-1H-indole-2-carbonyl)-4,4-dimethylpyrrolidine-2-carboxamido)-3-((S)-2-oxopyrrolidin-3-yl)propanoate (75 mg, 153.39 mol) in THE (2 mL) was added LiBH₄ (13 mg, 613.54 mol) at 0° C. The mixture was stirred at 25° C. for 1 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The title compound (70 mg, crude) was obtained as a white solid. ¹H NMR (400 MHz, CDCl₃) δ ppm 9.96-9.58 (m, 1H), 9.02-8.10 (m, 1H), 7.63-7.39 (m, 1H), 7.32-7.26 (m, 1H), 7.18-6.92 (m, 2H), 6.44-5.72 (m, 1H), 4.92-4.65 (m, 1H), 3.83-3.71 (m, 3H), 3.70-3.63 (m, 1H), 3.58-3.47 (m, 1H), 3.39-3.16 (m, 2H), 2.74-2.52 (m, 1H), 2.49-2.32 (m, 1H), 2.15-1.88 (m, 3H), 1.87-1.72 (m, 2H), 1.67-1.58 (m, 1H), 1.30-0.94 (m, 6H).

### Step 8: (S)-1-(7-chloro-1H-indole-2-carbonyl)-4,4-dimethyl-N—((S)-1-oxo-3-((S)-2-oxopyrrolidin-3-yl)propan-2-yl)pyrrolidine-2-carboxamide

To a solution of (S)-1-(7-chloro-1H-indole-2-carbonyl)-N—((S)-1-hydroxy-3-((S)-2-oxopyrrolidin-3-yl)propan-2-yl)-4,4-dimethylpyrrolidine-2-carboxamide (50 mg, 108.47 μmol,) in DCM (2 mL) was added DMP (92 mg, 216.94 mol). The mixture was stirred at 25° C. for 10 min. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 17%-57%, 11 min). Compound 9 (5 mg, 9.94% yield, 99.01% purity) was obtained as a whilte solid. ¹H NMR (400 MHz, CD₃OD) δ ppm 7.68-7.44 (m, 1H), 7.34-7.21 (m, 1H), 7.15-6.86 (m, 2H), 4.76-4.66 (m, 1H), 4.61-4.26 (m, 1H), 4.07-3.91 (m, 1H), 3.81-3.68 (m, 1H), 3.67-3.42 (m, 1H), 2.97-2.61 (m, 1H), 2.49-1.96 (m, 3H), 1.95-1.69 (m, 2H), 1.68-1.52 (m, 1H), 1.50-1.31 (m, 1H), 1.31-1.00 (m, 6H). ESI-MS [M+H]⁺ calc'd for C₂₃H₂₇ClN₄O₄, 459.17; found, 459.3.

### Example 10: (S)-6-(1H-indole-2-carbonyl)-N—((S)-1-oxo-3-((S)-2-oxopyrrolidin-3-yl)propan-2-yl)-6-azaspiro[3.4]octane-7-carboxamide (Compound 10)

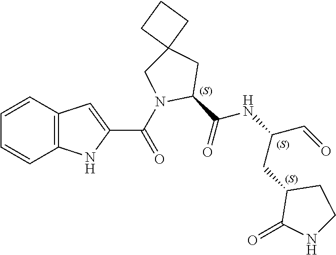

### Step 1: (S)-tert-butyl-7-(((S)-1-methoxy-1-oxo-3-((S)-2-oxopyrrolidin-3-yl)propan-2-yl)carbamoyl)-6-azaspiro[3.4]octane-6-carboxylate

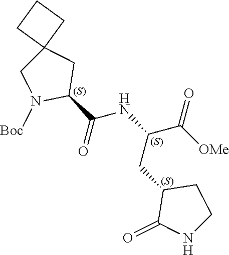

To a solution of (S)-6-(tert-butoxycarbonyl)-6-azaspiro[3.4]octane-7-carboxylic acid (450 mg, 1.76 mmol) in DCM (7 mL) and DIEA (682 mg, 5.28 mmol) was added dropwise T₃P (1.68 g, 2.64 mmol, 50% purity in EtOAc) at 0° C. After addition, the mixture was stirred at the same temperature for 30 min, and then (S)-methyl 2-amino-3-((S)-2-oxopyrrolidin-3-yl)propanoate (391 mg, 1.76 mmol, HCl) in DCM (5 mL) was added at 25° C. The resulting mixture was stirred at 25° C. for 11.5 hrs. The reaction mixture was diluted with DCM (30 mL). The combined organic layers were washed with 5% H₃PO₄ (10 mL), sat.aq. NaHCO₃ (10 mL), dried over anhydrous Na₂SO₄, filtered and concentrated to dryness. The title compound (740 mg, 46.66% yield) was obtained as a yellow solid which was used in the next step without further purification. ESI-MS [M+H]⁺ calc'd for C₂₁H₃₃N₃O₆, 424.24, found 424.2.

### Step 2: (S)-methyl-3-((S)-2-oxopyrrolidin-3-yl)-2-((S)-6-azaspiro[3.4]octane-7-carboxamido)propanoate

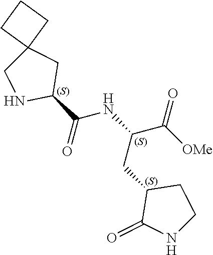

A mixture of (S)-tert-butyl 7-(((S)-1-methoxy-1-oxo-3-(2-oxoimidazolidin-1-yl)propan-2-yl)carbamoyl)-6-azaspiro[3.4]octane-6-carboxylate (700 mg, 1.65 mmol) in HCl/dioxane (4M, 10 mL) was degassed and purged with N₂ for 3 times, and then the mixture was stirred at 25° C. for 1 hr under N₂ atmosphere. The mixture was concentrated to give crude product. The title compound (590 mg, crude, HCl) was obtained as a white solid, which was used to next step directly without further purification.

### Step 3: (S)-methyl-2-((S)-6-(1H-indole-2-carbonyl)-6-azaspiro[3.4]octane-7-carboxamido)-3-((S)-2-oxopyrrolidin-3-yl)propanoate

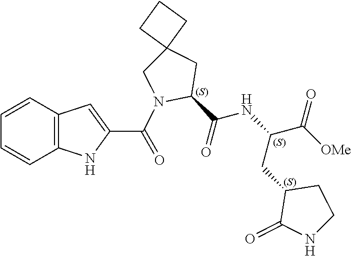

To a mixture of (S)-methyl 3-((S)-2-oxopyrrolidin-3-yl)-2-((S)-6-azaspiro[3.4]octane-7-carboxamido)propanoate (590 mg, 1.64 mmol, HCl) and 1H-indole-2-carboxylic acid (264 mg, 1.64 mmol) in DCM (10 mL) were added DIEA (635 mg, 4.92 mmol) and HATU (1.25 g, 3.28 mmol) at 0° C. The mixture was stirred at 25° C. for 2 hr. The mixture was diluted with sat.aq. NH₄Cl (20 mL), and extracted with DCM (20 mL×3). The combined organic layers were dried over Na₂SO₄, filtered and concentrated to crude product. The residue was purified by column chromatography (SiO₂, DCM/MeOH=5% to 10%). The title compound (336 mg, 40.41% yield) was obtained as a yellow solid. ¹H NMR (400 MHz, CD₃OD) δ ppm 7.71-7.43 (m, 2H), 7.29-7.20 (m, 1H), 7.14-6.80 (m, 2H), 4.70-4.52 (m, 2H), 4.12-3.98 (m, 1H), 3.78-3.69 (m, 3H), 3.00-2.71 (m, 1H), 2.65-2.33 (m, 3H), 2.26-1.92 (m, 10H), 1.91-1.75 (m, 2H).

### Step 4: (S)—N—((S)-1-hydroxy-3-((S)-2-oxopyrrolidin-3-yl)propan-2-yl)-6-(1H-indole-2-carbonyl)-6-azaspiro[3.4]octane-7-carboxamide

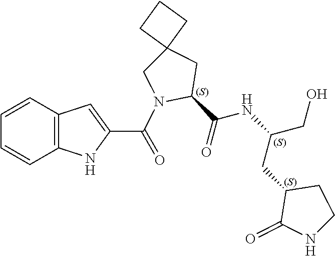

A mixture of (S)-methyl 2-((S)-6-(1H-indole-2-carbonyl)-6-azaspiro[3.4]octane-7-carboxamido)-3-((S)-2-oxopyrrolidin-3-yl)propanoate (120 mg, 257.22 mol) and LiBH₄ (16 mg, 771.66 mol) in THE (3 mL) was degassed and purged with N₂ for 3 times at 0° C., and then the mixture was stirred at 0° C. for 1 hr under N₂ atmosphere. The reaction was quenched by water (20 mL). The reaction mixture was extracted with DCM (20 mL×3). The combined organic phase was dried over anhydrous Na₂SO₄ and concentrated to dryness. The title compound (67 mg, 59.40% yield) was obtained as a yellow solid. ¹H NMR (400 MHz, CD₃OD) δ ppm 7.77-6.68 (m, 5H), 4.19-3.96 (m, 1H), 3.95-3.85 (m, 1H), 3.83-3.51 (m, 2H), 3.49-3.25 (m, 3H), 2.91-2.56 (m, 1H), 2.52-2.15 (m, 2H), 2.13-1.74 (m, 8H), 1.72-1.34 (m, 4H).

### Step 5: (S)-6-(1H-indole-2-carbonyl)-N—((S)-1-oxo-3-((S)-2-oxopyrrolidin-3-yl)propan-2-yl)-6-azaspiro[3.4]octane-7-carboxamide

To a solution of (S)—N—((S)-1-hydroxy-3-((S)-2-oxopyrrolidin-3-yl)propan-2-yl)-6-(1H-indole-2-carbonyl)-6-azaspiro[3.4]octane-7-carboxamide (60 mg, 136.82 mol) in DCM (2 mL) was added DMP (174 mg, 410.47 mol) at 25° C. The mixture was stirred at 25° C. for 0.5 hr. The reaction mixture was filtered. And the filtrate was concentrated directly to give crude product. The residue was purified by prep-HPLC (column: Phenomenex Gemini-NX C18 75×30 mm×3 μm; mobile phase: [water (10 mM NH₄HCO₃)-ACN]; B %: 15%-45%, 10 min). Compound 10 (5 mg, 8.07% yield, 96.43% purity) was obtained as a white solid. ¹H NMR (400 MHz, CD₃OD) δ ppm 7.59-7.37 (m, 1H), 7.35-7.28 (m, 1H), 7.16-7.06 (m, 1H), 7.01-6.69 (m, 2H), 4.57-4.21 (m, 2H), 4.01-3.80 (m, 2H), 3.72-3.55 (m, 1H), 2.81-2.22 (m, 3H), 2.11-1.82 (m, 8H), 1.62-1.45 (m, 1H), 1.35-1.30 (m, 1H), 1.25-1.16 (m, 1H). ESI-MS [M+H]⁺ calc'd for C₂₄H₂₈N₄O₄, 437.21, found 437.4.

### Example 11: (1R,2S,5S)-3-(1H-indole-2-carbonyl)-6,6-dimethyl-N—((S)-1-oxo-3-((S)-2-oxopiperidin-3-yl)propan-2-yl)-3-azabicyclo[3.1.0]hexane-2-carboxamide (Compound 11)

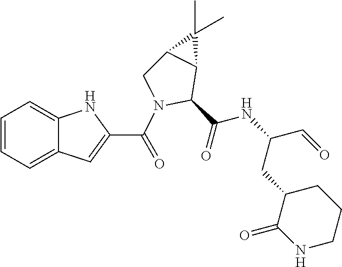 [SMILES: CC1(C)[C@@H]2[C@@H](C(=O)N[C@H](C=O)C[C@@H]3CCCNC3=O)N(C(=O)c3cc4ccccc4[nH]3)C[C@@H]21]

### Step 1: (1R,2S,5)-tert-butyl 2-(((S)-1-methoxy-1-oxo-3-((S)-2-oxopiperidin-3-yl)propan-2-yl)carbamoyl)-6,6-dimethyl-3-azabicyclo[3.1.0]hexane-3-carboxylate

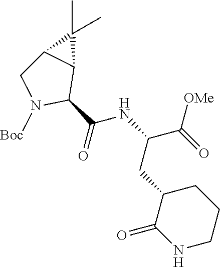 [SMILES: COC(=O)[C@H](C[C@@H]1CCCNC1=O)NC(=O)[C@@H]1[C@@H]2[C@H](CN1C(=O)OC(C)(C)C)C2(C)C]

To a solution of (1R,2S,5S)-3-(tert-butoxycarbonyl)-6,6-dimethyl-3-azabicyclo[3.1.0]hexane-2-carboxylic acid (300 mg, 1.18 mmol) and DIEA (607 mg, 4.70 mmol) in DCM (5 mL) was added dropwise T₃P (1.12 g, 1.76 mmol, 50% purity in EtOAc) at 0° C. After addition, the mixture was stirred at the same temperature for 30 min, and then (S)-methyl 2-amino-3-((S)-2-oxopiperidin-3-yl)propanoate (278 mg, 1.18 mmol, HCl) in DCM (5 mL) was added at 25° C. The resulting mixture was stirred at 25° C. for 10 hrs. The reaction mixture was diluted with H₂O (10 mL). The reaction mixture was extracted with DCM (10 mL×3). The combined organic phase was washed with 5% H₃PO₄ (10 mL×2), sat.aq.NaHCO₃ (10 mL×2) and brine (10 mL), dried over anhydrous Na₂SO₄, filtered and concentrated to dryness. The title compound (416 mg) was obtained as a white solid. ESI-MS [M+H]⁺ calc'd for C₂₂H₃₅N₃O₆, 438.26, found 438.3.

### Step 2: (S)-methyl 2-((1R,2S,5S)-6,6-dimethyl-3-azabicyclo[3.1.0]hexane-2-carboxamido)-3-((S)-2-oxopiperidin-3-yl)propanoate hydrochloride

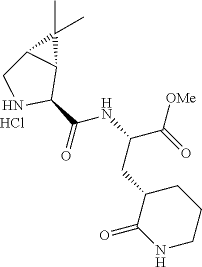 [SMILES: COC(=O)[C@H](C[C@@H]1CCCNC1=O)NC(=O)[C@H]1NC[C@H]2[C@@H]1C2(C)C ; Cl]

A mixture of (1R,2S,5S)-tert-butyl 2-(((S)-1-methoxy-1-oxo-3-((S)-2-oxopiperidin-3-yl)propan-2-yl)carbamoyl)-6,6-dimethyl-3-azabicyclo[3.1.0]hexane-3-carboxylate (416 mg, 950.80 mol) in HCl/dioxane (4 M, 10 mL) was degassed and purged with N₂ for 3 times, and then the mixture was stirred at 25° C. for 1 hr under N₂ atmosphere. The mixture was concentrated to give crude product. The title compound (355 mg, crude, HCl) was obtained as a white solid, which was used to next step directly.

### Step 3: (S)-methyl 2-((1R,2S,5S)-3-(1H-indole-2-carbonyl)-6,6-dimethyl-3-azabicyclo[3.1.0]hexane-2-carboxamido)-3-((S)-2-oxopiperidin-3-yl)propanoate

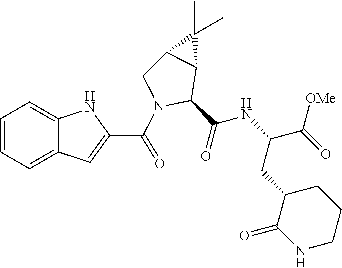 [SMILES: COC(=O)[C@H](C[C@@H]1CCCNC1=O)NC(=O)[C@@H]1[C@@H]2[C@H](CN1C(=O)c1cc3ccccc3[nH]1)C2(C)C]

To a solution of (S)-methyl 2-((1R,2S,5S)-6,6-dimethyl-3-azabicyclo[3.1.0]hexane-2-carboxamido)-3-((S)-2-oxopiperidin-3-yl)propanoate hydrochloride (355 mg, 949.52 μmol, HCl) in DCM (5 mL) was added 1H-indole-2-carboxylic acid (153 mg, 949.52 mol) and DIEA (490 mg, 3.80 mmol). And then HATU (469 mg, 1.23 mmol) was added slowly at 0° C. The resulting mixture was stirred at 25° C. for 10 h. Water (15 mL) was added to the reaction mixture. The mixture was extracted with DCM (10 mL×3). The combined organic phase was washed with sat.aq.NH₄Cl (10 mL×2), dried over anhydrous Na₂SO₄, filtered and concentrated to dryness. The residue was purified by prep-HPLC (column: Phenomenex Gemini-NX C18 75×30 mm×3 μm; mobile phase: [water (10 mM NH₄HCO₃)-ACN]; B %: 23%-63%, 11 min). The title compound (233 mg) was obtained as a white solid. ESI-MS [M+H]⁺ calc'd for C₂₆H₃₂N₄O₅, 481.24, found 481.3.

### Step 4: (1R,2S,5S)—N—((S)-1-hydroxy-3-((S)-2-oxopiperidin-3-yl)propan-2-yl)-3-(1H-indole-2-carbonyl)-6,6-dimethyl-3-azabicyclo[3.1.0]hexane-2-carboxamide

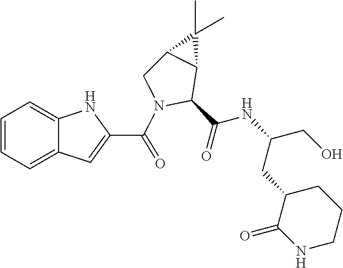 [SMILES: CC1(C)[C@@H]2[C@@H](C(=O)N[C@H](CO)C[C@@H]3CCCNC3=O)N(C(=O)c3cc4ccccc4[nH]3)C[C@@H]21]

To a mixture of (S)-methyl 2-((1R,2S,5S)-3-(1H-indole-2-carbonyl)-6,6-dimethyl-3-azabicyclo[3.1.0]hexane-2-carboxamido)-3-((S)-2-oxopiperidin-3-yl)propanoate (233 mg, 484.86 mol) in THE (5 mL) was added LiBH₄ (31 mg, 1.45 mmol) in one portion at 0° C. under N₂. The mixture was stirred at 25° C. for 1 h. Water (10 mL) was added to the mixture. The aqueous layer was extracted with DCM (10 mL×2). The combined organic layers were washed with brine (10 mL), dried over anhydrous Na₂SO₄, filtered and concentrated under reduced pressure. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 16%-56%, 11 min). The title compound (118 mg, 261.91 mol) was obtained as a white solid. ¹H NMR (400 MHz, CDCl₃) δ ppm 9.56 (s, 1H), 7.88 (d, J=7.2 Hz, 1H), 7.75-7.60 (m, 1H), 7.47-7.27 (m, 2H), 7.19-7.05 (m, 1H), 6.99-6.84 (m, 1H), 5.91 (s, 1H), 4.72-4.64 (m, 1H), 4.27 (dd, J=2.8, 9.6 Hz, 1H), 4.07-3.91 (m, 2H), 3.75 (dd, J=3.6, 11.6 Hz, 1H), 3.59-3.42 (m, 1H), 3.31-3.19 (m, 2H), 2.48-2.33 (m, 1H), 2.24-1.95 (m, 3H), 1.83-1.74 (m, 3H), 1.71-1.67 (m, 1H), 1.58-1.41 (m, 2H), 1.12-1.04 (m, 3H), 0.95-0.87 (m, 3H).

### Step 5: (1R,2S,5S)-3-(1H-indole-2-carbonyl)-6,6-dimethyl-N—((S)-1-oxo-3-((S)-2-oxopiperidin-3-yl)propan-2-yl)-3-azabicyclo[3.1.0]hexane-2-carboxamide

To a solution of (1R,2S,5S)—N—((S)-1-hydroxy-3-((S)-2-oxopiperidin-3-yl)propan-2-yl)-3-(1H-indole-2-carbonyl)-6,6-dimethyl-3-azabicyclo[3.1.0]hexane-2-carboxamide (60 mg, 132.58 μmol.) in DCM (6 mL) was added DMP (112 mg, 265.17 μmol, 82.09 uL.) at 25° C. The mixture was stirred at 25° C. for 1 hr. The residue was purified by prep-HPLC (column: Phenomenex Gemini-NX C18 75×30 mm×3 μm; mobile phase: [water (10 mM NH₄HCO₃)-ACN]; B %: 20%-50%, 10 min). Compound 11 was obtained as a brown solid (9.5 mg, 15.26% yield, 95.972% purity). ¹H NMR (400 MHz, CD₃OD) δ ppm 7.70-7.51 (m, 1H), 7.48-7.37 (m, 1H), 7.22 (q, J=7.2 Hz, 1H), 7.11-6.89 (m, 2H), 5.04-4.87 (m, 1H), 4.63-4.31 (m, 2H), 4.04-3.96 (m, 1H), 3.88-3.75 (m, 1H), 3.05-2.73 (m, 1H), 2.42-2.00 (m, 2H), 1.83-1.66 (m, 3H), 1.63-1.47 (m, 2H), 1.35-1.23 (m, 1H), 1.15-0.96 (m, 7H). ESI-MS [M+H]⁺ calc'd for C₂₅H₃₀N₄O₄, 451.23, found 451.4.

### Example 12: (1R,2S,5S)-3-((S)-3,3-dimethyl-2-(2,2,2-trifluoroacetamido) butanoyl)-6,6-dimethyl-N—((S)-1-oxo-3-((S)-2-oxopyrrolidin-3-yl)propan-2-yl)-3-azabicyclo[3.1.0]hexane-2-carboxamide (Compound 12)

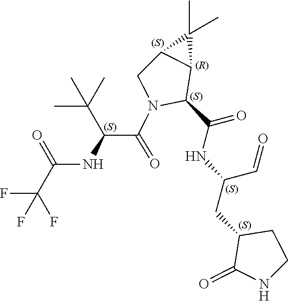

Compound 12 was synthesized by the same procedure as Compound 1. ¹H NMR (400 MHz, CD₃OD) δ ppm 4.53-4.41 (m, 1H), 4.38-4.26 (m, 1H), 4.08-3.72 (m, 3H), 3.29-3.12 (m, 2H), 2.73-2.49 (m, 1H), 2.43-2.24 (m, 1H), 2.11-1.90 (m, 1H), 1.83-1.67 (m, 1H), 1.65-1.37 (m, 3H), 1.16-0.85 (m, 15H). ESI-MS [M+H]⁺ calc'd for C₂₃H₃₃F₃N₄O₅, 503.53; found, 503.5.

### Example 13: (1R,2S,5S)-3-(4,7-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 13)

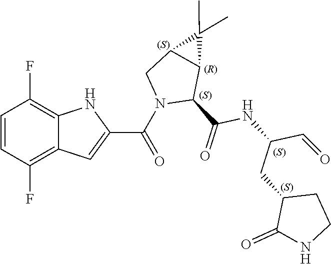

Step 1: ethyl 4,7-difluoro-1H-indole-2-carboxylate

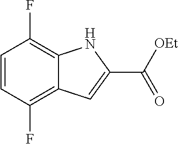 [SMILES: CCOC(=O)c1cc2c(F)ccc(F)c2[nH]1]

To a solution of (2,5-difluorophenyl)hydrazine (1.0 g, 5.54 mmol, HCl) in toluene (20 mL) was added 4-methylbenzenesulfonic acid (3.4 g, 19.74 mmol) and ethyl 2-oxopropanoate (643 mg, 5.54 mmol, 612.38 μL) at 25° C. The mixture was stirred at 120° C. for 12 hr. Water (30 mL) was added and the reaction mixture was adjusted to pH~7 by sat.aq.NaHCO₃. The resulting mixture was extracted with EtOAc (50 mL×3). The combined organic phase was washed with water (50 mL), dried over anhydrous Na₂SO₄, filtered and concentrated to dryness. The residue was purified by flash silica gel chromatography (Eluent of 0-5% EtOAc in PE). The title compound (144 mg, 11.55% yield) was obtained as a yellow solid. ¹H NMR (400 MHz, CDCl₃) δ ppm 9.12 (s, 1H), 7.30 (t, J=2.4 Hz, 1H), 6.99-6.85 (m, 1H), 6.70 (dt, J=3.2, 8.8 Hz, 1H), 4.44 (q, J=7.2 Hz, 2H), 1.43 (t, J=7.2 Hz, 3H).

### Step 2: 4, 7-difluoro-1H-indole-2-carboxylic acid

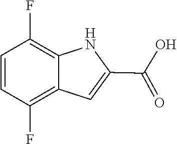 [SMILES: O=C(O)c1cc2c(F)ccc(F)c2[nH]1]

To a solution of ethyl 4,7-difluoro-1H-indole-2-carboxylate (120 mg, 532.88 mol) in MeOH (1 mL) and H₂O (1 mL) was added LiOH·H₂O (45 mg, 1.07 mmol). And then the mixture was stirred at 50° C. for 1 hr. Water (10 mL) was added and the reaction mixture was adjusted to pH~5 by aq. HCl (1 N). The resulting mixture was extracted with DCM (10 mL×3). The combined organic layers were washed with water (10 mL), dried over anhydrous Na₂SO₄, filtered and concentrated to dryness. The title compound (75 mg, crude) was obtained as a yellow solid. ¹H NMR (400 MHz, CD₃OD) δ ppm 7.18 (d, J=3.2 Hz, 1H), 6.98-6.85 (m, 1H), 6.68 (td, J=3.2, 9.6 Hz, 1H).

### Step 3: (S)-methyl 2-((1R,2S,5S)-3-(4,7-difluoro-1H-indole-2-carbonyl)-6,6-dimethyl-3-azabicyclo[3.1.0]hexane-2-carboxamido)-3-((S)-2-oxopyrrolidin-3-yl)propanoate

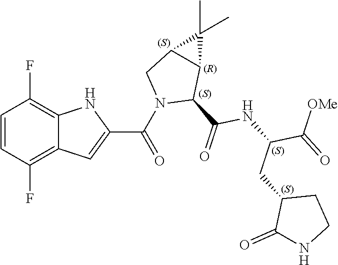

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (91 mg, 253.63 μmol, HCl), 4,7-difluoro-1H-indole-2-carboxylic acid (50 mg, 253.63 μmol,) and DIEA (98 mg, 760.89 mol) in DCM (3 mL) was added slowly HATU (193 mg, 507.26 mol) at 0° C. The resulting mixture was stirred at 25° C. for 10 hr. The reaction mixture was concentrated. Sat.aq.NH₄Cl (10 mL) was added to the residue. The resulting mixture was extracted with DCM (20 mL×3). The combined organic phase was washed with water (10 mL), dried over anhydrous Na₂SO₄, filtered and concentrated to dryness. The residue was purified by flash silica gel chromatography (Eluent of 0~94% EtOAc in PE). The title compound (150 mg, 92.60% yield) was obtained as a white solid. ¹H NMR (400 MHz, CDCl₃) δ ppm 10.46-9.79 (m, 1H), 9.00-7.93 (m, 1H), 7.03-6.91 (m, 1H), 6.90-6.79 (m, 1H), 6.70-6.56 (m, 1H), 6.31-6.11 (m, 1H), 4.77-4.64 (m, 1H), 4.34-4.22 (m, 1H), 4.04-3.86 (m, 1H), 3.72 (s, 3H), 3.62-3.46 (m, 1H), 3.42-3.27 (m, 2H), 2.69-2.52 (m, 1H), 2.48-2.29 (m, 2H), 2.00-1.89 (m, 1H), 1.87-1.75 (m, 1H), 1.72-1.55 (m, 2H), 1.13-0.89 (m, 6H).

### Step 4: (1R,2S,5S)-3-(4,7-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

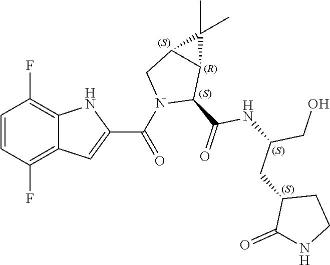

To a solution of (S)-methyl 2-((1R,2S,5S)-3-(4,7-difluoro-1H-indole-2-carbonyl)-6,6-dimethyl-3-azabicyclo[3.1.0]hexane-2-carboxamido)-3-((S)-2-oxopyrrolidin-3-yl)propanoate (150 mg, 298.50 mol) in THE (3 mL) was added LiBH₄ (50 mg, 2.30 mmol) at 0° C. The mixture was stirred at 25° C. for 1 h. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 15%-48%, 10 min). The title compound (20 mg, 13.92% yield) was obtained as a white solid. ¹H NMR (400 MHz, CD₃OD) δ ppm 7.13 (d, J=2.8 Hz, 1H), 6.96-6.86 (m, 1H), 6.76-6.61 (m, 1H), 4.32 (dd, J=5.6, 10.4 Hz, 1H), 4.09-3.98 (m, 1H), 3.94 (d, J=10.8 Hz, 1H), 3.86-3.74 (m, 1H), 3.56 (d, J=5.6 Hz, 1H), 3.50-3.42 (m, 1H), 3.29-3.25 (m, 1H), 2.75-2.53 (m, 1H), 2.44-2.29 (m, 1H), 2.05-1.68 (m, 3H), 1.67-1.27 (m, 3H), 1.18-0.92 (m, 6H).

### Step 5: (1R,2S,5S)-3-(4,7-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(4,7-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (20 mg, 42.15 mol) in DCM (4 mL) was added DMP (36 mg, 84.30 μmol, 26.10 L). The mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 12%-42%, 10 min). Compound 13 (5.4 mg, 26.07% yield, 96.16% purity) was obtained as a white solid. ¹H NMR (400 MHz, CD₃OD) δ ppm 7.17-6.85 (m, 2H), 6.75-6.59 (m, 1H), 4.54-4.38 (m, 1H), 4.35-3.96 (m, 2H), 3.96-3.89 (m, 1H), 3.85-3.69 (m, 1H), 2.69-2.27 (m, 2H), 2.09-1.68 (m, 3H), 1.65-1.29 (m, 3H), 1.21-0.92 (m, 6H). ESI-MS [M+H]⁺ calc'd for C₂₄H₂₆F₂N₄O₄, 473.19, found 473.4.

### Example 14: (1R,2S,5S)-3-(4-ethyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 14)

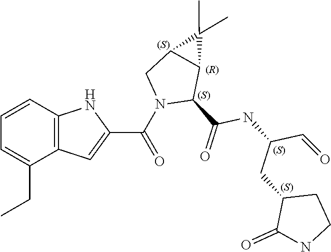

### Step 1: methyl 4-vinyl-1H-indole-2-carboxylate

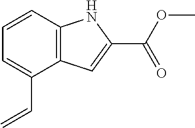

To a solution of methyl 4-bromo-1H-indole-2-carboxylate (2 g, 7.87 mmol) and 4,4,5,5-tetramethyl-2-vinyl-1,3,2-dioxaborolane (2.4 g, 15.74 mmol, 2.67 mL) in dioxane (16 mL) and H₂O (4 mL) was added Cs₂CO₃ (7.7 g, 23.61 mmol) and Pd(dppf)Cl₂ (576 mg, 787.15 mol) at 25° C. Then the mixture was stirred at 100° C. for 10 hours under N₂ atmosphere. The reaction mixture was filtered. Sat.aq. NH₄Cl (10 mL) was added to the filtrate. The aqueous phase was extracted with DCM (10 mL×2). The combined organic phase was washed with brine (10 mL×2), dried over anhydrous Na₂SO₄, filtered and concentrated to dryness. The residue was purified by flash silica gel chromatography (Eluent of 0~20% EtOAc/PE). The title compound (1 g, 56.82% yield) was obtained as a white solid.

¹H NMR (400 MHz, CDCl₃) δ ppm 8.90 (s, 1H), 7.48-7.40 (m, 1H), 7.35-7.28 (m, 2H), 7.19-7.05 (m, 1H), 5.94 (d, J=17.6 Hz, 1H), 5.44 (dd, J=1.2, 11.2 Hz, 1H), 3.96 (s, 3H).

### Step 2: methyl 4-ethyl-1H-indole-2-carboxylate

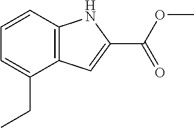

To a solution of methyl 4-vinyl-1H-indole-2-carboxylate (600 mg, 2.98 mmol) in THE (15 mL) was added wet Pd/C (635 mg, 10% purity) under N₂ atmosphere. The suspension was degassed and purged with H₂ for 3 times. The reaction mixture was stirred under H₂ (15 psi) at 25° C. for 2 hrs. The reaction mixture was filtered through the celites. The filtrate was concentrated to dryness. The title compound (500 mg, 74.26% yield) was obtained as a white solid which was used to the next step without further purification. ¹H NMR (400 MHz, CDCl₃) δ ppm 8.93 (br, 1H), 7.31-7.27 (m, 2H), 7.25-7.13 (m, 1H), 7.02-6.94 (m, 1H), 3.95 (s, 3H), 2.94 (q, J=7.6 Hz, 2H), 1.35 (t, J=7.6 Hz, 3H)

Step 3: 4-ethyl-1H-indole-2-carboxylic acid

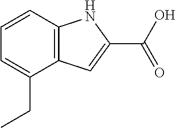

To a solution of methyl 4-ethyl-1H-indole-2-carboxylate (500 mg, 2.46 mmol) in THE (5 mL) and H₂O (5 mL) was added LiOH·H₂O (206 mg, 4.92 mmol) at 25° C. The mixture was stirred at 25° C. for 10 hrs. The reaction was diluted with water (10 mL). The resulting mixture was extracted with DCM (5 mL×3). The aqueous phase was acidified to pH~4 with aq. HCl (2M). The resulting suspension was filtered. The filter cake was then dried under vacuum. The title compound (300 mg, 58.00% yield) was obtained as a pink solid which was used in the next step without further purification. ¹H NMR (400 MHz, CDCl₃) δ ppm 8.88 (brs, 1H), 7.44 (d, J=2.0 Hz, 1H), 7.32-7.27 (m, 2H), 7.05-6.93 (m, 1H), 2.97 (q, J=7.6 Hz, 2H), 1.37 (t, J=7.6 Hz, 3H).

### Step 4: (S)-methyl 2-((1R,2S,5S)-3-(4-ethyl-1H-indole-2-carbonyl)-6,6-dimethyl-3-azabicyclo[3.1.0]hexane-2-carboxamido)-3-((S)-2-oxopyrrolidin-3-yl)propanoate

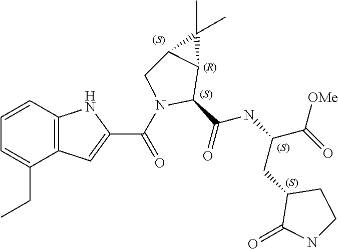

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (150 mg, 416.84 μmol, HCl) and 4-ethyl-1H-indole-2-carboxylic acid (79 mg, 416.84 mol) in DCM (5 mL) was added DIEA (162 mg, 1.25 mmol) at 0° C. The mixture was stirred for 5 min. And HATU (317 mg, 833.69 mol) was added into the above mixture at 0° C. The mixture was stirred at 25° C. for 10 hrs. 5% phosphoric acid solution (10 mL) was added to the reaction mixture. The mixture was extracted with DCM (10 mL×3). Then the combined organic phase was washed with sat.aq NaHCO₃ (20 mL), brine (20 mL), dried over anhydrous Na₂SO₄, filtered and concentrated to dryness. The residue was purified by prep-HPLC (Column: Phenomenex Gemini-NX C18 75×30 mm×3 μm; mobile phase: [water (0.05% NH₃H₂O+10 mM NH₄HCO₃)-ACN]; B %: 26%-66%, 11 min). The title compound (70 mg, 30.56% yield) was obtained as a white solid.

¹H NMR (400 MHz, CDCl₃) δ ppm 9.54-9.32 (m, 1H), 8.45-7.59 (m, 1H), 7.18-7.07 (m, 2H), 6.98-6.76 (m, 2H), 5.96-5.59 (m, 1H), 4.73-4.61 (m, 1H), 4.57-4.29 (m, 1H), 4.21-4.03 (m, 1H), 4.01-3.83 (m, 1H), 3.68-3.38 (m, 3H), 3.27-3.07 (m, 2H), 2.94-2.75 (m, 2H), 2.51-2.39 (m, 1H), 2.35-2.20 (m, 1H), 2.20-2.08 (m, 1H), 1.90-1.71 (m, 2H), 1.69-1.61 (m, 3H), 1.32-1.24 (m, 3H), 1.05-0.96 (m, 3H), 0.91-0.78 (m, 3H).

### Step 5: (1R,2S,5S)-3-(4-ethyl-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

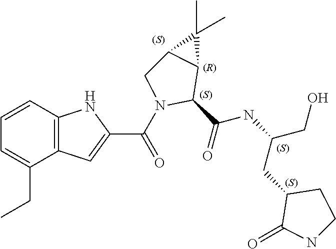

To a solution of (S)-methyl 2-((1R,2S,5S)-3-(4-ethyl-1H-indole-2-carbonyl)-6,6-dimethyl-3-azabicyclo[3.1.0]hexane-2-carboxamido)-3-((S)-2-oxopyrrolidin-3-yl)propanoate (70 mg, 141.53 mol) in THE (2 mL) was added LiBH₄ (30 mg, 1.38 mmol) at 0° C. Then the mixture was stirred at 25° C. for 2 hrs under N₂ atmosphere. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The reaction mixture was concentrated directly. The title compound (55 mg, 74.96% yield, 90% purity) was obtained as a white solid which was used in the next step without further purification. ¹H NMR (400 MHz, CDCl₃) δ ppm 9.73 (s, 1H), 8.19-7.72 (m, 1H), 7.39-7.29 (m, 1H), 7.23-7.17 (m, 1H), 7.01-6.84 (m, 2H), 4.69-4.56 (m, 1H), 4.21-4.09 (m, 1H), 4.11-3.79 (m, 3H), 3.78-3.71 (m, 2H), 3.57-3.47 (m, 1H), 3.33-3.18 (m, 2H), 2.97-2.82 (m, 2H), 2.41 (s, 2H), 2.15-2.00 (m, 1H), 1.71-1.58 (m, 2H), 1.35-1.28 (m, 3H), 1.08 (s, 3H), 0.94-0.88 (m, 3H).

### Step 6: (1R,2S,5S)-3-(4-ethyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(4-ethyl-1H-indole-2-carbonyl)-N—((S)-1-hydroxy-3—((S)-2-oxopyrrolidin-3-yl)propan-2-yl)-6,6-dimethyl-3-azabicyclo[3.1.0]hexane-2-carboxamide (50 mg, 107.16 mol) in DCM (2 mL) was added NaHCO₃ (18.0 mg, 214.33 mol) and DMP (136 mg, 321.49 mol) in one portion at 25° C. The mixture was stirred at 25° C. for 1 hr. The mixture was diluted with MeOH (3 mL) and then filtered. The filtrate was concentrated in vacuo. The residue was purified by prep-HPLC (column: Phenomenex Gemini-NX C18 75×30 mm×3 μm; mobile phase: [water (10 mM NH₄HCO₃)-ACN]; B %: 18%-58%, 11 min). Compound 14 (16.2 mg, 32.25% yield, 99.105% purity) was obtained as a whilte solid. ¹H NMR (400 MHz, CD₃OD) δ ppm 7.26 (t, J=8.0 Hz, 1H), 7.19-7.11 (m, 1H), 7.11-6.83 (m, 2H), 5.03-4.89 (m, 1H), 4.81-4.63 (m, 1H), 4.53-4.29 (m, 1H), 4.05-3.93 (m, 1H), 3.79-3.65 (m, 1H), 3.29-3.22 (m, 1H), 2.99-2.79 (m, 2H), 2.68-2.28 (m, 1H), 2.13-1.98 (m, 1H), 1.91-1.69 (m, 2H), 1.68-1.50 (m, 2H), 1.40-1.20 (m, 4H), 1.18-0.95 (m, 6H). ESI-MS [M+H]⁺ calc'd for C₂₆H₃₂N₄O₄, 465.24; found, 465.4.

### Example 15: (1R,2S,5S)-3-(4-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 15)

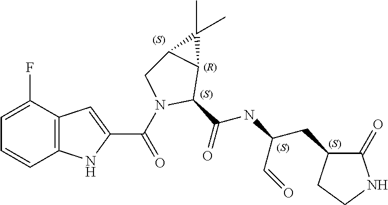

### Step 1: (S)-methyl 2-((1R,2S,5S)-3-(4-fluoro-1H-indole-2-carbonyl)-6,6-dimethyl-3-azabicyclo[3.1.0]hexane-2-carboxamido)-3-((S)-2-oxopyrrolidin-3-yl)propanoate

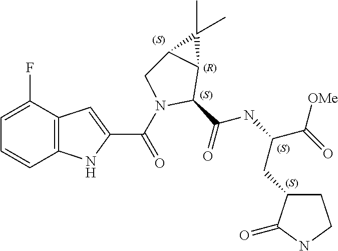

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (254 mg, 705.85 μmol, HCl) and 4-fluoro-1H-indole-2-carboxylic acid (126 mg, 705.85 mol) in DCM (5 mL) was added DIPEA (365 mg, 2.82 mmol) dropwise and HATU (403 mg, 1.06 mmol) slowly at 0° C. Then the mixture was stirred at 25° C. for 3 hours. Saturated aq. NaHCO₃ (10 mL) was added and the mixture was extracted with DCM (10 mL×3). The combined organic phase was washed with brine (10 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 23%-63%, 11 min). The title compound (190 mg, 53.25% yield) was obtained as a white solid. ¹H NMR (400 MHz, CD₃OD) δ ppm 7.28-7.22 (m, 1H), 7.21-7.14 (m, 1H), 7.12-6.89 (m, 1H), 6.74-6.69 (m, 1H), 4.85-4.78 (m, 1H), 4.63-4.52 (m, 1H), 4.41-4.25 (m, 1H), 4.04-3.94 (m, 1H), 3.84-3.67 (m, 3H), 3.35-3.31 (m, 2H), 3.07-2.30 (m, 2H), 2.23 (s, 1H), 1.90-1.67 (m, 2H), 1.64-1.43 (m, 2H), 1.20-0.92 (m, 6H).

### Step 2: (1R,2S,5S)-3-(4-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

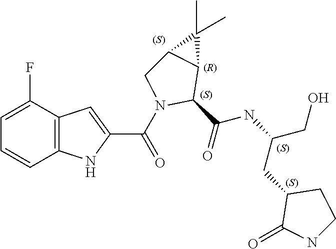

To a solution of (S)-methyl 2-((1R,2S,5S)-3-(4-fluoro-1H-indole-2-carbonyl)-6,6-dimethyl-3-azabicyclo[3.1.0]hexane-2-carboxamido)-3-((S)-2-oxopyrrolidin-3-yl)propanoate (160 mg, 330.22 mol) in THE (5 mL) was added LiBH₄ (22 mg, 990.67 mol) slowly at 0° C. Then the mixture was stirred at 25° C. for 1 hour. Saturated aq. NH₄Cl (10 mL) was added to the reaction mixture. Then the mixture was extracted with DCM (5 mL×3). The combined organic phase was washed with brine (5 mL×2), dried over anhydrous Na₂SO₄, filtered and concentrated in vacuo. The title compound (170 mg, crude) was obtained as a white solid. ESI-MS [M+H]⁺ calc'd for C₂₄H₂₉FN₄O₄, 457.22; found, 457.2.

### Step 3: (1R,2S,5S)-3-(4-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(4-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (96 mg, 210.29 mol) in DCM (4 mL) was added DMP (268 mg, 630.88 mol) slowly. Then the mixture was stirred at 25° C. for 1 hour. The reaction mixture was filtered. Saturated NaHCO₃ (5 mL) was added to the filtrate and the mixture was extracted with DCM (5 mL×3). The combined organic phase was washed with brine (4 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 17%-57%, 11 min). Compound 15 (26.1 mg, 27.01% yield, 98.913% purity) was obtained as a white solid. ¹H NMR (400 MHz, CD₃OD) δ ppm 7.29-7.22 (m, 1H), 7.22-7.13 (m, 1H), 7.12-6.91 (m, 1H), 6.73 (m, 1H), 4.63-4.40 (m, 1H), 4.36-3.70 (m, 3H), 3.29-3.24 (m, 1H), 2.97-2.58 (m, 1H), 2.45-2.28 (m, 1H), 2.09-1.33 (m, 6H), 1.16-0.95 (m, 6H). ESI-MS [M+H]⁺ calc'd for C₂₄H₂₇FN₄O₄, 455.20; found, 455.1.

### Example 16: (1R,2S,5S)-3-(2-(4,4-difluorocyclohexyl)acetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 16)

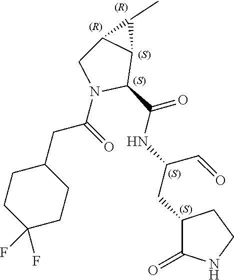

### Step 1: (S)-methyl 2-((1R,2S,5S)-3-(2-(4,4-difluorocyclohexyl)acetyl)-6,6-dimethyl-3-azabicyclo[3.1.0]hexane-2-carboxamido)-3-((S)-2-oxopyrrolidin-3-yl)propanoate

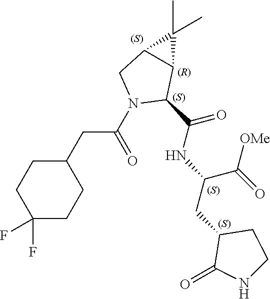

To a solution of 2-(4,4-difluorocyclohexyl)acetic acid (100 mg, 561.24 mol) in DCM (2 mL) was added DIEA (290 mg, 2.24 mmol) and T₃P (535 mg, 841.86 μmol, 50% purity in EtOAc) dropwise at 0° C. The mixture was stirred at 0° C. for 20 mins. Then (S)-methyl 2-((1R,2S,5S)-6,6-dimethyl-3-azabicyclo[3.1.0]hexane-2-carboxamido)-3-((S)-2-oxopyrrolidin-3-yl)propanoate (213 mg, 591.92 μmol, HCl) in DCM (2 mL) was added in one portion at 0° C. The mixture was stirred at 25° C. for 10 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: Xtimate C18 150×40 mm×10 μm; mobile phase: [water (0.05% HCl)-ACN]; B %: 25%-55%, 10 min). The title compound (86 mg, 18.97% yield) was obtained as a white solid. ¹H NMR (400 MHz, CDCl₃) δ ppm 8.68-7.56 (m, 1H), 6.81-6.69 (m, 1H), 6.45-6.23 (m, 1H), 4.69-4.51 (m, 1H), 4.46-4.13 (m, 1H), 4.00-3.83 (m, 1H), 3.81-3.69 (m, 3H), 3.56-3.30 (m, 3H), 2.45-2.41 (m, 1H), 2.32-1.63 (m, 13H), 1.61-1.46 (m, 2H), 1.35-1.22 (m, 2H), 1.12-0.87 (m, 6H).

### Step 2: (1R,2S,5S)-3-(2-(4,4-difluorocyclohexyl)acetyl)-N—((S)-1-hydroxy-3-((S)-2-oxopyrrolidin-3-yl)propan-2-yl)-6,6-dimethyl-3-azabicyclo[3.1.0]hexane-2-carboxamide

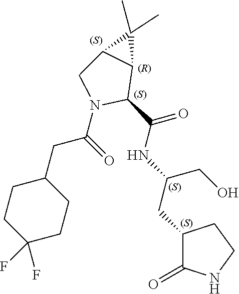

To a solution of (S)-methyl 2-((1R,2S,5S)-3-(2-(4,4-difluorocyclohexyl)acetyl)-6,6-dimethyl-3-azabicyclo[3.1.0]hexane-2-carboxamido)-3-((S)-2-oxopyrrolidin-3-yl)propanoate (85 mg, 175.78 mol) in THE (6 mL) was added LiBH₄ (12 mg, 527.35 mol) at 0° C. The mixture was stirred at 0° C. for 1 hr. H₂O (15 mL) was added to the reaction mixture. The resulting mixture was extracted with DCM (15 mL×3). The combined organic phase was washed with water (15 mL) and brine (15 mL), dried over anhydrous Na₂SO₄, filtered and concentrated to dryness. The title compound (75 mg, 36.07% yield) was obtained as yellow oil. ¹H NMR (400 MHz, CDCl₃) δ ppm 8.19-7.45 (m, 1H), 6.06-5.67 (m, 1H), 4.46-3.19 (m, 8H), 2.53-2.39 (m, 1H), 2.31-2.12 (m, 2H), 2.08-1.98 (m, 3H), 1.89-1.64 (m, 8H), 1.54-1.48 (m, 1H), 1.33-1.25 (m, 3H), 1.12-0.79 (m, 6H).

### Step 3: (1R,2S,5S)-3-(2-(4,4-difluorocyclohexyl)acetyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(2-(4,4-difluorocyclohexyl)acetyl)-N—((S)-1-hydroxy-3-((S)-2-oxopyrrolidin-3-yl)propan-2-yl)-6,6-dimethyl-3-azabicyclo[3.1.0]hexane-2-carboxamide (65 mg, 142.69 mol) in DCM (3 mL) was added DMP (182 mg, 428.07 mol) at 25° C. The reaction mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 20%-50%, 10 min) and prep-HPLC (column: Phenomenex Gemini-NX C18 75×30 mm×3 μm; mobile phase: [water (10 mM NH₄HCO₃)-ACN]; B %: 28%-58%, 10 min). Compound 16 (2.5 mg, 3.70% yield, 95.679% purity) was obtained as a white solid. ¹H NMR (400 MHz, CD₃OD) δ ppm 4.53-4.26 (m, 2H), 4.00-3.69 (m, 2H), 3.69-3.54 (m, 1H), 3.29-3.22 (m, 1H), 2.65-2.52 (m, 1H), 2.35-2.12 (m, 3H), 2.04-1.94 (m, 3H), 1.89-1.69 (m, 5H), 1.63-1.42 (m, 3H), 1.37-1.12 (m, 3H), 1.11-0.93 (m, 6H). ESI-MS [M+H]⁺ calc'd for C₂₃H₃₃F₂N₃O₄, 454.24; found, 454.3.

### Example 17: (1R,2S,5S)-6,6-dimethyl-N—((S)-1-oxo-3-((S)-2-oxopyrrolidin-3-yl)propan-2-yl)-3-(1-phenylcyclopentanecarbonyl)-3-azabicyclo[3.1.0]hexane-2-carboxamide (Compound 17)

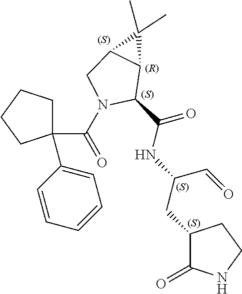

### Step 1: (S)-methyl 2-((1R,2S,5S)-6,6-dimethyl-3-(1-phenylcyclopentanecarbonyl)-3-azabicyclo[3.1.0]hexane-2-carboxamido)-3-((S)-2-oxopyrrolidin-3-yl)propanoate

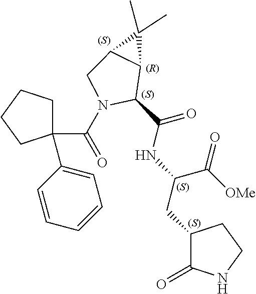

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (220 mg, 611.37 μmol, HCl), 1-phenylcyclopentanecarboxylic acid (116 mg, 611.37 mol) and DIEA (237 mg, 1.83 mmol) in DCM (4 mL) was added slowly HATU (465 mg, 1.22 mmol) at 0° C. The resulting mixture was stirred at 25° C. for 5 hr. Sat.aq.NH₄Cl (20 mL) was added to the reaction mixture. The resulting mixture was extracted with DCM (40 mL×3). The combined organic layers were dried over anhydrous Na₂SO₄, filtered and concentrated to dryness. The residue was purified by prep-HPLC (column: Phenomenex C18 75×30 mm×3 μm; mobile phase: [water (NH₃H₂O+NH₄HCO₃)-ACN]; B %: 31%-61%, 10 min). The title compound (45 mg, 12.97% yield) was obtained as a white solid. ¹H NMR (400 MHz, CDCl₃) δ ppm 7.31-7.22 (m, 6H), 5.67 (brs, 1H), 4.70-4.60 (m, 1H), 4.34 (s, 1H), 3.75 (s, 3H), 3.38-3.36 (m, 1H), 3.27-3.20 (m, 1H), 2.95 (d, J=10.8 Hz, 1H), 2.64-2.09 (m, 7H), 1.97-1.87 (m, 3H), 1.66-1.53 (m, 3H), 1.41-1.19 (m, 3H), 0.90 (s, 3H), 0.60 (s, 3H).

### Step 2: (1R,2S,5S)—N—((S)-1-hydroxy-3-((S)-2-oxopyrrolidin-3-yl)propan-2-yl)-6,6-dimethyl-3-(1-phenylcyclopentanecarbonyl)-3-azabicyclo[3.1.0]hexane-2-carboxamide

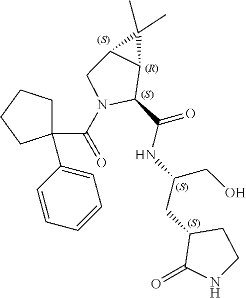

To a solution of (S)-methyl 2-((1R,2S,5S)-6,6-dimethyl-3-(1-phenylcyclopentanecarbonyl)-3-azabicyclo[3.1.0]hexane-2-carboxamido)-3-((S)-2-oxopyrrolidin-3-yl)propanoate (40 mg, 80.71 mol) in THE (3 mL) was added LiBH₄ (5 mg, 242.13 mol) at 0° C. The mixture was stirred at 25° C. for 1 h. The reaction was quenched with water (10 mL) drop-wise. The resulting mixture was extracted with DCM (10 mL×3). The combined organic phase was washed with water (5 mL), dried over anhydrous Na₂SO₄, filtered and concentrated to dryness. The title compound (40 mg, crude) was obtained as a white solid. ¹H NMR (400 MHz, CDCl₃) δ ppm 7.36-7.27 (m, 2H), 7.25-7.13 (m, 4H), 6.22-5.87 (m, 1H), 4.43-4.23 (m, 1H), 4.10-4.01 (m, 1H), 3.87-3.73 (m, 1H), 3.65-3.52 (m, 1H), 3.45-3.21 (m, 3H), 2.95 (d, J=10.8 Hz, 1H), 2.57-2.23 (m, 4H), 2.22-1.99 (m, 2H), 1.98-1.83 (m, 2H), 1.79-1.56 (m, 5H), 1.37-1.29 (m, 1H), 1.27-1.22 (m, 1H), 0.90 (s, 3H), 0.57 (s, 3H).

### Step 3: (1R,2S,5S)-6,6-dimethyl-N—((S)-1-oxo-3-((S)-2-oxopyrrolidin-3-yl)propan-2-yl)-3-(1-phenylcyclopentanecarbonyl)-3-azabicyclo[3.1.0]hexane-2-carboxamide

To a solution of (1R,2S,5S)—N—((S)-1-hydroxy-3-((S)-2-oxopyrrolidin-3-yl)propan-2-yl)-6,6-dimethyl-3-(1-phenylcyclopentanecarbonyl)-3-azabicyclo[3.1.0]hexane-2-carboxamide (40 mg, 85.54 mol) in DCM (8 mL) was added DMP (109 mg, 256.63 mol). The mixture was stirred at 25° C. for 2 hr. The reaction mixture was filtered. The filtrate was concentrated to dryness. The residue was purified by prep-HPLC (column: Phenomenex C18 250×50 mm×10 μm; mobile phase: [water (NH₄HCO₃)-ACN]; B %: 25%-55%, 10 min). Compound 17 (2.1 mg, 4.30 μmol, 5.03% yield, 95.427% purity) was obtained as a white solid. ¹H NMR (400 MHz, CD₃OD) δ ppm 7.36-7.29 (m, 2H), 7.28-7.20 (m, 3H), 4.52-4.28 (m, 1H), 4.08-3.87 (m, 1H), 3.44-3.37 (m, 1H), 3.35-3.25 (m, 2H), 2.92 (d, J=10.4 Hz, 1H), 2.73-2.60 (m, 1H), 2.46-2.33 (m, 2H), 2.32-1.99 (m, 3H), 1.89-1.71 (m, 4H), 1.69-1.49 (m, 3H), 1.37-1.24 (m, 2H), 0.91 (s, 3H), 0.58 (s, 3H). ESI-MS [M+H]⁺ calc'd for C₂₇H₃₅N₃O₄, 466.26; found, 466.2.

### Example 18: (1R,2S,5S)-3-(6,7-dimethyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 18)

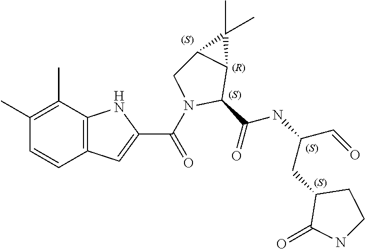

### Step 1: (S)-methyl 2-((1R,2S,5S)-3-(6,7-dimethyl-1H-indole-2-carbonyl)-6,6-dimethyl-3-azabicyclo[3.1.0]hexane-2-carboxamido)-3-((S)-2-oxopyrrolidin-3-yl)propanoate

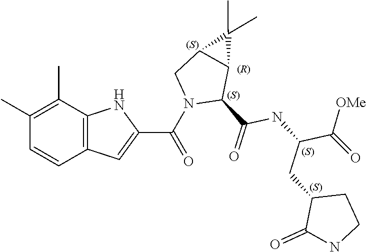

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (165 mg, 458.53 mol HCl), 6,7-dimethyl-1H-indole-2-carboxylic acid (87 mg, 458.53 mol) and DIEA (178 mg, 1.38 mmol) in DCM (3 mL) was added HATU (349 mg, 917.06 mol) slowly at 0° C. The mixture was stirred at 25° C. for 3 hr. Sat.aq.NH₄Cl (20 mL) was added and the resulting mixture was extracted with DCM (30 mL×3). The combined organic phase was washed with water (20 mL), dried over anhydrous Na₂SO₄, filtered and concentrated to dryness. The residue was purified by prep-HPLC (column: Xtimate C18 150×40 mm×10 μm; mobile phase: [water (10 mM NH₄HCO₃)-ACN]; B %: 30%-60%, 10 min). The title compound (100 mg, 40.35% yield) was obtained as a white solid. ¹H NMR (400 MHz, CDCl₃) δ ppm 9.29-9.11 (m, 1H), 8.62-7.86 (m, 1H), 7.44-7.28 (m, 1H), 6.99-6.91 (m, 1H), 6.91-6.77 (m, 1H), 6.28-5.87 (m, 1H), 4.78-4.66 (m, 1H), 4.63-4.36 (m, 1H), 4.24-4.07 (m, 1H), 4.02-3.89 (m, 1H), 3.77-3.47 (m, 3H), 3.34-3.17 (m, 2H), 2.56-2.45 (m, 1H), 2.40-2.28 (m, 6H), 2.25-2.08 (m, 1H), 2.02-1.92 (m, 1H), 1.89-1.72 (m, 2H), 1.70-1.36 (m, 2H), 1.16-0.78 (m, 6H).

### Step 2: (1R,2S,5S)-3-(6,7-dimethyl-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

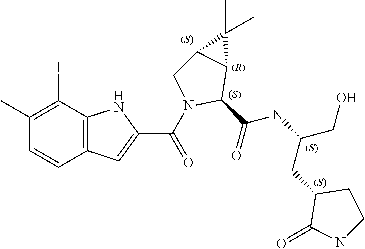

To a solution of (S)-methyl 2-((1R,2S,5S)-3-(6,7-dimethyl-1H-indole-2-carbonyl)-6,6-dimethyl-3-azabicyclo[3.1.0]hexane-2-carboxamido)-3-((S)-2-oxopyrrolidin-3-yl)propanoate (94 mg, 190.06 mol) in THE (2 mL) was added LiBH₄ (9 mg, 433.24 mol) at 0° C. The mixture was stirred at 25° C. for 1 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The title compound (90 mg, crude) was obtained as a white solid, which was used in the next step without further purification. ¹H NMR (400 MHz, CDCl₃) δ ppm 9.46-9.21 (m, 1H), 8.10-7.81 (m, 1H), 7.44-7.32 (m, 1H), 7.00-6.76 (m, 2H), 6.01-5.74 (m, 1H), 4.73-4.57 (m, 1H), 4.30-4.22 (m, 1H), 4.08-3.90 (m, 2H), 3.78-3.74 (m, 1H), 3.62-3.46 (m, 1H), 3.31-3.16 (m, 2H), 2.57-2.46 (m, 1H), 2.42-2.33 (m, 6H), 2.13-1.91 (m, 2H), 1.89-1.74 (m, 2H), 1.73-1.51 (m, 3H), 1.14-0.76 (m, 6H)

Step 3: (1R,2S,5S)-3-(6,7-dimethyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(6,7-dimethyl-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (80 mg, 171.46 mol) in DCM (3 mL) was added DMP (218 mg, 514.39 mol). The mixture was stirred at 25° C. for 10 min. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 21%-61%, 11 min). Compound 18 (30 mg, 60.94 μmol, 35.54% yield, 96.36% purity) was obtained as a white solid. ¹H NMR (400 MHz, CD₃OD) δ ppm 7.42-7.21 (m, 1H), 7.05-6.89 (m, 1H), 6.89-6.83 (m, 1H), 5.01-4.88 (m, 1H), 4.70-4.48 (m, 1H), 4.43-4.23 (m, 1H), 4.07-3.97 (m, 1H), 3.82-3.70 (m, 1H), 3.29-3.20 (m, 1H), 2.92-2.51 (m, 1H), 2.42 (s, 3H), 2.37 (d, J=6.0 Hz, 3H), 2.26-1.97 (m, 1H), 1.93-1.77 (m, 1H), 1.76-1.60 (m, 2H), 1.59-1.51 (m, 1H), 1.37-1.27 (m, 1H), 1.18-0.92 (m, 6H). ESI-MS [M+H]⁺ calc'd for C₂₆H₃₂N₄O₄, 465.24; found, 465.4.

### Example 19: (1R,2S,5S)-3-(6-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 19)

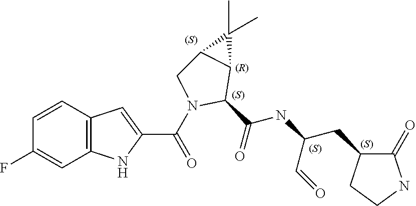

### Step 1: (S)-methyl 2-((1R,2S,5S)-3-(6-fluoro-1H-indole-2-carbonyl)-6,6-dimethyl-3-azabicyclo[3.1.0]hexane-2-carboxamido)-3-((S)-2-oxopyrrolidin-3-yl)propanoate

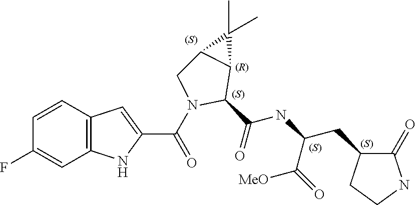

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (254 mg, 705.85 μmol, HCl) and 6-fluoro-1H-indole-2-carboxylic acid (126 mg, 705.85 mol) in DCM (5 mL) was added DIPEA (365 mg, 2.82 mmol) dropwise and HATU (402 mg, 1.06 mmol) slowly at 0° C. Then the mixture was stirred at 25° C. for 3 hours. Saturated aq. NaHCO₃ (10 mL) was added to the reaction mixture and the mixture was extracted with DCM (6 mL×3). The combined organic phase was washed with brine (10 mL×2), dried over anhydrous Na₂SO₄, filtered and concentrated to dryness. The residue was purified by prep-HPLC (column: Phenomenex Gemini-NX C18 75×30 mm×3 μm; mobile phase: [water (10 mM NH₄HCO₃)-ACN]; B %: 23%-63%, 11 min). The title compound (190 mg, 54.09% yield) was obtained as a white solid. ¹H NMR (400 MHz, CD₃OD) δ ppm 7.68-7.49 (m, 1H), 7.16-6.81 (m, 3H), 4.87-4.81 (m, 1H), 4.64-4.53 (m, 1H), 4.41-4.23 (m, 1H), 4.05-3.94 (m, 1H), 3.75-3.67 (m, 3H), 3.33-3.32 (m, 2H), 3.06-2.64 (m, 1H), 2.55-2.30 (m, 1H), 2.25-1.97 (m, 1H), 1.95-1.72 (m, 2H), 1.69-1.41 (m, 2H), 1.17-0.97 (m, 6H).

### Step 2: (1R,2S,5S)-3-(6-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

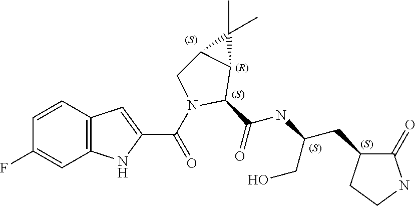

To a solution of (S)-methyl 2-((1R,2S,5S)-3-(6-fluoro-1H-indole-2-carbonyl)-6,6-dimethyl-3-azabicyclo[3.1.0]hexane-2-carboxamido)-3-((S)-2-oxopyrrolidin-3-yl)propanoate (160 mg, 330.22 mol) in THE (5 mL) was added LiBH₄ (22 mg, 990.67 mol) slowly at 0° C. Then the mixture was stirred at 25° C. for 1 hour. Saturated aq.NH₄Cl (10 mL) was added to the reaction mixture. Then the mixture was extracted with DCM (5 mL×3). The combined organic phase was washed with brine (5 mL×2), dried over anhydrous Na₂SO₄, filtered and concentrated in vacuo. The title compound (190 mg, crude) was obtained as a white solid. ESI-MS [M+H]⁺ calc'd for C₂₄H₂₉FN₄O₄, 457.22; found, 457.2.

### Step 3: (1R,2S,5S)-3-(6-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(6-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (96 mg, 210.29 mol) in DCM (4 mL) was added DMP (268 mg, 630.88 mol) slowly. Then the mixture was stirred at 25° C. for 1 hour. The reaction mixture was filtered. Saturated aq.NaHCO₃ (10 mL) was added to the filtrate and the mixture was extracted with DCM (5 mL×3). The combined organic phase was washed with brine (4 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 17%-57%, 11 min). Compound 19 (30 mg, 64.66 μmol, 30.75% yield) was obtained as a white solid. ¹H NMR (400 MHz, CD₃OD) δ ppm 7.69-7.46 (m, 1H), 7.16-6.78 (m, 3H), 4.65-4.38 (m, 1H), 4.33-3.70 (m, 3H), 3.28-3.23 (m, 1H), 2.99-2.57 (m, 1H), 2.41-2.27 (m, 1H), 2.10-1.32 (m, 6H), 1.16-0.95 (m, 6H). ESI-MS [M+H]⁺ calc'd for C₂₄H₂₇FN₄O₄, 455.20; found, 455.1.

### Example 20: (1R,2S,5S)-3-(4,6-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 20)

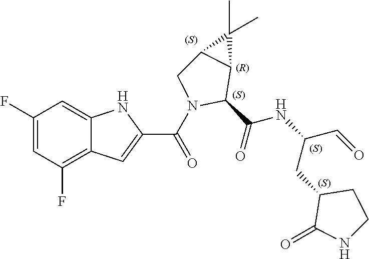

### Step 1: (S)-methyl 2-((1R,2S,5S)-3-(4,6-difluoro-1H-indole-2-carbonyl)-6,6-dimethyl-3-azabicyclo[3.1.0]hexane-2-carboxamido)-3-((S)-2-oxopyrrolidin-3-yl)propanoate

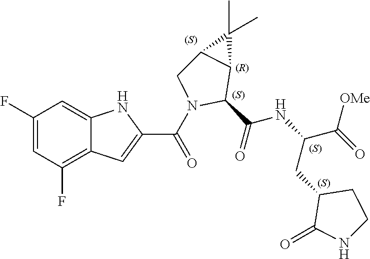

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate hydrochloride (168 mg, 466.86 mol, HCl) in DCM (5 mL) was added DIEA (181.0 mg, 1.40 mmol) and 4,6-difluoro-1H-indole-2-carboxylic acid (74 mg, 373.49 μmol) slowly at 25° C. Then HATU (355 mg, 933.72 mol) was added to the mixture at 0° C. The resulting mixture was stirred at 25° C. for 10 h. Water (12 mL) was added and the reaction was extracted with DCM (30 mL×2). The combined organic phase was washed with sat.aq.NH₄Cl (20 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 24%-64%, 11 min). The title compound (73.1 mg, 30.24% yield) was obtained as a white solid. ¹H NMR (400 MHz, CDCl₃) δ ppm 9.98-9.60 (m, 1H), 9.14-7.80 (m, 1H), 7.02-6.75 (m, 2H), 6.68-6.49 (m, 1H), 6.02-5.53 (m, 1H), 4.70-4.64 (m, 1H), 4.62-4.38 (m, 1H), 4.28-4.19 (m, 1H), 4.15-3.90 (m, 1H), 3.80-3.64 (m, 3H), 3.45-3.22 (m, 2H), 2.58-2.34 (m, 2H), 2.25-2.09 (m, 1H), 2.00-1.77 (m, 2H), 1.74-1.64 (m, 2H), 1.14-1.05 (m, 3H), 0.98-0.89 (m, 3H).

### Step 2: (1R,2S,5S)-3-(4,6-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

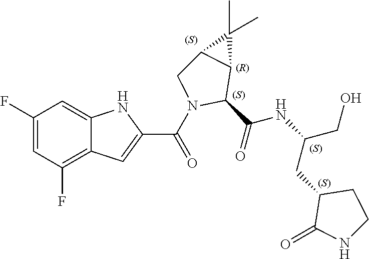

To a solution of (S)-methyl 2-((1R,2S,5S)-3-(4,6-difluoro-1H-indole-2-carbonyl)-6,6-dimethyl-3-azabicyclo[3.1.0]hexane-2-carboxamido)-3-((S)-2-oxopyrrolidin-3-yl)propanoate (73 mg, 145.27 mol) in THE (3 mL) at 0° C. was added LiBH₄ (10 mg, 435.81 mol) in one portion. The mixture was stirred at 25° C. for 1 hour. The residue was poured into water (10 mL). The aqueous phase was extracted with DCM (10 mL×3). The combined organic phase was washed with brine (5 mL), dried with anhydrous Na₂SO₄, filtered and concentrated under reduced pressure. The title compound (93.3 mg, crude) was obtained as a white solid, which was used in the next step without further purification. ¹H NMR (400 MHz, CDCl₃) δ ppm 10.28 (brs, 1H), 8.00 (d, J=7.2 Hz, 1H), 7.03-6.82 (m, 2H), 6.65-6.53 (m, 1H), 5.86 (brs, 1H), 4.84-4.56 (m, 1H), 4.33-4.24 (m, 1H), 4.10-4.01 (m, 1H), 3.97-3.90 (m, 1H), 3.79-3.72 (m, 1H), 3.63-3.52 (m, 1H), 3.33-3.19 (m, 2H), 2.58-2.46 (m, 1H), 2.42-2.30 (m, 1H), 2.20-2.08 (m, 1H), 1.84-1.65 (m, 3H), 1.62-1.53 (m, 2H), 1.06 (s, 3H), 0.98-0.85 (m, 3H).

### Step 3: (1R,2S,5S)-3-(4,6-difluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

A mixture of (1R,2S,5S)-3-(4,6-difluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (70 mg, 147.52 mol) in DCM (3 mL) was added DMP (188 mg, 442.57 μmol) and then the mixture was stirred at 25° C. for 1 h under N₂ atmosphere. The mixture was diluted with MeOH (3 mL) and filtered. The filtrate was concentrated in vacuo. The residue was purified by prep-HPLC (column: Phenomenex Gemini-NX C18 75×30 mm×3 μm; mobile phase: [water (10 mM NH₄HCO₃)-ACN]; B %: 10%-60%, 15 min) to give Compound 20 (6.5 mg, 12.94 μmol, 8.77% yield, 94.061% purity) as a white solid. ¹H NMR (400 MHz, CD₃OD) δ ppm 7.14-6.86 (m, 2H), 6.73-6.56 (m, 1H), 4.62-4.26 (m, 2H), 4.09-3.70 (m, 3H), 3.07-2.26 (m, 3H), 2.07-1.71 (m, 3H), 1.61-1.31 (m, 3H), 1.14-0.97 (m, 6H). ESI-MS [M+H]⁺ calc'd for C₂₄H₂₆F₂N₄O₄, 473.19; found, 473.4.

### Example 21: (1R,2S,5S)-3-(6-chloro-7-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 21)

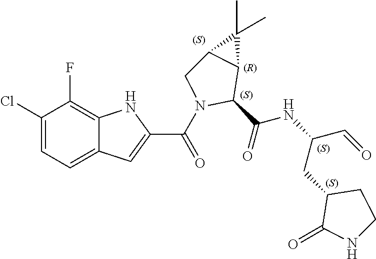

### Step 1: ethyl 6-chloro-7-fluoro-1H-indole-2-carboxylate

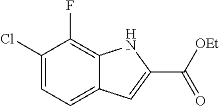 [SMILES: CCOC(=O)c1cc2ccc(Cl)c(F)c2[nH]1]

To a solution of (3-chloro-2-fluorophenyl) hydrazine (500 mg, 2.54 mmol, HCl) in toluene (10 mL) was added 4-methylbenzenesulfonic acid (1.31 g, 7.61 mmol) and ethyl 2-oxopropanoate (295 mg, 2.54 mmol) at 25° C. The mixture was stirred at 120° C. for 12 hr. Water (30 mL) was added to the mixture and the reaction mixture was adjusted to pH~7 by sat.aq.NaHCO₃. The resulting mixture was extracted with EtOAc (10 mL×3). The combined organic phase was washed with water (10 mL), dried over anhydrous Na₂SO₄, filtered and concentrated in vacuo. The residue was purified by flash silica gel chromatography (Eluent of 0-5% EtOAc in PE). The title compound (192 mg, 31.31% yield) was obtained as a yellowed solid. ¹H NMR (400 MHz, CDCl₃) δ ppm 9.09 (s, 1H), 7.39 (d, J=8.4 Hz, 1H), 7.22 (t, J=2.8 Hz, 1H), 7.12 (dd, J=2.8, 8.4 Hz, 1H), 4.44 (q, J=7.2 Hz, 2H), 1.44 (t, J=7.2 Hz, 3H)

### Step 2: 6-chloro-7-fluoro-1H-indole-2-carboxylic acid

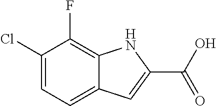 [SMILES: O=C(O)c1cc2ccc(Cl)c(F)c2[nH]1]

To a solution of ethyl 6-chloro-7-fluoro-1H-indole-2-carboxylate (190 mg, 786.28 mol) in THE (2 mL) and H₂O (2 mL) was added LiOH·H₂O (66 mg, 1.57 mmol). And then the mixture was stirred at 50° C. for 1 hr. Water (30 mL) was added to the mixture and the reaction mixture was adjusted to pH~5 by aq. HCl (1 N). The resulting mixture was extracted with DCM (20 mL×3). The combined organic phase was washed with water (10 mL), dried over anhydrous Na₂SO₄, filtered and concentrated. The title compound (130 mg, crude) was obtained as a yellow solid. ¹H NMR (400 MHz, CD₃OD) δ ppm 7.41 (d, J=8.4 Hz, 1H), 7.18 (d, J=3.2 Hz, 1H), 7.07 (dd, J=6.4, 8.4 Hz, 1H).

### Step 3: (S)-methyl 2-((1R,2S,5S)-3-(6-chloro-7-fluoro-1H-indole-2-carbonyl)-6,6-dimethyl-3-azabicyclo[3.1.0]hexane-2-carboxamido)-3-((S)-2-oxopyrrolidin-3-yl)propanoate

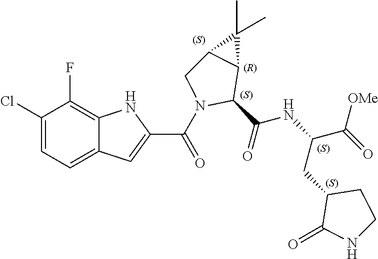

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (168 mg, 468.18 μmol, HCl), 6-chloro-7-fluoro-1H-indole-2-carboxylic acid (100 mg, 468.18 μmol,) and DIEA (182 mg, 1.40 mmol) in DCM (3 mL) was added slowly HATU (356 mg, 936.36 mol) at 0° C. The resulting mixture was stirred at 25° C. for 10 hr. Sat.aq.NH₄Cl (10 mL) was added. The resulting mixture was extracted with DCM (20 mL×3). The combined organic phase was washed with water (10 mL), dried over anhydrous Na₂SO₄, filtered and concentrated to dryness. The residue was purified by prep-HPLC (column: Phenomenex Gemini-NX C18 75×30 mm×3 μm; mobile phase: [water (0.05% NH₃H₂O+l0 mM NH₄HCO₃)-ACN]; B %: 25%-65%, 11 min). The title compound (53 mg, 20.98% yield) was obtained as a white solid.

¹H NMR (400 MHz, CDCl₃) δ ppm 9.95-9.53 (m, 1H), 8.81-8.06 (m, 1H), 7.40-7.28 (m, 1H), 7.17-6.99 (m, 1H), 6.98-6.37 (m, 1H), 5.92-5.61 (m, 1H), 4.86-4.64 (m, 1H), 4.57-4.35 (m, 1H), 4.26-4.08 (m, 1H), 4.01-3.84 (m, 1H), 3.79-3.52 (m, 3H), 3.42-3.25 (m, 2H), 2.63-2.30 (m, 2H), 2.24-2.04 (m, 1H), 2.02-1.81 (m, 2H), 1.78-1.68 (m, 1H), 1.46-1.21 (m, 1H), 1.14-0.82 (m, 6H).

### Step 4: (1R,2S,5S)-3-(6-chloro-7-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

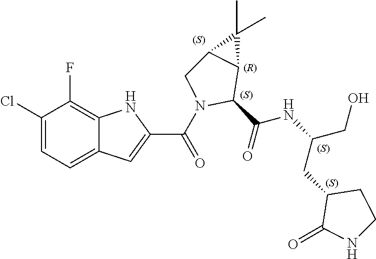

To a solution of (S)-methyl 2-((1R,2S,5S)-3-(6-chloro-7-fluoro-1H-indole-2-carbonyl)-6,6-dimethyl-3-azabicyclo[3.1.0]hexane-2-carboxamido)-3-((S)-2-oxopyrrolidin-3-yl)propanoate (50 mg, 96.35 mol) in THE (3 mL) was added LiBH₄ (30 mg, 1.38 mmol) at 0° C. The mixture was stirred at 25° C. for 1 h. The reaction was quenched with water (10 mL) drop-wise. The resulting mixture was extracted with DCM (10 mL×3). The combined organic phase was washed with water (10 mL), dried over anhydrous Na₂SO₄, filtered and concentrated. The title compound (33 mg, crude) was obtained as a white solid. ¹H NMR (400 MHz, CDCl₃) δ ppm 10.55-9.96 (m, 1H), 8.44-8.13 (m, 1H), 7.42-7.29 (m, 1H), 7.16-7.01 (m, 1H), 6.99-6.84 (m, 1H), 6.46-5.86 (m, 1H), 4.88-4.64 (m, 1H), 4.40-4.21 (m, 1H), 4.10-3.96 (m, 1H), 3.91-3.73 (m, 2H), 3.71-3.56 (m, 1H), 3.51-3.48 (m 1H), 3.42-3.20 (m, 2H), 2.68-2.46 (m, 1H), 2.45-2.29 (m, 1H), 2.20-1.96 (m, 1H), 1.66-1.55 (m, 2H), 1.45-1.39 (m, 1H), 1.30-1.21 (m, 1H), 1.12-0.82 (m, 6H).

### Step 5: (1R,2S,5S)-3-(6-chloro-7-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(6-chloro-7-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (30 mg, 61.11 mol) in DCM (6 mL) was added DMP (52 mg, 122.21 mol). The mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 28%-58%, 10 min). Compound 21 (1 mg, 3.27% yield, 97.76% purity) was obtained as a white solid. ¹H NMR (400 MHz, CD₃OD) δ ppm 7.49-7.28 (m, 1H), 7.16-6.88 (m, 2H), 5.01-4.91 (m, 1H), 4.52-4.28 (m, 1H), 4.02-3.70 (m, 2H), 3.29-2.97 (m, 2H), 2.70-2.43 (m, 1H), 2.40-1.98 (m, 1H), 1.86-1.29 (m, 5H), 1.18-0.92 (m, 6H). ESI-MS [M+H]⁺ calc'd for C₂₄H₂₆ClFN₄O₄, 489.16, found 489.3.

### Example 22 Synthesis of Compound 22

### Step 1: methyl 2-(3-oxocyclohexyl)acetate

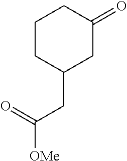 [SMILES: COC(=O)CC1CCCC(=O)C1]

A mixture of methyl 2-(3-hydroxycyclohexyl)acetate (500 mg, 2.90 mmol), PCC (1.88 g, 8.71 mmol) and silica gel (1.88 g) in DCM (20 mL) was degassed and purged with N₂ for 3 times, and then the mixture was stirred at 25° C. for 2 hr under N₂ atmosphere. The mixture was filtered and the filtrate was concentrated to give crude product. The residue was purified by flash silica gel chromatography (Eluent of 25% Ethyl acetate/Petroleum ether). The title compound (450 mg, 91.07% yield) was obtained as a colorless oil. ¹H NMR (400 MHz, CDCl₃) δ ppm 3.70 (s, 3H), 2.51-2.34 (m, 4H), 2.32-2.22 (m, 2H), 2.16-2.02 (m, 2H), 2.00-1.92 (m, 1H), 1.78-1.64 (m, 1H), 1.52-1.36 (m, 1H).

### Step 2: methyl 2-(3,3-difluorocyclohexyl)acetate

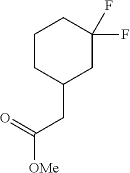 [SMILES: COC(=O)CC1CCCC(F)(F)C1]

To a mixture of methyl 2-(3-oxocyclohexyl)acetate (450 mg, 2.64 mmol) in DCM (15 mL) was added DAST (2.98 g, 18.51 mmol, 2.45 mL) dropwise at 0° C. The mixture was stirred at 25° C. for 12 hr. The mixture was quenched with saturated aqueous NaHCO₃ (60 mL) at 0° C., and extracted with DCM (30 mL×3). The organic layer was dried over Na₂SO₄ and concentrated to give crude product. The residue was purified by flash silica gel chromatography (Eluent of 5% Ethyl acetate/Petroleum ether). The title compound (450 mg, 88.56% yield) was obtained as a colorless oil. ¹H NMR (400 MHz, CDCl₃) δ ppm 3.61 (s, 3H), 3.01-2.61 (m, 1H), 2.28-2.19 (m, 2H), 2.17-1.91 (m, 3H), 1.77-1.63 (m, 2H), 1.48-1.27 (m, 1H), 1.18-0.92 (m, 2H).

### Step 3: 2-(3,3-difluorocyclohexyl)acetic acid

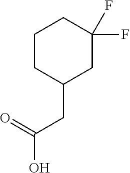 [SMILES: O=C(O)CC1CCCC(F)(F)C1]

A mixture of methyl 2-(3,3-difluorocyclohexyl)acetate (250 mg, 1.30 mmol) and LiOH·H₂O (163 mg, 3.90 mmol) in H₂O (2 mL) and THE (2 mL) was degassed and purged with N₂ for 3 times, and then the mixture was stirred at 25° C. for 4 hr under N₂ atmosphere. The reaction mixture was concentrated. Water (10 mL) was added and the reaction mixture was then adjusted to pH~4 by aqueous HCl (2 N). The resulting mixture was extracted with DCM (30 mL×10). The combined organic phase was washed with brine (30 mL), dried over anhydrous Na₂SO₄, filtered and concentrated to dryness. The title compound (140 mg, crude) was obtained as yellow oil, which was used to next step directly. ¹H NMR (400 MHz, CDCl₃) δ ppm 2.42-2.28 (m, 2H), 2.25-2.13 (m, 2H), 1.89-1.76 (m, 2H), 1.72-1.36 (m, 3H), 1.36-0.81 (m, 2H).

### Step 4: (2S)-methyl 2-((1R,2S,5S)-3-(2-(3,3-difluorocyclohexyl)acetyl)-6,6-dimethyl-3-azabicyclo[3.1.0]hexane-2-carboxamido)-3-((S)-2-oxopyrrolidin-3-yl)propanoate

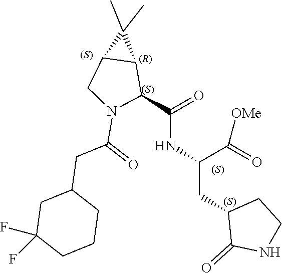

To a mixture of (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 708.63 μmol, HCl) and 2-(3,3-difluorocyclohexyl)acetic acid (126 mg, 708.63 mol) in DCM (5 mL) was added DIEA (366 mg, 2.83 mmol) and HATU (538 mg, 1.42 mmol) at 0° C. The mixture was stirred at 25° C. for 12 hr. The reaction mixture was quenched with saturated aqueous NH₄Cl (10 mL), and extracted with EtOAc (10 mL×2). The organic phase was washed with brine (10 mL×3), dried over Na₂SO₄, filtered and concentrated to give crude product. The crude product was purified by prep-HPLC (column: Welch Ultimate XB—CN 250×50×10 μm; mobile phase: [Heptane-EtOH (0.1% NH₃H₂O)]; B %: 10%-37%, 10 min). The title compound (120 mg, 27.67% yield) was obtained as a white solid. ESI-MS [M+H]⁺ calc'd for C₂₄H₃₅F₂N₃O₅, 484.25; found, 484.3.

### Step 5: (1R,2S,5S)-3-(2-(3,3-difluorocyclohexyl)acetyl)-N—((S)-1-hydroxy-3-((S)-2-oxopyrrolidin-3-yl)propan-2-yl)-6,6-dimethyl-3-azabicyclo[3.1.0]hexane-2-carboxamide

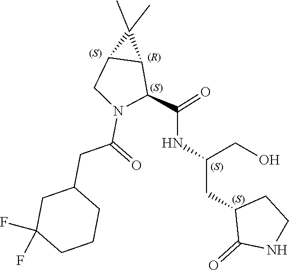

To a solution of (2S)-methyl 2-((1R,2S,5S)-3-(2-(3,3-difluorocyclohexyl)acetyl)-6,6-dimethyl-3-azabicyclo[3.1.0]hexane-2-carboxamido)-3-((S)-2-oxopyrrolidin-3-yl)propanoate (120 mg, 248.17 mol) in THE (2 mL) was added LiBH₄ (90 mg, 4.13 mmol) at 0° C. The mixture was stirred at 0° C. for 1 hr. The mixture was quenched with H₂O (10 mL), extracted with DCM (10 mL×3). The combined organic layer was dried over anhydrous Na₂SO₄, filtered and concentrated to dryness. The title compound (90 mg, 47.77% yield) was obtained which was used in the next step without further purification. ESI-MS [M+H]⁺ calc'd for C₂₅H₃₃N₃O₄, 456.26; found, 456.4.

### Step 6: (1R,2S,5S)-3-(2-(3,3-difluorocyclohexyl)acetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 22)

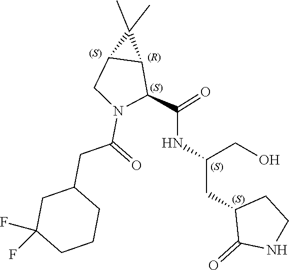

A mixture of (1R,2S,5S)-3-(2-(3,3-difluorocyclohexyl)acetyl)-N—((S)-1-hydroxy-3-((S)-2-oxopyrrolidin-3-yl)propan-2-yl)-6,6-dimethyl-3-azabicyclo[3.1.0]hexane-2-carboxamide (90 mg, 197.57 mol) and DMP (251 mg, 592.71 mol) in DCM (2 mL) was degassed and purged with N₂ for 3 times, and then the mixture was stirred at 25° C. for 0.5 hr under N₂ atmosphere. The mixture was filtered and the filtrate was concentrated to give crude product. The residue was purified by prep-HPLC (column: Phenomenex C18 75×30 mm×3 m; mobile phase: [water (NH₄HCO₃)-ACN]; B %: 20%-50%, 10 min). Compound 22 (9.1 mg, 9.45% yield, 93% purity) was obtained as a white solid. ¹H NMR (400 MHz, CD₃OD) δ ppm 4.58-4.46 (m, 1H), 4.38-4.28 (m, 1H), 4.08-3.84 (m, 2H), 3.77-3.55 (m, 1H), 3.30-3.25 (m, 1H), 2.79-2.51 (m, 1H), 2.44-1.87 (m, 8H), 1.83-1.24 (m, 9H), 1.09 (s, 3H), 1.02 -0.95 (m, 3H). ESI-MS [M+H]⁺ calc'd for C₂₃H₃₃F₂N₃O₄, 454.24; found, 454.4.

### Example 23: (1R,2S,5S)-6,6-dimethyl-N—((S)-1-oxo-3-((S)-2-oxopyrrolidin-3-yl)propan-2-yl)-3-(2-phenylacetyl)-3-azabicyclo[3.1.0]hexane-2-carboxamide (Compound 23)

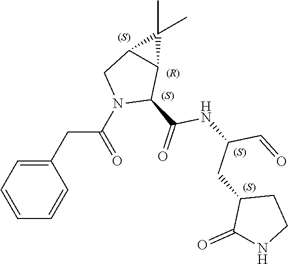

### Step 1: (S)-methyl 2-((1R,2S,5S)-6,6-dimethyl-3-(2-(4-methylcyclohexyl)acetyl)-3-azabicyclo[3.1.0]hexane-2-carboxamido)-3-((S)-2-oxopyrrolidin-3-yl)propanoate

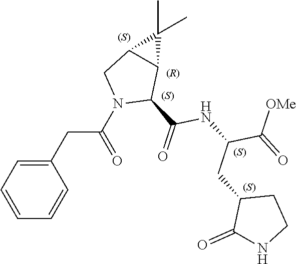

To a mixture of (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 708.63 μmol, HCl) and 2-phenylacetic acid (100 mg, 734.50 mol) in DCM (5 mL) was added DIEA (366 mg, 2.83 mmol) and HATU (538 mg, 1.42 mmol) at 0° C. The mixture was stirred at 25° C. for 12 hr. The reaction mixture was diluted with DCM (20 mL). The combined organic phase was washed with 5% H₃PO₄ (10 mL) and sat.aq. NaHCO₃ (10 mL), dried over anhydrous Na₂SO₄, filtered and concentrated. The crude product was purified by prep-HPLC (column: Xtimate C18 100×30 mm×10 μm; mobile phase: [water (10 mM NH₄HCO₃)-ACN]; B %: 10%-50%, 40 min). The title compound (70 mg, 20.81% yield) was obtained as a yellow solid. ¹H NMR (400 MHz, CD₃OD) δ ppm 7.37-7.18 (m, 5H), 4.59-4.53 (m, 1H), 4.35-4.32 (m, 1H), 3.94-3.88 (m, 1H), 3.79-3.74 (m, 3H), 3.71-3.57 (m, 3H), 3.30-3.22 (m, 2H), 2.70-2.57 (m, 1H), 2.39-2.28 (m, 1H), 2.22-2.13 (m, 1H), 1.88-1.74 (m, 2H), 1.60-1.43 (m, 2H), 1.09-1.04 (m, 3H), 0.88-0.75 (m, 3H).

### Step 2: (1R,2S,5S)—N—((S)-1-hydroxy-3-((S)-2-oxopyrrolidin-3-yl)propan-2-yl)-6,6-dimethyl-3-(2-phenylacetyl)-3-azabicyclo[3.1.0]hexane-2-carboxamide

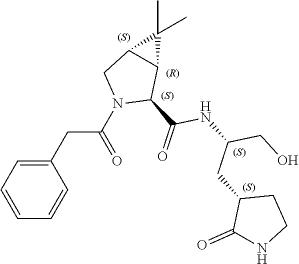

To a mixture of (S)-methyl 2-((1R,2S,5S)-6,6-dimethyl-3-(2-phenylacetyl)-3-azabicyclo[3.1.0]hexane-2-carboxamido)-3-((S)-2-oxopyrrolidin-3-yl)propanoate (70 mg, 158.54 mol) in THE (5 mL) was added LiBH₄ (10 mg, 475.63 mol) at 0° C. under N₂. The mixture was stirred at 0° C. for 1 hr. The mixture was quenched with H₂O (10 mL), and extracted with DCM (5 mL×3). The combined organic layer was dried over anhydrous Na₂SO₄, filtered and concentrated in vacuo. The crude product was purified by prep-HPLC (column: Welch Ultimate XB—NH₂ 250 mm×100 mm×10 μm; mobile phase: [Heptane-EtOH (0.1% NH₃H₂O)]; B %: 5%-40%, 15 min). The title compound (10 mg, 13.73% yield, 90% purity) was obtained as a white solid. ESI-MS [M+H]⁺ calc'd for C₂₃H₃₁N₃O₄, 414.23; found, 414.4.

### Step 3: (1R,2S,5S)-6,6-dimethyl-N—((S)-1-oxo-3-((S)-2-oxopyrrolidin-3-yl)propan-2-yl)-3-(2-phenylacetyl)-3-azabicyclo[3.1.0]hexane-2-carboxamide

A mixture of (1R,2S,5S)—N—((S)-1-hydroxy-3-((S)-2-oxopyrrolidin-3-yl)propan-2-yl)-6,6-dimethyl-3-(2-phenylacetyl)-3-azabicyclo[3.1.0]hexane-2-carboxamide (10 mg, 24.18 mol), DMP (30 mg, 72.55 mol) in DCM (2 mL) was degassed and purged with N₂ for 3 times. And the mixture was stirred at 25° C. for 0.5 hr under N₂ atmosphere. The mixture was filtered and the filtrate was concentrated to give crude product. The residue was purified by prep-HPLC (column: Phenomenex Gemini-NX C18 75×30 mm×3 μm; mobile phase: [water (10 mM NH₄HCO₃)-ACN]; B %: 15%-45%, 10 min). Compound 23 (1.7 mg, 4.01 μmol, 16.57% yield, 97% purity) was obtained as a white solid. ¹H NMR (400 MHz, CD₃OD) δ ppm 7.33-7.17 (m, 5H), 4.54-4.25 (m, 2H), 4.04-3.85 (m, 2H), 3.80-3.45 (m, 4H), 2.63-2.51 (m, 1H), 2.37-2.25 (m, 1H), 2.08-1.96 (m, 1H), 1.79-1.40 (m, 4H), 1.06-1.00 (m, 3H), 0.82-0.75 (m, 3H). ESI-MS [M+H]⁺ calc'd for C₂₄H₃₇N₃O₄, 412.22; found, 412.4.

### Example 24: (1R,2S,5S)-3-(5-fluoro-1H-indole-2-carbonyl)-N-[(1S)-1-formyl-2-[(3S)-2-oxopyrrolidin-3-yl]ethyl]-6,6-dimethyl-3-azabicyclo[3.1.0]hexane-2-carboxamide (Compound 24)

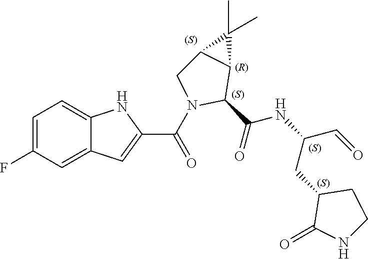

Compound 24 was synthesized by the same procedure as Compound 1. ¹H NMR (400 MHz, CD₃OD) δ ppm 7.44-7.37 (m, 1H), 7.35-7.15 (m, 1H), 7.08-6.84 (m, 2H), 5.00-4.89 (m, 1H), 4.65-4.56 (m, 1H), 4.54-4.25 (m, 1H), 4.02-3.71 (m, 2H), 2.98-2.59 (m, 1H), 2.44-2.29 (m, 1H), 2.10-1.83 (m, 1H), 1.81-1.68 (m, 2H), 1.66-1.55 (m, 1H), 1.53-1.25 (m, 2H), 1.20-0.88 (m, 6H). ESI-MS [M+H]⁺ calc'd for C₂₄H₂₇FN₄O₄, 455.20; found, 455.4.

### Example 25: (1R,2S,5S)-3-(7-chloro-1H-indole-2-carbonyl)-6,6-dimethyl-N—((S)-1-oxo-3-((S)-2-oxopiperidin-3-yl)propan-2-yl)-3-azabicyclo[3.1.0]hexane-2-carboxamide (Compound 25)

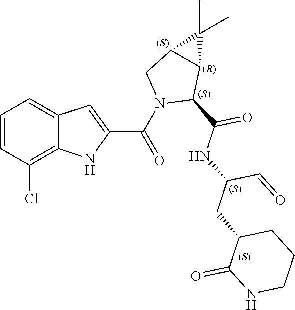

### Step 1: (S)-methyl 2-((1R,2S,5S)-3-(7-chloro-1H-indole-2-carbonyl)-6,6-dimethyl-3-azabicyclo[3.1.0]hexane-2-carboxamido)-3-((S)-2-oxopiperidin-3-yl)propanoate

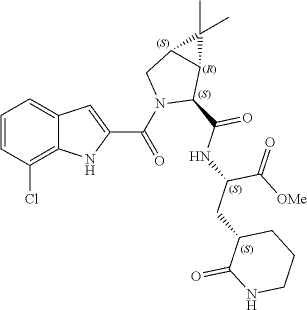

To a solution of 7-chloro-1H-indole-2-carboxylic acid (148 mg, 754.27 mol) in DCM (5 mL) was added T₃P (719 mg, 1.13 mmol, 50% purity in EtOAc) and DIEA (389 mg, 3.02 mmol) dropwise at 0° C. The mixture was stirred at 0° C. for 20 mins. Then (S)-methyl 2-((1R,2S,5S)-6,6-dimethyl-3-azabicyclo[3.1.0]hexane-2-carboxamido)-3-((S)-2-oxopiperidin-3-yl)propanoate (282 mg, 754.27 μmol, HCl) in DCM (2 mL) was added in one portion at 0° C. The mixture was stirred at 25° C. for 10 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: Phenomenex Gemini-NX C18 75×30 mm×3 μm; mobile phase: [water (10 mM NH₄HCO₃)-ACN]; B %: 28%-58%, 10 min). The title compound (45 mg, 9.05% yield) was obtained as a white solid. ¹H NMR (400 MHz, CDCl₃) δ ppm 11.37-7.82 (m, 1H), 7.65-7.50 (m, 1H), 7.33-7.27 (m, 2H), 7.17-6.93 (m, 2H), 6.36-5.91 (m, 1H), 4.94-4.47 (m, 2H), 4.42-4.17 (m, 1H), 4.14-3.87 (m, 1H), 3.80-3.47 (m, 3H), 3.38-3.06 (m, 2H), 2.52-2.14 (m, 2H), 2.10-1.39 (m, 7H), 1.16-0.88 (m, 6H)

### Step 2: (1R,2S,5S)-3-(7-chloro-1H-indole-2-carbonyl)-N—((S)-1-hydroxy-3-((S)-2-oxopiperidin-3-yl)propan-2-yl)-6,6-dimethyl-3-azabicyclo[3.1.0]hexane-2-carboxamide

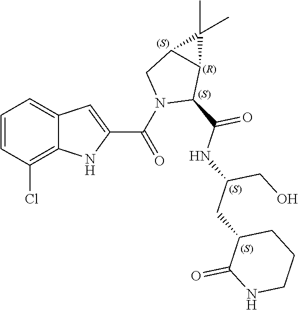

To a solution of (S)-methyl 2-((1R,2S,5S)-3-(7-chloro-1H-indole-2-carbonyl)-6,6-dimethyl-3-azabicyclo[3.1.0]hexane-2-carboxamido)-3-((S)-2-oxopiperidin-3-yl)propanoate (45 mg, 87.38 mol) in THE (6 mL) was added LiBH₄ (7 mg, 321.40 mol) dropwise at 0° C. The mixture was stirred at 0° C. for 1 hr. H₂O (10 mL) was added to the reaction mixture. The resulting mixture was extracted with DCM (5 mL×3). The combined organic phase was washed with brine (5 mL), water (5 mL), dried over anhydrous Na₂SO₄, filtered and concentrated to dryness. The title compound (50 mg, 77.57% yield) was obtained as a white solid.

¹H NMR (400 MHz, CDCl₃) δ ppm 10.16-8.17 (m, 1H), 7.63-7.43 (m, 1H), 7.34-7.28 (m, 1H), 7.13-6.89 (m, 2H), 6.02 (s, 1H), 4.72-4.53 (m, 1H), 4.28-3.92 (m, 2H), 3.84-3.68 (m, 3H), 3.61-3.45 (m, 1H), 3.38-2.83 (m, 2H), 2.44-2.25 (m, 1H), 2.15-2.01 (m, 1H), 1.80-1.71 (m, 3H), 1.65-1.52 (m, 3H), 1.26 (m, 1H), 1.15-0.83 (m, 6H).

### Step 3: (1R,2S,5S)-3-(7-chloro-1H-indole-2-carbonyl)-6,6-dimethyl-N—((S)-1-oxo-3—((S)-2-oxopiperidin-3-yl)propan-2-yl)-3-azabicyclo[3.1.0]hexane-2-carboxamide

To a solution of (1R,2S,5S)-3-(7-chloro-1H-indole-2-carbonyl)-N—((S)-1-hydroxy-3-((S)-2-oxopiperidin-3-yl)propan-2-yl)-6,6-dimethyl-3-azabicyclo[3.1.0]hexane-2-carboxamide (45 mg, 92.40 mol) in DCM (3 mL) was added DMP (118 mg, 277.21 mol) at 25° C. The reaction mixture was stirred at 25° C. for 1 hr. The reaction mixture was filtered. The filtrate was concentrated to dryness. The residue was purified by prep-HPLC (column: Phenomenex C18 250×50 mm×10 μm; mobile phase: [water (NH₄HCO₃)-ACN]; B %: 25%-55%, 10 min). Compound 25 (3.5 mg, 7.31% yield, 93.58% purity) was obtained as a white solid. ¹H NMR (400 MHz, CD₃OD) δ ppm 7.67-7.47 (m, 1H), 7.27 (t, J=8.0 Hz, 1H), 7.17-6.93 (m, 2H), 5.03-4.89 (m, 1H), 4.81-4.64 (m, 1H), 4.54-4.27 (m, 1H), 4.11-3.92 (m, 1H), 3.90-3.74 (m, 1H), 3.26-3.21 (m, 1H), 3.04-2.79 (m, 1H), 2.53-2.15 (m, 1H), 2.13-1.91 (m, 1H), 1.87-1.58 (m, 3H), 1.57-1.22 (m, 3H), 1.19-0.92 (m, 6H). ESI-MS [M+H]⁺ calc'd for C₂₅H₂₉ClN₄O₄, 485.19; found, 485.3.

### Example 26: (1S,3aR,6aS)-2-(1H-indole-2-carbonyl)-N—((S)-1-oxo-3-((S)-2-oxopyrrolidin-3-yl)propan-2-yl)octahydrocyclopenta[c]pyrrole-1-carboxamide (Compound 26)

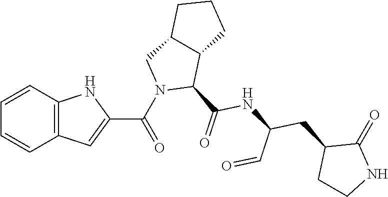 [SMILES: O=C[C@H](C[C@@H]1CCNC1=O)NC(=O)[C@@H]1[C@H]2CCC[C@H]2CN1C(=O)c1cc2ccccc2[nH]1]

### Step 1: (1S,3aR,6aS)-tert-butyl 1-(((S)-1-methoxy-1-oxo-3-((S)-2-oxopyrrolidin-3-yl)propan-2-yl)carbamoyl)hexahydrocyclopenta[c]pyrrole-2(1H)-carboxylate

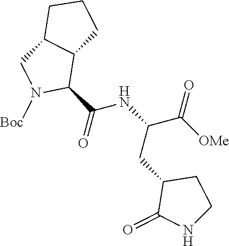 [SMILES: COC(=O)[C@H](C[C@@H]1CCNC1=O)NC(=O)[C@@H]1[C@H]2CCC[C@H]2CN1C(=O)OC(C)(C)C]

To a solution of (1S,3aR,6aS)-2-(tert-butoxycarbonyl)octahydrocyclopenta[c]pyrrole-1-carboxylic acid (400 mg, 1.57 mmol) in DCM (5 mL) and DIPEA (607 mg, 4.70 mmol) was added dropwise T₃P (1.50 g, 2.35 mmol, 50% purity in EtOAc) at 0° C. After addition, the mixture was stirred at the same temperature for 30 min, and then (S)-methyl 2-amino-3-((S)-2-oxopyrrolidin-3-yl)propanoate (380 mg, 1.71 mmol, HCl) in DCM (5 mL) was added at 25° C. The resulting mixture was stirred at 25° C. for 11.5 hrs. The reaction mixture was diluted with DCM (20 mL). The combined organic phase was washed with 5% H₃PO₄ (10 mL) and sat.aq. NaHCO₃ (10 mL), dried over anhydrous Na₂SO₄, filtered and concentrated to dryness. The title compound (573 mg, 57.00% yield) was obtained as a yellow oil. ¹H NMR (400 MHz, CD₃OD) δ ppm 4.60-4.45 (m, 1H), 4.12-3.99 (m, 1H), 3.80-3.71 (m, 4H), 3.68-3.64 (m, 1H), 3.30-3.21 (m, 2H), 2.78-2.60 (m, 2H), 2.54-2.32 (m, 1H), 2.25-1.95 (m, 2H), 1.94-1.58 (m, 7H), 1.53-1.41 (m, 10H)

### Step 2: (S)-methyl 2-((1S,3aR,6aS)-octahydrocyclopenta[c]pyrrole-1-carboxamido)-3-((S)-2-oxopyrrolidin-3-yl)propanoate

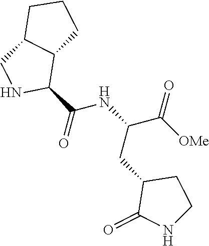 [SMILES: COC(=O)[C@H](C[C@@H]1CCNC1=O)NC(=O)[C@H]1NC[C@@H]2CCC[C@@H]21]

A mixture of (1S,3aR,6aS)-tert-butyl 1-(((S)-1-methoxy-1-oxo-3-((S)-2-oxopyrrolidin-3-yl)propan-2-yl)carbamoyl)hexahydrocyclopenta[c]pyrrole-2(1H)-carboxylate (200 mg, 472.25 mol) in HCl/dioxane (4M, 3 mL) was degassed and purged with N₂ for 3 times, and then the mixture was stirred at 25° C. for 1 hr under N₂ atmosphere. The mixture was concentrated to give crude product. The title compound (130 mg, crude) was obtained as a white solid.

### Step 3: (S)-methyl 2-((1S,3aR,6aS)-2-(1H-indole-2-carbonyl)octahydrocyclopenta[c]pyrrole-1-carboxamido)-3-((S)-2-oxopyrrolidin-3-yl)propanoate

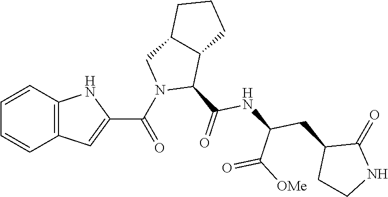 [SMILES: COC(=O)[C@H](C[C@@H]1CCNC1=O)NC(=O)[C@@H]1[C@H]2CCC[C@H]2CN1C(=O)c1cc2ccccc2[nH]1]

To a mixture of (S)-methyl 2-((1S,3aR,6aS)-octahydrocyclopenta[c]pyrrole-1-carboxamido)-3-((S)-2-oxopyrrolidin-3-yl)propanoate (130 mg, 402.00 mol) and 1H-indole-2-carboxylic acid (65 mg, 402.00 mol) in DCM (2 mL) was added HATU (305 mg, 803.99 mol) and DIEA (155 mg, 1.21 mmol, 210 μL) at 0° C. The mixture was stirred at 25° C. for 2 hr. The mixture was diluted with sat.aq. NH₄Cl (10 mL), and extracted with DCM (20 mL×3). The combined organic layer was dried over Na₂SO₄, filtered and concentrated to give crude product. The crude product was purified by prep-HPLC (column: Phenomenex Gemini-NX C18 75×30 mm×3 μm; mobile phase: [water (0.05% NH₃H₂O+10 mM NH₄HCO₃)-ACN]; B %: 21%-51%, 11 min). The title compound (76 mg, 39.31% yield) was obtained as a white solid. ¹H NMR (400 MHz, CD₃OD) δ ppm 7.73-7.53 (m, 1H), 7.46 (d, J=8.4 Hz, 1H), 7.29-7.20 (m, 1H), 7.13-6.82 (m, 2H), 4.64-4.01 (m, 3H), 3.91-3.48 (m, 4H), 3.06-2.70 (m, 3H), 2.56-1.43 (m, 12H).

### Step 4: (1S,3aR,6aS)—N—((S)-1-hydroxy-3-((S)-2-oxopyrrolidin-3-yl)propan-2-yl)-2-(1H-indole-2-carbonyl)octahydrocyclopenta[c]pyrrole-1-carboxamide

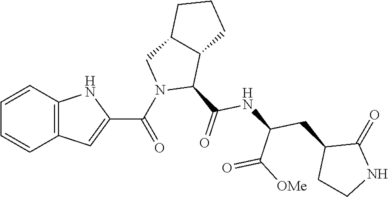 [SMILES: COC(=O)[C@H](C[C@@H]1CCNC1=O)NC(=O)[C@@H]1[C@H]2CCC[C@H]2CN1C(=O)c1cc2ccccc2[nH]1]

To a solution of (S)-methyl 2-((1S,3aR,6aS)-2-(1H-indole-2-carbonyl)octahydrocyclopenta[c]pyrrole-1-carboxamido)-3-((S)-2-oxopyrrolidin-3-yl)propanoate (70 mg, 150.04 mol) in THE (5 mL) was added LiBH₄ (71 mg, 3.26 mmol) at 0° C. The mixture was stirred at 0° C. for 1 hr. The mixture was quenched with H₂O (5 mL), and extracted with DCM (5 mL×3). The combined organic layer was dried over anhydrous Na₂SO₄, filtered and concentrated to dryness. The title compound (25 mg, 19.00% yield) was obtained as a white solid. ESI-MS [M+H]⁺ calc'd for C₂₄H₃₀N₄O₄, 439.23; found, 439.2.

### Step 5: (1S,3aR,6aS)-2-(1H-indole-2-carbonyl)-N—((S)-1-oxo-3-((S)-2-oxopyrrolidin-3-yl)propan-2-yl)octahydrocyclopenta[c]pyrrole-1-carboxamide

To a mixture of (1S,3aR,6aS)—N—((S)-1-hydroxy-3-((S)-2-oxopyrrolidin-3-yl)propan-2-yl)-2-(1H-indole-2-carbonyl)octahydrocyclopenta[c]pyrrole-1-carboxamide (25 mg, 57.01 mol) in DCM (2 mL) was added DMP (72 mg, 171.03 mol). The mixture was stirred at 25° C. for 0.5 hr. The mixture was filtered and the filtrate was concentrated to give crude product. The crude product was purified by prep-HPLC (column: Phenomenex Gemini-NX C18 75×30 mm×3 μm; mobile phase: [water (10 mM NH₄HCO₃)-ACN]; B %: 17%-47%, 10 min). Compound 26 (2 mg, 4.44 μmol, 7.80% yield, 97% purity) was obtained as a yellow solid. ¹H NMR (400 MHz, CD₃OD) δ ppm 7.72-7.52 (m, 1H), 7.45 (d, J=8.4 Hz, 1H), 7.24 (t, J=7.6 Hz, 1H), 7.12-6.85 (m, 2H), 4.60-4.26 (m, 2H), 4.14-3.62 (m, 3H), 3.03-2.26 (m, 5H), 2.09-2.02 (m, 1H), 1.98-1.85 (m, 3H), 1.80-1.70 (m, 2H), 1.66-1.54 (m, 2H), 1.41-1.30 (m, 1H). ESI-MS [M+H]⁺ calc'd for C₂₄H₂₈N₄O₄, 437.21; found, 437.2.

### Example 27: (1R,2S,5S)-3-(5H-[1,3]dioxolo[4,5-f]indole-6-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 27)

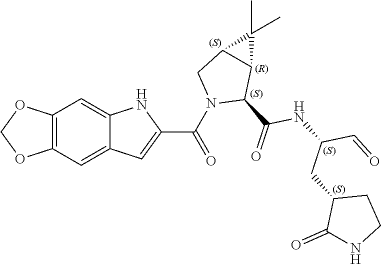

### Step 1: (S)-methyl 2-((1R,2S,5S)-3-(5H-[1,3]dioxolo[4,5-f]indole-6-carbonyl)-6,6-dimethyl-3-azabicyclo[3.1.0]hexane-2-carboxamido)-3-((S)-2-oxopyrrolidin-3-yl)propanoate

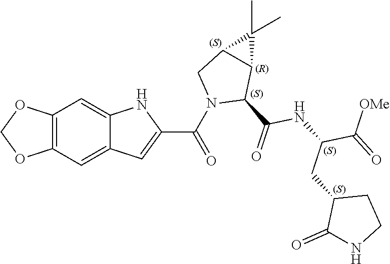

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (150 mg, 463.84 μmol, HCl) and DIEA (180 mg, 1.39 mmol) in DCM (10 mL) was added 5H-[1,3]dioxolo[4,5-f]indole-6-carboxylic acid (95 mg, 463.84 mol). After addition, HATU (353 mg, 927.68 mol) was added at 0° C. The mixture was stirred at 25° C. for 12 hr. The reaction mixture was poured into sat.aq NH₄Cl (20 mL). The resulting mixture was extracted with EtOAc (20 mL×2). The combined organic phase was washed with brine (30 mL), dried over anhydrous Na₂SO₄, filtered and concentrated to dryness. The title compound (224 mg, crude) was obtained as a brown solid. ESI-MS [M+H]⁺ calc'd for C₂₆H₃₀N₄O₇, 511.21; found, 511.2.

### Step 2: (1R,2S,5S)-3-(5H-[1,3]dioxolo[4,5-f]indole-6-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

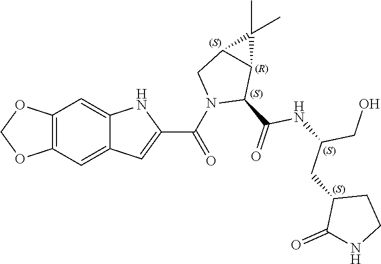

To a solution of (S)-methyl 2-((1R,2S,5S)-3-(5H-[1,3]dioxolo[4,5-f]indole-6-carbonyl)-6,6-dimethyl-3-azabicyclo[3.1.0]hexane-2-carboxamido)-3-((S)-2-oxopyrrolidin-3-yl)propanoate (200 mg, 391.74 mol) in THE (3 mL) was added LiBH₄ (10 mg, 459.14 mol) at 0° C. Then the mixture was stirred at 0° C. for 1 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 14%-54%, 11 min). The title compound (70 mg, 35.67% yield, 96.31% purity) was obtained as a white solid. ¹H NMR (400 MHz, CDCl₃) δ ppm 9.75-9.63 (m, 1H), 8.10-7.71 (m, 1H), 7.00-6.90 (m, 1H), 6.87-6.71 (m, 2H), 6.00-5.92 (m, 2H), 5.92-5.82 (m, 1H), 4.70-4.56 (m, 1H), 4.26-4.17 (m, 1H), 4.07-3.98 (m, 1H), 3.90 (d, J=10.0 Hz, 1H), 3.81-3.72 (m, 1H), 3.58-3.44 (m, 1H), 3.30-3.16 (m, 2H), 2.60-2.47 (m, 1H), 2.38-2.28 (m, 1H), 2.13-2.06 (m, 1H), 1.78-1.68 (m, 1H), 1.68-1.62 (m, 1H), 1.62-1.56 (m, 1H), 1.55-1.39 (m, 1H), 1.15-0.75 (m, 6H).

### Step 3: (1R,2S,5S)-3-(5H-[1,3]dioxolo[4,5-f]indole-6-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(5H-[1,3]dioxolo[4,5-f]indole-6-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (40 mg, 82.90 mol) in DCM (10 mL) was added DMP (105 mg, 248.69 mol, 77 μL) at 25° C. The mixture was stirred at 25° C. for 1 hr. The reaction mixture was filtered and the filtrate was concentrated to dryness directly. The residue was purified by prep-HPLC (column: Phenomenex Gemini-NX C18 75×30 mm×3 μm; mobile phase: [water (10 mM NH₄HCO₃)-ACN]; B %:10%-60%, 15 min). Compound 27 (3.8 mg, 6.91 μmol, 8.33% yield, 87.36% purity) was obtained as a light yellow solid. ¹H NMR (400 MHz, CD₃OD) δ ppm 7.06-6.93 (m, 1H), 6.91-6.83 (m, 1H), 6.83-6.44 (m, 1H), 6.06-5.81 (m, 2H), 4.99-4.92 (m, 1H), 4.69-4.56 (m, 1H), 4.52-4.30 (m, 1H), 4.29-3.89 (m, 2H), 3.87-3.66 (m, 1H), 2.70-2.44 (m, 1H), 2.41-2.26 (m, 1H), 2.10-1.89 (m, 1H), 1.75-1.65 (m, 1H), 1.63-1.47 (m, 2H), 1.43-1.28 (m, 1H), 1.17-0.93 (m, 6H). ESI-MS [M+H]⁺ calc'd for C₂₅H₂₈N₄O₆, 481.20; found, 481.4.

### Example 28: (1R,2S,5S)-3-(7-chloro-4-methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 28)

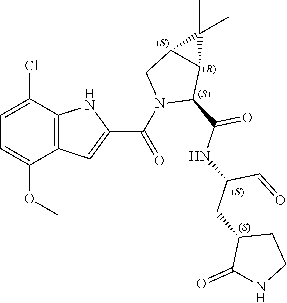

### Step 1: (2-chloro-5-methoxyphenyl)hydrazine

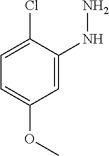

To a solution of 2-chloro-5-methoxyaniline (2 g, 12.69 mmol) in HCl (12 M, 10.00 mL) was added dropwise a solution of NaNO₂ (1.31 g, 19.04 mmol) in H₂O (10 mL) in at 0° C. After addition, the mixture was stirred at 0° C. for 30 min, and then SnCl₂ (4.81 g, 25.38 mmol) was added slowly at 0° C. The resulting mixture was stirred at 0° C. for 2 hr. The reaction mixture was filtered. The filtrate was adjusted to pH~10 with aq. NaOH (6 M). The resulting mixture was extracted with DCM (100 mL×3). The combined organic phase was washed with water (50 mL), dried over anhydrous Na₂SO₄, filtered and concentrated to dryness. The residue was purified by flash silica gel chromatography (Eluent of 0~16% EtOAc in PE). The title compound (860 mg, 34.66% yield) was obtained as a yellow solid. H NMR (400 MHz, CDCl₃) δ ppm 7.14 (d, J=8.4 Hz, 1H), 6.72 (d, J=2.8 Hz, 1H), 6.31 (dd, J=2.8, 8.8 Hz, 1H), 3.82 (s, 3H).

### Step 2: ethyl 7-chloro-4-methoxy-1H-indole-2-carboxylate

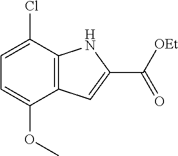 [SMILES: CCOC(=O)c1cc2c(OC)ccc(Cl)c2[nH]1]

To a solution of (2-chloro-5-methoxyphenyl)hydrazine (860 mg, 4.98 mmol) in toluene (10 mL) was added TsOH (2.57 g, 14.95 mmol) and ethyl 2-oxopropanoate (579 mg, 4.98 mmol) at 25° C. The mixture was stirred at 120° C. for 12 hr. Water (20 mL) was added and the reaction mixture was adjusted to pH~7 by sat.aq. NaHCO₃. The resulting mixture was extracted with EtOAc (50 mL×3). The combined organic phase was washed with water (20 mL), dried over anhydrous Na₂SO₄, filtered and concentrated to dryness. The residue was purified by flash silica gel chromatography (Eluent of 0~5% EtOAc in PE). The title compound (80 mg, 6.33% yield) was obtained as a white solid. ¹H NMR (400 MHz, CDCl₃) δ ppm 8.97 (s, 1H), 7.35 (d, J=2.4 Hz, 1H), 7.21 (d, J=8.0 Hz, 1H), 6.44 (d, J=8.0 Hz, 1H), 4.41 (q, J=7.2 Hz, 2H), 3.94 (s, 3H), 1.41 (t, J=7.2 Hz, 3H).

### Step 3: 7-chloro-4-methoxy-1H-indole-2-carboxylic acid

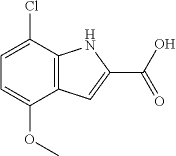

To a solution of ethyl 7-chloro-4-methoxy-1H-indole-2-carboxylate (80 mg, 315.36 mol) in THE (1 mL) and H₂O (1 mL) was added LiOH·H₂O (26 mg, 630.71 mol). And then the mixture was stirred at 25° C. for 10 hr. Water (10 mL) was added and the reaction mixture was adjusted to pH~5 by aq. HCl (1 N). The resulting mixture was extracted with DCM (10 mL×3). The combined organic phase was washed with water (10 mL), dried over anhydrous Na₂SO₄, filtered and concentrated to dryness. The title compound (70 mg, crude) was obtained as a yellow solid. ¹H NMR (400 MHz, CD₃OD) δ ppm 7.24 (s, 1H), 7.19 (d, J=8.0 Hz, 1H), 6.52 (d, J=8.4 Hz, 1H), 3.93 (s, 3H).

### Step 4: (S)-methyl 2-((1R,2S,5S)-3-(7-chloro-4-methoxy-1H-indole-2-carbonyl)-6,6-dimethyl-3-azabicyclo[3.1.0]hexane-2-carboxamido)-3-((S)-2-oxopyrrolidin-3-yl)propanoate

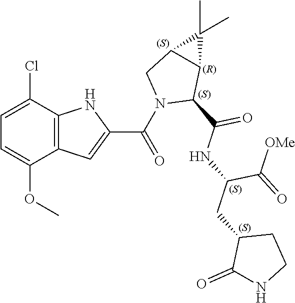

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (112 mg, 310.25 μmol, HCl), 7-chloro-4-methoxy-1H-indole-2-carboxylic acid (70 mg, 310.25 μmol,) and DIEA (120 mg, 930.74 mol) in DCM (3 mL) was added slowly HATU (236 mg, 620.49 mol) at 0° C. The resulting mixture was stirred at 25° C. for 3 hr. The reaction mixture was concentrated. Sat.aq.NH₄Cl (10 mL) was added to the residue. The resulting mixture was extracted with DCM (20 mL×3). The combined organic phase was washed with water (10 mL), dried over anhydrous Na₂SO₄, filtered and concentrated to dryness. The residue was purified by prep-HPLC (column: Phenomenex C18 75×30 mm×3 μm; mobile phase: [water (NH₃H₂O+NH₄HCO₃)-ACN]; B %: 26%-50%, 9 min). The title compound (50 mg, 27.90% yield) was obtained as a white solid. ¹H NMR (400 MHz, CDCl₃) δ ppm 9.62-9.35 (m, 1H), 8.29-7.91 (m, 1H), 7.21-7.12 (m, 1H), 7.11-6.92 (m, 1H), 6.47-6.35 (m, 1H), 5.85-5.67 (m, 1H), 4.82-4.63 (m, 1H), 4.60-4.37 (m, 1H), 4.31-4.09 (m, 1H), 4.08-3.84 (m, 4H), 3.80-3.50 (m, 3H), 3.41-3.04 (m, 2H), 2.66-2.50 (m, 1H), 2.49-2.12 (m, 2H), 2.11-1.81 (m, 4H), 1.15-0.83 (m, 6H).

### Step 5: (1R,2S,5S)-3-(7-chloro-4-methoxy-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

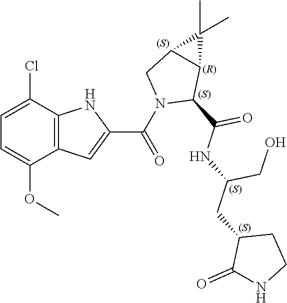

To a solution of (S)-methyl 2-((1R,2S,5S)-3-(7-chloro-4-methoxy-1H-indole-2-carbonyl)-6,6-dimethyl-3-azabicyclo[3.1.0]hexane-2-carboxamido)-3-((S)-2-oxopyrrolidin-3-yl)propanoate (50 mg, 94.16 mol) in THE (3 mL) was added LiBH₄ (50 mg, 2.30 mmol) at 0° C. The mixture was stirred at 25° C. for 1 h. The reaction was quenched with water (10 mL) drop-wise. The resulting mixture was extracted with DCM (10 mL×3). The combined organic phase was washed with water (5 mL), dried over anhydrous Na₂SO₄, filtered and concentrated. The title compound (30 mg, 41.81% yield) was obtained as a white solid. ¹H NMR (400 MHz, CDCl₃) δ ppm 9.83-9.29 (m, 1H), 8.24-7.78 (m, 1H), 7.23-7.12 (m, 1H), 7.12-6.93 (m, 1H), 6.54-6.34 (m, 1H), 5.90-5.62 (m, 1H), 4.85-4.57 (m, 1H), 4.55-4.23 (m, 1H), 4.22-3.81 (m, 6H), 3.80-3.68 (m, 1H), 3.64-3.47 (m, 1H), 3.37-3.30 (m, 1H), 2.65-2.16 (m, 2H), 2.09-1.94 (m, 2H), 1.72-1.53 (m, 3H), 1.12-0.82 (m, 6H).

### Step 6: (1R,2S,5S)-3-(7-chloro-4-methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-chloro-4-methoxy-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (30 mg, 59.64 mol) in DCM (6 mL) was added DMP (76 mg, 178.93 mol). The mixture was stirred at 25° C. for 0.5 hr. The reaction mixture was filtered and the filtrate was concentrated. The residue was purified by prep-HPLC (column: Phenomenex C18 75×30 mm×3 μm; mobile phase: [water (NH₄HCO₃)-ACN]; B %: 22%-52%, 10 min). Compound 28 (5.4 mg, 17.69% yield, 97.902% purity) was obtained as a white solid. ¹H NMR (400 MHz, CD₃OD) δ ppm 7.26-6.93 (m, 2H), 6.59-6.41 (m, 1H), 5.04-4.94 (m, 1H), 4.53-4.25 (m, 1H), 4.06-3.97 (m, 1H), 3.95-3.85 (m, 3H), 3.83-3.69 (m, 1H), 3.27-2.86 (m, 1H), 2.70-2.30 (m, 1H), 2.28-1.88 (m, 2H), 1.81-1.48 (m, 3H), 1.43-1.32 (m, 1H), 1.21-0.90 (m, 6H). ESI-MS [M+H]⁺ calc'd for C₂₅H₂₉ClN₄O₅, 501.18, found 501.3.

### Example 29: (1R,2S,5S)-6,6-dimethyl-N—((S)-1-oxo-3-((S)-2-oxopyrrolidin-3-yl)propan-2-yl)-3-(7-(trifluoromethyl)-1H-indole-2-carbonyl)-3-azabicyclo[3.1.0]hexane-2-carboxamide (Compound 29)

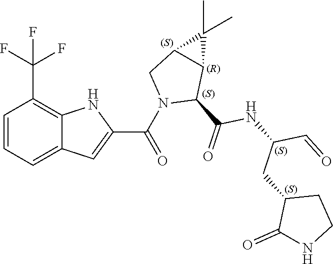

### Step 1: (S)-methyl 2-((1R,2S,5S)-6,6-dimethyl-3-(7-(trifluoromethyl)-1H-indole-2-carbonyl)-3-azabicyclo[3.1.0]hexane-2-carboxamido)-3-((S)-2-oxopyrrolidin-3-yl)propanoate

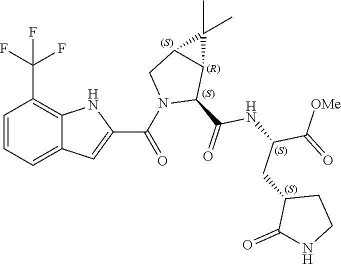

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 708.63 μmol, HCl) in DCM (4 mL) was added DIEA (274 mg, 2.13 mmol) and 7-(trifluoromethyl)-1H-indole-2-carboxylic acid (163 mg, 708.63 mol) dropwise at 0° C. Then HATU (539 mg, 1.42 mmol) was added in one portion at 0° C. The mixture was stirred at 25° C. for 10 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: Phenomenex Gemini-NX C18 75×30 mm×3 μm; mobile phase: [water (10 mM NH₄HCO₃)-ACN]; B %: 30%-60%, 10 min). The title compound (95 mg, 23.80% yield) was obtained as a white solid. ¹H NMR (400 MHz, CDCl₃) δ ppm 9.88-9.53 (m, 1H), 8.70-8.21 (m, 1H), 7.90-7.52 (m, 2H), 7.25-6.98 (m, 2H), 5.94-5.58 (m, 1H), 4.75-4.33 (m, 2H), 4.25-3.85 (m, 2H), 3.78-3.48 (m, 3H), 3.44-3.22 (m, 2H), 2.66-2.31 (m, 2H), 2.03-1.92 (m, 1H), 1.69-1.49 (m, 4H), 1.14-0.88 (m, 6H).

### Step 2: (1R,2S,5S)—N—((S)-1-hydroxy-3-((S)-2-oxopyrrolidin-3-yl)propan-2-yl)-6,6-dimethyl-3-(7-(trifluoromethyl)-1H-indole-2-carbonyl)-3-azabicyclo[3.1.0]hexane-2-carboxamide

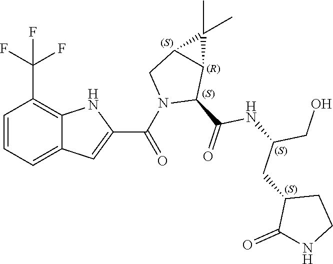

To a solution of (S)-methyl 2-((1R,2S,5S)-6,6-dimethyl-3-(7-(trifluoromethyl)-1H-indole-2-carbonyl)-3-azabicyclo[3.1.0]hexane-2-carboxamido)-3-((S)-2-oxopyrrolidin-3-yl)propanoate (95 mg, 177.73 mol) in THE (6 mL) was added LiBH₄ (12 mg, 533.18 mol) dropwise at 0° C. The mixture was stirred at 0° C. for 1 hr. H₂O (15 mL) was added to the reaction mixture. The resulting mixture was extracted with DCM (15 mL×3). The combined organic phase was washed with brine (15 mL), water (15 mL), dried over anhydrous Na₂SO₄, filtered and concentrated to dryness. The title compound (104 mg, crude) was obtained as a white solid. ¹H NMR (400 MHz, CDCl₃) δ ppm 10.27-9.51 (m, 1H), 8.48-8.20 (m, 1H), 7.91-7.52 (m, 2H), 7.24-7.00 (m, 2H), 5.73 (brs, 1H), 4.74-3.92 (m, 3H), 3.78-3.74 (m, 2H), 3.63-3.47 (m, 2H), 3.44-3.23 (m, 2H), 2.67-2.27 (m, 2H), 2.03-1.84 (m, 3H), 1.75-1.51 (m, 2H), 1.10-0.89 (m, 6H).

### Step 3: (1R,2S,5S)-6,6-dimethyl-N—((S)-1-oxo-3-((S)-2-oxopyrrolidin-3-yl)propan-2-yl)-3-(7-(trifluoromethyl)-1H-indole-2-carbonyl)-3-azabicyclo[3.1.0]hexane-2-carboxamide

To a solution of (1R,2S,5S)—N—((S)-1-hydroxy-3-((S)-2-oxopyrrolidin-3-yl)propan-2-yl)-6,6-dimethyl-3-(7-(trifluoromethyl)-1H-indole-2-carbonyl)-3-azabicyclo[3.1.0]hexane-2-carboxamide (100 mg, 197.43 mol) in DCM (6 mL) was added DMP (252 mg, 592.28 mol) at 25° C. The reaction mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 26%-56%, 10 min). Compound 29 (29.9 mg, 29.44% yield, 98.07% purity) was obtained as a white solid. ¹H NMR (400 MHz, CD₃OD) δ ppm 7.99-7.80 (m, 1H), 7.61-7.53 (m, 1H), 7.29-7.01 (m, 2H), 5.01-4.89 (m, 1H), 4.54-3.97 (m, 3H), 3.93-3.36 (m, 2H), 2.96-2.57 (m, 1H), 2.41-1.68 (m, 4H), 1.66-1.52 (m, 2H), 1.30 (s, 1H), 1.16-0.97 (m, 6H). ESI-MS [M+H]⁺ calc'd for C₂₅H₂₇F₃N₄O₄, 505.20; found, 505.4.

### Example 30: (1R,2S,5S)-6,6-dimethyl-N—((S)-1-oxo-3-((S)-2-oxopyrrolidin-3-yl)propan-2-yl)-3-(4H-thieno[3,2-b]pyrrole-5-carbonyl)-3-azabicyclo[3.1.0]hexane-2-carboxamide (Compound 30)

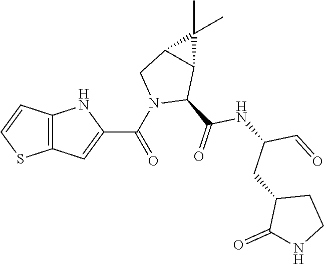 [SMILES: CC1(C)[C@@H]2[C@@H](C(=O)N[C@H](C=O)C[C@@H]3CCNC3=O)N(C(=O)c3cc4sccc4[nH]3)C[C@@H]21]

### Step 1: (S)-methyl 2-((1R,2S,5S)-6,6-dimethyl-3-(4H-thieno[3,2-b]pyrrole-5-carbonyl)-3-azabicyclo[3.1.0]hexane-2-carboxamido)-3-((S)-2-oxopyrrolidin-3-yl)propanoate

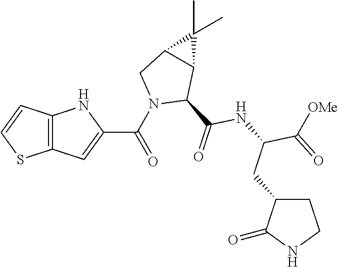 [SMILES: COC(=O)[C@H](C[C@@H]1CCNC1=O)NC(=O)[C@@H]1[C@@H]2[C@H](CN1C(=O)c1cc3sccc3[nH]1)C2(C)C]

To a mixture of 4H-thieno[3,2-b]pyrrole-5-carboxylic acid (75 mg, 444.63 mol) and DIEA (229 mg, 1.78 mmol) in DCM (5 mL) was added T₃P (424 mg, 666.95 μmol, 50% purity in EtOAc) at 0° C. After addition, the reaction mixture was stirred at the same temperature for 30 min. The above solution was added to a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (160 mg, 444.63 μmol, HCl) in DCM (2 mL) at 0° C. Then the resulting mixture was degassed and purged with N₂ for 3 times, and the mixture was stirred at 25° C. for 10 hours under N₂ atmosphere. The reaction mixture was concentrated. To the residue was added water (10 mL). The resulting mixture was extracted with DCM (10 mL×3). The combined organic phase was washed with brine (10 mL), dried over anhydrous Na₂SO₄, filtered and concentrated to dryness. The residue was purified by prep-HPLC (column: Xtimate C18 150×40 mm×10 μm; mobile phase: [water (0.225% FA)-ACN]; B %: 30%-60%, 10 min). The title compound (70 mg, 28.98% yield) was obtained as a white solid. ¹H NMR (400 MHz, CDCl₃) δ ppm 9.74-9.39 (m, 1H), 8.53-7.52 (m, 1H), 7.23-7.13 (m, 1H), 6.98-6.70 (m, 2H), 6.04-5.68 (m, 1H), 4.64-4.26 (m, 2H), 4.14-3.99 (m, 1H), 3.91-3.81 (m, 1H), 3.70-3.48 (m, 3H), 3.36-3.16 (m, 2H), 2.59-2.26 (m, 2H), 2.16-2.04 (m, 1H), 2.02-1.70 (m, 2H), 1.62-1.54 (m, 2H), 1.05-0.99 (m, 3H), 0.84 (s, 3H)

### Step 2: (1R,2S,5S)—N—((S)-1-hydroxy-3-((S)-2-oxopyrrolidin-3-yl)propan-2-yl)-6,6-dimethyl-3-(4H-thieno[3,2-b]pyrrole-5-carbonyl)-3-azabicyclo[3.1.0]hexane-2-carboxamide

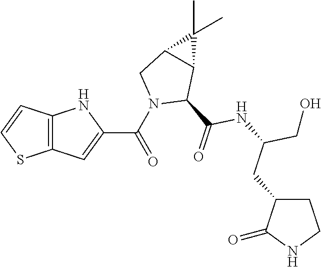 [SMILES: CC1(C)[C@@H]2[C@@H](C(=O)N[C@H](CO)C[C@@H]3CCNC3=O)N(C(=O)c3cc4sccc4[nH]3)C[C@@H]21]

To a solution of (S)-methyl 2-((1R,2S,5S)-6,6-dimethyl-3-(4H-thieno[3,2-b]pyrrole-5-carbonyl)-3-azabicyclo[3.1.0]hexane-2-carboxamido)-3-((S)-2-oxopyrrolidin-3-yl)propanoate (70 mg, 148.13 mol) in THE (5 mL) was added LiBH₄ (10 mg, 444.39 mol) at 0° C. Then the mixture was stirred at 25° C. for 2 hours under N₂ atmosphere. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The crude product was used in the next step directly. The title compound (47 mg, 67.80% yield) was obtained as a white solid. ¹H NMR (400 MHz, CDCl₃) δ ppm 10.12-9.70 (m, 1H), 8.06-7.58 (m, 1H), 7.22-7.15 (m, 1H), 6.94-6.71 (m, 2H), 5.96-5.68 (m, 1H), 4.53 (s, 1H), 4.20-4.09 (m, 1H), 3.98-3.75 (m, 2H), 3.65-3.52 (m, 1H), 3.50-3.35 (m, 1H), 3.28-3.04 (m, 2H), 2.46-2.35 (m, 1H), 2.30-2.18 (m, 1H), 2.03-1.89 (m, 1H), 1.70-1.65 (m, 1H), 1.61-1.41 (m, 3H), 0.99 (s, 3H), 0.87-0.76 (m, 3H)

Step 3: (1R,2S,5S)-6,6-dimethyl-N—((S)-1-oxo-3-((S)-2-oxopyrrolidin-3-yl)propan-2-yl)-3-(4H-thieno[3,2-b]pyrrole-5-carbonyl)-3-azabicyclo[3.1.0]hexane-2-carboxamide

To a solution of (1R,2S,5S)—N—((S)-1-hydroxy-3-((S)-2-oxopyrrolidin-3-yl)propan-2-yl)-6,6-dimethyl-3-(4H-thieno[3,2-b]pyrrole-5-carbonyl)-3-azabicyclo[3.1.0]hexane-2-carboxamide (40 mg, 89.98 mol) in DCM (6 mL) was added DMP (77 mg, 179.96 mol) at 25° C. The reaction mixture was stirred at 25° C. for 0.5 hr. The reaction mixture was filtered and the filtrate was concentrated. The residue was purified by prep-HPLC (column: Phenomenex Gemini-NX C18 75×30 mm×3 μm; mobile phase: [water (10 mM NH₄HCO₃)-ACN]; B %: 15%-45%, 8 min). Compound 30 (3.5 mg, 8.65% yield, 98.413% purity) was obtained as a white solid. ¹H NMR (400 MHz, CD₃OD) δ ppm 7.37-7.25 (m, 1H), 7.06-6.84 (m, 2H), 4.61-4.40 (m, 1H), 4.26-3.73 (m, 3H), 3.28-2.98 (m, 2H), 2.69-2.27 (m, 2H), 2.06-1.93 (m, 1H), 1.80-1.60 (m, 2H), 1.59-1.39 (m, 2H), 1.16-0.92 (m, 6H). ESI-MS [M+H]⁺ calc'd for C₂₂H₂₆N₄O₄S, 443.17, found 443.4.

### Example 31: (S)-5-(4-Methoxy-1H-indole-2-carbonyl)-N—((S)-1-oxo-3-((S)-2-oxopyrrolidin-3-yl)propan-2-yl)-5-azaspiro[2.4]heptane-6-carboxamide (Compound 31)

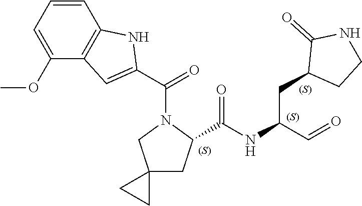

Compound 31 was synthesized by the same procedure as Compound 2. ¹H NMR (400 MHz, CDCl₃) δ ppm 9.69-9.53 (m, 1H), 9.53-9.44 (m, 1H), 8.66-8.39 (m, 1H), 7.24-7.18 (m, 1H), 7.07-6.97 (m, 2H), 6.50 (m, 1H), 5.88-5.61 (m, 1H), 5.09-4.90 (m, 1H), 4.44-4.25 (m, 1H), 3.96 (s, 3H), 3.94-3.88 (m, 2H), 3.35-3.23 (m, 2H), 2.67-2.31 (m, 3H), 2.30-2.21 (m, 1H), 2.17-2.09 (m, 1H), 1.98-1.91 (m, 2H), 0.72-0.63 (m, 4H). ESI-MS [M+H]⁺ calc'd for C₂₄H₂₈N₄O₅, 453.21; found, 453.2.

### Example 32: (1R,2S,5S)-3-(7-cyano-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 32)

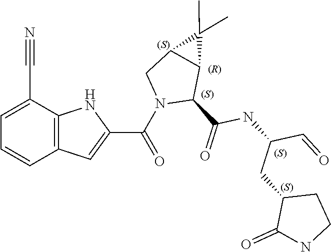

### Step 1: (S)-methyl 2-((1R,2S,5S)-3-(7-cyano-1H-indole-2-carbonyl)-6,6-dimethyl-3-azabicyclo[3.1.0]hexane-2-carboxamido)-3-((S)-2-oxopyrrolidin-3-yl)propanoate

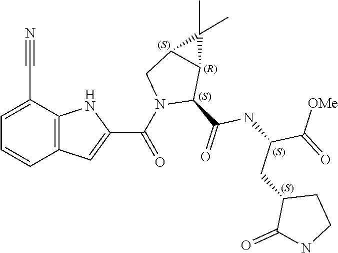

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (250 mg, 694.74 μmol, HCl), 7-cyano-1H-indole-2-carboxylic acid (129 mg, 694.74 mol), and DIEA (269 mg, 2.08 mmol) in DCM (4 mL) was added HATU (528 mg, 1.39 mmol) slowly at 0° C. The mixture was stirred at 25° C. for 3 hr. The reaction mixture was diluted with 5% H₃PO₄ (30 mL) and was extracted with DCM (30 mL×2). The combined organic layer was washed with 5% H₃PO₄ (20 mL×3), sat.aq.NaHCO₃ (20 mL×4), water (20 mL), and brine (20 mL), and dried over anhydrous Na₂SO₄, filtered and concentrated to dryness. The residue was purified by prep-HPLC (column: Xtimate C18 150×40 mm×10 μm; mobile phase: [water (10 mM NH₄HCO₃)-ACN]; B %: 15%-45%, 10 min). The title compound (150 mg, 43.04% yield) was obtained as a white solid. ¹H NMR (400 MHz, CDCl₃) δ ppm 11.84 (br s, 1H), 9.39 (br d, J=3.6 Hz, 1H), 7.97-7.86 (m, 1H), 7.65-7.53 (m, 1H), 7.23-7.12 (m, 2H), 7.05-6.95 (m, 1H), 4.95 (s, 1H), 4.28-4.20 (m, 1H), 4.15-4.05 (m, 1H), 3.73 (s, 3H), 3.52-3.44 (m, 2H), 3.42-3.37 (m, 1H), 2.73-2.61 (m, 1H), 2.56-2.43 (m, 1H), 2.12-1.99 (m, 2H), 1.80-1.76 (m, 1H), 1.72-1.69 (m, 1H), 1.33-1.25 (m, 1H), 1.01 (s, 3H), 0.89 (s, 3H).

### Step 2: (1R,2S,5S)-3-(7-cyano-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

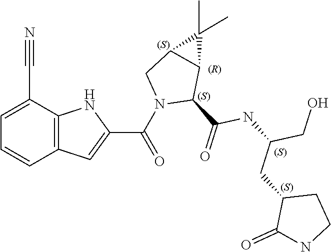

To a solution of (S)-methyl 2-((1R,2S,5S)-3-(7-cyano-1H-indole-2-carbonyl)-6,6-dimethyl-3-azabicyclo[3.1.0]hexane-2-carboxamido)-3-((S)-2-oxopyrrolidin-3-yl)propanoate (145 mg, 294.99 mol) in THE (3 mL) was added LiBH₄ (26 mg, 1.18 mmol) at 0° C. The mixture was stirred at 25° C. for 1 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated to dryness. The title compound (103 mg, crude) was obtained as a white solid.

¹H NMR (400 MHz, DMSO-d) δ ppm 8.02 (d, J=7.2 Hz, 1H), 7.98-7.78 (m, 1H), 7.76-7.65 (m, 1H), 7.62-7.48 (m, 1H), 7.37-7.14 (m, 2H), 7.11-6.87 (m, 1H), 4.75-4.68 (m, 1H), 4.25-4.12 (m, 1H), 3.87-3.71 (m, 2H), 3.68-3.54 (m, 2H), 3.17-2.81 (m, 2H), 2.40-2.30 (m, 1H), 2.23-2.07 (m, 1H), 1.64-1.50 (m, 2H), 1.49-1.32 (m, 3H), 1.21-1.14 (m, 1H), 1.08-1.02 (m, 3H), 0.99-0.80 (m, 3H).

### Step 3: (1R,2S,5S)-3-(7-cyano-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-cyano-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (50 mg, 107.87 mol) in DCM (5 mL) was added DMP (137 mg, 323.61 mol). The mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 14%-54%, 11 min). Compound 32 (15 mg, 29.08% yield, 96.51% purity) was obtained as a white solid. ¹H NMR (400 MHz, CD₃OD) δ ppm 8.07-7.83 (m, 1H), 7.73-7.58 (m, 1H), 7.26-6.99 (m, 2H), 4.67-4.60 (m, 1H), 4.42-4.26 (m, 1H), 4.09-3.97 (m, 1H), 3.94-3.67 (m, 2H), 3.28-2.92 (m, 1H), 2.71-2.28 (m, 2H), 2.12-1.99 (m, 1H), 1.82-1.52 (m, 4H), 1.22-0.92 (m, 6H). ESI-MS [M+H]⁺ calc'd for C₂₅H₂₇N₅O₄, 462.21; found, 462.1.

### Example 33: (1R,2S,5S)-6,6-Dimethyl-3-(7-methyl-1H-indole-2-carbonyl)-N—((S)-1-oxo-3-((S)-2-oxopyrrolidin-3-yl)propan-2-yl)-3-azabicyclo[3.1.0]hexane-2-carboxamide (Compound 33)

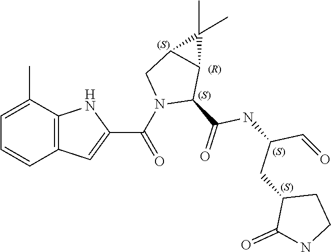

Compound 33 was synthesized by the same procedure as Compound 1. ¹H NMR (400 MHz, CD₃OD) δ ppm 7.54-7.32 (m, 1H), 7.09-6.87 (m, 3H), 5.02-4.96 (m, 0.5H), 4.68-4.62 (m, 0.5H), 4.53-4.27 (m, 1H), 4.08-3.42 (m, 3H), 3.29-3.23 (m, 1H), 2.90-2.55 (m, 1H), 2.51 (s, 3H), 2.40-2.29 (m, 1H), 2.24-1.97 (m, 1H), 1.89-1.67 (m, 2H), 1.62-1.50 (m, 1H), 1.41-1.26 (m, 1H), 1.17-0.93 (m, 6H). ESI-MS [M+H]⁺ calc'd for C₂₅H₃₀N₄O₄, 451.23; found, 451.4.

### Example 34: (1R,2S,5S)-3-(4-isopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 34)

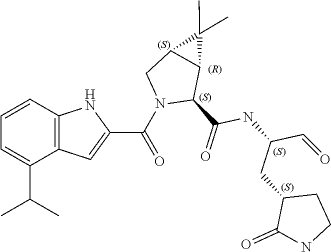

### Step 1: methyl 4-(prop-1-en-2-yl)-1H-indole-2-carboxylate

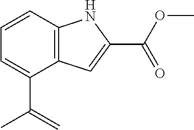 [SMILES: C=C(C)c1cccc2[nH]c(C(=O)OC)cc12]

To a solution of methyl 4-bromo-1H-indole-2-carboxylate (490 mg, 1.93 mmol) and potassium trifluoro(prop-1-en-2-yl)borate (571 mg, 3.86 mmol) in dioxane (8 mL) and H₂O (2 mL) was added Cs₂CO₃ (1.89 g, 5.79 mmol) and Pd(dppf)Cl₂ (141 mg, 192.85 mol). Then the mixture was stirred at 100° C. for 10 hours under N₂ atmosphere. The reaction mixture was filtered. Saturated aq.NH₄Cl (20 mL) was added to the filtrate and the mixture was extracted with DCM (20 mL×2). The combined organic phase was washed with brine (20 mL×2), dried over anhydrous Na₂SO₄, filtered and concentrated in vacuo. The residue was purified by flash silica gel chromatography (eluent of 0-16% PE/EtOAc). The title compound (410 mg, 96.27% yield) was obtained as a white solid. ESI-MS [M+H]⁺ calc'd for C₁₃H₁₃NO₂, 216.09; found, 216.1.

### Step 2: methyl 4-isopropyl-1H-indole-2-carboxylate

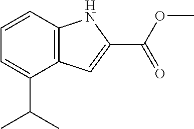 [SMILES: COC(=O)c1cc2c(C(C)C)cccc2[nH]1]

To a solution of methyl 4-(prop-1-en-2-yl)-1H-indole-2-carboxylate (400 mg, 1.86 mmol) in MeOH (10 mL) was added wet Pd/C (0.09 g, 10% purity) slowly at 25° C. Then the mixture was degassed and purged with H₂ for 3 times. The mixture was stirred at 25° C. for 10 hours under H₂ atmosphere. The reaction mixture was filtered and the cake was washed with MeOH (5 mL×2). The combined filtrate was concentrated in vacuo. The title compound (220 mg, 54.49% yield) was obtained as a white solid. ¹H NMR (400 MHz, CDCl₃) δ ppm 8.85 (s, 1H), 7.34 (d, J=2.4 Hz, 1H), 7.29-7.26 (m, 2H), 7.03-7.01 (m, 1H), 3.40-3.32 (m, 1H), 1.38 (d, J=7.2 Hz, 1H)

### Step 3: 4-isopropyl-1H-indole-2-carboxylic acid

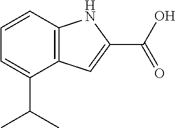

To a solution of methyl 4-isopropyl-1H-indole-2-carboxylate (220 mg, 1.01 mmol) in THF (5 mL) and H₂O (1 mL) was added LiOH·H₂O (85 mg, 2.03 mL) in one portion. Then the mixture was stirred at 25° C. for 10 hours. Water (10 mL) was added. Then the mixture was washed with DCM (5 mL×2). The aqueous phase was adjusted to pH=3 with HCl (1N). The resulting mixture was extracted with DCM (6 mL×3). The combined organic phase was washed with brine (10 mL), dried over anhydrous Na₂SO₄, filtered and concentrated in vacuo. The title compound (220 mg, crude) was obtained as a light brown solid. ESI-MS [M+H]⁺ calc'd for C₁₂H₁₃NO₂, 204.09; found, 204.1.

### Step 4: (S)-methyl 2-((1R,2S,5S)-3-(4-isopropyl-1H-indole-2-carbonyl)-6,6-dimethyl-3-azabicyclo[3.1.0]hexane-2-carboxamido)-3-((S)-2-oxopyrrolidin-3-yl)propanoate

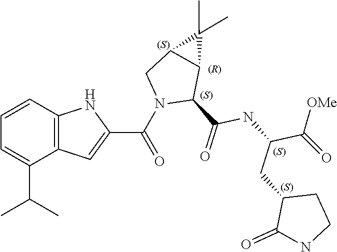

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (169 mg, 469.64 μmol, HCl) and 4-isopropyl-1H-indole-2-carboxylic acid (95 mg, 469.64 mol) in DCM (5 mL) was added DIPEA (243 mg, 1.88 mmol) dropwise and HATU (268 mg, 704.47 mol) slowly at 0° C. Then the mixture was stirred at 25° C. for 3 hours. Saturated aq. NaHCO₃ (10 mL) was added and the mixture was extracted with DCM (10 mL×3). The combined organic phase was washed with brine (10 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 29%-69%, 11 min). The title compound (140 mg, 53.15% yield) was obtained as a white solid. ¹H NMR (400 MHz, CD₃OD) δ ppm 7.30-7.23 (m, 1H), 7.19 (t, J=7.8 Hz, 1H), 7.15-7.07 (m, 1H), 6.96-6.90 (m, 1H), 4.62-4.54 (m, 1H), 4.37-4.29 (m, 1H), 4.08-3.97 (m, 1H), 3.77-3.60 (m, 3H), 3.48-3.36 (m, 1H), 3.30-3.27 (m, 3H), 2.97-2.62 (m, 1H), 2.48-2.30 (m, 1H), 2.25-1.95 (m, 1H), 1.91-1.61 (m, 3H), 1.60-1.26 (m, 7H), 1.18-0.97 (m, 6H).

### Step 5: (1R,2S,5S)—N—((S)-1-hydroxy-3-((S)-2-oxopyrrolidin-3-yl)propan-2-yl)-3-(4-isopropyl-1H-indole-2-carbonyl)-6,6-dimethyl-3-azabicyclo[3.1.0]hexane-2-carboxamide

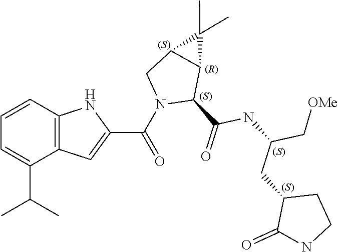

To a solution of (S)-methyl 2-((1R,2S,5S)-3-(4-isopropyl-1H-indole-2-carbonyl)-6,6-dimethyl-3-azabicyclo[3.1.0]hexane-2-carboxamido)-3-((S)-2-oxopyrrolidin-3-yl)propanoate (140 mg, 275.26 mol) in THE (5 mL) was added LiBH₄ (70 mg, 3.21 mmol) slowly at 0° C. Then the mixture was stirred at 0° C. for 1 hour. Saturated aq. NH₄Cl (10 mL) was added to the reaction mixture dropwise. Then the aqueous phase was extracted with DCM (10 mL×3). The combined organic phase was washed with brine (10 mL×2), dried over anhydrous Na₂SO₄, filtered and concentrated in vacuo. The title compound (170 mg, crude) was obtained as a light yellow solid. ESI-MS [M+H]⁺ calc'd for C₂₇H₃₆N₄O₄, 481.27; found, 481.3.

### Step 6: (1R,2S,5S)-3-(4-isopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)—N—((S)-1-hydroxy-3-((S)-2-oxopyrrolidin-3-yl)propan-2-yl)-3-(4-isopropyl-1H-indole-2-carbonyl)-6,6-dimethyl-3-azabicyclo[3.1.0]hexane-2-carboxamide (80 mg, 166.46 mol) in DCM (4 mL) was added DMP (212 mg, 499.38 mol) slowly. Then the mixture was stirred at 25° C. for 30 minutes. The reaction mixture was filtered. Saturated aq. NaHCO₃ (5 mL) was added to the filtrate and the mixture was extracted with DCM (4 mL×2). The combined organic phase was washed with brine (5 mL), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 24%-64%, 11 min). Compound 34 (9 mg, 10.98% yield, 97.228% purity) was obtained as a white solid. ¹H NMR (400 MHz, CD₃OD) δ ppm 7.29-7.15 (m, 2H), 7.14-6.87 (m, 2H), 4.68-4.48 (m, 1H), 4.40-4.30 (m, 1H), 4.10-3.72 (m, 2H), 3.51-3.32 (m, 2H), 3.27-3.21 (m, 1H), 2.88-2.57 (m, 1H), 2.41-2.12 (m, 1H), 2.10-1.50 (m, 5H), 1.40-1.28 (m, 6H), 1.18-0.95 (m, 6H). ESI-MS [M+H]⁺ calc'd for C₂₇H₃₄N₄O₄, 479.26; found, 479.1.

### Example 35 (S)—N—((S)-4-Amino-3,4-dioxo-1-((S)-2-oxopyrrolidin-3-yl)butan-2-yl)-5-(1H-indole-2-carbonyl)-5-azaspiro[2.4]heptane-6-carboxamide (Compound 35)

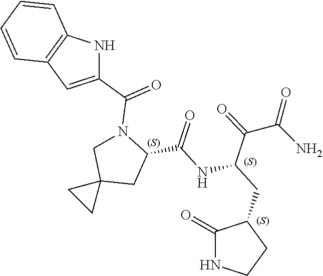

Compound 35 was synthesized by the same procedure as Compound 50. ¹H NMR (400 MHz, CD₃OD) δ ppm 7.69-7.50 (m, 1H), 7.48-7.40 (m, 1H), 7.27-7.17 (m, 1H), 7.10-6.82 (m, 2H), 5.35-4.89 (m, 1H), 4.69-4.46 (m, 1H), 4.41-4.07 (m, 1H), 4.06-3.82 (m, 1H), 3.79-3.62 (m, 1H), 2.98-2.60 (m, 1H), 2.58-2.34 (m, 1H), 2.32-2.13 (m, 1H), 2.11-1.89 (m, 2H), 1.88-1.49 (m, 2H), 1.46-1.15 (m, 1H), 0.81-0.57 (m, 4H). ESI-MS [M+H]⁺ calc'd for C₂₄H₂₇N₅O₅, 466.20; found, 466.4.

### Example 36: (1R,2S,5S)-3-(7-Fluoro-4-methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 36)

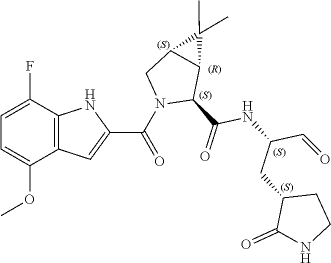

Compound 36 was synthesized by the same procedure as Compound 1. ¹H NMR (400 MHz, CD₃OD) δ ppm 7.14-6.96 (m, 1H), 6.92-6.80 (m, 1H), 6.44-6.30 (m, 1H), 4.65-4.62 (m, 1H), 4.53-4.26 (m, 1H), 4.06-3.95 (m, 1H), 3.93-3.82 (m, 3H), 3.80-3.71 (m, 1H), 3.38-3.33 (m, 1H), 3.29-3.24 (m, 1H), 2.93-2.17 (m, 2H), 2.15-1.73 (m, 2H), 1.70-1.41 (m, 2H), 1.40-1.32 (m, 1H), 1.18-0.91 (m, 6H). ESI-MS [M+H]⁺ calc'd for C₂₅H₂₉FN₄O₅, 485.21; found, 485.4.

### Example 37: (1R,2S,5S)-6,6-dimethyl-N—((S)-1-oxo-3-((S)-2-oxopyrrolidin-3-yl)propan-2-yl)-3-((R)-2-phenylpropanoyl)-3-azabicyclo[3.1.0]hexane-2-carboxamide (Compound 37)

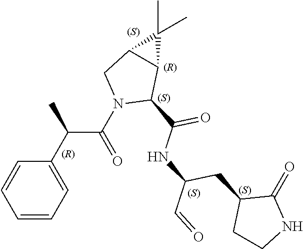

### Step 1: (S)-methyl 2-((1R,2S,5S)-6,6-dimethyl-3-((R)-2-phenylpropanoyl)-3-azabicyclo[3.1.0]hexane-2-carboxamido)-3-((S)-2-oxopyrrolidin-3-yl)propanoate

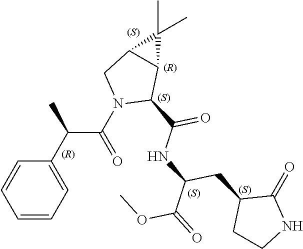

To a solution of (R)-2-phenylpropanoic acid (70 mg, 469.64 mol) and DIEA (242 mg, 1.88 mmol) in DCM (5 mL) was added dropwise T₃P (448 mg, 704.47 μmol, 50% purity in EtOAc) dropwise at 0° C. After addition, the mixture was stirred at the same temperature for 30 min, and then (S)-methyl 2-((1R,2S,5S)-6,6-dimethyl-3-azabicyclo[3.1.0]hexane-2-carboxamido)-3-((S)-2-oxopyrrolidin-3-yl)propanoate (169 mg, 469.64 μmol, HCl) was added at 25° C. The resulting mixture was stirred at 25° C. for 11.5 hrs. The reaction mixture was diluted with DCM (30 mL). The combined organic phase was washed with 5% H₃PO₄ (15 mL×2), sat.aq. NaHCO₃ (15 mL×2), dried over anhydrous Na₂SO₄, filtered and concentrated in vacuo. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 19%-59%, 11 min). The title compound (38 mg, 16.48% yield) was obtained as a white solid. ¹H NMR (400 MHz, CDCl₃) δ ppm 7.60 (d, J=7.2 Hz, 1H), 7.27-7.22 (m, 5H), 5.50 (s, 1H), 4.65-4.58 (m, 1H), 4.30 (s, 1H), 3.87-3.80 (m, 1H), 3.77 (s, 3H), 3.70-3.60 (m, 1H), 3.40-3.33 (m, 3H), 2.67-2.44 (m, 2H), 2.27-2.16 (m, 1H), 2.02-1.84 (m, 2H), 1.45-1.39 (m, 5H), 0.94 (s, 3H), 0.41 (s, 3H).

### Step 2: (1R,2S,5S)—N—((S)-1-hydroxy-3-((S)-2-oxopyrrolidin-3-yl)propan-2-yl)-6,6-dimethyl-3-((R)-2-phenylpropanoyl)-3-azabicyclo[3.1.0]hexane-2-carboxamide

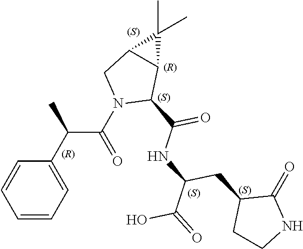

To a mixture of methyl (S)-methyl 2-((1R,2S,5S)-6,6-dimethyl-3-((R)-2-phenylpropanoyl)-3-azabicyclo[3.1.0]hexane-2-carboxamido)-3-((S)-2-oxopyrrolidin-3-yl)propanoate (35 mg, 76.83 mol) in THE (2 mL) was added LiBH₄ (5 mg, 230.49 mol) at 0° C. under N₂. The mixture was stirred at 0° C. for 1 hr. The mixture was quenched with H₂O (2 mL), extracted with DCM (5 mL×3). The combined organic layer was dried over anhydrous Na₂SO₄, filtered and concentrated in vacuo. The residue was used to next step directly. The title compound (36 mg, 83.29% yield) was obtained as a white solid. ESI-MS [M+H]⁺ calc'd for C₂₄H₃₃N₃O₄ 428.25, found 428.2.

### Step 3: (1R,2S,5S)-6,6-dimethyl-N—((S)-1-oxo-3-((S)-2-oxopyrrolidin-3-yl)propan-2-yl)-3-((R)-2-phenylpropanoyl)-3-azabicyclo[3.1.0]hexane-2-carboxamide

To a mixture of (1R,2S,5S)—N—((S)-1-hydroxy-3-((S)-2-oxopyrrolidin-3-yl)propan-2-yl)-6,6-dimethyl-3-((R)-2-phenylpropanoyl)-3-azabicyclo[3.1.0]hexane-2-carboxamide (30 mg, 70.17 mol) in DCM (2 mL) was added DMP (89 mg, 210.51 mol). The mixture was stirred at 25° C. for 0.5 hr. The mixture was filtered and the filtrate was concentrated to give crude product. The residue was purified by prep-HPLC (column: Phenomenex Gemini-NX C18 75×30 mm×3 μm; mobile phase: [water (10 mM NH₄HCO₃)-ACN]; B %: 24%-54%, 10 min). Compound 37 (8.6 mg, 28.51% yield) was obtained as a white solid. ¹H NMR (400 MHz, CD₃OD) δ ppm 7.35-7.22 (m, 5H), 4.63-4.48 (m, 1H), 4.27-4.22 (m, 1H), 4.08-3.98 (m, 1H), 3.95-3.87 (m, 1H), 3.81 (q, J=6.8 Hz, 1H), 3.31-3.29 (m, 2H), 2.74-2.61 (m, 1H), 2.47-2.32 (m, 1H), 2.11-1.99 (m, 1H), 1.82-1.54 (m, 2H), 1.47-1.38 (m, 2H), 1.38-1.33 (m, 3H), 0.97-0.92 (m, 3H), 0.46-0.38 (m, 3H). ESI-MS [M+H]⁺ calc'd for C₂₄H₃₁N₃O₄ 426.23, found 426.2.

### Example 38: (2S,4S)-4-Cyclohexyl-1-(4-fluoro-1H-indole-2-carbonyl)-N—((S)-1-oxo-3-((S)-2-oxopyrrolidin-3-yl)propan-2-yl)pyrrolidine-2-carboxamide (Compound 38)

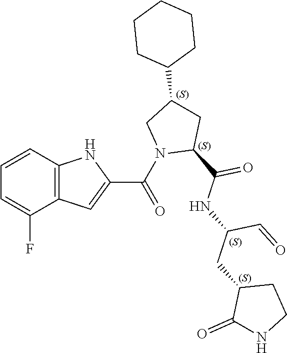

Compound 38 was synthesized by the same procedure as Compound 2.

¹H NMR (400 MHz, CD₃OD) δ ppm 7.32-7.25 (m, 1H), 7.24-7.15 (m, 1H), 7.12-6.87 (m, 1H), 6.84-6.68 (m, 1H), 5.17-4.91 (m, 1H), 4.85-4.24 (m, 3H), 4.19-3.92 (m, 1H), 3.92-3.56 (m, 1H), 3.11-2.32 (m, 3H), 2.24-1.94 (m, 3H), 1.85-1.49 (m, 7H), 1.38-0.97 (m, 6H). ESI-MS [M+H]⁺ calc'd for C₂₇H₃₃FN₄O₄, 497.25; found, 497.4.

### Example 39: (1R,2S,5S)-3-(7-fluoro-4-isopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 39)

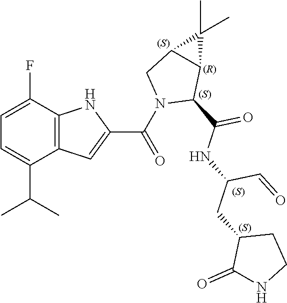

### Step 1: 2-fluoro-5-(prop-1-en-2-yl)aniline

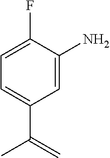

To a solution of 5-bromo-2-fluoroaniline (5.0 g, 26.3 mmol) and 4,4,5,5-tetramethyl-2-(prop-1-en-2-yl)-1,3,2-dioxaborolane (5.31 g, 78.9 mmol) in dioxane (60 mL) and H₂O (12 mL) was added Cs₂CO₃ (25.7 g, 78.9 mmol) and Pd(dppf)Cl₂ (963 mg, 1.32 mmol). The mixture was stirred at 100° C. for 12 hr under N₂. H₂O (20 mL) was added. The reaction mixture was filtered and the cake was washed with DCM (10 mL×2). The filtrate was extracted with DCM (20 mL×3). The combined organic phase was dried over anhydrous Na₂SO₄, filtered and concentrated in vacuo. The residue was purified by flash silica gel chromatography (eluent of 0~20% PE/EtOAc). The title compound (2.88 g, 72.40% yield) was obtained as yellow oil. ¹H NMR (400 MHz, CDCl₃) δ ppm 6.99-6.86 (m, 2H), 6.82-6.75 (m, 1H), 5.26 (s, 1H), 5.02 (s, 1H), 2.10 (s, 3H).

### Step 2: 2-fluoro-5-isopropylaniline

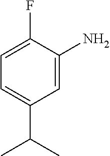

To a solution of 2-fluoro-5-(prop-1-en-2-yl)aniline (2.88 g, 19.1 mmol) in MeOH (50 mL) was added Pd/C (1.0 g, 10% purity). The mixture was stirred at 20° C. under H₂ (30 psi) for 10 hr. The reaction mixture was filtered and the filtrate was concentrated to give a crude product. The title compound (2.1 g, 71.96% yield) was obtained as red oil. ¹H NMR (400 MHz, CDCl₃) δ ppm 6.85-6.76 (m, 1H), 6.64 (dd, J=8.8, 2.0 Hz, 1H), 6.55-6.51 (m, 1H), 2.86-2.69 (m, 1H), 1.20 (d, J=6.8 Hz, 6H).

### Step 3: (2-fluoro-5-isopropylphenyl)hydrazine

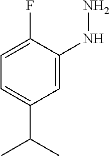

To a solution of 2-fluoro-5-isopropylaniline (1 g, 6.53 mmol) in HCl (12 M, 10 mL) was added a solution of NaNO₂ (675 mg, 9.79 mmol) in H₂O (10 mL) at 0° C. The mixture was stirred at 0° C. for 0.5 hr. SnCl₂·2H₂O (2.95 g, 13.1 mmol) was added and the mixture was stirred at 0° C. for 2 h. The reaction mixture was adjusted to pH~1 by NaOH (6 M). The resulting mixture was extracted with EtOAc (10 mL×3). The combined organic phase was washed with water (10 mL), dried over anhydrous Na₂SO₄, filtered and concentrated. The residue was purified by flash silica gel chromatography (Eluent of 0~10% EtOAc in PE). The title compound (671 mg, 61.11% yield) was obtained as a white solid. ¹H NMR (400 MHz, CDCl₃) δ ppm 7.04-6.97 (m, 1H), 6.90-6.82 (m, 1H), 6.52-6.39 (m, 2H), 3.98 (s, 2H), 2.34-2.26 (m, 1H), 1.16 (d, J=6.8 Hz, 6H).

### Step 4: ethyl 7-fluoro-4-isopropyl-1H-indole-2-carboxylate

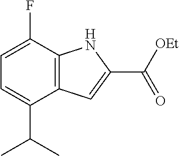 [SMILES: CCOC(=O)c1cc2c(C(C)C)ccc(F)c2[nH]1]

To a solution of (2-fluoro-5-isopropylphenyl)hydrazine (671 mg, 3.99 mmol) in toluene (5 mL) was added ethyl 2-oxopropanoate (926 mg, 7.89 mmol) and TsOH (2.06 g, 12.0 mmol) at 25° C. The mixture was stirred at 120° C. for 12 hr. Water (10 mL) was added and the reaction mixture was adjusted to pH~7 by sat.aq.NaHCO₃. The resulting mixture was extracted with EtOAc (10 mL×3). The combined organic phase was washed with water (10 mL), dried over anhydrous Na₂SO₄, filtered and concentrated in vacuo. The residue was purified by flash silica gel chromatography (Eluent of 0~10% EtOAc in PE. The title compound (251 mg, 25.24% yield) was obtained as an off-white solid. ¹H NMR (400 MHz, CDCl₃) δ ppm 9.02 (br s, 1H), 7.26 (dd, J=2.4, 3.2 Hz, 1H), 6.93-6.85 (m, 1H), 6.84-6.78 (m, 1H), 4.36 (q, J=7.2 Hz, 2H), 3.33-3.13 (m, 1H), 1.36 (t, J=7.2 Hz, 3H)

### Step 5: 7-fluoro-4-isopropyl-1H-indole-2-carboxylic acid

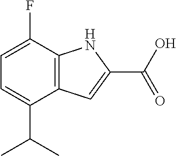 [SMILES: CC(C)c1ccc(F)c2[nH]c(C(=O)O)cc12]

To a solution of ethyl 7-fluoro-4-isopropyl-1H-indole-2-carboxylate (200 mg, 802.31 mol) in THE (2 mL) and H₂O (2 mL) was added LiOH·H₂O (67 mg, 1.60 mmol). And the mixture was stirred at 25° C. for 10 hr. Water (10 mL) was added and the reaction mixture was adjusted to pH~5 by aq. HCl (1 N). The resulting mixture was extracted with DCM (20 mL×3). The combined organic phase was washed with brine (10 mL), dried over anhydrous Na₂SO₄, filtered and concentrated in vauco. The title compound (100 mg, crude) was obtained as a yellow solid. ¹H NMR (400 MHz, CD₃OD) δ ppm 7.29 (d, J=3.2 Hz, 1H), 6.96-6.82 (m, 2H), 3.39-3.32 (m, 1H), 1.35 (d, J=7.2 Hz, 6H).

### Step 6: (S)-methyl 2-((1R,2S,5S)-3-(7-fluoro-4-isopropyl-1H-indole-2-carbonyl)-6,6-dimethyl-3-azabicyclo[3.1.0]hexane-2-carboxamido)-3-((S)-2-oxopyrrolidin-3-yl)propanoate

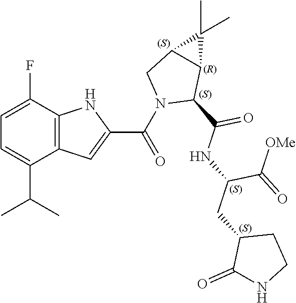

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (163 mg, 452.02 μmol, HCl), 7-fluoro-4-isopropyl-1H-indole-2-carboxylic acid (100 mg, 452.02 mol) and DIEA (175 mg, 1.36 mmol) in DCM (3 mL) was added slowly HATU (344 mg, 904.05 mol) at 0° C. The resulting mixture was stirred at 25° C. for 3 hr. Sat.aq.NH₄Cl (10 mL) was added to the residue. The resulting mixture was extracted with DCM (20 mL×3). The combined organic phase was washed with water (10 mL), dried over anhydrous Na₂SO₄, filtered and concentrated in vacuo. The residue was purified by prep-HPLC (column: Phenomenex C18 75×30 mm×3 μm; mobile phase: [water (NH₃H₂O+NH₄HCO₃)-ACN]; B %: 34%-64%, 10 min). The title compound (45 mg, 12.87% yield) was obtained as a white solid. ¹H NMR (400 MHz, CDCl₃) δ ppm 9.79 (s, 1H), 8.62-7.90 (m, 1H), 7.08-6.97 (m, 1H), 6.97-6.76 (m, 2H), 6.19-5.80 (m, 1H), 4.78-4.64 (m, 1H), 4.60-4.37 (m, 1H), 4.36-4.12 (m, 1H), 4.07-3.90 (m, 1H), 3.77-3.40 (m, 3H), 3.38-3.07 (m, 3H), 2.67-2.29 (m, 2H), 2.26-2.12 (m, 1H), 1.94-1.72 (m, 4H), 1.40-1.26 (m, 6H), 1.15-0.86 (m, 6H).

### Step 7: (1R,2S,5S)-3-(7-fluoro-4-isopropyl-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

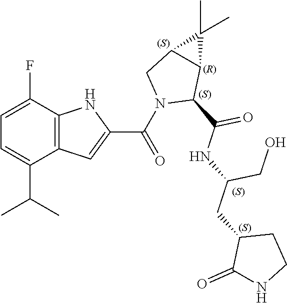

To a solution of (S)-methyl 2-((1R,2S,5S)-3-(7-fluoro-4-isopropyl-1H-indole-2-carbonyl)-6,6-dimethyl-3-azabicyclo[3.1.0]hexane-2-carboxamido)-3-((S)-2-oxopyrrolidin-3-yl)propanoate (45 mg, 85.45 mol) in THE (3 mL) was added LiBH₄ (6 mg, 256.36 mol) at 0° C. The mixture was stirred at 25° C. for 1 h. The reaction was quenched with water (10 mL) drop-wise. The resulting mixture was extracted with DCM (10 mL×3). The combined organic phase was washed with water (10 mL), dried over anhydrous Na₂SO₄, filtered and concentrated. The title compound (50 mg, crude) was obtained as a white solid. ¹H NMR (400 MHz, CDCl₃) δ ppm 10.17-9.43 (m, 1H), 9.03-7.87 (m, 1H), 7.10-6.75 (m, 3H), 6.56-5.67 (m, 1H), 5.04-4.60 (m, 1H), 4.57-4.22 (m, 1H), 4.18-3.77 (m, 3H), 3.65-3.49 (m, 1H), 3.44-3.21 (m, 3H), 2.61-2.48 (m, 1H), 2.45-2.31 (m, 1H), 2.13-1.97 (m, 1H), 1.85-1.75 (m, 2H), 1.49-1.25 (m, 8H), 1.12-0.85 (m, 6H).

### Step 8: (1R,2S,5S)-3-(7-fluoro-4-isopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-fluoro-4-isopropyl-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (50 mg, 100.28 mol) in DCM (5 mL) was added DMP (128 mg, 300.85 mol). The mixture was stirred at 25° C. for 0.5 hr. The reaction mixture was filtered. The filtrate was concentrated to dryness. The residue was purified by prep-HPLC (column: Phenomenex C18 75×30 mm×3 μm; mobile phase: [water (NH₄HCO₃)-ACN]; B %: 28%-58%, 10 min). Compound 39 (7.7 mg, 14.88% yield, 96.239% purity) was obtained as a white solid. ¹H NMR (400 MHz, CD₃OD) δ ppm 7.15 (d, J=3.2 Hz, 1H), 6.96-6.79 (m, 2H), 4.42-4.29 (m, 1H), 4.09-3.94 (m, 1H), 3.94-3.66 (m, 1H), 3.50-3.34 (m, 1H), 3.29-3.12 (m, 3H), 2.96-2.56 (m, 1H), 2.43-2.26 (m, 1H), 2.13-1.91 (m, 1H), 1.86-1.67 (m, 2H), 1.66-1.50 (m, 2H), 1.49-1.23 (m, 6H), 1.22-0.86 (m, 6H). ESI-MS [M+H]⁺ calc'd for C₂₇H₃₃FN₄O₄, 497.25; found, 497.4.

### Example 40: (1R,2S,5S)-6,6-Dimethyl-N—((S)-1-oxo-3-((S)-2-oxopyrrolidin-3-yl)propan-2-yl)-3-(2-(5,6,7,8-tetrahydronaphthalen-2-yl)acetyl)-3-azabicyclo[3.1.0]hexane-2-carboxamide (Compound 40)

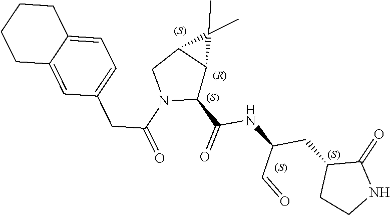

Compound 40 was synthesized by the same procedure as Compound 1. ¹H NMR (400 MHz, CD₃OD) δ ppm 7.06-6.75 (m, 3H), 4.53-4.42 (m, 1H), 4.35-4.22 (m, 1H), 4.04-3.72 (m, 2H), 3.59-3.50 (m, 2H), 3.27-3.10 (m, 2H), 2.81-2.66 (m, 4H), 2.65-2.50 (m, 1H), 2.37-2.22 (m, 1H), 2.08-1.91 (m, 1H), 1.80-1.76 (m, 3H), 1.74-1.22 (m, 5H), 1.11-0.65 (m, 6H). ESI-MS [M+H]⁺ calc'd for C₂₇H₃₅N₃O₄, 466.26; found, 466.4.

### Example 41: (1R,2S,5S)-3-(4-Cyclopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 41)

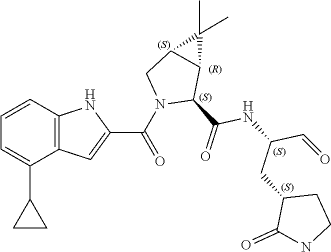

Compound 41 was synthesized by the same procedure as Compound 1. ¹H NMR (400 MHz, CD₃OD) δ ppm 7.25-7.04 (m, 3H), 6.68-6.55 (m, 1H), 5.05-4.91 (m, 1H), 4.78-4.63 (m, 1H), 4.53-4.32 (m, 1H), 4.05-3.94 (m, 1H), 3.89-3.67 (m, 1H), 3.29-3.23 (m, 1H), 2.82-2.57 (m, 1H), 2.41-2.25 (m, 1H), 2.20-2.04 (m, 1H), 1.88-1.70 (m, 2H), 1.61-1.51 (m, 1H), 1.44-1.20 (m, 2H), 1.17-1.11 (m, 4H), 1.07-1.00 (m, 2H), 1.05-0.98 (m, 2H), 0.92-0.58 (m, 2H). ESI-MS [M+H]⁺ calc'd for C₂₇H₃₂N₄O₄, 477.24; found, 477.4.

### Example 42: (1R,2S,5S)-3-(6-Chloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 42)

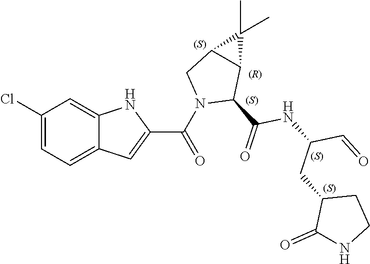

Compound 42 was synthesized by the same procedure as Compound 1. ¹H NMR (400 MHz, CD₃OD) δ ppm 7.66-7.47 (m, 1H), 7.46-7.42 (m, 1H), 7.09-6.87 (m, 2H), 4.99-4.89 (m, 1H), 4.53-4.37 (m, 1H), 4.33-3.94 (m, 2H), 3.81-3.69 (m, 1H), 3.29-3.22 (m, 1H), 3.01-2.57 (m, 1H), 2.42-2.28 (m, 1H), 2.11-1.83 (m, 1H), 1.82-1.66 (m, 2H), 1.62-1.52 (m, 1H), 1.47-1.32 (m, 1H), 1.18-0.88 (m, 6H). ESI-MS [M+H]⁺ calc'd for C₂₄H₂₇ClN₄O₄, 471.17; found, 471.4.

### Example 43: (1R,2S,5S)-3-(4-Chloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 43)

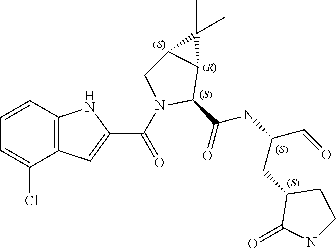

Compound 43 was synthesized by the same procedure as Compound 1. ¹H NMR (400 MHz, CD₃OD) δ ppm 7.38 (dd, J=4.4, 8.4 Hz, 1H), 7.22-7.14 (m, 1H), 7.12-6.89 (m, 2H), 5.02-4.89 (m, 1H), 4.55-4.39 (m, 1H), 4.36-3.92 (m, 2H), 3.83-3.68 (m, 1H), 3.29-3.22 (m, 1H), 2.92-2.20 (m, 2H), 2.11-1.93 (m, 1H), 1.86-1.51 (m, 3H), 1.44-1.26 (m, 1H), 1.17-0.95 (m, 6H). ESI-MS [M+H]⁺ calc'd for C₂₄H₂₇ClN₄O₄, 471.17; found, 471.3.

### Example 44: (1R,2S,5S)-3-(5,7-Dichloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 44)

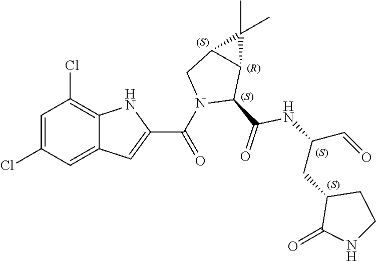

Compound 44 was synthesized by the same procedure as Compound 83. ¹H NMR (400 MHz, CD₃OD) δ ppm 7.66-7.48 (m, 1H), 7.32-7.26 (m, 1H), 7.11-6.88 (m, 1H), 4.96-4.88 (m, 1H), 4.54-4.37 (m, 1H), 4.31-4.01 (m, 1H), 3.90-3.71 (m, 1H), 3.28-2.98 (m, 1H), 2.67-2.49 (m, 1H), 2.38-1.98 (m, 1H), 1.86-1.23 (m, 6H), 1.18-0.95 (m, 6H). ESI-MS [M+H]⁺ calc'd for C₂₄H₂₆Cl₂N₄O₄, 505.13; found, 505.3.

### Example 45: (1R,2S,5S)-3-(1-(3-Chlorophenyl) cyclopropanecarbonyl)-N—((S)-4-(cyclopropylamino)-3,4-dioxo-1-((S)-2-oxopyrrolidin-3-yl)butan-2-yl)-6,6-dimethyl-3-azabicyclo[3.1.0]hexane-2-carboxamide (Compound 45)

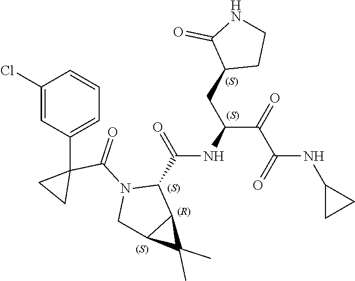

Compound 45 was synthesized by the same procedure as Compound 119. ¹H NMR (400 MHz, CD₃OD) δ ppm 7.40-7.09 (m, 4H), 4.62-4.11 (m, 2H), 3.67-3.54 (m, 1H), 3.41-3.32 (m, 2H), 3.29-3.15 (m, 1H), 2.80-2.55 (m, 2H), 2.53-2.30 (m, 1H), 2.14-1.57 (m, 3H), 1.48-1.40 (m, 2H), 1.39-1.08 (m, 4H), 1.05-0.92 (m, 3H), 0.85-0.46 (m, 7H). ESI-MS [M+H]⁺ calc'd for C₂₉H₃₅ClN₄O₅, 555.23; found, 555.4.

### Example 46: (S)-5-(7-cyano-1H-indole-2-carbonyl)-N—((S)-1-oxo-3-((S)-2-oxopyrrolidin-3-yl)propan-2-yl)-5-azaspiro[2.4]heptane-6-carboxamide (Compound 46)

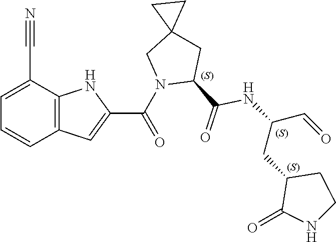

### Step 1: (S)-methyl 2-((S)-5-(7-cyano-1H-indole-2-carbonyl)-5-azaspiro[2.4]heptane-6-carboxamido)-3-((S)-2-oxopyrrolidin-3-yl)propanoate

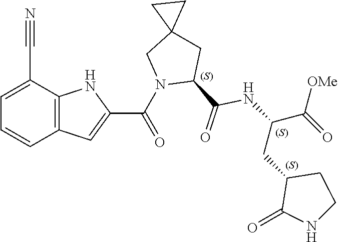

To a solution of (S)-methyl 3-((S)-2-oxopyrrolidin-3-yl)-2-((S)-5-azaspiro[2.4]heptane-6-carboxamido)propanoate (170 mg, 491.58 μmol, HCl) and DIEA (191 mg, 1.47 mmol) in DCM (10 mL) was added 7-cyano-1H-indole-2-carboxylic acid (92 mg, 491.58 mol). After addition, HATU (374 mg, 983.17 mol) was added at 0° C. The mixture was stirred at 25° C. for 12 hr. The reaction mixture was poured into sat.aq NH₄Cl (10 mL). The resulting mixture was extracted with EtOAc (10 mL×2). The combined organic phase was washed with brine (10 mL), dried over anhydrous Na₂SO₄, filtered and concentrated to dryness. The title compound (480 mg, crude) was obtained as a light yellow solid. ESI-MS [M+H]⁺ calc'd. for C₂₅H₂₇N₅O₅, 478.20, found 478.1.

### Step 2: (S)-5-(7-cyano-1H-indole-2-carbonyl)-N—((S)-1-hydroxy-3-((S)-2-oxopyrrolidin-3-yl)propan-2-yl)-5-azaspiro[2.4]heptane-6-carboxamide

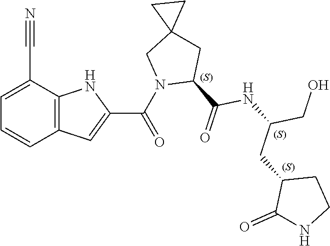

To a solution of (S)-methyl 2-((S)-5-(7-cyano-1H-indole-2-carbonyl)-5-azaspiro[2.4]heptane-6-carboxamido)-3-((S)-2-oxopyrrolidin-3-yl)propanoate (230 mg, 481.66 mol) in THE (3 mL) was added LiBH₄ (40 mg, 1.84 mmol) at 0° C. Then the mixture was stirred at 25° C. for 10 hr. The reaction was quenched with water (10 mL) drop-wise. The resulting mixture was extracted with DCM (10 mL×3). The combined organic phase was washed with water (10 mL), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 10%-40%, 10 min). The title compound (28 mg, 12.74% yield) was obtained as a white solid. ¹H NMR (400 MHz CDCl₃) δ ppm 12.07 (s, 1H), 9.27 (d, J=3.6 Hz, 1H), 7.92 (d, J=8.0 Hz, 1H), 7.61-7.53 (d, J=6.8 Hz, 1H), 7.18 (t, J=7.6 Hz, 1H), 7.13 (s, 1H), 7.04-6.98 (m, 1H), 5.13 (q, J=4.4 Hz, 1H), 3.93-3.84 (m, 1H), 3.82-3.70 (m, 2H), 3.64-3.42 (m, 3H), 3.28 (d, J=10.0 Hz, 1H), 2.62-2.44 (m, 2H), 2.33-2.24 (m, 1H), 2.10-1.87 (m, 3H), 1.78-1.72 (m, 1H), 0.75-0.38 (m, 4H).

### Step 3: (S)-5-(7-cyano-1H-indole-2-carbonyl)-N—((S)-1-oxo-3-((S)-2-oxopyrrolidin-3-yl)propan-2-yl)-5-azaspiro[2.4]heptane-6-carboxamide

To a solution of (S)-5-(7-cyano-1H-indole-2-carbonyl)-N—((S)-1-hydroxy-3-((S)-2-oxopyrrolidin-3-yl)propan-2-yl)-5-azaspiro[2.4]heptane-6-carboxamide (24 mg, 53.39 mol) in DCM (4 mL) was added DMP (68 mg, 160.18 mol) at 25° C. The mixture was stirred at 25° C. for 1 hr. The residue was purified by prep-HPLC (column: Phenomenex Gemini-NX C18 75×30 mm×3 μm; mobile phase: [water (10 mM NH₄HCO₃)-ACN]; B %: 9%-39%, 10 min). Compound 46 (1.8 mg, 7.33% yield, 97.33% purity) was obtained as a white solid.

¹H NMR (400 MHz, CD₃OD) δ ppm 8.10-7.80 (m, 1H), 7.73-7.61 (m, 1H), 7.28-6.99 (m, 2H), 4.52-4.29 (m, 1H), 4.11-3.68 (m, 3H), 3.30-3.00 (m, 2H), 2.72-2.37 (m, 2H), 2.28-1.97 (m, 3H), 1.88-1.57 (m, 2H), 0.84-0.56 (m, 4H). ESI-MS [M+H]⁺ calc'd. for C₂₄H₂₅N₅O₄, 448.19, found 448.4.

### Example 47: (S)-5-(1H-Indole-2-carbonyl)-N—((S)-1-oxo-3-((S)-2-oxopyrrolidin-3-yl)propan-2-yl)-5-azaspiro[2.4]heptane-6-carboxamide (Compound 47)

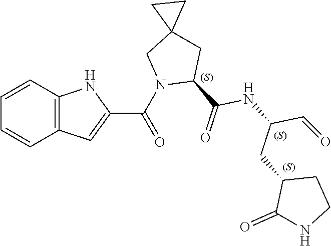

Compound 47 was synthesized by the same procedure as Compound 2. ¹H NMR (400 MHz, CD₃OD) δ ppm 7.69-7.51 (m, 1H), 7.44 (d, J=8.4 Hz, 1H), 7.29-7.16 (m, 1H), 7.11-6.85 (m, 2H), 5.27-5.11 (m, 1H), 4.52-4.31 (m, 1H), 4.11-3.83 (m, 2H), 3.79-3.63 (m, 1H), 2.76-2.31 (m, 2H), 2.28-2.13 (m, 1H), 2.12-1.95 (m, 2H), 1.94-1.69 (m, 1H), 1.68-1.31 (m, 2H), 0.87-0.54 (m, 4H). ESI-MS [M+H]⁺ calc'd for C₂₃H₂₆N₄O₄, 423.20; found, 423.4.

### Example 48: (1R,2S,5S)-6,6-dimethyl-3-(2-methyl-2-phenylpropanoyl)-N—((S)-1-oxo-3-((S)-2-oxopyrrolidin-3-yl)propan-2-yl)-3-azabicyclo[3.1.0]hexane-2-carboxamide (Compound 48)

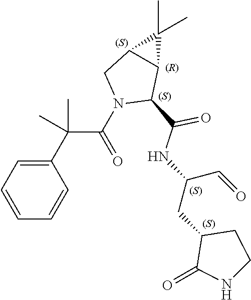

### Step 1: (S)-methyl 2-((1R,2S,5S)-6,6-dimethyl-3-(2-methyl-2-phenylpropanoyl)-3-azabicyclo[3.1.0]hexane-2-carboxamido)-3-((S)-2-oxopyrrolidin-3-yl)propanoate

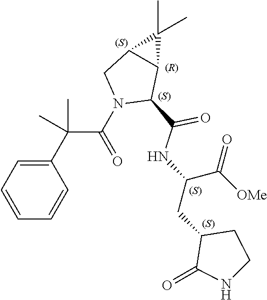

To a solution of 2-methyl-2-phenylpropanoic acid (127 mg, 778.94 mol) in DCM (3 mL) was added (S)-methyl 2-((1R,2S,5S)-6,6-dimethyl-3-azabicyclo[3.1.0]hexane-2-carboxamido)-3-((S)-2-oxopyrrolidin-3-yl)propanoate (229 mg, 708.13 μmol, HCl), DIEA (389 mg, 3.02 mmol) and HATU (269 mg, 708.13 mol). The mixture was stirred at 20° C. for 5 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: C18-6 100×30 mm×5 μm; mobile phase: [water (FA)-ACN]; B %: 42%-72%, 8 min). The title compound (71 mg, 19.97% yield) was obtained as yellow oil. ¹H NMR (400 MHz, CDCl₃) δ ppm 7.33-7.30 (m, 2H), 7.24-7.21 (m, 2H), 7.19-7.12 (m, 1H), 5.78 (s, 1H), 4.70-4.61 (m, 1H), 4.38 (s, 1H), 3.76 (s, 3H), 3.46-3.30 (m, 2H), 3.10-3.01 (m, 1H), 2.81 (d, J=10.8 Hz, 1H), 2.62-2.46 (m, 2H), 2.22-2.15 (m, 1H), 2.00-1.89 (m, 2H), 1.60-1.47 (m, 6H), 1.36 (d, J=7.6 Hz, 1H), 1.25-1.19 (m, 1H), 0.91 (s, 3H), 0.72 (s, 3H).

### Step 2: (1R,2S,5S)—N—((S)-1-hydroxy-3-((S)-2-oxopyrrolidin-3-yl)propan-2-yl)-6,6-dimethyl-3-(2-methyl-2-phenylpropanoyl)-3-azabicyclo[3.1.0]hexane-2-carboxamide

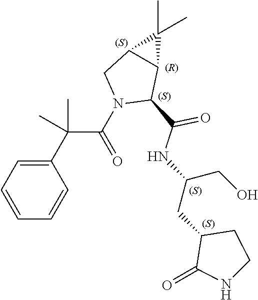

To a solution of (S)-methyl 2-((1R,2S,5S)-6,6-dimethyl-3-(2-methyl-2-phenylpropanoyl)-3-azabicyclo[3.1.0]hexane-2-carboxamido)-3-((S)-2-oxopyrrolidin-3-yl)propanoate (60 mg, 127.78 mol) in THE (3 mL) was added LiBH₄ (13 mg, 638.88 mol) at 0° C. The mixture was stirred at 0° C. for 1 hr. The mixture was quenched with H₂O (2 mL), extracted with DCM (5 mL×3). The combined organic layer was dried over anhydrous Na₂SO₄, filtered and concentrated in vacuo. The residue was used to next step directly. The title compound (40 mg, 42.54% yield) was obtained as a white solid. ESI-MS [M+H]⁺ calc'd for C₂₅H₃₅N₃O₄, 442.26; found, 442.3.

### Step 3: (1R,2S,5S)-6,6-dimethyl-3-(2-methyl-2-phenylpropanoyl)-N—((S)-1-oxo-3-((S)-2-oxopyrrolidin-3-yl)propan-2-yl)-3-azabicyclo[3.1.0]hexane-2-carboxamide

A mixture of (1R,2S,5S)—N—((S)-1-hydroxy-3-((S)-2-oxopyrrolidin-3-yl)propan-2-yl)-6,6-dimethyl-3-(2-methyl-2-phenylpropanoyl)-3-azabicyclo[3.1.0]hexane-2-carboxamide (40 mg, 90.59 mol), DMP (115 mg, 271.76 mol) in DCM (2 mL) was degassed and purged with N₂ for 3 times, and then the mixture was stirred at 25° C. for 0.5 hr under N₂ atmosphere. The mixture was filtered and the filtrate was concentrated. The residue was purified by prep-HPLC (column: Phenomenex C18 75×30 mm×3 μm; mobile phase: [water (NH₄HCO₃)-ACN]; B %: 24%-54%, 10 min). Compound 48 (1.2 mg, 2.83% yield, 94% purity) was obtained as a white solid. ¹H NMR (400 MHz, CD₃OD) δ ppm 7.42-7.22 (m, 5H), 4.52-4.38 (m, 1H), 4.12-3.92 (m, 1H), 3.39-3.36 (m, 1H), 3.23-3.14 (m, 1H), 2.85-2.79 (m, 1H), 2.77-2.62 (m, 1H), 2.50-2.36 (m, 1H), 2.15-2.00 (m, 1H), 1.87-1.74 (m, 1H), 1.71-1.59 (m, 1H), 1.54 (s, 3H), 1.48 (s, 3H), 1.41-1.24 (m, 3H), 0.96 (s, 3H), 0.75 (s, 3H). ESI-MS [M+H]⁺ calc'd for C₂₅H₃₃N₃O₄, 440.25; found, 440.3.

### Example 49: (1R,2S,5S)-3-(7-chloro-4-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 49)

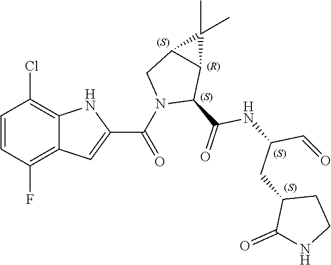

### Step 1: ethyl 7-chloro-4-fluoro-1H-indole-2-carboxylate

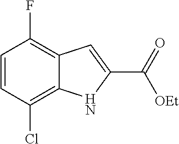 [SMILES: CCOC(=O)c1cc2c(F)ccc(Cl)c2[nH]1]

To a solution of (2-chloro-5-fluorophenyl)hydrazine (2 g, 10.15 mmol, HCl) in toluene (15 mL) was added ethyl 2-oxopropanoate (1.18 g, 10.15 mmol, 1.12 mL) and TsOH (5.24 g, 30.45 mmol) at 25° C. The mixture was stirred at 120° C. for 12 hr. Water (20 mL) was added and the reaction mixture was adjusted to pH~7 by sat.aq. NaHCO₃. The resulting mixture was extracted with EtOAc (10 mL×3). The combined organic phase was washed with water (10 mL), dried over anhydrous Na₂SO₄, filtered and concentrated. The residue was purified by flash silica gel chromatography (Eluent of 0~9% Ethylacetate/Petroleum ether). The title compound (200 mg, 2.72% yield) was obtained as a yellow solid. ¹H NMR (400 MHz, CDCl₃) δ ppm 9.07 (br s, 1H), 7.34-7.31 (m, 1H), 7.26-7.22 (m, 1H), 6.83-6.75 (m, 1H), 4.45 (q, J=7.2 Hz, 2H), 1.40 (t, J=7.2 Hz, 3H).

### Step 2: 7-chloro-4-fluoro-1H-indole-2-carboxylic acid

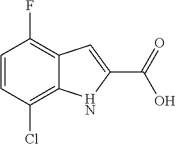 [SMILES: O=C(O)c1cc2c(F)ccc(Cl)c2[nH]1]

To a solution of ethyl 7-chloro-4-fluoro-1H-indole-2-carboxylate (300 mg, 1.24 mmol) in THF (5 mL) and H₂O (5 mL) was added LiOH·H₂O (105 mg, 2.48 mmol) in one portion. The mixture was stirred at 50° C. for 1 hr. The reaction mixture was adjusted to pH~3 with aq. HCl (1 N). H₂O (15 mL) was added to the reaction mixture. The resulting mixture was extracted with DCM (20 mL×3). The combined organic phase was washed with brine (20 mL), water (20 mL), dried over anhydrous Na₂SO₄, filtered and concentrated in vacuo. The title compound (230 mg, 81.13% yield) was obtained as a yellow solid. ¹H NMR (400 MHz, DMSO-d) δ ppm 13.33 (br s, 1H), 12.38 (br s, 1H), 7.34-7.29 (m, 1H), 7.20 (d, J=2.4 Hz, 1H), 6.95-6.87 (m, 1H).

### Step 3: (S)-methyl 2-((1R,2S,5S)-3-(7-chloro-4-fluoro-1H-indole-2-carbonyl)-6,6-dimethyl-3-azabicyclo[3.1.0]hexane-2-carboxamido)-3-((S)-2-oxopyrrolidin-3-yl)propanoate

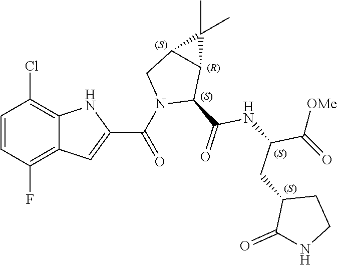

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (255 mg, 708.63 μmol, HCl) in DCM (5 mL) was added DIEA (275 mg, 2.13 mmol) and 7-chloro-4-fluoro-1H-indole-2-carboxylic acid (152 mg, 708.63 mol) at 0° C. Then HATU (539 mg, 1.42 mmol) was added in one portion at 0° C. The mixture was stirred at 25° C. for 10 hr. The reaction mixture was poured into water (20 mL). The resulting mixture was extracted with DCM (15 mL×3). The combined organic phase was washed with sat.aq.NaHCO₃ (15 mL×2) and brine (15 mL), dried over anhydrous Na₂SO₄, filtered and concentrated to dryness. The residue was purified by prep-HPLC (column: Xtimate C18 150×40 mm×10 μm; mobile phase: [water (10 mM NH₄HCO₃)-ACN]; B %: 30%-60%, 15 min). The title compound (108.5 mg, 28.63% yield) was obtained as a brown solid. ¹H NMR (400 MHz, CDCl₃) δ ppm 9.75-8.96 (m, 1H), 8.17-7.53 (m, 1H), 7.23-7.13 (m, 1H), 7.08-6.91 (m, 1H), 6.80-6.52 (m, 1H), 5.84-5.53 (m, 1H), 4.97-4.29 (m, 2H), 4.27-3.82 (m, 2H), 3.78-3.48 (m, 3H), 3.42-3.29 (m, 2H), 2.63-2.10 (m, 3H), 2.03-1.82 (m, 2H), 1.71-1.65 (m, 1H), 1.52-1.25 (m, 1H), 1.12-0.91 (m, 6H).

### Step 4: (1R,2S,5S)-3-(7-chloro-4-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide

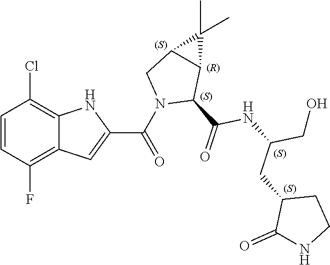

To a solution of (S)-methyl 2-((1R,2S,5S)-3-(7-chloro-4-fluoro-1H-indole-2-carbonyl)-6,6-dimethyl-3-azabicyclo[3.1.0]hexane-2-carboxamido)-3-((S)-2-oxopyrrolidin-3-yl)propanoate (107 mg, 206.18 mol) in THE (8 mL) was added LiBH₄ (13 mg, 618.54 mol) dropwise at 0° C. The mixture was stirred at 0° C. for 1 hr. H₂O (15 mL) was added to the reaction mixture. The resulting mixture was extracted with DCM (15 mL×3). The combined organic phase was washed with brine (15 mL), water (15 mL), dried over anhydrous Na₂SO₄, filtered and concentrated. The title compound (110 mg, 88.39% yield) was obtained as a white solid. ¹H NMR (400 MHz, CDCl₃) δ ppm 10.05-9.44 (m, 1H), 8.67-7.73 (m, 1H), 7.26-7.14 (m, 1H), 7.10-6.97 (m, 1H), 6.90-6.49 (m, 1H), 5.77-5.46 (m, 1H), 4.95-4.48 (m, 1H), 4.40-4.23 (m, 1H), 4.07-3.94 (m, 1H), 3.86-3.81 (m, 1H), 3.78-3.75 (m, 1H), 3.60-3.52 (m, 1H), 3.41-3.22 (m, 2H), 2.63-2.27 (m, 2H), 2.15-1.97 (m, 1H), 1.74-1.64 (m, 2H), 1.44 (s, 1H), 1.33-1.20 (m, 1H), 1.11-0.91 (m, 6H).

### Step 5: (1R,2S,5S)-3-(7-chloro-4-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(7-chloro-4-fluoro-1H-indole-2-carbonyl)-N—((S)-1-hydroxy-3-((S)-2-oxopyrrolidin-3-yl)propan-2-yl)-6,6-dimethyl-3-azabicyclo[3.1.0]hexane-2-carboxamide (55 mg, 112.03 mol) in DCM (5 mL) was added DMP (143 mg, 336.08 mol) at 25° C. The reaction mixture was stirred at 25° C. for 1 hr. The reaction mixture was filtered. The filtrate was concentrated. The residue was purified by prep-HPLC (column: Phenomenex Gemini-NX C18 75×30 mm×3 μm; mobile phase: [water (10 mM NH₄HCO₃)-ACN]; B %: 25%-55%, 10 min). Compound 49 (8.2 mg, 14.88% yield, 99.412% purity) was obtained as a white solid. ¹H NMR (400 MHz, CD₃OD) δ ppm 7.38-7.17 (m, 1H), 7.16-6.94 (m, 1H), 6.82-6.57 (m, 1H), 4.98-4.85 (m, 1H), 4.54-4.27 (m, 1H), 4.08-3.92 (m, 1H), 3.90-3.73 (m, 1H), 3.27-2.97 (m, 2H), 2.69-2.50 (m, 1H), 2.40-2.28 (m, 1H), 2.14-1.98 (m, 1H), 1.85-1.51 (m, 4H), 1.18-0.97 (m, 6H). ESI-MS [M+H]⁺ calc'd for C₂₄H₂₆ClFN₄O₄, 489.16; found, 489.4.

### Example 50: (1R, 2S, 5S)—N—((S)-4-amino-3, 4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide (Compound 50)

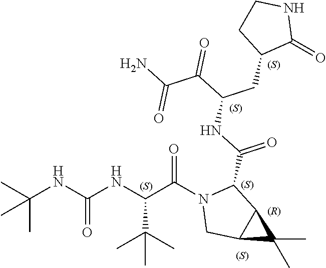

### Step 1: (1R,2S,5S)-methyl 3-((S)-2-(3-(tert-butyl)ureido)-3,3-dimethylbutanoyl)-6,6-dimethyl-3-azabicyclo[3.1.0]hexane-2-carboxylate

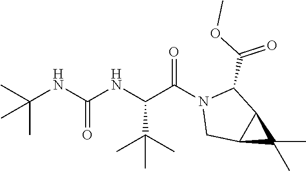 [SMILES: COC(=O)[C@@H]1[C@@H]2[C@H](CN1C(=O)[C@@H](NC(=O)NC(C)(C)C)C(C)(C)C)C2(C)C]

To a solution of (S)-2-(3-(tert-butyl)ureido)-3,3-dimethylbutanoic acid (2 g, 8.68 mmol) in DCM (30 mL) and DIPEA (4.49 g, 34.74 mmol) was added T₃P (8.29 g, 13.03 mmol, 7.75 mL, 50% purity in EtOAc) at 0° C. After addition, the mixture was stirred at this temperature for 30 min, and then (1R,2S,5S)-methyl 6,6-dimethyl-3-azabicyclo[3.1.0]hexane-2-carboxylate hydrochloride (2.14 g, 10.42 mmol) was added to the mixture. The resulting mixture was stirred at 25° C. for 10 hr. The reaction mixture was diluted with EtOAc (150 mL) and 5% H₃PO₄ (150 mL) for 30 min. The aqueous phase was extracted with EtOAc (100 mL). The combined organic layer was washed with 5% H₃PO₄ (150 mL×3), sat.aq.NaHCO₃ (150 mL×4), water (150 mL), and brine (150 mL), and dried over anhydrous Na₂SO₄, filtered and concentrated. Crude product (3.29 g, crude) was obtained as a yellow solid. ¹H NMR (400 MHz, DMSO-d₆) δ ppm 5.95 (s, 1H), 5.89 (d, J=10.0 Hz, 1H), 4.21-4.11 (m, 2H), 4.02 (d, J=10.4 Hz, 1H), 3.80-3.72 (m, 1H), 3.64 (s, 3H), 1.55-1.47 (m, 1H), 1.43-1.36 (m, 1H), 1.16 (s, 9H), 0.99 (s, 3H), 0.90 (s, 9H), 0.82 (s, 3H)

Step 2: (1R,2S,5S)-3-((S)-2-(3-(tert-butyl)ureido)-3,3-dimethylbutanoyl)-6,6-dimethyl-3-azabicyclo[3.1.0]hexane-2-carboxylic acid

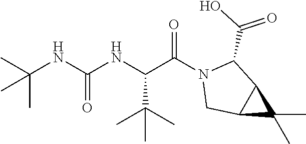 [SMILES: CC(C)(C)NC(=O)N[C@H](C(=O)N1C[C@H]2[C@@H]([C@H]1C(=O)O)C2(C)C)C(C)(C)C]

To a solution of (1R,2S,5S)-methyl 3-((S)-2-(3-(tert-butyl)ureido)-3,3-dimethylbutanoyl)-6,6-dimethyl-3-azabicyclo[3.1.0]hexane-2-carboxylate (1 g, 2.62 mmol) in THF (5 mL) and H₂O (5 mL) was added LiOH·H₂O (220 mg, 5.24 mmol). The mixture was stirred at 25° C. for 1 hr. The reaction was extracted with ethyl acetate (5 mL×2). The water phase was then adjusted to pH=4 by aq. HCl (1 N). The resulting mixture was extracted with DCM (10 mL×3). The combined organic phase was washed with water (10 mL), dried over anhydrous Na₂SO₄, filtered and concentrated. The title compound (1.03 g, 97.31% yield) was obtained as a white solid. ¹H NMR (400 MHz, DMSO-d₆) δ ppm 5.96 (s, 1H), 5.89 (d, J=10.0 Hz, 1H), 4.17-4.08 (m, 3H), 3.99 (d, J=10.0 Hz, 1H), 3.78-3.71 (m, 1H), 1.50-1.45 (m, 1H), 1.37 (d, J=8.0 Hz, 1H), 1.16 (s, 9H), 1.00 (s, 3H), 0.91 (s, 9H), 0.81 (s, 3H)

### Step 3: tert-butyl ((S)-1-hydroxy-3-((S)-2-oxopyrrolidin-3-yl)propan-2-yl)carbamate

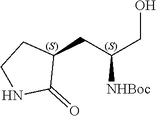

To a solution of (S)-methyl 2-((tert-butoxycarbonyl)amino)-3-((S)-2-oxopyrrolidin-3-yl)propanoate (5 g, 17.46 mmol) in THF (50 mL) was added LiBH₄ (1.85 g, 84.93 mmol) at 0° C. Then the mixture was stirred at 25° C. for 3 hrs. Water (200 mL) was added to the mixture. The aqueous layer was extracted with ethyl acetate (200 mL×5). The combined organic layers were washed with brine (200 mL), dried over anhydrous Na₂SO₄, filtered and concentrated under reduced pressure. The product (4.5 g, 99.76% yield) as a white solid was used for next step directly without further purification. ¹H NMR (400 MHz, CDCl₃) δ ppm 5.85 (s, 1H), 5.45 (s, 1H), 3.73 (s, 1H), 3.66-3.55 (m, 2H), 3.41-3.28 (m, 2H), 2.58-2.34 (m, 2H), 1.89-1.77 (m, 2H), 1.68-1.57 (m, 1H), 1.44 (s, 9H).

### Step 4: tert-butyl ((S)-1-oxo-3-((S)-2-oxopyrrolidin-3-yl)propan-2-yl)carbamate

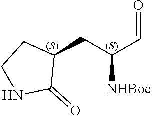

To a solution of tert-butyl ((S)-1-hydroxy-3-((S)-2-oxopyrrolidin-3-yl)propan-2-yl)carbamate (2.5 g, 9.68 mmol) in DCM (25 mL) was added SO₃·Py (9.24 g, 58.07 mmol) and TEA (5.88 g, 58.07 mmol) and DMSO (7.56 g, 96.78 mmol). Then the mixture was stirred at 25° C. for 1 h. Water (100 mL) was added to the mixture. The aqueous layer was extracted with DCM (100 mL×2). The combined organic layers were washed with brine (100 mL), dried over anhydrous Na₂SO₄, filtered and concentrated under reduced pressure. The residue was purified by flash chromatography on silica gel (MeOH in DCM 0-5%) to give the title compound (2.4 g, 96.76% yield) as a black brown oil. ¹H NMR (400 MHz, CDCl₃) δ ppm 9.55 (s, 1H), 8.61 (d, J=4.0 Hz, 1H), 5.54-5.41 (m, 1H), 4.20-4.13 (m, 1H), 3.45 (s, 2H), 2.04-2.01 (m, 5H), 1.43 (s, 9H).

### Step 5: tert-butyl ((2S)-1-cyano-1-hydroxy-3-((S)-2-oxopyrrolidin-3-yl)propan-2-yl)carbamate

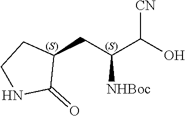

tert-butyl ((S)-1-oxo-3-((S)-2-oxopyrrolidin-3-yl)propan-2-yl)carbamate (2.4 g, 9.36 mmol) was dissolved in MeOH (15 mL) and cooled to 0° C. The solution of NaHSO₃ (1.46 g, 14.05 mmol) dissolved in H₂O (22 mL) was added to the above mixture. Then the mixture was stirred at 25° C. for 5 hrs. NaCN (550 mg, 11.22 mmol) was dissolved in H₂O (5 mL) and added with EtOAc (50 mL) to the above mixture. The reaction was stirred at 25° C. for 5 hrs. Water (100 mL) was added to the mixture. The aqueous layer was extracted with ethyl acetate (100 mL×2). The combined organic layers were washed with brine (100 mL), dried over anhydrous Na₂SO₄, filtered and concentrated under reduced pressure. The residue was purified by flash chromatography on silica gel (EtOAc in Petroleum ether=0-5%) to give the title compound (1.3 g, 49.00% yield) as a colorless oil. 1H NMR (400 MHz, CDCl₃) δ ppm 6.02-5.87 (m, 1H), 4.75-4.45 (m, 1H), 4.01-3.85 (m, 1H), 3.44-3.30 (m, 2H), 2.60-2.28 (m, 2H), 2.15-1.81 (m, 3H), 1.50-1.38 (m, 9H)

### Step 6: tert-butyl ((2S)-4-amino-3-hydroxy-4-oxo-1-((S)-2-oxopyrrolidin-3-yl)butan-2-yl)carbamate

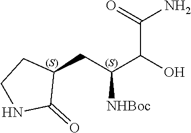

To a solution of tert-butyl ((2S)-1-cyano-1-hydroxy-3-((S)-2-oxopyrrolidin-3-yl)propan-2-yl)carbamate (200 mg, 705.91 mol) in DMSO (2 mL) was added K₂CO₃ (97 mg, 705.91 mol). H₂O₂ (140 mg, 1.24 mmol, 119 μL, 30% purity) was added to the above mixture at 10° C. dropwise. Then the mixture was stirred at 25° C. for 2 hrs. Saturated Na₂SO₃ (2 mL) was added to the mixture. The reaction mixture was filtered. The combined filtrate was concentrated under reduce pressure. The residue was purified by prep-HPLC (column: 3_Phenomenex Luna C18 75×30 mm×3 μm; mobile phase: [water (0.05% HCl)-ACN]; B %: 0%-25%, 40 min) to give the title compound (200 mg, 94.02% yield) as a white solid. ESI-MS [M+H]⁺ calc'd for C₁₃H₂₃N₃O₅, 302.2, found 302.4.

### Step 7: (3S)-3-amino-2-hydroxy-4-((S)-2-oxopyrrolidin-3-yl)butanamide

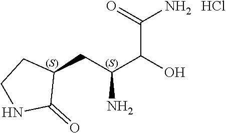

To a solution of tert-butyl N-[(1S)-3-amino-2-hydroxy-3-oxo-1-[[(3S)-2-oxopyrrolidin-3-yl]methyl]propyl]carbamate (150 mg, 497.78 mol) in MeOH (4 mL) was added HCl/dioxane (4 M, 124.44 L). The resulting mixture was stirred at 25° C. for 1 hour. The reaction mixture was concentrated under reduce pressure to give a residue. The product (118 mg, 99.73% yield, HCl) as a white solid was used for next step directly without further purification.

### Step 8: (1R,2S,5S)—N-((2S)-4-amino-3-hydroxy-4-oxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide

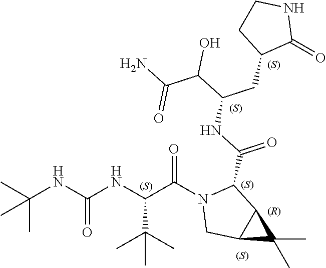

To a solution of (3S)-3-amino-2-hydroxy-4-((S)-2-oxopyrrolidin-3-yl)butanamide (118 mg, 496.46 μmol, HCl) in DCM (2 mL) was added DIEA (192 mg, 1.49 mmol) and (1R,2S,5S)-3-((S)-2-(3-(tert-butyl)ureido)-3,3-dimethylbutanoyl)-6,6-dimethyl-3-azabicyclo[3.1.0]hexane-2-carboxylic acid (146 mg, 397.17 μmol) slowly at 25° C. And then a solution of HATU (377 mg, 992.92 mol) in DCM (2 mL) was added dropwise at 0° C. The resulting mixture was stirred at 25° C. for 16 hrs. The reaction mixture was concentrated under reduce pressure to give a residue. The residue was purified by prep-HPLC (column: 3_Phenomenex Luna C18 75×30 mm×3 μm; mobile phase: [water (0.05% HCl)-ACN]; B %: 10%-40%, 40 min) to give the title compound (60 mg, 21.95% yield) as a white solid. 1H NMR (400 MHz, CD₃OD) δ ppm 4.33-4.22 (m, 2H), 4.09-3.89 (m, 3H), 3.30-3.17 (m, 2H), 2.70-2.54 (m, 1H), 2.47-2.10 (m, 2H), 1.89-1.50 (m, 3H), 1.49-1.30 (m, 2H), 1.29-1.22 (m, 9H), 1.08-1.03 (m, 3H), 1.03-0.95 (m, 9H), 0.95-0.90 (m, 3H).

### Step 9: (1R, 2S, 5S)—N—((S)-4-amino-3, 4-dioxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide

To a solution of (1R,2S,5S)—N-((2S)-4-amino-3-hydroxy-4-oxo-1-((S)-2-oxopyrrolidin-3-yl)butan-2-yl)-3-((S)-2-(3-(tert-butyl)ureido)-3,3-dimethylbutanoyl)-6,6-dimethyl-3-azabicyclo[3.1.0]hexane-2-carboxamide (60 mg, 108.95 mol) in DCM (1 mL) was added Dess-Martin (139 mg, 326.86 mol). Then the mixture was stirred at 25° C. for 16 h. The reaction was concentrated under reduce pressure to give a residue. The residue was purified by prep-HPLC (column: 3_Phenomenex Luna C18 75×30 mm×3 μm; mobile phase: [water (0.05% HCl)-ACN]; B %: 10%-50%, 40 min) to give Compound 50 (13.9 mg, 23.25% yield) as a white solid. ¹H NMR (400 MHz, CD₃OD) δ ppm 4.38-4.18 (m, 3H), 4.07-3.88 (m, 2H), 3.28-3.14 (m, 2H), 2.65-2.52 (m, 1H), 2.44-2.25 (m, 1H), 2.15-1.96 (m, 1H), 1.81-1.42 (m, 4H), 1.29-1.22 (m, 9H), 1.07-0.93 (m, 15H). ESI-MS [M+H]⁺ calc'd for C₂₇H₄₄N₆O₆, 549.3, found 549.5.

### Example 51: (S)-1-(4-Fluoro-1H-indole-2-carbonyl)-4,4-dimethyl-N—((S)-1-oxo-3-((S)-2-oxopyrrolidin-3-yl)propan-2-yl)pyrrolidine-2-carboxamide (Compound 51)

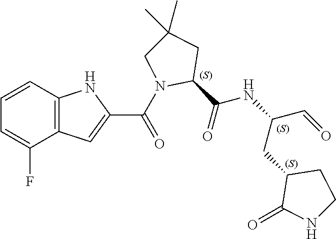

Compound 51 was synthesized by the same procedure as Compound 1. ¹H NMR (400 MHz, CD₃OD) δ ppm 7.28-7.23 (m, 1H), 7.21-7.12 (m, 1H), 7.07-6.88 (m, 1H), 6.81-6.66 (m, 1H), 4.58 (s, 1H), 4.50-4.28 (m, 1H), 4.06-3.76 (m, 2H), 3.73-3.41 (m, 1H), 2.96-2.63 (m, 1H), 2.46-1.98 (m, 3H), 1.94-1.52 (m, 3H), 1.42-1.29 (m, 1H), 1.27-1.00 (m, 6H). ESI-MS [M+H]⁺ calc'd for C₂₃H₂₇FN₄O₄, 443.20, found 443.1.

### Example 52: (1R,2S,5S)-6,6-dimethyl-3-(2-(4-methylcyclohexyl)acetyl)-N—((S)-1-oxo-3-((S)-2-oxopyrrolidin-3-yl)propan-2-yl)-3-azabicyclo[3.1.0]hexane-2-carboxamide (Compound 52)

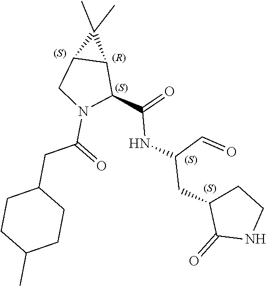

### Step 1: (S)-methyl 2-((1R,2S,5S)-6,6-dimethyl-3-(2-(4-methylcyclohexyl)acetyl)-3-azabicyclo[3.1.0]hexane-2-carboxamido)-3-((S)-2-oxopyrrolidin-3-yl)propanoate

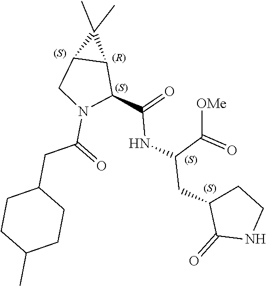

To a solution of (S)-methyl 2-((1R,2S,5S)-6,6-dimethyl-3-azabicyclo[3.1.0]hexane-2-carboxamido)-3-((S)-2-oxopyrrolidin-3-yl)propanoate (165 mg, 458.53 μmol, HCl), 2-(4-methylcyclohexyl)acetic acid (72 mg, 458.53 μmol,) and DIEA (178 mg, 1.38 mmol) in DCM (3 mL) was added HATU (349 mg, 917.06 mol) slowly at 0° C. The resulting mixture was stirred at 25° C. for 10 hr. The resulting suspension was diluted with DCM (10 mL). The mixture was washed with sat.aq.NH₄Cl (10 mL), dried over anhydrous Na₂SO₄, filtered and concentrated in vacuo. The residue was purified by flash silica gel chromatography (Eluent of 0~100% EtOAc in PE). The title compound (267 mg, 30.78% yield) was obtained as a yellow oil. ¹H NMR (400 MHz, CDCl₃) δ ppm 10.83-10.31 (m, 1H), 8.81-7.31 (m, 1H), 4.64-4.37 (m, 1H), 4.35-4.07 (m, 1H), 3.95-3.79 (m, 1H), 3.79-3.70 (m, 3H), 3.70-3.62 (m, 1H), 3.56-3.44 (m, 1H), 3.42-3.30 (m, 1H), 2.65-2.40 (m, 2H), 2.38-2.28 (m, 1H), 2.25-2.08 (m, 3H), 2.06-1.97 (m, 1H), 1.93-1.81 (m, 1H), 1.76-1.58 (m, 3H), 1.57-1.54 (m, 5H), 1.39-1.15 (m, 3H), 1.09-0.81 (m, 9H).

### Step 2: (1R,2S,5S)—N—((S)-1-hydroxy-3-((S)-2-oxopyrrolidin-3-yl)propan-2-yl)-6,6-dimethyl-3-(2-(4-methylcyclohexyl)acetyl)-3-azabicyclo[3.1.0]hexane-2-carboxamide

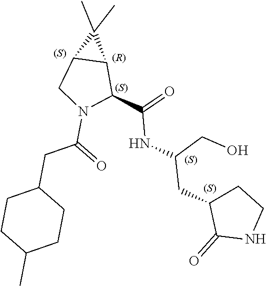

To a solution of (S)-methyl 2-((1R,2S,5S)-6,6-dimethyl-3-(2-(4-methylcyclohexyl)acetyl)-3-azabicyclo[3.1.0]hexane-2-carboxamido)-3-((S)-2-oxopyrrolidin-3-yl)propanoate (260 mg, 563.27 mol) in THE (3 mL) was added LiBH₄ (70 mg, 3.21 mmol) at 0° C. The mixture was stirred at 25° C. for 1 h. The reaction was quenched with water (10 mL) drop-wise. The resulting mixture was extracted with DCM (10 mL×3). The combined organic phase was washed with water (10 mL), dried over anhydrous Na₂SO₄, filtered and concentrated in vacuo. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 21%-61%, 11 min). The title compound (30 mg, 7.92% yield) was obtained as a white solid. ¹H NMR (400 MHz, CD₃OD) δ ppm 8.15-7.98 (m, 1H), 4.38-4.18 (m, 1H), 4.07-3.84 (m, 2H), 3.76-3.45 (m, 4H), 3.29-3.20 (m, 1H), 2.71-2.56 (m, 1H), 2.40-2.14 (m, 3H), 2.08-1.87 (m, 2H), 1.81-1.39 (m, 11H), 1.37-1.22 (m, 2H), 1.07 (s, 3H), 0.97-0.83 (m, 6H).

### Step 3: (1R,2S,5S)-6,6-dimethyl-3-(2-(4-methylcyclohexyl)acetyl)-N—((S)-1-oxo-3-((S)-2-oxopyrrolidin-3-yl)propan-2-yl)-3-azabicyclo[3.1.0]hexane-2-carboxamide

To a solution of (1R,2S,5S)—N—((S)-1-hydroxy-3-((S)-2-oxopyrrolidin-3-yl)propan-2-yl)-6,6-dimethyl-3-(2-(4-methylcyclohexyl)acetyl)-3-azabicyclo[3.1.0]hexane-2-carboxamide (20 mg, 46.13 mol) in DCM (4 mL) was added DMP (59 mg, 138.38 mol). The mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 26%-56%, 10 min). Compound 52 (7.0 mg, 34.87% yield, 99.156% purity) was obtained as a white solid. ¹H NMR (400 MHz, CD₃OD) δ ppm 4.54-4.33 (m, 1H), 4.32-4.20 (m, 1H), 4.08-3.66 (m, 2H), 3.65-3.50 (m, 1H), 3.29-3.20 (m, 1H), 2.68-1.81 (m, 6H), 1.80-1.62 (m, 4H), 1.61-1.38 (m, 5H), 1.36-1.17 (m, 2H), 1.12-0.83 (m, 11H). ESI-MS [M+H]⁺ calc'd for C₂₄H₃₇N₃O₄, 432.28; found, 432.4.

### Example 53: (1R,2S,5S)-3-(2-cyclohexylacetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 53)

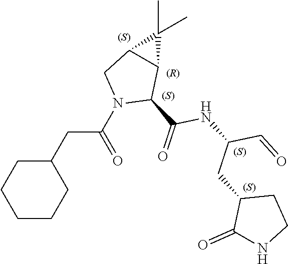

### Step 1: (S)-methyl 2-((1R,2S,aS)-3-(2-cyclohexylacetyl)-6,6-dimethyl-3-azabicyclo[3.1.0]hexane-2-carboxamido)-3-((S)-2-oxopyrrolidin-3-yl)propanoate

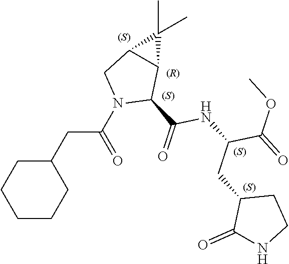

To a solution of 2-cyclohexylacetic acid (101 mg, 708.63 μmol) and DIEA (367 mg, 2.83 mmol) in DCM (4 mL) was added T₃P (677 mg, 1.06 mmol, 50% purity in EtOAc) dropwise at 0° C. After addition, the mixture was stirred at 0° C. for 20 mins. Then (S)-methyl-2-((1R,2 S, 5S)-6,6-dimethyl-3-azabicyclo [3.1.0]hexane-2-carboxamido)-3-((S)-2-oxopyrrolidin-3-yl)propanoate (255 mg, 708.63 μmol, HCl) in DCM (4 mL) was added in one portion at 0° C. The mixture was stirred at 25° C. for 10 hr. The reaction mixture was poured into water (20 mL). The resulting mixture was extracted with DCM (15 mL×3). The combined organic phase was washed with 5% H₃PO₄ (15 mL×2), sat.aq.NaHCO₃ (15 mL×2) and brine (15 mL), dried over anhydrous Na₂SO₄, filtered and concentrated to dryness. The residue was purified by prep-HPLC (column: Xtimate C18 150×40 mm×10 μm; mobile phase: [water (0.05% HCl)-ACN]; B %: 30%-60%, 10 min). The title compound (175 mg, 37.16% yield) was obtained as colorless oil. ¹H NMR (400 MHz, CDCl₃) δ ppm 8.41-7.43 (m, 1H), 6.28-5.67 (m, 1H), 4.61-4.38 (m, 1H), 4.33-4.15 (m, 1H), 3.86-3.77 (m, 1H), 3.75-3.67 (m, 3H), 3.49 (d, J=10.4 Hz, 1H), 3.41-3.28 (m, 2H), 2.58-2.36 (m, 2H), 2.19-2.09 (m, 2H), 1.91-1.79 (m, 2H), 1.76-1.59 (m, 8H), 1.58-1.45 (m, 2H), 1.38-1.08 (m, 4H), 1.07-1.01 (m, 3H), 0.93-0.89 (m, 3H).

### Step 2: (1R,2S,5S)-3-(2-cyclohexylacetyl)-N—((S)-1-hydroxy-3-((S)-2-oxopyrrolidin-3-yl)propan-2-yl)-6,6-dimethyl-3-azabicyclo[3.1.0]hexane-2-carboxamide

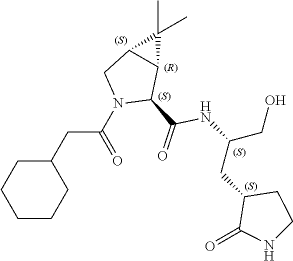

To a solution of (S)-methyl 2-((1R,2S,5S)-3-(2-cyclohexylacetyl)-6,6-dimethyl-3-azabicyclo[3.1.0]hexane-2-carboxamido)-3-((S)-2-oxopyrrolidin-3-yl)propanoate (85 mg, 189.92 mol) in THE (6 mL) was added LiBH₄ (12 mg, 569.75 mol) at 0° C. The mixture was stirred at 0° C. for 1 hr. H₂O (10 mL) was added. The resulting mixture was extracted with DCM (15 mL×3). The combined organic phase was washed with brine (25 mL), water (25 mL), dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 22%-52%, 10 min). The title compound (45 mg, 29.91% yield, 52.95% purity) was obtained as a white solid. ¹H NMR (400 MHz, CDCl₃) δ ppm 7.82-7.44 (m, 1H), 6.34-6.00 (m, 1H), 4.27-4.16 (m, 1H), 4.14-3.95 (m, 1H), 3.93-3.81 (m, 1H), 3.77-3.67 (m, 1H), 3.63-3.43 (m, 3H), 3.39-3.27 (m, 2H), 2.58-2.32 (m, 2H), 2.20-1.98 (m, 3H), 1.90-1.54 (m, 8H), 1.53-1.37 (m, 2H), 1.31-1.08 (m, 3H), 1.07-0.79 (m, 8H).

### Step 3: (1R,2S,5S)-3-(2-cyclohexylacetyl)-6,6-dimethyl-N—((S)-1-oxo-3-((S)-2-oxopyrrolidin-3-yl)propan-2-yl)-3-azabicyclo[3.1.0]hexane-2-carboxamide

To a solution of (1R,2S,5S)-3-(2-cyclohexylacetyl)-N—((S)-1-hydroxy-3-((S)-2-oxopyrrolidin-3-yl)propan-2-yl)-6,6-dimethyl-3-azabicyclo[3.1.0]hexane-2-carboxamide (40 mg, 95.34 mol) in DCM (3 mL) was added DMP (81 mg, 190.68 mol) at 25° C. The reaction mixture was stirred at 25° C. for 0.5 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 25%-55%, 10 min). Compound 53 (5 mg, 12.08% yield, 96.212% purity) was obtained as a white solid. ¹H NMR (400 MHz, CD₃OD) δ ppm 4.52-4.33 (m, 1H), 4.30-3.61 (m, 3H), 3.60-3.53 (m, 1H), 3.29-3.26 (m, 1H), 2.66-2.27 (m, 2H), 2.23-1.89 (m, 3H), 1.83-1.62 (m, 7H), 1.61-1.39 (m, 3H), 1.33-1.13 (m, 3H), 1.11-0.81 (m, 8H). ESI-MS [M+H]⁺ calc'd for C₂₃H₃₅N₃O₄, 418.26 found 418.4.

### Example 54: (1R,2S,5S)-3-(2-Cyclohexylacetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 54)

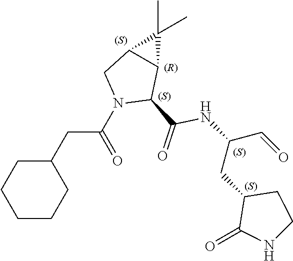

Compound 54 was synthesized by the same procedure as Compound 1. ¹H NMR (400 MHz, CD₃OD) δ ppm 7.53-7.23 (m, 2H), 7.12-6.85 (m, 1H), 4.53-4.38 (m, 1H), 4.33-4.23 (m, 1H), 4.06-3.91 (m, 2H), 3.85-3.70 (m, 1H), 3.28-3.23 (m, 1H), 2.74-2.57 (m, 1H), 2.52-2.27 (m, 1H), 2.08-1.83 (m, 1H), 1.80-1.67 (m, 2H), 1.63-1.50 (m, 2H), 1.48-1.36 (m, 1H), 1.17-0.93 (m, 6H). ESI-MS [M+H]⁺ calc'd for C₂₄H₂₆F₂N₄O₄, 473.19, found 473.2.

### Example 55: (1R,2S,5S)-3-(2-(Azepan-1-yl)acetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 55)

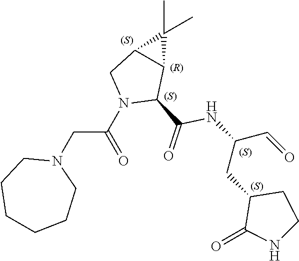

Compound 55 was synthesized by the same procedure as Compound 1. ¹H NMR (400 MHz, CD₃OD) δ ppm 4.53-4.45 (m, 1H), 4.32-3.83 (m, 3H), 3.76-3.35 (m, 3H), 3.28-2.99 (m, 2H), 2.81-2.69 (m, 3H), 2.67-2.27 (m, 3H), 2.07-1.93 (m, 1H), 1.72-1.42 (m, 11H), 1.11-0.96 (m, 6H). ESI-MS [M+H]⁺ calc'd for C₂₃H₃₆N₄O₄, 451.27, found 451.3.

### Example 56: (1R,2S,5S)-3-(6-Methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 56)

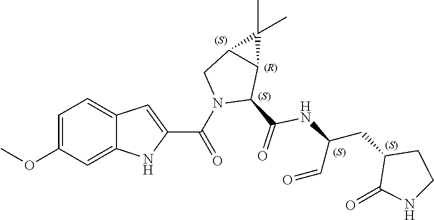

Compound 56 was synthesized by the same procedure as Compound 1. ¹H NMR (400 MHz, CD₃OD) δ ppm 7.55-7.34 (m, 1H), 7.03-6.82 (m, 2H), 6.71 (ddd, J=2.4, 8.8, 11.2 Hz, 1H), 4.52-4.38 (m, 1H), 4.30-3.93 (m, 2H), 3.82 (d, J=3.6 Hz, 3H), 3.79-3.73 (m, 1H), 3.29-3.23 (m, 1H), 2.95-2.23 (m, 2H), 2.04-1.86 (m, 1H), 1.80-1.25 (m, 5H), 1.15-0.93 (m, 6H). ESI-MS [M+H]⁺ calc'd for C₂₅H₃₀N₄O₅, 467.22, found 467.4.

### Example 57: (S)-1-(1H-Indole-2-carbonyl)-4,4-dimethyl-N—((S)-1-oxo-3-((S)-2-oxopyrrolidin-3-yl)propan-2-yl)pyrrolidine-2-carboxamid (Compound 57)

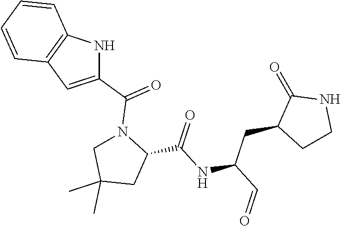 [SMILES: CC1(C)C[C@@H](C(=O)N[C@H](C=O)C[C@@H]2CCNC2=O)N(C(=O)c2cc3ccccc3[nH]2)C1]

Compound 57 was synthesized by the same procedure as Compound 2. ¹H NMR (400 MHz, CDCl₃) δ_{H}=9.56-9.43 (m, 2H), 8.57-8.38 (m, 1H), 7.66 (d, J=8.0 Hz, 1H), 7.42 (d, J=8.8 Hz, 1H), 7.33-7.27 (m, 1H), 7.13 (t, J=7.6 Hz, 1H), 6.93 (s, 1H), 5.87-5.67 (m, 1H), 4.80 (t, J=9.2 Hz, 1H), 4.43-4.29 (m, 1H), 3.89-3.82 (m, 1H), 3.80-3.73 (m, 1H), 3.39-3.25 (m, 2H), 2.69-2.51 (m, 1H), 2.48-2.32 (m, 1H), 2.23-1.90 (m, 5H), 1.26-1.20 (m, 3H), 1.05 (s, 3H). ESI-MS [M+H]⁺ calc'd for C₂₃H₂₈N₄O₄, 425.21, found 425.1.

### Example 58: (S)-1-(7-Cyano-1H-indole-2-carbonyl)-4,4-dimethyl-N—((S)-1-oxo-3-((S)-2-oxopyrrolidin-3-yl)propan-2-yl)pyrrolidine-2-carboxamide (Compound 58)

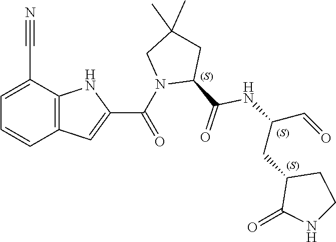

Compound 58 was synthesized by the same procedure as Compound 2. ¹H NMR (400 MHz, CD₃OD) δ ppm 8.02-7.83 (m, 1H), 7.69-7.60 (m, 1H), 7.27-6.96 (m, 2H), 5.16-4.87 (m, 1H), 4.76-4.66 (m, 1H), 4.51-4.28 (m, 1H), 4.07-3.41 (m, 3H), 3.01-2.62 (m, 1H), 2.41-2.26 (m, 1H), 2.14-2.00 (m, 1H), 1.93-1.80 (m, 1H), 1.79-1.67 (m, 1H), 1.65-1.50 (m, 1H), 1.46-1.27 (m, 1H), 1.26-0.98 (m, 6H). ESI-MS [M+H]⁺ calc'd for C₂₄H₂₇N₅O₄, 450.21, found 450.4.

### Example 59: (1R,2S,5S)-3-(4-Cyano-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 59)

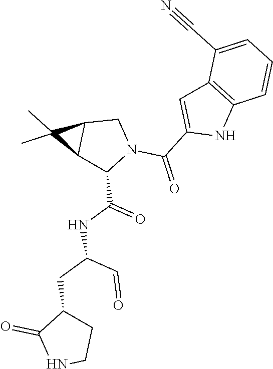 [SMILES: CC1(C)[C@@H]2[C@@H](C(=O)N[C@H](C=O)C[C@@H]3CCNC3=O)N(C(=O)c3cc4c(C#N)cccc4[nH]3)C[C@@H]21]

Compound 59 was synthesized by the same procedure as Compound 1. ¹H NMR (400 MHz, CD₃OD) δ ppm 7.81-7.71 (m, 1H), 7.59-7.46 (m, 1H), 7.41-7.30 (m, 1H), 7.18-6.93 (m, 1H), 4.85-4.69 (m, 1H), 4.55-4.29 (m, 1H), 4.09-3.66 (m, 3H), 3.30-3.23 (m, 1H), 2.97-2.58 (m, 1H), 2.48-2.29 (m, 1H), 2.10-1.73 (m, 2H), 1.66-1.25 (m, 3H), 1.20-0.95 (m, 6H). ESI-MS [M+H]⁺ calc'd for C₂₅H₂₇N₅O₄, 462.2, found 462.3.

### Example 60: (1R,2S,5S)-3-(1H-Indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 60)

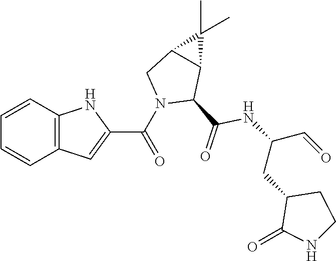 [SMILES: CC1(C)[C@@H]2[C@@H](C(=O)N[C@H](C=O)C[C@@H]3CCNC3=O)N(C(=O)c3cc4ccccc4[nH]3)C[C@@H]21]

Compound 60 was synthesized by the same procedure as Compound 1. ¹H NMR (400 MHz, CD₃OD) δ ppm 7.66 (d, J=8.0 Hz, 1H), 7.53 (d, J=8.0 Hz, 1H), 7.46-7.40 (m, 1H), 7.22 (q, J=7.2 Hz, 1H), 7.10-6.99 (m, 2H), 6.94-6.88 (m, 1H), 5.03-4.90 (m, 1H), 4.65-4.63 (m, 1H), 4.52-4.38 (m, 1H), 4.35-4.27 (m, 1H), 4.07-3.94 (m, 2H), 3.80-3.67 (m, 1H), 2.91-2.80 (m, 1H), 2.70-2.57 (m, 1H), 2.42-2.29 (m, 1H), 2.26-2.14 (m, 1H), 2.10-1.98 (m, 1H), 1.95-1.79 (m, 1H), 1.74-1.68 (m, 1H), 1.64-1.53 (m, 1H), 1.44-1.26 (m, 1H), 1.17-1.06 (m, 4H), 0.99-0.96 (m, 1H). ESI-MS [M+H]⁺ calc'd for C₂₄H₂₈N₄O₄, 437.21, found 437.0.

### Example 61: (2S,4S)-4-Cyclohexyl-1-(1H-indole-2-carbonyl)-N—((S)-1-oxo-3-((S)-2-oxopyrrolidin-3-yl)propan-2-yl)pyrrolidine-2-carboxamide (Compound 61)

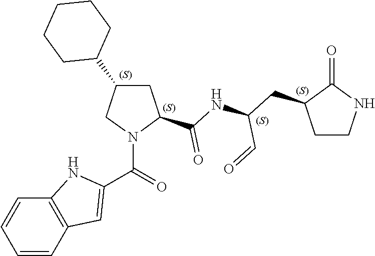

Compound 61 was synthesized by the same procedure as Compound 2. ¹H NMR (400 MHz, CD₃OD) δ ppm 7.71-7.49 (m, 1H), 7.48-7.39 (m, 1H), 7.28-7.15 (m, 1H), 7.12-6.83 (m, 2H), 5.10 (d, J=6.0 Hz, 1H), 4.75-4.53 (m, 1H), 4.39 (d, J=3.2 Hz, 1H), 4.20-3.89 (m, 1H), 3.86-3.54 (m, 1H), 3.07-2.55 (m, 1H), 2.51-2.25 (m, 2H), 2.25-1.86 (m, 4H), 1.77 (d, J=10.0 Hz, 4H), 1.71-1.54 (m, 2H), 1.53-1.39 (m, 1H), 1.38-1.18 (m, 4H), 1.15-0.94 (m, 2H). ESI-MS [M+H]⁺ calc'd for C₂₇H₃₄N₄O₄, 479.26, found 479.4.

### Example 62: (S)-1-(7-Fluoro-1H-indole-2-carbonyl)-4,4-dimethyl-N—((S)-1-oxo-3-((S)-2-oxopyrrolidin-3-yl)propan-2-yl)pyrrolidine-2-carboxamide (Compound 62)

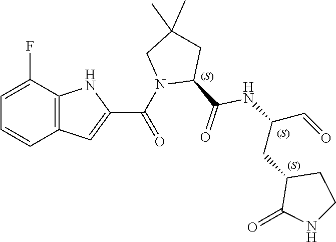

Compound 62 was synthesized by the same procedure as Compound 2. ¹H NMR (400 MHz, CD₃OD) δ ppm 7.43-7.17 (m, 1H), 7.01-6.74 (m, 3H), 4.40-4.18 (m, 1H), 3.97-3.80 (m, 1H), 3.75-3.31 (m, 3H), 2.85-2.53 (m, 1H), 2.35-1.88 (m, 3H), 1.84-1.70 (m, 1H), 1.70-1.58 (m, 1H), 1.56-1.39 (m, 1H), 1.27-1.17 (m, 1H), 1.15-0.92 (m, 6H. ESI-MS [M+H]⁺ calc'd for C₂₃H₂₇FN₄O₄, 443.20, found 443.1.

### Example 63: (1S,3aR,6aS)-2-(2-(2,4-Dichlorophenoxy)acetyl)-N—((S)-1-oxo-3-((S)-2-oxopyrrolidin-3-yl)propan-2-yl)octahydrocyclopenta[c]pyrrole-1-carboxamide (Compound 63)

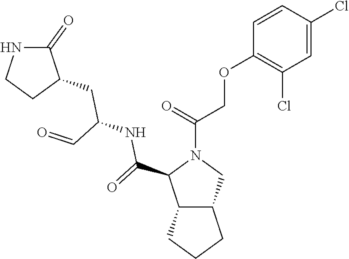 [SMILES: O=C[C@H](C[C@@H]1CCNC1=O)NC(=O)[C@@H]1[C@H]2CCC[C@H]2CN1C(=O)COc1ccc(Cl)cc1Cl]

Compound 63 was synthesized by the same procedure as Compound 2. ¹H NMR (400 MHz, CD₃OD) δ ppm 7.34-7.28 (m, 1H), 7.16-7.08 (m, 1H), 6.89-6.81 (m, 1H), 4.41-4.10 (m, 2H), 3.90-3.68 (m, 2H), 3.45-3.34 (m, 1H), 3.08-2.73 (m, 3H), 2.67-2.37 (m, 2H), 2.10-2.00 (m, 1H), 1.95-1.76 (m, 3H), 1.75-1.15 (m, 7H). ESI-MS [M+H]⁺ calc'd for C₂₃H₂₇C₁₂N₃O₅, 496.13, found 496.1.

### Example 64: (1R,2S,5S)-3-(2-(2,2-Difluorocyclohexyl)acetyl)-6,6-dimethyl-N—((S)-1-oxo-3-((S)-2-oxopyrrolidin-3-yl)propan-2-yl)-3-azabicyclo[3.1.0]hexane-2-carboxamide (Compound 64)

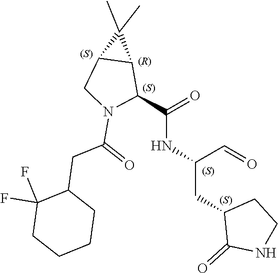

Compound 64 was synthesized by the same procedure as Compound 1. ¹H NMR (400 MHz, CD₃OD) δ ppm 4.66-4.15 (m, 3H), 3.91-3.57 (m, 2H), 3.55-3.43 (m, 1H), 2.68-2.29 (m, 2H), 2.27-2.13 (m, 2H), 1.98-1.86 (m, 2H), 1.81-1.31 (m, 10H), 1.28-1.03 (m, 2H), 1.00-0.96 (m, 3H), 0.90-0.84 (m, 3H). ESI-MS [M+H]⁺ calc'd for C₂₃H₃₃F₂N₃O₄, 454.24, found 454.5.

### Example 65: (1R,2S,5S)-3-(4-Methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 65)

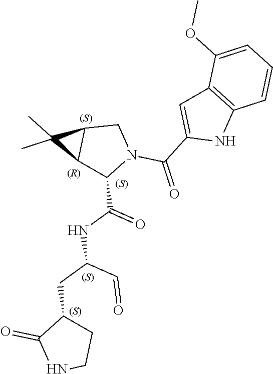

Compound 65 was synthesized by the same procedure as Compound 1. ¹H NMR (400 MHz, CD₃OD) δ ppm 9.66-9.24 (m, 1H), 8.32 (d, J=9.6 Hz, 1H), 7.22-6.91 (m, 3H), 6.55-6.37 (m, 1H), 5.09-4.90 (m, 1H), 4.83-4.59 (m, 1H), 4.52-4.26 (m, 1H), 4.05-3.74 (m, 5H), 3.30-3.22 (m, 1H), 2.46-2.19 (m, 1H), 2.10-1.91 (m, 1H), 1.86-1.49 (m, 3H), 1.48-1.22 (m, 2H), 1.18-0.94 (m, 6H). ESI-MS [M+H]⁺ calc'd for C₂₅H₃₀N₄O₅, 467.2, found 467.4.

### Example 66: (1R,2S,5S)-3-(4-Isopropoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 66)

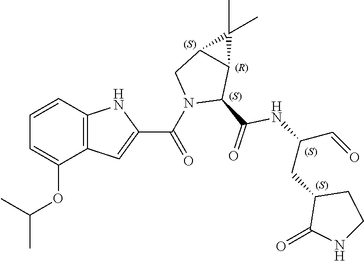

Compound 66 was synthesized by the same procedure as Compound 1. ¹H NMR (400 MHz, CD₃OD) δ ppm 7.07-6.98 (m, 1H), 6.97-6.90 (m, 1H), 6.89-6.80 (m, 1H), 6.37 (d, J=7.6 Hz, 1H), 4.70-4.62 (m, 1H), 4.61-4.38 (m, 3H), 4.32-4.16 (m, 1H), 3.98-3.86 (m, 1H), 3.70-3.56 (m, 1H), 3.19-3.14 (m, 1H), 2.73-2.46 (m, 1H), 2.31-1.79 (m, 2H), 1.72-1.38 (m, 3H), 1.36-1.20 (m, 6H), 1.20-0.97 (m, 5H), 0.87 (s, 1H). ESI-MS [M+H]⁺ calc'd for C₂₇H₃₄N₄O₅, 495.25; found, 495.3.

### Example 67: (1R,2S,5S)-3-((R)-2-Cyclohexylpropanoyl)-6,6-dimethyl-N—((S)-1-oxo-3-((S)-2-oxopyrrolidin-3-yl)propan-2-yl)-3-azabicyclo[3.1.0]hexane-2-carboxamide (Compound 67)

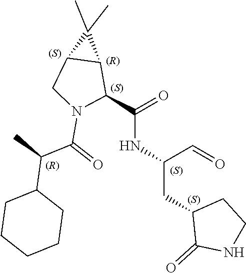

Compound 67 was synthesized by the same procedure as Compound 1. ¹H NMR (400 MHz, CD₃OD) δ ppm 4.60-4.48 (m, 1H), 4.40-4.26 (m, 1H), 4.06-3.88 (m, 2H), 3.70-3.63 (m, 1H), 3.31-3.27 (m, 1H), 2.70-2.56 (m, 1H), 2.50-2.27 (m, 2H), 2.09-1.96 (m, 1H), 1.95-1.73 (m, 3H), 1.71-1.42 (m, 7H), 1.32-1.15 (m, 3H), 1.09 (s, 3H), 1.07-0.88 (m, 8H). ESI-MS [M+H]⁺ calc'd for C₂₄H₃₇N₃O₄, 432.28; found, 432.5.

### Example 68: (1R,2S,5S)-3-(5-Methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 68)

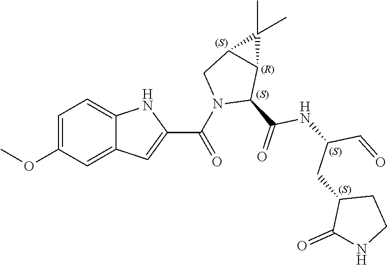

Compound 68 was synthesized by the same procedure as Compound 1. ¹H NMR (400 MHz, CD₃OD) δ ppm 7.35-7.28 (m, 1H), 7.14-6.98 (m, 1H), 6.98-6.83 (m, 2H), 5.02-4.89 (m, 1H), 4.64-4.39 (m, 1H), 4.32-3.92 (m, 2H), 3.80 (d, J=10.8 Hz, 3H), 3.78-3.69 (m, 1H), 3.29-3.22 (m, 1H), 2.92-2.57 (m, 1H), 2.40-2.17 (m, 1H), 2.06-1.90 (m, 1H), 1.79-1.68 (m, 1H), 1.62-1.52 (m, 1H), 1.50-1.24 (m, 2H), 1.17-0.88 (m, 6H). ESI-MS [M+H]⁺ calc'd for C₂₅H₃₀N₄O₅, 467.22; found, 467.4.

### Example 69: (1R,2S,5S)-3-(5-Chloro-7-methyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 69)

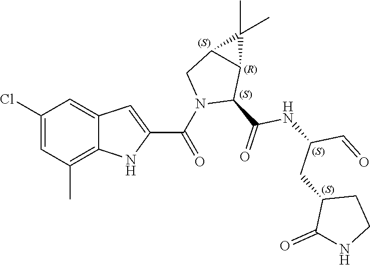

Compound 69 was synthesized by the same procedure as Compound 1. ¹H NMR (400 MHz, CD₃OD) δ ppm 7.51-7.33 (m, 1H), 7.06-6.82 (m, 2H), 4.99-4.88 (m, 1H), 4.54-4.38 (m, 1H), 4.32-3.96 (m, 2H), 3.95-3.73 (m, 1H), 2.99-2.58 (m, 1H), 2.50 (s, 3H), 2.44-2.29 (m, 1H), 2.07-1.82 (m, 1H), 1.80-1.70 (m, 1H), 1.64-1.50 (m, 2H), 1.40-1.29 (m, 1H), 1.19-0.89 (m, 7H). ESI-MS [M+H]⁺ calc'd for C₂₅H₂₉ClN₄O₄, 485.19; found, 485.4.

### Example 70: (1R,2S,5S)-3-(4-Ethoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 70)

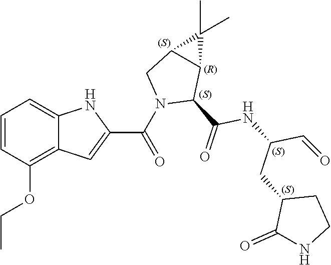

Compound 70 was synthesized by the same procedure as Compound 1. ¹H NMR (400 MHz, CD₃OD) δ ppm 7.17-6.94 (m, 3H), 6.53-6.40 (m, 1H), 4.68-4.55 (m, 1H), 4.52-4.26 (m, 1H), 4.23-4.07 (m, 2H), 4.06-3.94 (m, 1H), 3.83-3.63 (m, 1H), 3.37-3.32 (m, 1H), 3.28-3.22 (m, 1H), 2.81-2.30 (m, 2H), 2.21-1.91 (m, 2H), 1.83-1.67 (m, 1H), 1.62-1.27 (m, 5H), 1.17-0.91 (m, 6H). ESI-MS [M+H]⁺ calc'd for C₂₆H₃₂N₄O₅, 481.24; found, 481.1.

### Example 71: (2S,4S)-4-cyclohexyl-1-(7-fluoro-1H-indole-2-carbonyl)-N—((S)-1-oxo-3-((S)-2-oxopyrrolidin-3-yl)propan-2-yl)pyrrolidine-2-carboxamide (Compound 71)

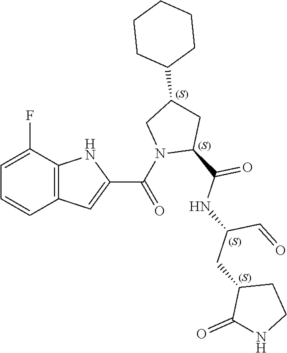

### Step 1: (2S,4S)-1-tert-butoxycarbonyl-4-cyclohexyl-pyrrolidine-2-carboxylic acid

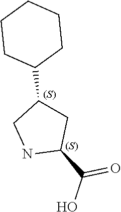

To a solution of (2S,4S)-4-cyclohexylpyrrolidine-2-carboxylic acid (2.0 g, 10.14 mmol) and K₂CO₃ (4.9 g, 35.49 mmol) in THE (15 mL) was added H₂O (15 mL). After addition, the mixture was stirred at 0° C. for 5 min, and then tert-butoxycarbonyl tert-butyl carbonate (4.43 g, 20.28 mmol) was added dropwise at 0° C. The resulting mixture was stirred at 25° C. for 12 h. Water (10 mL) was added to the reaction mixture. The reaction mixture was then adjusted to pH~5 by aq. HCl (2M). The resulting mixture was extracted with EtOAc (20 mL×3). The combined organic phase was washed with brine (20 mL), dried over anhydrous Na₂SO₄, filtered and concentrated. The title compound (3.9 g, crude) was obtained as a white solid. ¹H NMR (400 MHz, CDCl₃) δ ppm 4.45-4.22 (m, 1H), 3.80-3.52 (m, 1H), 3.02-2.87 (m, 1H), 2.43-2.11 (m, 1H), 2.06-1.86 (m, 1H), 1.78-1.57 (m, 6H), 1.48-1.36 (m, 9H), 1.24-1.06 (m, 4H), 1.04-0.85 (m, 2H).

### Step 2: (2S,4S)-tert-butyl 4-cyclohexyl-2-(((S)-1-methoxy-1-oxo-3-((S)-2-oxopyrrolidin-3-yl)propan-2-yl)carbamoyl)pyrrolidine-1-carboxylate

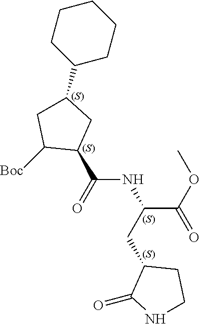

To a solution of (2S,4S)-1-(tert-butoxycarbonyl)-4-cyclohexylpyrrolidine-2-carboxylic acid (1 g, 3.36 mmol) and DIEA (1.74 g, 13.45 mmol) in DCM (10 mL) was added dropwise T₃P (3.21 g, 5.04 mmol, 50% purity in EtOAc) at 0° C. After addition, the mixture was stirred at the same temperature for 30 min, and then (S)-methyl 2-amino-3-((S)-2-oxopyrrolidin-3-yl)propanoate (748.7 mg, 3.36 mmol, HCl) was added dropwise at 25° C. The resulting mixture was stirred at 25° C. for 10 h. Water (10 mL) was added and the reaction was extracted with DCM (10 mL×3). The combined organic phase was washed with 5% H₃PO₄ (5 mL×2) and sat aq. NaHCO₃ (5 mL×2), dried over anhydrous Na₂SO₄, filtered and concentrated to dryness. The title compound (1.89 g, 73.65% yield) was obtained as a yellow solid. ¹H NMR (400 MHz, CDCl₃) δ ppm 7.92-7.36 (m, 1H), 6.90-6.19 (m, 1H), 4.64-4.41 (m, 1H), 4.37-4.15 (m, 1H), 3.77-3.65 (m, 3H), 3.64-3.52 (m, 1H), 3.40-3.24 (s, 2H), 3.08-2.82 (m, 1H), 2.49-2.12 (m, 4H), 1.92-1.75 (m, 3H), 1.74-1.57 (m, 6H), 1.49-1.37 (m, 9H), 1.30-1.05 (m, 4H), 0.95 (s, 2H).

### Step 3: (S)-methyl 2-((2S,4S)-4-cyclohexylpyrrolidine-2-carboxamido)-3-((S)-2-oxopyrrolidin-3-yl)propanoate hydrochloride

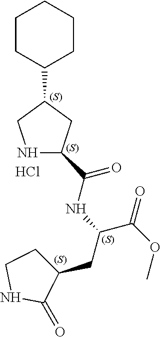

A solution of (2S,4S)-tert-butyl 4-cyclohexyl-2-(((S)-1-methoxy-1-oxo-3-((S)-2-oxopyrrolidin-3-yl)propan-2-yl)carbamoyl)pyrrolidine-1-carboxylate (300 mg, 644.36 mol) in HCl/dioxane (3 mL) was stirred at 25° C. for 1 h. The reaction mixture was concentrated directly. The title compound (258 mg, crude, HCl) was obtained as a yellow solid, which was used to next step without further purification.

### Step 5: (S)-methyl 2-((2S,4S)-4-cyclohexyl-1-(7-fluoro-1H-indole-2-carbonyl)pyrrolidine-2-carboxamido)-3-((S)-2-oxopyrrolidin-3-yl)propanoate

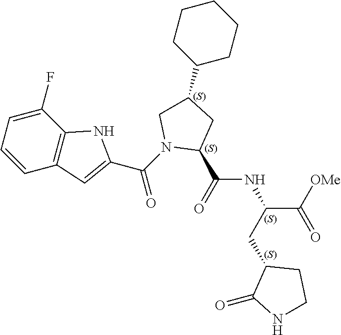

To a mixture of (S)-methyl 2-((2S,4S)-4-cyclohexylpyrrolidine-2-carboxamido)-3-((S)-2-oxopyrrolidin-3-yl)propanoate (258 mg, 641.91 mol HCl) and 7-fluoro-1H-indole-2-carboxylic acid (115 mg, 641.91 mol) in DCM (5 mL) was added DIEA (248 mg, 1.93 mmol) and HATU (488 mg, 1.28 mmol) at 0° C. The mixture was stirred at 25° C. for 12 hrs. The mixture was diluted with DCM (20 mL), and washed with sat.aq. NH₄Cl (10 mL×2). The organic layer was dried over Na₂SO₄, filtered and concentrated in vacuo. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 30%-70%, 11 min). The solvent after prep-HPLC purification was extracted with DCM (30 mL×3). The combined organic layer was dried over Na₂SO₄, filtered and concentrated to dryness. The title compound (60 mg, 13.14% yield) was obtained as a yellow oil. ESI-MS [M+H]⁺ calc'd for C₂₈H₃₅FN₄O₅, 527.26; found, 527.2.

### Step 6: (2S,4S)-4-cyclohexyl-1-(7-fluoro-1H-indole-2-carbonyl)-N—((S)-1-hydroxy-3-((S)-2-oxopyrrolidin-3-yl)propan-2-yl)pyrrolidine-2-carboxamide

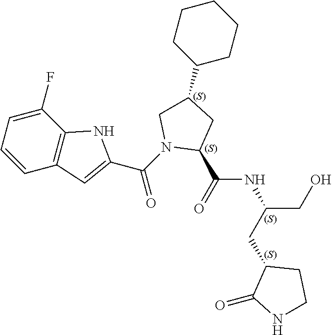

To a mixture of (S)-methyl 2-((2S,4S)-4-cyclohexyl-1-(7-fluoro-1H-indole-2-carbonyl)pyrrolidine-2-carboxamido)-3-((S)-2-oxopyrrolidin-3-yl)propanoate (60 mg, 113.94 mol) in THE (3 mL) was added LiBH₄ (50 mg, 2.30 mmol, 2.30 mL) at 0° C. under N₂. The mixture was stirred at 0° C. for 1 hr. The mixture was quenched with H₂O (10 mL), and extracted with DCM (5 mL×3). The combined organic layer was dried over anhydrous Na₂SO₄, filtered and concentrated in vacuo. The title compound (60 mg, 68.65% yield) was obtained as a white solid. ESI-MS [M+H]⁺ calc'd for C₂₇H₃₅FN₄O₄, 499.26; found, 499.3.

### Step 7: (2S,4S)-4-cyclohexyl-1-(7-fluoro-1H-indole-2-carbonyl)-N—((S)-1-oxo-3-((S)-2-oxopyrrolidin-3-yl)propan-2-yl)pyrrolidine-2-carboxamide

To a mixture of (2S,4S)-4-cyclohexyl-1-(7-fluoro-1H-indole-2-carbonyl)-N—((S)-1-hydroxy-3-((S)-2-oxopyrrolidin-3-yl)propan-2-yl)pyrrolidine-2-carboxamide (50 mg, 100.28 mol) in DCM (2 mL) was added DMP (85 mg, 200.57 mol). The mixture was stirred at 25° C. for 0.5 hr. The mixture was quenched with H₂O (3 mL), and extracted with DCM:MeOH (10:1, 5 mL×3). The combined organic layer was dried over anhydrous Na₂SO₄, filtered and concentrated in vacuo. The residue was purified by prep-HPLC (column: Phenomenex Gemini-NX C18 75×30 mm×3 μm; mobile phase: [water (10 mM NH₄HCO₃)-ACN]; B %: 27%-67%, 11 min). Compound 71 (8.3 mg, 15.18% yield, 91.1% purity) was obtained as a white solid. ¹H NMR (400 MHz, CD₃OD) δ ppm 7.60-7.28 (m, 1H), 7.17-6.88 (m, 3H), 4.73 (br d, J=8.8 Hz, 1H), 4.55-4.37 (m, 1H), 4.32-4.10 (m, 1H), 4.05-3.72 (m, 1H), 3.67-3.47 (m, 1H), 3.11-2.93 (m, 1H), 2.69-2.52 (m, 1H), 2.48-2.23 (m, 2H), 2.22-1.90 (m, 3H), 1.88-1.57 (m, 6H), 1.55-1.17 (m, 5H), 1.16-0.95 (m, 2H). ESI-MS [M+H]⁺ calc'd for C₂₇H₃₃FN₄O₄, 497.25; found, 497.3.

### Example 72: (1R,2S,5S)-6,6-Dimethyl-3-(6-methyl-1H-indole-2-carbonyl)-N—((S)-1-oxo-3-((S)-2-oxopyrrolidin-3-yl)propan-2-yl)-3-azabicyclo[3.1.0]hexane-2-carboxamide (Compound 72)

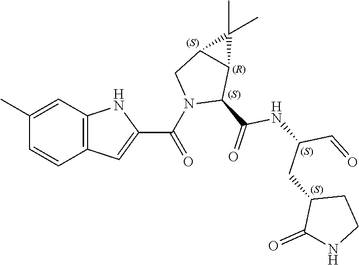

Compound 72 was synthesized by the same procedure as Compound 1. ¹H NMR (400 MHz, CD₃OD) δ ppm 7.58-7.37 (m, 1H), 7.28-7.18 (m, 1H), 7.07-6.89 (m, 1H), 6.89-6.81 (m, 1H), 4.54-4.37 (m, 1H), 4.32-3.93 (m, 2H), 3.83-3.67 (m, 1H), 3.29-3.21 (m, 1H), 2.94-2.56 (m, 1H), 2.42 (d, J=5.2 Hz, 3H), 2.39-2.18 (m, 1H), 2.10-1.66 (m, 3H), 1.64-1.25 (m, 3H), 1.19-0.92 (m, 6H). ESI-MS [M+H]⁺ calc'd for C₂₅H₃₀N₄O₄, 451.23; found, 451.4.

### Example 73: (1R,2S,5S)-3-(4,4-Dimethylpentanoyl)-6,6-dimethyl-N—((S)-1-oxo-3-((S)-2-oxopyrrolidin-3-yl)propan-2-yl)-3-azabicyclo[3.1.0]hexane-2-carboxamide (Compound 73)

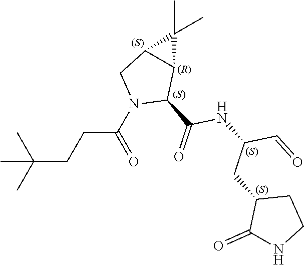

Compound 73 was synthesized by the same procedure as Compound 1. ¹H NMR (400 MHz, CD₃OD) δ ppm 4.53-4.44 (m, 1H), 4.35-4.24 (m, 1H), 4.02-3.66 (m, 2H), 3.64-3.47 (m, 1H), 3.29-3.24 (m, 1H), 2.62-2.08 (m, 4H), 2.06-1.94 (m, 1H), 1.87-1.65 (m, 1H), 1.61-1.38 (m, 5H), 1.10-0.95 (m, 6H), 0.93-0.86 (m, 9H). ESI-MS [M+H]⁺ calc'd for C₂₂H₃₅N₃O₄, 406.26; found, 406.4.

### Example 74: (1R,2S,5S)-3-(6-Cyano-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 74)

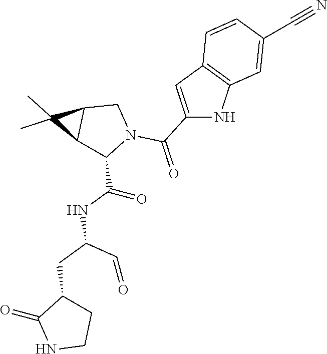 [SMILES: CC1(C)[C@@H]2[C@@H](C(=O)N[C@H](C=O)C[C@@H]3CCNC3=O)N(C(=O)c3cc4ccc(C#N)cc4[nH]3)C[C@@H]21]

Compound 74 was synthesized by the same procedure as Compound 1. ¹H NMR (400 MHz, CD₃OD) δ ppm 7.87-7.68 (m, 2H), 7.37-7.25 (m, 1H), 7.21-6.96 (m, 1H), 4.53-4.38 (m, 1H), 4.35-4.00 (m, 1H), 4.00-3.92 (m, 1H), 3.80-3.66 (m, 1H), 3.30-3.21 (m, 1H), 3.00-2.57 (m, 1H), 2.41-2.23 (m, 1H), 2.11-1.77 (m, 1H), 1.77-1.69 (m, 1H), 1.68-1.54 (m, 2H), 1.54-1.23 (m, 2H), 1.16-0.96 (m, 6H). ESI-MS [M+H]⁺ calc'd for C₂₅H₂₇N₅O₄, 462.21; found, 462.4.

### Example 75: (2S,4S)-1-(7-cyano-1H-indole-2-carbonyl)-4-cyclohexyl-N—((S)-1-oxo-3-((S)-2-oxopyrrolidin-3-yl)propan-2-yl)pyrrolidine-2-carboxamide (Compound 75)

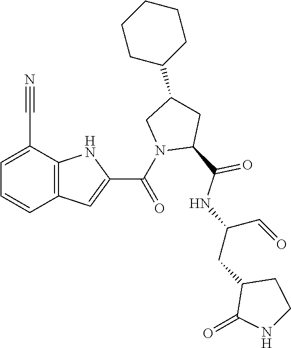

Step 1: methyl 7-bromo-1H-indole-2-carboxylate

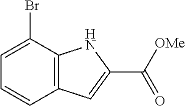 [SMILES: COC(=O)c1cc2cccc(Br)c2[nH]1]

To a solution of 7-bromo-1H-indole-2-carboxylic acid (1.00 g, 4.17 mmol) in MeOH (12 mL) was added SOCl₂ (1.5 g, 12.50 mmol) at 0° C. The mixture was stirred at 50° C. for 12 hr. The reaction mixture was concentrated in vacuo. The residue was dissolved in water (20 mL) and then extracted with EtOAc (40 mL×3). The combined organic phase was washed with water (30 mL), dried over anhydrous Na₂SO₄, filtered and concentrated. The title compound (1.17 g, crude) was obtained as a white solid. ¹H NMR (400 MHz, CDCl₃) δ ppm 8.97 (s, 1H), 7.64 (d, J=8.0 Hz, 1H), 7.80 (d, J=8.0 Hz, 1H), 7.28 (d, J=2.4 Hz, 1H), 7.04 (t, J=8.0 Hz, 1H), 3.97 (s, 3H).

### Step 2: methyl 7-cyano-1H-indole-2-carboxylate

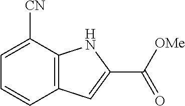 [SMILES: COC(=O)c1cc2cccc(C#N)c2[nH]1]

A mixture of methyl 7-bromo-1H-indole-2-carboxylate (500 mg, 1.97 mmol) and CuCN (352 mg, 3.94 mmol, 859.76 μL) in NMP (10 mL) was degassed and purged with N₂ for 3 times, and then the mixture was stirred at 180° C. for 2 hr under microwave. Water (15 mL) was added and the reaction mixture was adjusted to pH~10 by aq. NaOH (6 M). The resulting mixture was extracted with EtOAc (30 mL×3). The combined organic phase was washed with aq. NH₄Cl (20 mL×3), dried over anhydrous Na₂SO₄, filtered and concentrated in vacuo. The title compound (420 mg, crude) was obtained as a yellow solid. ¹H NMR (400 MHz, CDCl₃) δ ppm 9.49 (s, 1H), 7.94 (d, J=8.0 Hz, 1H), 7.66 (dd, J=0.8, 7.2 Hz, 1H), 7.30 (d, J=2.0 Hz, 1H), 7.23 (t, J=8.0 Hz, 1H), 3.99 (s, 3H).

### Step 3: 7-cyano-1H-indole-2-carboxylic acid

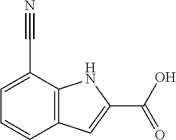 [SMILES: N#Cc1cccc2cc(C(=O)O)[nH]c12]

To a solution of methyl 7-cyano-1H-indole-2-carboxylate (420 mg, 2.10 mmol) in THE (4 mL) and H₂O (4 mL) was added LiOH·H₂O (352 mg, 8.39 mmol). The mixture was stirred at 25° C. for 10 hr. Water (10 mL) was added and the reaction mixture was adjusted to pH~5 by aq. HCl (1 N). The resulting mixture was extracted with EtOAc (30 mL×3). The combined organic phase was washed with brine (20 mL), dried over anhydrous Na₂SO₄, filtered and concentrated. The title compound (310 mg, crude) was obtained as a yellow solid. ¹H NMR (400 MHz, CD₃OD) δ ppm 7.98 (d, J=8.4 Hz, 1H), 7.68 (d, J=7.6 Hz, 1H), 7.28 (s, 1H), 7.25-7.16 (m, 1H).

### Step 4: (S)-methyl 2-((2S,4S)-1-(7-cyano-1H-indole-2-carbonyl)-4-cyclohexylpyrrolidine-2-carboxamido)-3-((S)-2-oxopyrrolidin-3-yl)propanoate

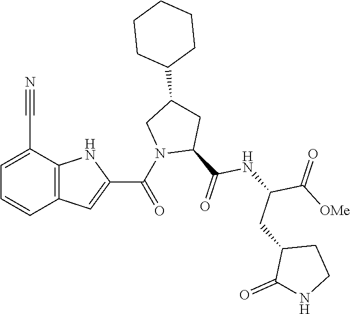 [SMILES: COC(=O)[C@H](C[C@@H]1CCNC1=O)NC(=O)[C@@H]1C[C@@H](C2CCCCC2)CN1C(=O)c1cc2cccc(C#N)c2[nH]1]

To a solution of compound (S)-methyl 2-((2S,4S)-4-cyclohexylpyrrolidine-2-carboxamido)-3-((S)-2-oxopyrrolidin-3-yl)propanoate (648 mg, 1.61 mmol, HCl), 7-cyano-1H-indole-2-carboxylic acid (300 mg, 1.61 mmol,) and DIEA (625 mg, 4.83 mmol) in DCM (10 mL) was added slowly HATU (1.23 g, 3.22 mmol) at 0° C. The resulting mixture was stirred at 25° C. for 3 hr. Sat.aq.NH₄Cl (30 mL) was added. The resulting mixture was extracted with DCM (50 mL×3). The combined organic phase was washed with water (20 mL), dried over anhydrous Na₂SO₄, filtered and concentrated in vacuo. The residue was purified by flash silica gel chromatography (Eluent of 0~96% EtOAc in PE). The title compound (500 mg, 26.72% yield) was obtained as a yellow solid. ¹H NMR (400 MHz, CDCl₃) δ ppm 11.55 (brs, 1H), 9.05 (brs, 1H), 8.05-7.81 (m, 1H), 7.66-7.47 (m, 1H), 7.24-6.94 (m, 2H), 5.17-4.69 (m, 1H), 4.65-4.17 (m, 1H), 4.09-3.96 (m, 1H), 3.79-3.73 (m, 3H), 3.60-3.37 (m, 3H), 2.79-2.24 (m, 8H), 2.16-2.07 (m, 1H), 1.96-1.56 (m, 5H), 1.23-0.79 (m, 5H).

### Step 5: (2S,4S)-1-(7-cyano-1H-indole-2-carbonyl)-4-cyclohexyl-N—((S)-1-hydroxy-3-((S)-2-oxopyrrolidin-3-yl)propan-2-yl)pyrrolidine-2-carboxamide

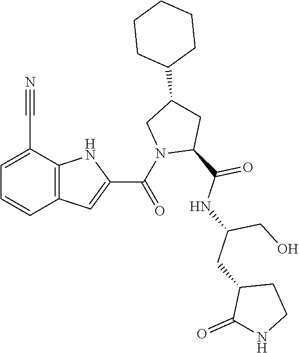 [SMILES: N#Cc1cccc2cc(C(=O)N3C[C@H](C4CCCCC4)C[C@H]3C(=O)N[C@H](CO)C[C@@H]3CCNC3=O)[nH]c12]

To a solution of (S)-methyl 2-((2S,4S)-1-(7-cyano-1H-indole-2-carbonyl)-4-cyclohexylpyrrolidine-2-carboxamido)-3-((S)-2-oxopyrrolidin-3-yl)propanoate (500 mg, 937.00 mol) in THE (5 mL) was added LiBH₄ (70 mg, 3.21 mmol) at 0° C. The mixture was stirred at 25° C. for 1 h. The reaction was quenched with water (15 mL) drop-wise. The resulting mixture was extracted with DCM (30 mL×3). The combined organic phase was washed with water (10 mL), dried over anhydrous Na₂SO₄, filtered and concentrated in vacuo. The residue was purified by prep-HPLC (column: Phenomenex C18 75×30 mm×3 m; mobile phase: [water (NH₃H₂O+NH₄HCO₃)-ACN]; B %: 27%-57%, 10 min). The title compound (30 mg, 4.88% yield) was obtained. ¹H NMR (400 MHz, CDCl₃) δ ppm 11.87 (brs, 1H), 9.01 (d, J=3.6 Hz, 1H), 7.97-7.61 (m, 1H), 7.61-7.36 (m, 1H), 7.19 (t, J=7.6 Hz, 1H), 6.99 (d, J=2.0 Hz, 1H), 5.12-4.82 (m, 1H), 4.68-4.37 (m, 1H), 4.13-3.99 (m, 1H), 3.96-3.84 (m, 1H), 3.79-3.56 (m, 2H), 3.53-3.28 (m, 2H), 3.05 (t, J=9.6 Hz, 1H), 2.59-2.45 (m, 1H), 2.43-2.27 (m, 1H), 2.08-1.46 (m, 10H), 1.34-1.03 (m, 5H), 1.00-0.80 (m, 2H).

### Step 6: (2S,4S)-1-(7-cyano-1H-indole-2-carbonyl)-4-cyclohexyl-N—((S)-1-oxo-3-((S)-2-oxopyrrolidin-3-yl)propan-2-yl)pyrrolidine-2-carboxamide

To a solution of (2S,4S)-1-(7-cyano-1H-indole-2-carbonyl)-4-cyclohexyl-N—((S)-1-hydroxy-3-((S)-2-oxopyrrolidin-3-yl)propan-2-yl)pyrrolidine-2-carboxamide (20 mg, 39.56 mol) in DCM (4 mL) was added DMP (50 mg, 118.67 mol). The mixture was stirred at 25° C. for 1 hr. The reaction mixture was filtered. The filtrate was concentrated. The residue was purified by prep-HPLC (column: Phenomenex C18 75×30 mm×3 μm; mobile phase: [water (NH₄HCO₃)-ACN]; B %: 28%-58%, 10 min). Compound 75 (4.3 mg, 20.48% yield, 94.895% purity) was obtained as a white solid. 1H NMR (400 MHz, CD₃OD) δ ppm 8.11-7.81 (m, 1H), 7.75-7.57 (m, 1H), 7.34-6.91 (m, 2H), 4.72 (br d, J=8.8 Hz, 1H), 4.54-4.35 (m, 1H), 4.29-4.10 (m, 1H), 4.07-3.72 (m, 1H), 3.67-3.45 (m, 1H), 3.29-3.02 (m, 1H), 2.73-2.51 (m, 1H), 2.48-1.92 (m, 4H), 1.81-0.99 (m, 14H). ESI-MS [M+H]⁺ calc'd for C₂₈H₃₃N₅O₄, 504.25; found, 504.4.

### Example 76: (S)-5-(4-Cyano-1H-indole-2-carbonyl)-N—((S)-1-oxo-3-((S)-2-oxopyrrolidin-3-yl)propan-2-yl)-5-azaspiro[2.4]heptane-6-carboxamide (Compound 76)

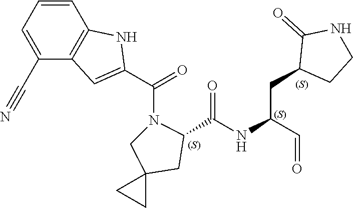

Compound 76 was synthesized by the same procedure as Compound 2. ¹H NMR (400 MHz, CD₃OD) δ ppm 7.80-7.73 (m, 1H), 7.55-7.47 (m, 1H), 7.39-7.31 (m, 1H), 7.12-6.94 (m, 1H), 4.84-4.78 (m, 1H), 4.51-4.36 (m, 1H), 4.06-3.93 (m, 1H), 3.89 (dd, J=6.0, 10.0 Hz, 1H), 3.85-3.66 (m, 1H), 2.71-2.51 (m, 1H), 2.47-2.34 (m, 1H), 2.28-2.18 (m, 1H), 2.13-1.98 (m, 2H), 1.95-1.36 (m, 3H), 0.83-0.59 (m, 4H). ESI-MS [M+H]⁺ calc'd for C₂₄H₂₅N₅O₄, 448.19; found, 448.4.

### Example 77: (1R,2S,5S)-3-(7-Fluoro-4-methyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 77)

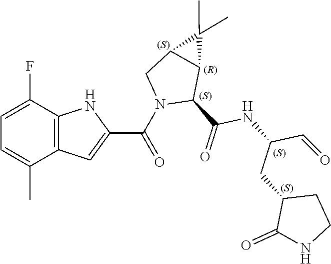

Compound 77 was synthesized by the same procedure as Compound 1. ¹H NMR (400 MHz, CD₃OD) δ ppm 7.14-6.73 (m, 3H), 4.56-3.93 (m, 3H), 3.83-3.64 (m, 1H), 2.94-2.59 (m, 1H), 2.56-2.45 (m, 3H), 2.42-1.98 (m, 2H), 1.91-1.26 (m, 6H), 1.20-0.99 (m, 6H). ESI-MS [M+H]⁺ calc'd for C₂₅H₂₉FN₄O₄, 469.22; found, 469.4.

### Example 78: (1R,2S,5S)-3-(4H-Furo[3,2-b]pyrrole-5-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 78)

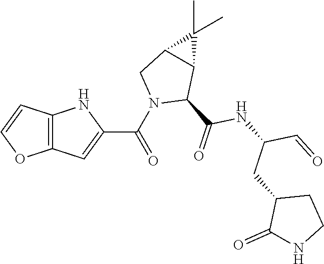 [SMILES: CC1(C)[C@@H]2[C@@H](C(=O)N[C@H](C=O)C[C@@H]3CCNC3=O)N(C(=O)c3cc4occc4[nH]3)C[C@@H]21]

Compound 78 was synthesized by the same procedure as Compound 1. ¹H NMR (400 MHz, CD₃OD) δ ppm 7.65-7.42 (m, 1H), 6.83-6.35 (m, 2H), 4.56-4.39 (m, 1H), 4.17 (m, 1H), 3.99-3.76 (m, 2H), 3.11-2.74 (m, 1H), 2.67-2.54 (m, 1H), 2.44-2.24 (m, 1H), 2.11-1.96 (m, 1H), 1.81-1.42 (m, 5H), 1.11-0.93 (m, 6H). ESI-MS [M+H]⁺ calc'd for C₂₂H₂₆N₄O₅, 427.19; found, 427.4.

### Example 79: (1R,2S,5S)-3-(2-Cyclohexylacetyl)-6,6-dimethyl-N—((S)-1-oxo-3-((S)-2-oxopiperidin-3-yl)propan-2-yl)-3-azabicyclo[3.1.0]hexane-2-carboxamide (Compound 79)

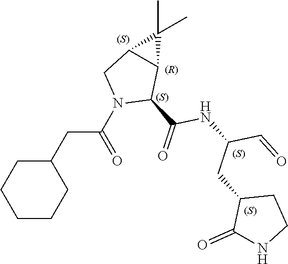

Compound 79 was synthesized by the same procedure as Compound 1. ¹H NMR (400 MHz, CD₃OD) δ ppm 4.82 (m, 1H), 4.55-4.22 (m, 2H), 4.07-3.65 (m, 2H), 3.62-3.54 (m, 1H), 3.29-3.23 (m, 2H), 2.43 (m, 1H), 2.22-2.05 (m, 3H), 1.85-1.62 (m, 8H), 1.56-1.38 (m, 3H), 1.32-1.13 (m, 3H), 1.13-0.79 (m, 8H). ESI-MS [M+H]⁺ calc'd for C₂₄H₃₇N₃O₄, 432.28; found, 432.4.

### Example 80: (1R,2S,5S)-6,6-Dimethyl-3-(5-(methylsulfonyl)-1H-indole-2-carbonyl)-N—((S)-1-oxo-3-((S)-2-oxopyrrolidin-3-yl)propan-2-yl)-3-azabicyclo[3.1.0]hexane-2-carboxamide (Compound 80)

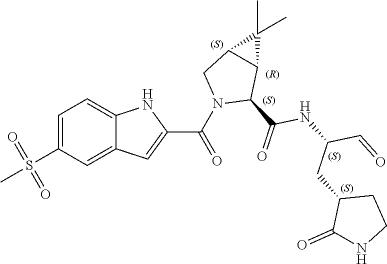

Compound 80 was synthesized by the same procedure as Compound 1. ¹H NMR (400 MHz, CD₃OD) δ ppm 8.40-8.19 (m, 1H), 7.76-7.68 (m, 1H), 7.67-7.59 (m, 1H), 7.31-7.05 (m, 1H), 4.64-4.38 (m, 1H), 4.36-3.94 (m, 2H), 3.82-3.68 (m, 1H), 3.36-3.32 (m, 1H), 3.28-3.23 (m, 1H), 3.15 (d, J=18.4 Hz, 3H), 3.00-2.58 (m, 1H), 2.50-2.29 (m, 1H), 2.12-1.75 (m, 1H), 1.72-1.30 (m, 4H), 1.19-0.96 (m, 6H). ESI-MS [M+H]⁺ calc'd for C₂₅H₃₀N₄O₆S, 515.19; found, 515.1.

### Example 81: (2S,4S)-1-(7-chloro-1H-indole-2-carbonyl)-4-cyclohexyl-N—((S)-1-oxo-3-((S)-2-oxopyrrolidin-3-yl)propan-2-yl)pyrrolidine-2-carboxamide (Compound 81)

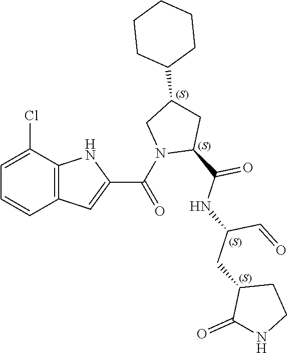

### Step 1: (u)-methyl 2-((2S,4S )-4-cyclohexylpyrrolidine-2-carboxamido)-3-((S)-2-oxopyrrolidin-3-yl)propanoate

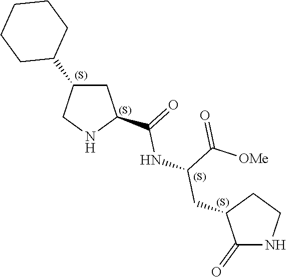

To a mixture of (2S,4S)-tert-butyl 4-cyclohexyl-2-(((S)-1-methoxy-1-oxo-3-((S)-2-oxopyrrolidin-3-yl) propan-2-yl) carbamoyl) pyrrolidine-1-carboxylate (350 mg, 751.75 μmol) in dioxane (4 mL) was added HCl/dioxane (4M, 4 mL). The mixture was stirred at 25° C. for 1 hr. The reaction mixture was concentrated to dryness. The title compound (300 mg, crude, HCl) was obtained as a white solid.

### Step 2: (S)-methyl 2-((2S,4S)-1-(7-chloro-1H-indole-2-carbonyl)-4-cyclohexylpyrrolidine-2-carboxamido)-3-((S)-2-oxopyrrolidin-3-yl)propanoate

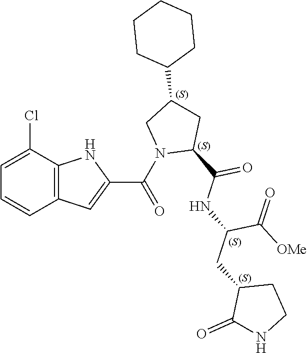

To a solution of (S)-methyl 2-((2S,4S)-4-cyclohexylpyrrolidine-2-carboxamido)-3-((S)-2-oxopyrrolidin-3-yl)propanoate (300 mg, 746.40 μmol, HCl), 7-chloro-1H-indole-2-carboxylic acid (146 mg, 746.40 mol) and DIEA (289 mg, 2.24 mmol) in DCM (5 mL) was added HATU (567 mg, 1.49 mmol) slowly at 0° C. The mixture was stirred at 25° C. for 3 hr. Sat.aq.NH₄C₁ (20 mL) was added. The resulting mixture was extracted with DCM (20 mL×3). The combined organic phase was washed with brine (20 mL), dried over anhydrous Na₂SO₄, filtered and concentrated to dryness. The residue was purified by flash silica gel chromatography (Eluent of 0~88% EtOAc in PE). The title compound (400 mg, 58.54% yield) was obtained as a yellow solid. ¹H NMR (400 MHz, CDCl₃) δ ppm 9.78-9.38 (m, 1H), 8.80-7.92 (m, 1H), 7.69-7.54 (m, 1H), 7.53-7.28 (m, 1H), 7.16-7.02 (m, 1H), 6.94 (s, 1H), 6.13-5.99 (m, 1H), 4.98-4.72 (m, 1H), 4.64-4.27 (m, 1H), 4.25-3.94 (m, 1H), 3.80-3.65 (m, 3H), 3.46-3.28 (m, 3H), 2.41-2.24 (m, 3H), 1.97-1.73 (m, 5H), 1.69-1.63 (m, 2H), 1.30-0.92 (m, 9H).

### Step 3: (2S,4S)-1-(7-chloro-1H-indole-2-carbonyl)-4-cyclohexyl-N—((S)-1-hydroxy-3-((S)-2-oxopyrrolidin-3-yl)propan-2-yl)pyrrolidine-2-carboxamide

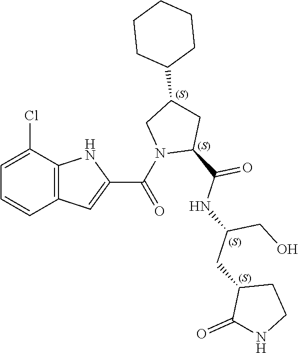

To a solution of (S)-methyl 2-((2S,4S)-1-(7-chloro-1H-indole-2-carbonyl)-4-cyclohexylpyrrolidine-2-carboxamido)-3-((S)-2-oxopyrrolidin-3-yl)propanoate (400 mg, 736.58 mol) in THF (4 mL) was added LiBH₄ (140 mg, 6.43 mmol) at 0° C. The mixture was stirred at 25° C. for 1 hr. The reaction was quenched with water (10 mL) dropwise. The resulting mixture was extracted with DCM (10 mL×3). The combined organic phase was washed with water (10 mL), dried over anhydrous Na₂SO₄, filtered and concentrated to dryness. The residue was purified by prep-HPLC (column: Xtimate C18 150×40 mm×10 μm; mobile phase: [water (0.05% NH₃H₂O)-ACN]; B %: 35%-65%, 10 min). The title compound (106 mg, 27.25% yield,) was obtained as a white solid. ¹H NMR (400 MHz, CDCl₃) δ ppm 9.85-9.46 (m, 1H), 8.37-7.94 (m, 1H), 7.64-7.44 (m, 1H), 7.31-7.27 (m, 1H), 7.10-7.03 (m, 1H), 7.00-6.77 (m, 1H), 5.92-5.59 (m, 1H), 4.94-4.65 (m, 1H), 4.22 (t, J=8.4 Hz, 1H), 4.09-3.91 (m, 1H), 3.82-3.73 (m, 1H), 3.63-3.52 (m, 1H), 3.42 (t, J=9.2 Hz, 1H), 3.35-3.24 (m, 2H), 2.60-2.24 (m, 5H), 2.08-2.02 (m, 1H), 1.91-1.74 (m, 3H), 1.69-1.57 (m, 3H), 1.30-0.91 (m, 7H).

### Step 4: (2S,4S)-1-(7-chloro-1H-indole-2-carbonyl)-4-cyclohexyl-N—((S)-1-oxo-3-((S)-2-oxopyrrolidin-3-yl)propan-2-yl)pyrrolidine-2-carboxamide

To a solution of (2S,4S)-1-(7-chloro-1H-indole-2-carbonyl)-4-cyclohexyl-N—((S)-1-hydroxy-3-((S)-2-oxopyrrolidin-3-yl)propan-2-yl)pyrrolidine-2-carboxamide (100 mg, 194.16 mol) in DCM (5 mL) was added DMP (165 mg, 388.32 mol). The mixture was stirred at 25° C. for 1 hr. The reaction mixture was filtered. The filtrate was concentrated to dryness. The residue was purified by prep-HPLC (column: Phenomenex Gemini-NX C18 75×30 mm×3 μm; mobile phase: [water (10 mM NH₄HCO₃)-ACN]; B %: 32%-62%, 10 min). Compound 81 (20.6 mg, 38.88 μmol, 20.03% yield, 96.84 purity) was obtained as a white solid. ¹H NMR (400 MHz, CD₃OD) δ ppm 7.71-7.42 (m, 1H), 7.32-7.21 (m, 1H), 7.15-6.87 (m, 2H), 4.71 (br d, J=8.4 Hz, 1H), 4.55-4.34 (m, 1H), 4.28-4.10 (m, 1H), 4.06-3.71 (m, 1H), 3.63-3.43 (m, 1H), 3.29-2.95 (m, 1H), 2.67-2.52 (m, 1H), 2.43-2.17 (m, 2H), 2.13-1.90 (m, 2H), 1.88-0.92 (m, 14H) ESI-MS [M+H]⁺ calc'd for C₂₇H₃₃ClN₄O₄, 513.22; found, 513.2.

### Example 82: (S)-5-(6-Cyano-1H-indole-2-carbonyl)-N—((S)-1-oxo-3-((S)-2-oxopyrrolidin-3-yl)propan-2-yl)-5-azaspiro[2.4]heptane-6-carboxamide (Compound 82)

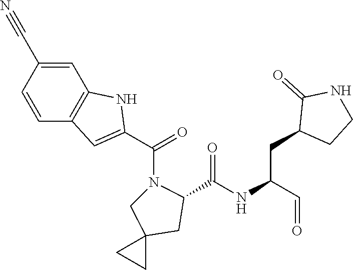 [SMILES: N#Cc1ccc2cc(C(=O)N3CC4(CC4)C[C@H]3C(=O)N[C@H](C=O)C[C@@H]3CCNC3=O)[nH]c2c1]

Compound 82 was synthesized by the same procedure as Compound 2. ¹H NMR (400 MHz, CD₃OD) δ ppm 7.86 (s, 1H), 7.83-7.68 (m, 1H), 7.36-7.27 (m, 1H), 7.13-6.95 (m, 1H), 4.84-4.75 (m, 2H), 4.51-4.35 (m, 1H), 4.10-3.93 (m, 1H), 3.89-3.81 (m, 1H), 3.78-3.68 (m, 1H), 3.12-2.62 (m, 1H), 2.59-2.37 (m, 1H), 2.27-1.97 (m, 2H), 1.90-1.71 (m, 1H), 1.67-1.39 (m, 2H), 0.80-0.60 (m, 4H). ESI-MS [M+H]⁺ calc'd for C₂₄H₂₅N₅O₄, 448.19; found, 448.3.

### Example 83: (1R,2S,5S)-6,6-dimethyl-N—((S)-1-oxo-3-((S)-2-oxopyrrolidin-3-yl)propan-2-yl)-3-(1H-pyrrolo[3,2-b]pyridine-2-carbonyl)-3-azabicyclo[3.1.0]hexane-2-carboxamide (Compound 83)

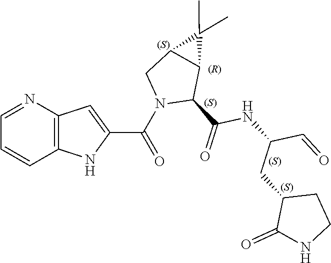

### Step 1: (S)-2-((tert-butoxycarbonyl)amino)-3-((S)-2-oxopyrrolidin-3-yl)propanoic acid

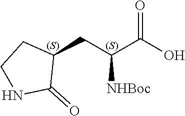

To a solution of (S)-methyl 2-((tert-butoxycarbonyl)amino)-3-((S)-2-oxopyrrolidin-3-yl)propanoate (5 g, 17.46 mmol) in THF (25 mL) and H₂O (25 mL) was added LiOH H₂O (1.5 g, 34.93 mmol) at 25° C. The mixture was stirred at 25° C. for 1 h. Water (15 mL) was added to the reaction mixture. The resulting mixture was extracted with EtOAc:PE (1:5) (10 mL). The water phase was then adjusted to pH~6 by aq. HCl (1 M) and then lyophilized. The title compound (6.1 g, crude,) was obtained as a white solid. ¹H NMR (400 MHz, DMSO-d₆) δ ppm 7.56 (s, 1H), 6.11 (d, J=7.2 Hz, 1H), 3.70-3.62 (m, 1H), 3.16-3.07 (m, 2H), 2.31-2.10 (m, 2H), 1.95-1.83 (m, 1H), 1.67-1.51 (m, 2H), 1.36 (s, 9H)

Step 2: tert-butyl ((S)-1-(methoxy(methyl)amino)-1-oxo-3-((S)-2-oxopyrrolidin-3-yl)propan-2-yl)carbamate

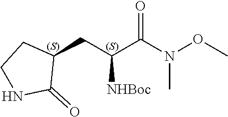

To a solution of (S)-2-((tert-butoxycarbonyl)amino)-3-((S)-2-oxopyrrolidin-3-yl)propanoic acid (5 g, 18.36 mmol) in DCM (40 mL) was added DIEA (9.5 g, 73.45 mmol, 12.79 mL) and T₃P (17.5 g, 27.54 mmol, 16.38 mL, 50% purity) at 0° C. After addition, the mixture was stirred at 0° C. for 5 min, and then N,O-dimethylhydroxylamine (2.7 g, 27.54 mmol, 1.5 eq, HCl) was added dropwise at 0° C. The resulting mixture was stirred at 25° C. for 12 hr. The reaction mixture was poured into water (20 mL). The resulting mixture was extracted with DCM (30 mL×2). The combined organic phase was washed with 5% H₃PO₄ (50 mL), sat.aq.NaHCO₃ (50 mL) and brine (30 mL), dried over anhydrous Na₂SO₄, filtered and concentrated to dryness. The title compound (3.2 g, 53.39% yield) was obtained as a light yellow solid. ¹H NMR (400 MHz, CDCl₃) δ ppm 5.95 (s, 1H), 5.39 (d, J=8.8 Hz, 1H), 4.75-4.62 (m, 1H), 3.77 (s, 3H), 3.40-3.30 (m, 2H), 3.20 (s, 3H), 2.57-2.43 (m, 2H), 2.16-2.05 (m, 1H), 1.92-1.62 (m, 2H), 1.42 (s, 9H).

### Step 3: (S)-2-amino-N-methoxy-N-methyl-3-((S)-2-oxopyrrolidin-3-yl)propanamide

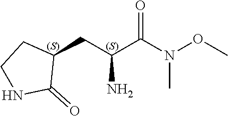

A mixture of tert-butyl ((S)-1-(methoxy(methyl)amino)-1-oxo-3-((S)-2-oxopyrrolidin-3-yl)propan-2-yl)carbamate (1.2 g, 3.81 mmol) in HCl/dioxane (12 mL) was stirred at 25° C. for 2 hrs. The reaction mixture was concentrated directly. The title compound (960 mg, 90.21% yield, HCl) was obtained as a white solid.

### Step 4: (1R,2S,5S)-tert-butyl 2-(((S)-1-(methoxy(methyl)amino)-1-oxo-3-((S)-2-oxopyrrolidin-3-yl)propan-2-yl)carbamoyl)-6,6-dimethyl-3-azabicyclo[3.1.0]hexane-3-carboxylate

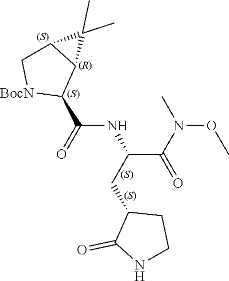

To a mixture of (1R,2S,5S)-3-(tert-butoxycarbonyl)-6,6-dimethyl-3-azabicyclo[3.1.0]hexane-2-carboxylic acid (950 mg, 3.72 mmol) in DCM (10 mL) was added T₃P (3.55 g, 5.58 mmol, 3.32 mL, 50% purity) and DIEA (1.92 g, 14.88 mmol, 2.59 mL) at 0° C. After addition, the reaction mixture was stirred at the same temperature for 5 min. To the above solution was added a solution of (S)-2-amino-N-methoxy-N-methyl-3-((S)-2-oxopyrrolidin-3-yl)propanamide (936.61 mg, 3.72 mmol, HCl) in DCM (4 mL) at 0° C. Then the resulting mixture was degassed and purged with N₂ for 3 times, and the mixture was stirred at 25° C. for 10 hrs under N₂ atmosphere. 5% phosphoric acid solution (10 mL) was added to the reaction mixture. The aqueous phase was extracted with DCM (10 mL×3). Then the combined organic phase was washed with sat.aq NaHCO₃ (20 mL), brine (20 mL), dried over anhydrous Na₂SO₄, filtered and concentrated. The crude product used into the next step without further purification. The title compound (1.2 g, 64.14% yield) was obtained as a white solid. ¹H NMR (400 MHz, CDCl₃) δ ppm 7.17-6.95 (m, 1H), 5.99-5.43 (m, 1H), 5.05-4.89 (m, 1H), 4.15-3.99 (m, 1H), 3.79 (d, J=9.6 Hz, 3H), 3.67-3.58 (m, 1H), 3.58-3.40 (m, 1H), 3.37-3.26 (m, 2H), 3.19 (d, J=5.6 Hz, 3H), 2.58-2.35 (m, 2H), 2.24-2.10 (m, 1H), 1.88-1.69 (m, 2H), 1.47-1.34 (m, 11H), 1.04-0.90 (m, 6H)

### Step 5: (1R,2S,5S)—N—((S)-1-(methoxy(methyl)amino)-1-oxo-3-((S)-2-oxopyrrolidin-3-yl)propan-2-yl)-6,6-dimethyl-3-azabicyclo[3.1.0]hexane-2-carboxamide

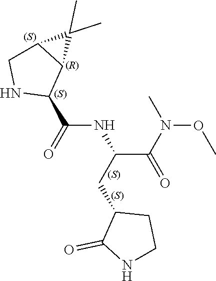

To a solution of (1R,2S,5S)-tert-butyl 2-(((S)-1-(methoxy(methyl)amino)-1-oxo-3-((S)-2-oxopyrrolidin-3-yl)propan-2-yl)carbamoyl)-6,6-dimethyl-3-azabicyclo[3.1.0]hexane-3-carboxylate (300 mg, 662.92 μmol) in dioxane (6 mL) was added HCl/dioxane (4 M, 6 mL). The mixture was stirred at 25° C. for 2 hr. The reaction mixture was concentrated directly. The title compound (280 mg, crude, HCl) was obtained as a white solid.

### Step 6: (1R,2S,5S)—N—((S)-1-(methoxy(methyl)amino)-1-oxo-3-((S)-2-oxopyrrolidin-3-yl)propan-2-yl)-6,6-dimethyl-3-(1H-pyrrolo[3,2-b]pyridine-2-carbonyl)-3-azabicyclo[3.1.0]hexane-2-carboxamide

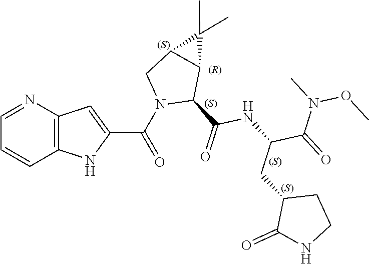

To a solution of 1H-pyrrolo[3,2-b]pyridine-2-carboxylic acid (115 mg, 709.37 μmol) and DIEA (275 mg, 2.13 mmol, 370.67 uL) in DCM (5 mL) was added (1R,2S,5S)—N—((S)-1-(methoxy(methyl)amino)-1-oxo-3-((S)-2-oxopyrrolidin-3-yl)propan-2-yl)-6,6-dimethyl-3-azabicyclo[3.1.0]hexane-2-carboxamide (250 mg, 709.37 μmol). After addition, HATU (540 mg, 1.42 mmol) was added at 0° C. The mixture was stirred at 25° C. for 3 hr. The reaction mixture was poured into sat.aq.NH₄Cl (20 mL). The resulting mixture was extracted with DCM (10 mL×3). The combined organic phase was washed with brine (20 mL), dried over anhydrous Na₂SO₄, filtered and concentrated to dryness. The residue was purified by prep-HPLC (column: Phenomenex Gemini-NX C18 75×30 mm×3 μm; mobile phase: [water (0.05% NH₃H₂O+10 mM NH₄HCO₃)-ACN]; B %: 5%-45%, 11 min). The title compound (150 mg, 284.44 μmol, 40.10% yield) was obtained as a white solid. ¹H NMR (400 MHz, CDCl₃) δ ppm 9.88-8.99 (m, 1H), 8.53-8.20 (m, 1H), 7.73-7.33 (m, 2H), 7.16-6.83 (m, 2H), 5.92-5.69 (m, 1H), 4.97-4.77 (m, 1H), 4.62-4.49 (m, 1H), 4.25-3.89 (m, 2H), 3.87-3.70 (m, 4H), 3.19-3.10 (m, 4H), 2.55-2.08 (m, 4H), 1.83-1.74 (m, 1H), 1.07-0.77 (m, 8H).

### Step 7: (1R,2S,5S)-6,6-dimethyl-N—((S)-1-oxo-3-((S)-2-oxopyrrolidin-3-yl)propan-2-yl)-3-(1H-pyrrolo[3,2-b]pyridine-2-carbonyl)-3-azabicyclo[3.1.0]hexane-2-carboxamide

To a solution of (1R,2S,5S)—N—((S)-1-(methoxy(methyl)amino)-1-oxo-3-((S)-2-oxopyrrolidin-3-yl)propan-2-yl)-6,6-dimethyl-3-(1H-pyrrolo[3,2-b]pyridine-2-carbonyl)-3-azabicyclo[3.1.0]hexane-2-carboxamide (70 mg, 140.97 μmol) in THE (3 mL) was added LiAlH₄ (2 M, 105 uL) at 0° C. The mixture was stirred at 25° C. for 12 hr. The reaction was quenched with sat.aq.NH₄Cl (1 mL) drop-wise, filtered and concentrated to dryness. The residue was purified by prep-HPLC (column: Phenomenex Gemini-NX C18 75×30 mm×3 μm; mobile phase: [water (0.05% NH3H2O+10 mM NH₄HCO₃)-ACN]; B %: 0%-40%, 15 min). Compound 83 (4 mg, 8.67 μmol, 6.15% yield, 94.80% purity) was obtained as a white solid. ¹H NMR (400 MHz, CD₃OD) δ ppm 8.45-8.33 (m, 1H), 7.94-7.84 (m, 1H), 7.33-7.22 (m, 1H), 7.19-6.95 (m, 1H), 4.53-3.70 (m, 4H), 3.29-3.23 (m, 1H), 2.95-2.58 (m, 1H), 2.45-2.29 (m, 1H), 2.13-1.96 (m, 1H), 1.89-1.68 (m, 2H), 1.67-1.47 (m, 2H), 1.34-0.97 (m, 7H). ESI-MS [M+H]⁺ calc'd. for C₂₃H₂₇N₅O₄ 438.21, found 456.2.

### Example 84: (S)—N—((S)-1-Oxo-3-((S)-2-oxopyrrolidin-3-yl)propan-2-yl)-6-(1-phenylcyclopropanecarbonyl)-6-azaspiro[3.4]octane-7-carboxamide (Compound 84)

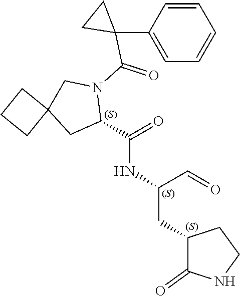

Compound 84 was synthesized by the same procedure as Compound 2. ¹H NMR (400 MHz, CD₃OD) δ ppm 7.40-7.16 (m, 5H), 4.86-4.43 (m, 2H), 4.35 (t, J=8.0 Hz, 1H), 4.04-3.88 (m, 1H), 3.57 (d, J=10.8 Hz, 1H), 3.26-3.16 (m, 1H), 2.78-2.57 (m, 1H), 2.47-2.23 (m, 2H), 2.10-1.98 (m, 1H), 1.95-1.67 (m, 7H), 1.67-1.49 (m, 3H), 1.35-1.12 (m, 3H). ESI-MS [M+H]⁺ calc'd for C₂₅H₃₁N₃O₄, 438.23; found, 438.4.

### Example 85: (1R,2S,5S)-6,6-Dimethyl-3-(4-methyl-1H-indole-2-carbonyl)-N—((S)-1-oxo-3-((S)-2-oxopyrrolidin-3-yl)propan-2-yl)-3-azabicyclo[3.1.0]hexane-2-carboxamide (Compound 85)

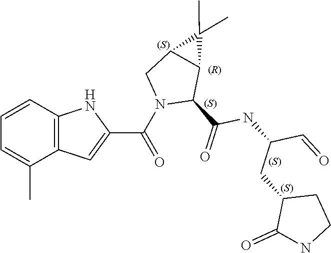

Compound 85 was synthesized by the same procedure as Compound 1. ¹H NMR (400 MHz, CD₃OD) δ ppm 7.29-7.21 (m, 1H), 7.16-7.10 (m, 1H), 7.09-6.89 (m, 1H), 6.89-6.80 (m, 1H), 4.67-4.61 (m, 1H), 4.52-4.30 (m, 1H), 4.03-3.66 (m, 2H), 3.29-3.23 (m, 1H), 2.82-2.59 (m, 1H), 2.58-2.45 (m, 3H), 2.39-1.82 (m, 2H), 1.81-1.66 (m, 2H), 1.64-1.19 (m, 3H), 1.18-0.94 (m, 6H). ESI-MS [M+H]⁺ calc'd for C₂₅H₃₀N₄O₄, 451.23; found, 451.2.

### Example 86: (1R,2S,5S)-6,6-Dimethyl-N—((S)-1-oxo-3-((S)-2-oxopyrrolidin-3-yl)propan-2-yl)-3-(2-(tetrahydro-2H-pyran-4-yl)acetyl)-3-azabicyclo[3.1.0]hexane-2-carboxamide (Compound 86)

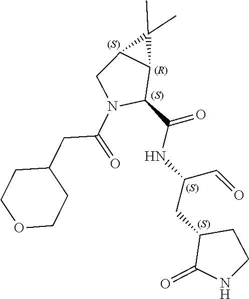

Compound 86 was synthesized by the same procedure as Compound 1. ¹H NMR (400 MHz, CD₃OD) δ ppm 4.56-4.43 (m, 1H), 4.37-4.25 (m, 1H), 4.03-3.66 (m, 4H), 3.64-3.55 (m, 1H), 3.45-3.35 (m, 2H), 3.28-3.22 (m, 1H), 2.72-2.51 (m, 1H), 2.44-2.14 (m, 3H), 2.07-1.96 (m, 2H), 1.91-1.40 (m, 6H), 1.37-1.17 (m, 2H), 1.13-0.92 (m, 6H). ESI-MS [M+H]⁺ calc'd for C₂₂H₃₃N₃O₅, 420.24; found, 420.2.

### Example 87: (S)-5-(7-Fluoro-1H-indole-2-carbonyl)-N—((S)-1-oxo-3-((S)-2-oxopyrrolidin-3-yl)propan-2-yl)-5-azaspiro[2.4]heptane-6-carboxamide (Compound 87)

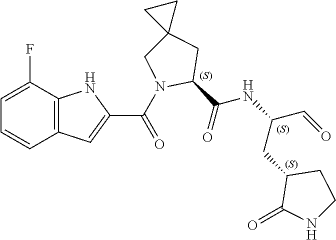

Compound 87 was synthesized by the same procedure as Compound 2. ¹H NMR (400 MHz, CD₃OD) δ ppm 7.53-7.29 (m, 1H), 7.12-6.86 (m, 3H), 4.65-4.31 (m, 2H), 4.12-3.64 (m, 3H), 2.72-2.34 (m, 2H), 2.30-2.15 (m, 1H), 2.14-1.91 (m, 2H), 1.90-1.72 (m, 1H), 1.70-1.52 (m, 1H), 1.51-1.26 (m, 1H), 0.85-0.56 (m, 4H). ESI-MS [M+H]⁺ calc'd for C₂₃H₂₅FN₄O₄, 441.19; found, 441.1.

### Example 88: (1R,2S,5S)-3-(7-Chloro-4-isopropyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 88)

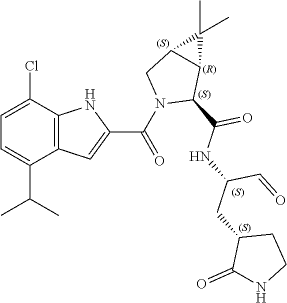

Compound 88 was synthesized by the same procedure as Compound 1. ¹H NMR (400 MHz, CD₃OD) δ ppm 7.29-6.83 (m, 3H), 4.70-4.65 (m, 1H), 4.41-4.26 (m, 1H), 4.11-3.94 (m, 1H), 3.93-3.70 (m, 1H), 3.51-3.34 (m, 1H), 3.28-3.22 (m, 1H), 2.97-2.52 (m, 1H), 2.44-2.28 (m, 1H), 2.24-1.96 (m, 1H), 1.85-1.67 (m, 2H), 1.66-1.44 (m, 2H), 1.42-1.24 (m, 7H), 1.22-0.90 (m, 6H). ESI-MS [M+H]⁺ calc'd for C₂₇H₃₃ClN₄O₄, 513.22; found, 513.1.

### Example 89: (1R,2S,5S)-3-(5-Chloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 89)

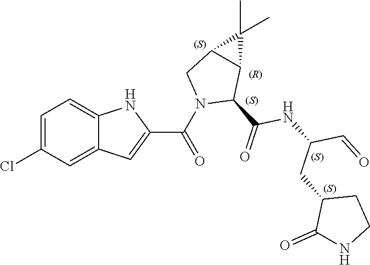

Compound 89 was synthesized by the same procedure as Compound 83. ¹H NMR (400 MHz, CD₃OD) δ ppm 7.69-7.48 (m, 1H), 7.45-7.37 (m, 1H), 7.23-7.15 (m, 1H), 7.07-6.83 (m, 1H), 5.04-4.94 (m, 1H), 4.53-4.38 (m, 1H), 4.32-3.95 (m, 2H), 3.80-3.64 (m, 1H), 2.99-2.56 (m, 1H), 2.43-2.28 (m, 1H), 2.10-1.91 (m, 1H), 1.90-1.75 (m, 1H), 1.74-1.67 (m, 1H), 1.64-1.30 (m, 3H), 1.19-0.85 (m, 6H). ESI-MS [M+H]⁺ calc'd for C₂₄H₂₇ClN₄O₄, 471.17; found, 471.4.

### Example 90: (1R,2S,5S)-3-(5-Chloro-1-methyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 90)

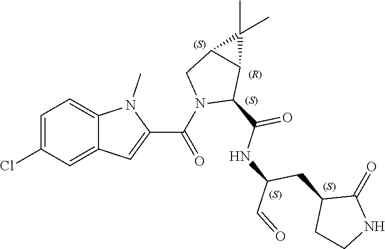

Compound 90 was synthesized by the same procedure as Compound 1. ¹H NMR (400 MHz, CD₃OD) δ ppm 7.63-7.50 (m, 1H), 7.45-7.38 (m, 1H), 7.26-7.18 (m, 1H), 6.85-6.60 (m, 1H), 4.77-4.60 (m, 1H), 4.54-4.33 (m, 1H), 4.11-3.94 (m, 1H), 3.81-3.73 (m, 3H), 3.72-3.64 (m, 1H), 3.59-3.37 (m, 1H), 3.09-2.56 (m, 2H), 2.45-1.97 (m, 1H), 1.82-1.61 (m, 1H), 1.61-1.54 (m, 2H), 1.41-1.25 (m, 2H), 1.23-1.03 (m, 6H). ESI-MS [M+H]⁺ calc'd for C₂₅H₂₉ClN₄O₄, 485.19; found, 485.4.

### Example 91: (1R,2S,5S)-3-(7-Methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 91)

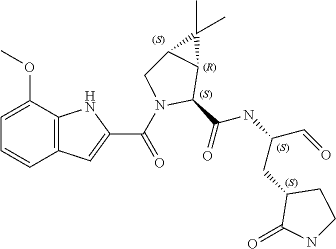

Compound 91 was synthesized by the same procedure as Compound 1. ¹H NMR (400 MHz, CD₃OD) δ ppm 7.28-7.09 (m, 1H), 7.07-6.84 (m, 2H), 6.79-6.68 (m, 1H), 5.02-4.93 (m, 1H), 4.65-4.56 (m, 1H), 4.54-4.37 (m, 1H), 4.33-4.01 (m, 1H), 3.98-3.94 (m, 3H), 3.83-3.68 (m, 1H), 2.97-2.53 (m, 1H), 2.42-2.26 (m, 1H), 2.12-1.82 (m, 1H), 1.81-1.67 (m, 2H), 1.65-1.28 (m, 3H), 1.20-0.93 (m, 6H). ESI-MS [M+H]⁺ calc'd for C₂₅H₃₀N₄O₅, 467.22; found, 467.4.

### Example 92: (1R,2S,5S)-3-((S)-2-Cyclohexylpropanoyl)-6,6-dimethyl-N—((S)-1-oxo-3-((S)-2-oxopyrrolidin-3-yl)propan-2-yl)-3-azabicyclo[3.1.0]hexane-2-carboxamide (Compound 92)

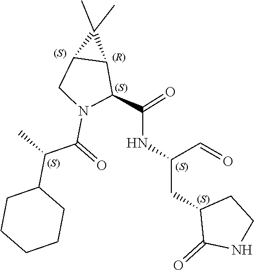

Compound 92 was synthesized by the same procedure as Compound 1. ¹H NMR (400 MHz, CD₃OD) δ ppm 4.52-4.39 (m, 1H), 4.33-4.07 (m, 1H), 4.04-3.67 (m, 2H), 3.64-3.51 (m, 1H), 3.28-3.24 (m, 1H), 2.70-1.93 (m, 4H), 1.89-1.61 (m, 6H), 1.60-1.37 (m, 4H), 1.29-1.11 (m, 3H), 1.10-1.05 (m, 3H), 1.05-0.79 (m, 8H). ESI-MS [M+H]⁺ calc'd for C₂₄H₃₇N₃O₄, 432.57; found, 432.4.

### Example 93: (1R,2S,5S)-6,6-Dimethyl-3-(5-methyl-1H-indole-2-carbonyl)-N—((S)-1-oxo-3-((S)-2-oxopyrrolidin-3-yl)propan-2-yl)-3-azabicyclo[3.1.0]hexane-2-carboxamide (Compound 93)

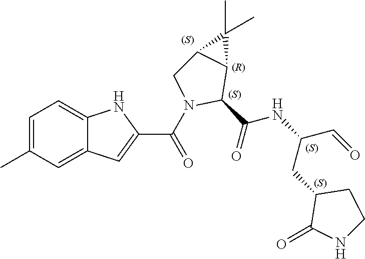

Compound 93 was synthesized by the same procedure as Compound 1. ¹H NMR (400 MHz, CD₃OD) δ ppm 7.45-7.27 (m, 2H), 7.10-7.02 (m, 1H), 6.99-6.80 (m, 1H), 5.02-4.89 (m, 1H), 4.82-4.61 (m, 1H), 4.52-4.25 (m, 1H), 4.06-3.95 (m, 1H), 3.79-3.43 (m, 1H), 3.29-3.19 (m, 1H), 2.89-2.55 (m, 1H), 2.39 (d, J=14.4 Hz, 3H), 2.35-2.13 (m, 1H), 2.09-1.88 (m, 1H), 1.78-1.66 (m, 1H), 1.63-1.52 (m, 1H), 1.46-1.22 (m, 2H), 1.16-0.94 (m, 6H). ESI-MS [M+H]⁺ calc'd for C₂₅H₃₀N₄O₄, 451.23; found, 451.4.

### Example 94: (1R,2S,5S)-3-(1-Hydroxycyclohexanecarbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 94)

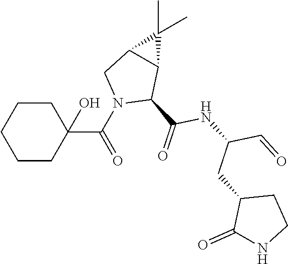 [SMILES: CC1(C)[C@@H]2[C@@H](C(=O)N[C@H](C=O)C[C@@H]3CCNC3=O)N(C(=O)C3(O)CCCCC3)C[C@@H]21]

Compound 94 was synthesized by the same procedure as Compound 1.

¹H NMR (400 MHz, CD₃OD) δ ppm 4.54-4.45 (m, 1H), 4.39-4.34 (m, 1H), 4.08-3.72 (m, 3H), 3.29-3.26 (m, 1H), 2.67-2.20 (m, 2H), 2.05-1.83 (m, 2H), 1.76-1.47 (m, 11H), 1.43-1.19 (m, 2H), 1.09-0.90 (m, 6H). ESI-MS [M+H]⁺ calc'd for C₂₂H₃₃N₃O₅, 420.24; found, 420.4.

### Example 95: (1R,2S,5S)-3-(3,3-Dimethylbutanoyl)-6,6-dimethyl-N—((S)-1-oxo-3-((S)-2-oxopyrrolidin-3-yl)propan-2-yl)-3-azabicyclo[3.1.0]hexane-2-carboxamide (Compound 95)

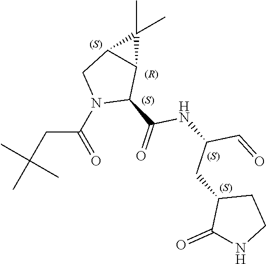

Compound 95 was synthesized by the same procedure as Compound 1. ¹H NMR (400 MHz, CD₃OD) δ ppm 4.57-4.35 (m, 1H), 4.34-4.21 (m, 1H), 4.05-3.88 (m, 2H), 3.71-3.54 (m, 1H), 3.29-3.22 (m, 1H), 2.71-2.29 (m, 2H), 2.28-2.08 (m, 2H), 2.07-1.67 (m, 2H), 1.63-1.34 (m, 3H), 1.12-0.94 (m, 15H). ESI-MS [M+H]⁺ calc'd for C₂₁H₃₃N₃O₄, 392.25; found, 392.3.

### Example 96: (1R,2S,5S)-3-(1,4-Dimethylindole-2-carbonyl)-N-[(1S)-1-formyl-2-[(3S)-2-oxopyrrolidin-3-yl]ethyl]-6,6-dimethyl-3-azabicyclo[3.1.0]hexane-2-carboxamide (Compound 96)

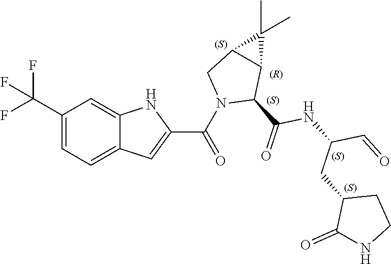

Compound 96 was synthesized by the same procedure as Compound 1. ¹H NMR (400 MHz, CD₃OD) δ ppm 7.94-7.66 (m, 2H), 7.37-7.23 (m, 1H), 7.20-6.96 (m, 1H), 4.66-4.49 (m, 1H), 4.46-4.22 (m, 1H), 4.09-3.70 (m, 2H), 2.98-2.58 (m, 1H), 2.53-2.26 (m, 1H), 2.23-1.95 (m, 1H), 1.92-1.69 (m, 2H), 1.67-1.49 (m, 2H), 1.47-1.21 (m, 2H), 1.18-0.94 (m, 6H). ESI-MS [M+H]⁺ calc'd for C₂₅H₂₇F₃N₄O₄, 505.20; found, 505.4.

### Example 97: (1R,2S,5S)-3-(4,6-Dichloro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 97)

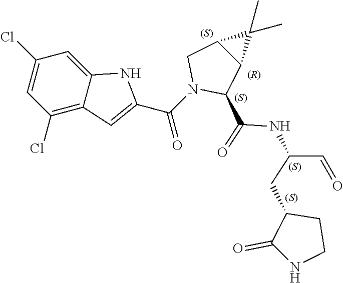

Compound 97 was synthesized by the same procedure as Compound 1. ¹H NMR (400 MHz, CD₃OD) δ ppm 7.45-7.39 (m, 1H), 7.15-7.09 (m, 1H), 7.08-6.87 (m, 1H), 4.65-4.57 (m, 1H), 4.53-4.39 (m, 1H), 4.35-3.92 (m, 2H), 3.83-3.68 (m, 1H), 3.02-2.55 (m, 1H), 2.43-2.26 (m, 1H), 2.06-1.90 (m, 1H), 1.85-1.72 (m, 1H), 1.72-1.51 (m, 2H), 1.50-1.26 (m, 2H), 1.20-0.91 (m, 6H). ESI-MS [M+H]⁺ calc'd for C₂₄H₂₆Cl₂N₄O₄, 505.13; found, 505.4.

### Example 98: (2S,4R)-4-fluoro-1-(1H-Indole-2-carbonyl)-N—((S)-1-oxo-3-((S)-2-oxopyrrolidin-3-yl)propan-2-yl)pyrrolidine-2-carboxamide (Compound 98)

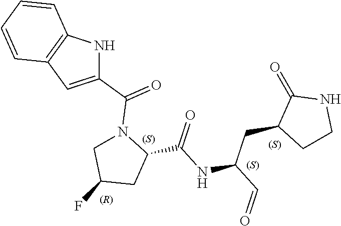

Compound 98 was synthesized by the same procedure as Compound 2. ¹H NMR (400 MHz, CD₃OD) δ ppm 7.68-7.52 (m, 1H), 7.44 (d, J=8.4 Hz, 1H), 7.23 (t, J=8.0 Hz, 1H), 7.11-7.04 (m, 1H), 7.04-6.89 (m, 1H), 5.52-5.20 (m, 1H), 4.36-3.57 (m, 4H), 3.29-3.23 (m, 1H), 2.84-2.57 (m, 2H), 2.39-2.00 (m, 3H), 1.85-1.54 (m, 2H), 1.39-1.16 (m, 1H). ESI-MS [M+H]⁺ calc'd for C₂₁H₂₃FN₄O₄, 415.43; found, 415.0.

### Example 99: (1R,2S,5S)—N—((S)-4-(Cyclopropylamino)-3,4-dioxo-1-((S)-2-oxopyrrolidin-3-yl)butan-2-yl)-6,6-dimethyl-3-(1-phenylcyclopentanecarbonyl)-3-azabicyclo[3.1.0]hexane-2-carboxamide (Compound 99)

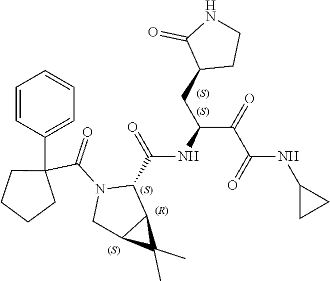

Compound 99 was synthesized by the same procedure as Compound 119. ¹H NMR (400 MHz, CD₃OD) δ ppm 7.39-7.29 (m, 2H), 7.29-7.15 (m, 3H), 4.65-4.12 (m, 2H), 3.50-3.34 (m, 2H), 3.30-3.16 (m, 1H), 3.02-2.86 (m, 1H), 2.84-2.59 (m, 2H), 2.54-2.35 (m, 2H), 2.34-2.17 (m, 2H), 2.15-1.73 (m, 5H), 1.71-1.46 (m, 3H), 1.37-1.18 (m, 2H), 0.92 (d, J=3.6 Hz, 3H), 0.82-0.65 (m, 2H), 0.65-0.50 (m, 5H). ESI-MS [M+H]⁺ calc'd for C₃₁H₄₀N₄O₅, 549.30; found, 549.5.

### Example 100: (1R,2S,5S)-3-(4-Fluoro-1-methyl-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 100)

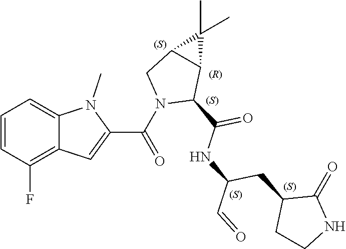

Compound 100 was synthesized by the same procedure as Compound 1. ¹H NMR (400 MHz, CD₃OD) δ ppm 7.29-7.13 (m, 2H), 6.93-6.64 (m, 2H), 4.83-4.73 (m, 1H), 4.55-4.32 (m, 1H), 4.14-3.98 (m, 1H), 3.84-3.74 (m, 3H), 3.73-3.47 (m, 2H), 3.29-3.19 (m, 1H), 3.07-2.79 (m, 1H), 2.69-2.31 (m, 1H), 2.13-1.68 (m, 1H), 1.64-1.49 (m, 3H), 1.42-1.28 (m, 1H), 1.22-1.13 (m, 3H), 1.12-1.03 (m, 3H). ESI-MS [M+H]⁺ calc'd for C₂₅H₂₉FN₄O₄, 469.22; found, 469.4.

### Example 101: (1R,2S,5S)-3-(5-Fluoro-6-methoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 101)

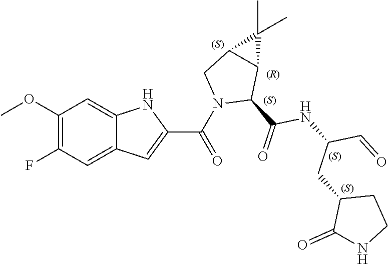

Compound 101 was synthesized by the same procedure as Compound 83. ¹H NMR (400 MHz, CD₃OD) δ ppm 7.35-7.14 (m, 1H), 7.08-6.80 (m, 2H), 4.59-4.37 (m, 1H), 4.30-3.93 (m, 2H), 3.89 (d, J=3.6 Hz, 3H), 3.82-3.67 (m, 1H), 3.29-3.22 (m, 2H), 2.64-2.34 (m, 1H), 2.10-1.67 (m, 3H), 1.66-1.35 (m, 3H), 1.17-0.89 (m, 6H). ESI-MS [M+H]⁺ calc'd for C₂₅H₂₉FN₄O₅, 485.21; found, 485.1.

### Example 102: (2S,4S)-1-(1H-Indole-2-carbonyl)-4-methoxy-N—((S)-1-oxo-3-((S)-2-oxopyrrolidin-3-yl)propan-2-yl)pyrrolidine-2-carboxamide (Compound 102)

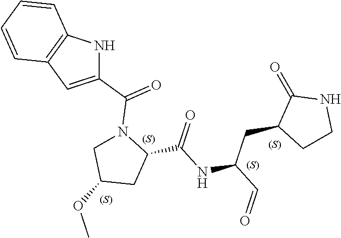

Compound 102 was synthesized by the same procedure as Compound 2. ¹H NMR (400 MHz, CD₃OD) δ ppm 7.72-7.52 (m, 1H), 7.44 (d, J=8.0 Hz, 1H), 7.27-7.16 (m, 1H), 7.13-6.84 (m, 2H), 4.76 (s, 1H), 4.52-4.31 (m, 1H), 4.22-3.75 (m, 4H), 3.36-3.30 (m, 3H), 2.67-2.18 (m, 4H), 2.08-1.34 (m, 4H). ESI-MS [M+H]⁺ calc'd for C₂₂H₂₆N₄O₅, 427.19; found, 427.1.

### Example 103: (2S,4R)-4-cyclohexyl-1-(1H-Indole-2-carbonyl)-N—((S)-1-oxo-3-((S)-2-oxopyrrolidin-3-yl)propan-2-yl)pyrrolidine-2-carboxamide (Compound 103)

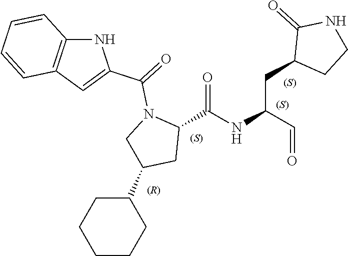

Compound 103 was synthesized by the same procedure as Compound 2. ¹H NMR (400 MHz, CD₃OD) δ ppm 7.70-7.49 (m, 1H), 7.47-7.30 (m, 1H), 7.26-7.16 (m, 1H), 7.11-6.79 (m, 2H), 4.66-4.52 (m, 2H), 4.37-4.17 (m, 1H), 4.12-3.84 (m, 1H), 3.78-3.56 (m, 1H), 3.43-3.33 (m, 1H), 2.90-2.60 (m, 1H), 2.55-2.34 (m, 1H), 2.24-1.98 (m, 2H), 1.90-1.72 (m, 5H), 1.71-1.55 (m, 3H), 1.39-1.26 (m, 4H), 1.23-0.99 (m, 3H). ESI-MS [M+H]⁺ calc'd for C₂₇H₃₄N₄O₄, 479.26; found, 479.1.

### Example 104: (1R,2S,5S)-6,6-Dimethyl-N—((S)-1-oxo-3-((S)-2-oxopyrrolidin-3-yl)propan-2-yl)-3-((S)-2-phenylpropanoyl)-3-azabicyclo[3.1.0]hexane-2-carboxamide (Compound 104)

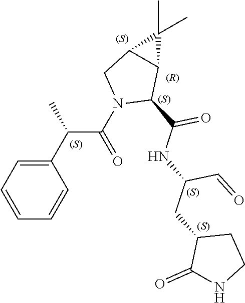

Compound 104 was synthesized by the same procedure as Compound 1. ¹H NMR (400 MHz, CD₃OD) δ ppm 7.54-6.91 (m, 5H), 4.57-4.30 (m, 1H), 4.21-3.84 (m, 2H), 3.82-3.62 (m, 1H), 3.59-3.34 (m, 2H), 3.27-3.10 (m, 1H), 2.68-2.22 (m, 2H), 2.10-1.52 (m, 3H), 1.52-1.37 (m, 2H), 1.37-1.27 (m, 3H), 1.13-0.30 (m, 6H). ESI-MS [M+H]⁺ calc'd for C₂₄H₃₁N₃O₄, 426.23; found, 426.4.

### Example 105: (1R,2S,5S)—N—((S)-4-(Cyclopropylamino)-3,4-dioxo-1-((S)-2-oxopyrrolidin-3-yl)butan-2-yl)-6,6-dimethyl-3-(2-(4-methylcyclohexyl)acetyl)-3-azabicyclo[3.1.0]hexane-2-carboxamide (Compound 105)

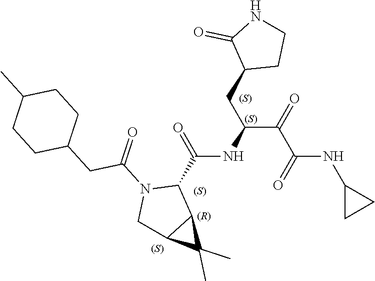

Compound 105 was synthesized by the same procedure as Compound 119. ¹H NMR (400 MHz, CD₃OD) δ ppm 4.65-4.09 (m, 2H), 3.99-3.83 (m, 1H), 3.69-3.53 (m, 1H), 3.40-3.32 (m, 1H), 3.30-3.16 (m, 2H), 2.81-2.39 (m, 2H), 2.37-1.83 (m, 5H), 1.80-1.57 (m, 4H), 1.54-1.37 (m, 5H), 1.35-1.21 (m, 2H), 1.13-1.04 (m, 3H), 1.00-0.84 (m, 7H), 0.82-0.66 (m, 2H), 0.65-0.50 (m, 2H). ESI-MS [M+H]⁺ calc'd for C₂₈H₄₂N₄O₅, 515.32; found, 515.5.

### Example 106: (1S,3S,4R,6S)-6-Fluoro-2-(1H-indole-2-carbonyl)-N—((S)-1-oxo-3-((S)-2-oxopyrrolidin-3-yl)propan-2-yl)-2-azabicyclo[2.2.1]heptane-3-carboxamide (Compound 106)

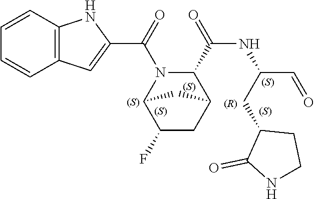

Compound 106 was synthesized by the same procedure as Compound 2. ¹H NMR (400 MHz, CD₃OD) δ ppm 7.76-7.39 (m, 2H), 7.30-7.17 (m, 1H), 7.14-6.78 (m, 2H), 5.17-4.96 (m, 1H), 4.53-4.27 (m, 1H), 4.12-3.33 (m, 2H), 3.18-2.78 (m, 2H), 2.65-2.36 (m, 1H), 2.35-2.09 (m, 2H), 2.08-1.57 (m, 5H), 1.57-1.28 (m, 2H). ESI-MS [M+H]⁺ calc'd for C₂₃H₂₅FN₄O₄, 441.19; found, 441.2.

### Example 107: (2S,4R)-1-(1H-Indole-2-carbonyl)-4-methyl-N—((S)-1-oxo-3-((S)-2-oxopyrrolidin-3-yl)propan-2-yl)pyrrolidine-2-carboxamide (Compound 107)

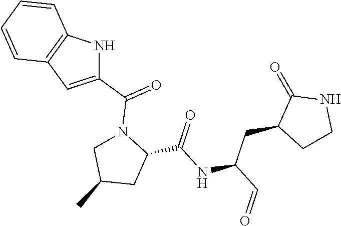 [SMILES: C[C@@H]1C[C@@H](C(=O)N[C@H](C=O)C[C@@H]2CCNC2=O)N(C(=O)c2cc3ccccc3[nH]2)C1]

Compound 107 was synthesized by the same procedure as Compound 2. ¹H NMR (400 MHz, CD₃OD) δ ppm 7.68-7.50 (m, 1H), 7.44 (br d, J=8.0 Hz, 1H), 7.26-7.18 (m, 1H), 7.10-6.84 (m, 2H), 4.85-4.66 (m, 1H), 4.65-4.46 (m, 1H), 4.40-4.18 (m, 1H), 4.16-3.89 (m, 1H), 3.84-3.50 (m, 1H), 2.98-2.53 (m, 2H), 2.45-2.16 (m, 2H), 2.14-1.71 (m, 3H), 1.67-1.33 (m, 2H), 1.13 (d, J=6.4 Hz, 3H). ESI-MS [M+H]⁺ calc'd for C₂₂H₂₆N₄O₄, 411.2; found, 411.0.

### Example 108: (S)-1-(1H-Indole-2-carbonyl)-4-methylene-N—((S)-1-oxo-3-((S)-2-oxopyrrolidin-3-yl)propan-2-yl)pyrrolidine-2-carboxamide (Compound 108)

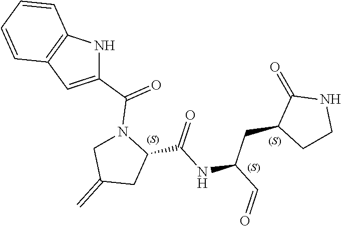

Compound 108 was synthesized by the same procedure as Compound 2. ¹H NMR (400 MHz, CD₃OD) δ ppm 7.71-7.52 (m, 1H), 7.44 (d, J=8.4 Hz, 1H), 7.23 (t, J=7.2 Hz, 1H), 7.13-6.87 (m, 2H), 5.27-5.08 (m, 2H), 4.75-4.64 (m, 1H), 4.48-4.36 (m, 1H), 4.05-3.68 (m, 1H), 3.29-3.23 (m, 1H), 3.09-2.91 (m, 1H), 2.87-2.57 (m, 2H), 2.53-2.26 (m, 1H), 2.05-1.89 (m, 1H), 1.80-1.35 (m, 3H). ESI-MS [M+H]⁺ calc'd for C₂₂H₂₄N₄O₄, 409.18; found, 409.0.

### Example 109: (2S,4R)-4-(2-Chlorobenzyl)-1-(1H-indole-2-carbonyl)-N—((S)-1-oxo-3-((S)-2-oxopyrrolidin-3-yl)propan-2-yl)pyrrolidine-2-carboxamide (Compound 109)

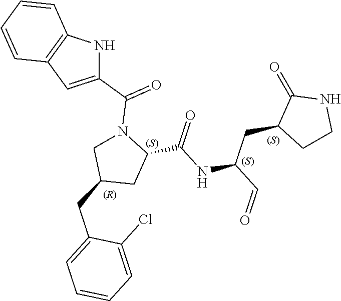

Compound 109 was synthesized by the same procedure as Compound 2. ¹H NMR (400 MHz, CD₃OD) δ ppm 7.69-7.52 (m, 1H), 7.50-7.30 (m, 3H), 7.29-7.17 (m, 3H), 7.11-6.87 (m, 2H), 4.49-4.16 (m, 1H), 4.06-3.89 (m, 1H), 3.85-3.71 (m, 1H), 3.58-3.44 (m, 1H), 3.34-3.25 (m, 2H), 3.04-2.84 (m, 3H), 2.78-2.56 (m, 1H), 2.44-2.27 (m, 1H), 2.14-1.71 (m, 3H), 1.61-1.29 (m, 2H). ESI-MS [M+H]⁺ calc'd for C₂₈H₂₉ClN₄O₄, 521.19; found, 521.3.

### Example 110: (1R,2S,5S)-6,6-Dimethyl-3-(3-methylcyclobutanecarbonyl)-N—((S)-1-oxo-3-((S)-2-oxopyrrolidin-3-yl)propan-2-yl)-3-azabicyclo[3.1.0]hexane-2-carboxamide (Compound 110)

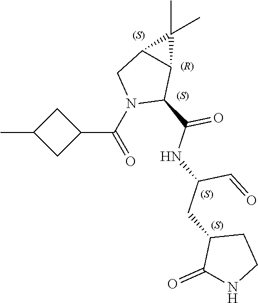

Compound 110 was synthesized by the same procedure as Compound 1. ¹H NMR (400 MHz, CD₃OD) δ ppm 4.60-4.44 (m, 1H), 4.35-4.15 (m, 1H), 4.03-3.88 (m, 1H), 3.85-3.75 (m, 1H), 3.74-3.47 (m, 1H), 3.18-2.96 (m, 1H), 2.68-2.48 (m, 1H), 2.45-2.21 (m, 4H), 2.13-1.79 (m, 3H), 1.77 (s, 2H), 1.59-1.48 (m, 2H), 1.48-1.40 (m, 1H), 1.14 (d, J=6.4 Hz, 1H), 1.11-0.99 (m, 5H), 0.95-0.90 (m, 3H). ESI-MS [M+H]⁺ calc'd for C₂₁H₃₁N₃O₄, 390.23; found, 390.4.

### Example 111: (1R,2S,5S)-3-(5-Ethoxy-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 111)

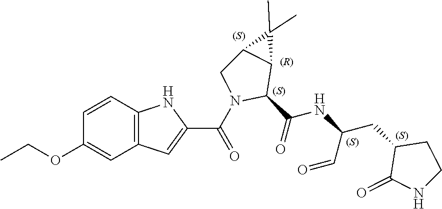

Compound 111 was synthesized by the same procedure as Compound 1. ¹H NMR (400 MHz, CD₃OD) δ ppm 7.31 (dd, J=6.8, 8.8 Hz, 1H), 7.13-6.97 (m, 1H), 6.96-6.81 (m, 2H), 4.59 (s, 1H), 4.52-4.38 (m, 1H), 4.31-3.92 (m, 4H), 3.83-3.72 (m, 1H), 3.28-3.20 (m, 1H), 2.94-2.55 (m, 1H), 2.41-2.20 (m, 1H), 2.03-1.86 (m, 1H), 1.77-1.68 (m, 1H), 1.66-1.48 (m, 2H), 1.40 (dt, J=0.8, 6.8 Hz, 3H), 1.37-1.30 (m, 1H), 1.16-0.95 (m, 6H). ESI-MS [M+H]⁺ calc'd for C₂₆H₃₂N₄O₅, 481.24; found, 481.5.

### Example 112: (2S,4R)-4-Cyano-1-(1H-indole-2-carbonyl)-N—((S)-1-oxo-3-((S)-2-oxopyrrolidin-3-yl)propan-2-yl)pyrrolidine-2-carboxamide (Compound 112)

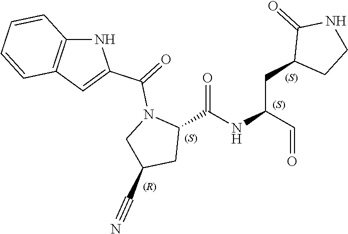

Compound 112 was synthesized by the same procedure as Compound 2. ¹H NMR (400 MHz, CD₃OD) δ ppm 7.77-7.49 (m, 1H), 7.44 (d, J=8.0 Hz, 1H), 7.23 (t, J=7.6 Hz, 1H), 7.13-6.82 (m, 2H), 4.56 (s, 1H), 4.50-4.35 (m, 1H), 4.33-4.13 (m, 1H), 3.97 (s, 1H), 3.86-3.56 (m, 1H), 3.53-3.34 (m, 1H), 3.02-2.20 (m, 4H), 2.12-1.69 (m, 2H), 1.68-1.16 (m, 2H). ESI-MS [M+H]⁺ calc'd for C₂₂H₂₃N₅O₄, 422.18; found, 422.4.

### Example 113: (2S,3aS,7aS)-1-(1H-Indole-2-carbonyl)-N—((S)-1-oxo-3-((S)-2-oxopyrrolidin-3-yl)propan-2-yl)octahydro-1H-indole-2-carboxamide (Compound 113)

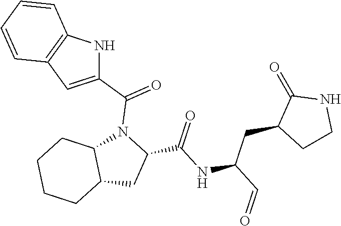 [SMILES: O=C[C@H](C[C@@H]1CCNC1=O)NC(=O)[C@@H]1C[C@@H]2CCCC[C@@H]2N1C(=O)c1cc2ccccc2[nH]1]

Compound 113 was synthesized by the same procedure as Compound 2. ¹H NMR (400 MHz, CD₃OD) δ ppm 7.69-7.47 (m, 1H), 7.42 (d, J=8.4 Hz, 1H), 7.20 (br t, J=7.6 Hz, 1H), 7.08-6.99 (m, 1H), 6.98-6.79 (m, 1H), 5.21-4.99 (m, 1H), 4.80-4.66 (m, 1H), 4.51-4.32 (m, 1H), 4.30-4.19 (m, 1H), 4.05-3.59 (m, 1H), 2.86-2.63 (m, 1H), 2.49-2.33 (m, 2H), 2.27-2.14 (m, 2H), 2.12-2.03 (m, 1H), 1.85-1.74 (m, 4H), 1.60-1.50 (m, 2H), 1.46-1.19 (m, 4H). ESI-MS [M+H]⁺ calc'd for C₂₅H₃₀N₄O₄, 451.23; found, 451.4.

### Example 114: (1R,2S,5S)-3-(5,6-dimethoxy-1H-Indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 114)

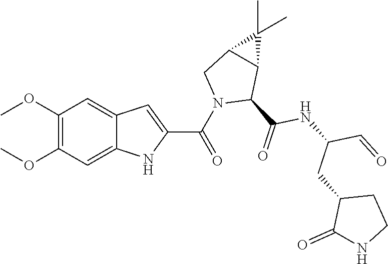 [SMILES: COc1cc2cc(C(=O)N3C[C@H]4[C@@H]([C@H]3C(=O)N[C@H](C=O)C[C@@H]3CCNC3=O)C4(C)C)[nH]c2cc1OC]

Compound 114 was synthesized by the same procedure as Compound 1. ¹H NMR (400 MHz, CD₃OD) δ ppm 7.18-6.73 (m, 3H), 5.01-4.88 (m, 1H), 4.65-4.22 (m, 2H), 4.05-3.94 (m, 1H), 3.89-3.79 (m, 6H), 3.29-3.17 (m, 1H), 2.95-2.56 (m, 1H), 2.40-2.21 (m, 1H), 2.14-1.83 (m, 2H), 1.81-1.26 (m, 4H), 1.16-0.93 (m, 6H). ESI-MS [M+H]⁺ calc'd for C₂₆H₃₂N₄O₆, 497.24; found, 497.4.

### Example 115: (S)-4,4-difluoro-1-(1H-Indole-2-carbonyl)-N—((S)-1-oxo-3-((S)-2-oxopyrrolidin-3-yl)propan-2-yl)pyrrolidine-2-carboxamide (Compound 115)

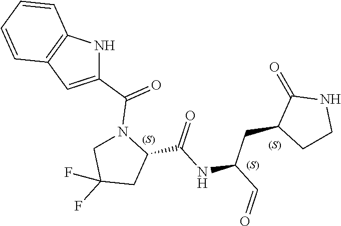

Compound 115 was synthesized by the same procedure as Compound 2. ¹H NMR (400 MHz, CD₃OD) δ ppm 7.77-7.51 (m, 1H), 7.43 (d, J=8.0 Hz, 1H), 7.23 (t, J=7.2 Hz, 1H), 7.12-6.83 (m, 2H), 3.87-3.86 (m, 1H), 4.51-3.66 (m, 4H), 2.95-2.25 (m, 4H), 2.08-1.32 (m, 4H). ESI-MS [M+H]⁺ calc'd for C₂₁H₂₂F₂N₄O₄, 433.16; found, 433.3.

### Example 116: N—((S)-1-((1R,2S,5S)-2-(((S)-4-amino-3,4-dioxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)-1H-indole-2-carboxamide (Compound 116)

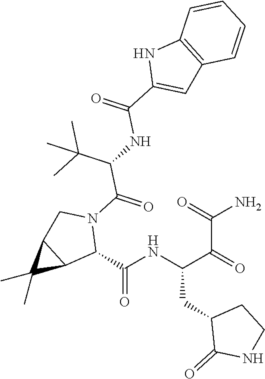 [SMILES: CC(C)(C)[C@H](NC(=O)c1cc2ccccc2[nH]1)C(=O)N1C[C@H]2[C@@H]([C@H]1C(=O)N[C@@H](C[C@@H]1CCNC1=O)C(=O)C(N)=O)C2(C)C]

### Step 1: (1R,2S,5S)-methyl 3-((S)-2-((tert-butoxycarbonyl)amino)-3,3-dimethylbutanoyl)-6,6-dimethyl-3-azabicyclo[3.1.0]hexane-2-carboxylate

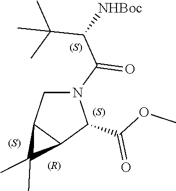

To a solution of (S)-2-((tert-butoxycarbonyl)amino)-3,3-dimethylbutanoic acid (1 g, 4.32 mmol) in DCM (15 mL) was added DIPEA (2.24 g, 17.29 mmol, 3.01 mL), and then T₃P (4.13 g, 6.49 mmol, 3.86 mL, 50% purity) was added at 0° C. The mixture was stirred at 0° C. for 1 hr. Then (1R,2S,5S)-methyl 6,6-dimethyl-3-azabicyclo[3.1.0]hexane-2-carboxylate hydrochloride (889 mg, 4.32 mmol, HCl) was added, the resulting mixture was stirred at 25° C. for 10 hr. The reaction mixture was diluted with EtOAc (50 mL) and 5% H₃PO₄ (50 mL) for 30 min. The aqueous phase was extracted with EtOAc (50 mL). The combined organic layer was washed with 5% H₃PO₄ (50 mL×3), sat.aq.NaHCO₃ (50 mL×4), water (50 mL×2) and brine (50 mL), and dried over anhydrous Na₂SO₄, filtered and concentrated. The residue was purified by flash silica gel chromatography (ISCO®; 12 g, SepaFlash® Silica Flash Column, eluent of 0~20% EtOAc/PE@30 mL/min). The title compound (630 mg, 37.40% yield) was obtained as colorless oil. ¹H NMR (400 MHz, DMSO-d₆) δ ppm 6.71 (d, J=9.2 Hz, 1H), 4.20 (s, 1H), 4.07-4.00 (m, 1H), 3.92 (d, J=10.0 Hz, 1H), 3.82-3.75 (m, 1H), 3.70-3.62 (m, 3H), 1.55-1.48 (m, 1H), 1.43-1.39 (m, 1H), 1.39-1.30 (m, 9H), 1.00 (s, 3H), 0.93 (s, 9H), 0.84 (s, 3H).

### Step 2: (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid

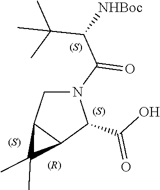

To a solution of (1R,2S,5S)-methyl 3-((S)-2-((tert-butoxycarbonyl)amino)-3,3-dimethylbutanoyl)-6,6-dimethyl-3-azabicyclo[3.1.0]hexane-2-carboxylate (630 mg, 1.65 mmol) in THF (3 mL) and H₂O (3 mL) was added LiOH·H₂O (138 mg, 3.29 mmol). The resulting mixture was stirred at 25° C. for 1 hr. To the reaction mixture was added 2M HCl until pH was adjusted to 5-6. The aqueous layer was extracted with ethyl acetate (50 mL×2). The combined organic layers were washed with brine (30 mL), dried over anhydrous Na₂SO₄, filtered and concentrated under reduced pressure to give the title compound (550 mg, 90.62% yield) as a white solid. ¹H NMR (400 MHz, CDCl₃) δ ppm 5.14 (d, J=10.4 Hz, 1H), 4.48 (s, 1H), 4.24 (d, J=10.4 Hz, 1H), 3.82 (dd, J=5.6, 10.4 Hz, 1H), 1.75 (d, J=7.6 Hz, 1H), 1.53-1.47 (m, 1H), 1.40 (s, 9H), 1.26 (t, J=7.2 Hz, 1H), 1.06 (s, 3H), 1.00 (s, 9H), 0.90 (s, 3H).

### Step 3: tert-butyl ((2S)-1-((1R,2S,5S)-2-(((2S)-4-amino-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)carbamate

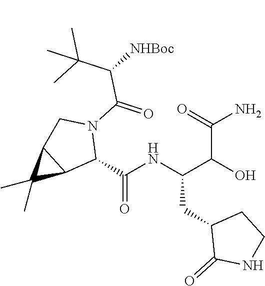

To a solution of (3S)-3-amino-2-hydroxy-4-((S)-2-oxopyrrolidin-3-yl)butanamide hydrochloride (100 mg, 496.96 mmol) in DCM (4 mL) was added DIEA (178 mg, 1.38 mmol, 240 uL) and (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid (135 mg, 366.75 mmol) slowly at 25° C. And then a solution of HATU (349 mg, 916.87 mmol) was added dropwise at 0° C. The resulting mixture was stirred at 25° C. for 16 hrs. The mixture was concentrated under reduce pressure to give a residue. The residue was purified by prep-HPLC (column: 3_Phenomenex Luna C18 75×30 mm×3 mm; mobile phase: [water (0.05% HCl)-ACN]; B %: 10%-60%, 40 min) to give the title compound (80 mg, 31.63% yield) as a white solid. ESI-MS [M+H]+ calc'd for C₂₇H₄₅N₅O₇, 552.3; found, 552.5.

### Step 4: (1R, 2S, 5S)-3-((S)-2-amino-3, 3-dimethylbutanoyl)-N-((2S)-4-amino-3-hydroxy-4-oxo-1-((S)-2-oxopyrrolidin-3-yl)butan-2-yl)-6,6-dimethyl-3-azabicyclo[3.1.0]hexane-2-carboxamide

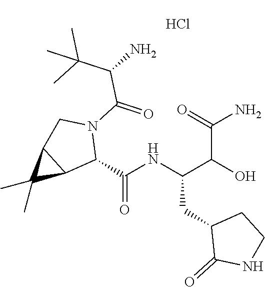

To a solution of tert-butyl ((2S)-1-((1R,2S,5S)-2-(((2S)-4-amino-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)carbamate (135 mg, 244.71 mmol) in MeOH (4 mL) was added HCl/dioxane (4 M, 3 mL). Then the mixture was stirred at 25° C. for 1 h. The reaction was concentrated under reduced pressure to give a residue. The title compound (119 mg, 99.65% yield, HCl) was obtained as a white solid.

### Step 5: N-((2S)-1-((1R,2S,5S)-2-(((2S)-4-amino-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)-1H-indole-2-carboxamide

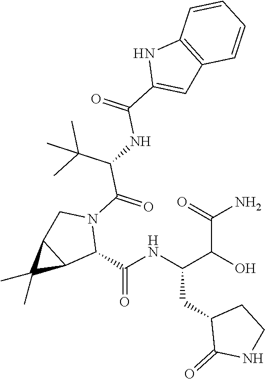 [SMILES: CC(C)(C)[C@H](NC(=O)c1cc2ccccc2[nH]1)C(=O)N1C[C@H]2[C@@H]([C@H]1C(=O)N[C@@H](C[C@@H]1CCNC1=O)C(O)C(N)=O)C2(C)C]

To a solution of (1R,2S,5S)-3-((S)-2-amino-3,3-dimethylbutanoyl)-N-((2S)-4-amino-3-hydroxy-4-oxo-1-((S)-2-oxopyrrolidin-3-yl)butan-2-yl)-6,6-dimethyl-3-azabicyclo[3.1.0]hexane-2-carboxamide hydrochloride (119 mg, 243.84 mmol, HCl) in DCM (2 mL) was added DIEA (95 mg, 731.53 mmol, 127 uL) and 1H-indole-2-carboxylic acid (31 mg, 195.07 mmol) slowly at 25° C. And HATU (185 mg, 487.69 mmol) was added to the above mixture dropwise at 0° C. The resulting mixture was stirred at 25° C. for 16 hrs. Water (10 mL) was added to the mixture. The aqueous layer was extracted with DCM (50 mL×2). The combined organic layers were concentrated under reduced pressure. The residue was purified by prep-HPLC (column: Phenomenex Gemini-NX C18 80×30 mm×5 mm; mobile phase: [water (0.05% HCl)-ACN]; B %: 35%-65%, 10 min) to give the title compound (80 mg, 55.17% yield) as a white solid. ¹H NMR (400 MHz, CD₃OD) δ ppm 7.61 (d, J=8.0 Hz, 1H), 7.47-7.34 (m, 1H), 7.27-7.13 (m, 2H), 7.10-7.01 (m, 1H), 4.82-4.75 (m, 1H), 4.51-4.26 (m, 2H), 4.16-3.84 (m, 3H), 3.79-3.53 (m, 1H), 3.29-3.21 (m, 1H), 2.79-2.10 (m, 3H), 1.97-1.53 (m, 3H), 1.51-1.36 (m, 3H), 1.36-1.24 (m, 1H), 1.13-1.04 (m, 9H), 1.03-0.86 (m, 3H).

### Step 6: N—((S)-1-((1R,2S,5S)-2-(((S)-4-amino-3,4-dioxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)-1H-indole-2-carboxamide

To a solution of N-((2S)-1-((1R,2S,5S)-2-(((2S)-4-amino-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)-1H-indole-2-carboxamide (50 mg, 84.08 μmol) in DCM (3 mL) was added NaHCO₃ (14 mg, 168.15 μmol, 6.54 uL). Then the mixture was stirred at 25° C. for 1 h. Dess-Martin (107 mg, 252.23 μmol, 78 uL) was added to the above mixture. Then the mixture was stirred at 25° C. for 1 hrs. Water (20 mL) was added to the mixture. The aqueous layer was extracted with DCM (50 mL×2). The combined organic layers were washed with brine (30 mL), dried over anhydrous Na₂SO₄, filtered and concentrated under reduced pressure. The residue was purified by prep-HPLC (column: Phenomenex Gemini-NX C18 75×30 mm×3 μm; mobile phase: [water (10 mM NH₄HCO₃)-ACN]; B %: 21%-61%, 4 min) to Compound 116 (6.0 mg, 12.04% yield) as an off-white solid. ¹H NMR (400 MHz, CD₃OD) δ ppm 7.61 (d, J=8.0 Hz, 1H), 7.43 (d, J=8.4 Hz, 1H), 7.26-7.19 (m, 1H), 7.17-7.13 (m, 1H), 7.10-7.02 (m, 1H), 4.82-4.78 (m, 1H), 4.44-4.15 (m, 2H), 4.15-3.92 (m, 2H), 3.31-3.24 (m, 2H), 2.85-1.69 (m, 4H), 1.66-1.25 (m, 3H), 1.16-1.01 (m, 12H), 0.98-0.87 (m, 3H). ESI-MS [M+H]+ calc'd for C₃₁H₄₀N₆O₆, 593.3; found, 593.5.

### Example 117: (1R,2S,5S)-3-(5-Chloro-4-fluoro-1H-indole-2-carbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 117)

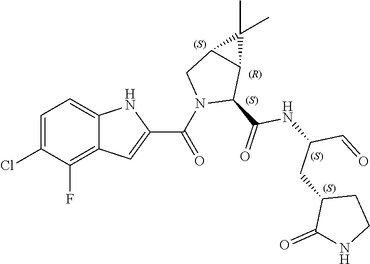

Compound 117 was synthesized by the same procedure as Compound 1. ¹H NMR (400 MHz, CD₃OD) δ ppm 7.30-7.18 (m, 2H), 7.13-6.88 (m, 1H), 4.97-4.88 (m, 1H), 4.53-4.39 (m, 1H), 4.34-4.01 (m, 1H), 4.00-3.74 (m, 2H), 3.28-3.22 (m, 1H), 3.04-2.29 (m, 2H), 2.07-1.86 (m, 1H), 1.77-1.53 (m, 3H), 1.47-1.29 (m, 1H), 1.18-0.93 (m, 6H). ESI-MS [M+H]⁺ calc'd for C₂₄H₂₆ClFN₄O₄, 489.17; found, 489.3.

### Example 118: (1R,2S,5S)-3-(1,4-Dimethylindole-2-carbonyl)-N-[(1S)-1-formyl-2-[(3S)-2-oxopyrrolidin-3-yl]ethyl]-6,6-dimethyl-3-azabicyclo[3.1.0]hexane-2-carboxamide (Compound 118)

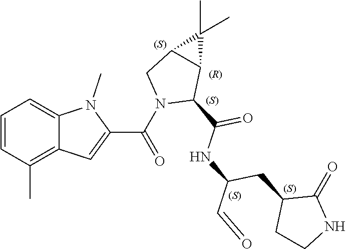

Compound 118 was synthesized by the same procedure as Compound 1. ¹H NMR (400 MHz, CD₃OD) δ ppm 7.27-7.09 (m, 2H), 6.95-6.65 (m, 2H), 4.53-4.32 (m, 1H), 4.17-4.02 (m, 1H), 3.83-3.62 (m, 4H), 3.56-3.46 (m, 1H), 2.99-2.87 (m, 1H), 2.69-2.58 (m, 1H), 2.55-2.44 (m, 3H), 2.12-1.71 (m, 1H), 1.67-1.53 (m, 3H), 1.28-1.04 (m, 8H), 0.98-0.83 (m, 1H). ESI-MS [M+H]⁺ calc'd for C₂₆H₃₂N₄O₄, 465.24; found, 465.4.

### Example 119: (1R,2S,5S)—N—((S)-4-(cyclopropylamino)-3,4-dioxo-1-((S)-2-oxopyrrolidin-3-yl)butan-2-yl)-6,6-dimethyl-3-(1-phenylcyclopropanecarbonyl)-3-azabicyclo[3.1.0]hexane-2-carboxamide (Compound 119)

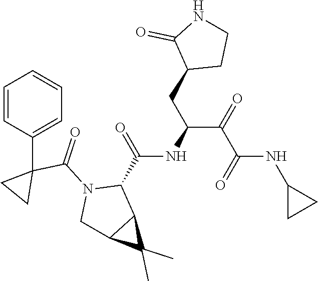 [SMILES: CC1(C)[C@@H]2[C@@H](C(=O)N[C@@H](C[C@@H]3CCNC3=O)C(=O)C(=O)NC3CC3)N(C(=O)C3(c4ccccc4)CC3)C[C@@H]21]

### Step 1: tert-butyl ((S)-1-hydroxy-3-((S)-2-oxopyrrolidin-3-yl)propan-2-yl)carbamate

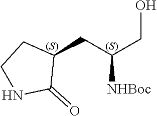

To a solution of (S)-methyl 2-((tert-butoxycarbonyl)amino)-3-((S)-2-oxopyrrolidin-3-yl)propanoate (10 g, 34.93 mmol) in THE (100 mL) was added LiBH₄ (2.28 g, 104.78 mmol) and LiBH₄ (2.57 g, 117.98 mmol) at 0° C. And then the mixture was stirred at 25° C. for 1 hr. The reaction was quenched with water (30 mL) drop-wise. The resulting mixture was extracted with DCM (100 ml×3). The combined organic phase was washed with water (50 mL) dried over anhydrous Na₂SO₄, filtered and concentrated. The title compound (9.19 g, crude) was obtained as a white solid. ¹H NMR (400 MHz, CDCl₃) δ ppm 6.42 (s, 1H), 3.78-3.66 (m, 1H), 3.66-3.53 (m, 2H), 3.40-3.28 (m, 2H), 3.27-3.06 (m, 1H), 2.57-2.33 (m, 2H), 2.02-1.90 (m, 1H), 1.88-1.75 (m, 1H), 1.67-1.54 (m, 1H), 1.43 (s, 9H).

### Step 2: tert-butyl ((S)-1-oxo-3-((S)-2-oxopyrrolidin-3-yl)propan-2-yl)carbamate

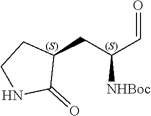

To a solution of tert-butyl ((S)-1-hydroxy-3-((S)-2-oxopyrrolidin-3-yl)propan-2-yl)carbamate (2 g, 7.74 mmol) in DCM (30 mL) was added SO₃·Py (7.39 g, 46.46 mmol), TEA (4.70 g, 46.46 mmol, 6.47 mL) and DMSO (6.05 g, 77.43 mmol, 6.05 mL) at 25° C. Then the mixture was stirred at 25° C. for 1 hr. The mixture was diluted with Water (100 mL), extracted with DCM (200 mL×3). The organic layer was dried over Na₂SO₄, filtered and concentrated to give crude product. The title compound (2 g, crude) was obtained as yellow oil.

### Step 3: isocyanocyclopropane

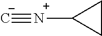

To a solution of cyclopropanamine (5 g, 87.57 mmol, 6.07 mL) in DCM (40 mL) was added a solution of NaOH (21.02 g, 525.45 mmol) in H₂O (40 mL). Then benzyl(triethyl)ammonium;chloride (399 mg, 1.75 mmol) and CHCl₃ (62.73 g, 525.45 mmol, 42.38 mL) were added respectively. The mixture was stirred at 25° C. for 12 hr. The organic layer was extracted with a syringe. The residue was directly purified by flash silica gel chromatography (Eluent of 0% MeOH in DCM). Eluent in a test tube with most pungent odour was used directly in the next step. Isocyanocyclopropane (6.6 g, crude) was obtained as yellow oil. ¹H NMR (400 MHz, CDCl₃) δ ppm 2.69 (tt, J=3.6, 7.6 Hz, 1H), 1.02-0.93 (m, 2H), 0.91-0.80 (m, 2H).

### Step 4: (3S)-3-((tert-butoxycarbonyl)amino)-1-(cyclopropylamino)-1-oxo-4-((S)-2-oxopyrrolidin-3-yl)butan-2-yl acetate

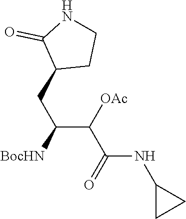 [SMILES: CC(=O)OC(C(=O)NC1CC1)[C@H](C[C@@H]1CCNC1=O)NC(=O)OC(C)(C)C]

To a solution of tert-butyl ((S)-1-oxo-3-((S)-2-oxopyrrolidin-3-yl)propan-2-yl)carbamate (2 g, 7.80 mmol) and isocyanocyclopropane (523.52 mg, 7.80 mmol) in DCM (25 mL) was added AcOH (937 mg, 15.61 mmol). Then the mixture was stirred at 25° C. for 12 hr. The reaction mixture was diluted with EtOAc (50 mL). The combined organic layer was washed with sat.aq. NaHCO₃ (20 mL), 1 M Na₂CO₃ (20 mL) and brine (20 mL), and dried over anhydrous Na₂SO₄, filtered and concentrated. The residue was purified by prep-HPLC (column: YMC Triart 30×150 mm×7 μm; mobile phase: [water (NH₃H₂O+NH₄HCO₃)-ACN]; B %: 2%-32%, 27 min). The title compound (120 mg, 3.76% yield) was obtained as a yellow solid. ¹H NMR (400 MHz, CDCl₃) δ ppm 6.58-6.31 (m, 1H), 6.12 (s, 1H), 5.61-4.98 (m, 2H), 4.30-3.97 (m, 1H), 3.44-3.19 (m, 2H), 2.67 (s, 1H), 2.50-2.29 (m, 2H), 2.20-2.11 (m, 3H), 2.07-1.91 (m, 1H), 1.90-1.74 (m, 1H), 1.61-1.49 (m, 1H), 1.41 (s, 9H), 0.86-0.66 (m, 2H), 0.51 (s, 2H).

### Step 5: (3S)-3-amino-1-(cyclopropylamino)-1-oxo-4-((S)-2-oxopyrrolidin-3-yl)butan-2-yl acetate

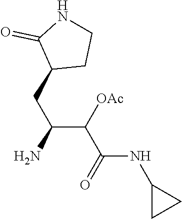 [SMILES: CC(=O)OC(C(=O)NC1CC1)[C@@H](N)C[C@@H]1CCNC1=O]

To a solution of (3S)-3-((tert-butoxycarbonyl)amino)-1-(cyclopropylamino)-1-oxo-4-((S)-2-oxopyrrolidin-3-yl)butan-2-yl acetate (60 mg, 156.48 mol) in DCM (1 mL) was added TFA (0.1 mL). The mixture was stirred at 25° C. for 1 hr. The reaction mixture was concentrated directly. The title compound (60 mg, crude, TFA) was obtained as yellow oil.

### Step 6: (3S)-1-(cyclopropylamino)-3-((1R,2S,5S)-6,6-dimethyl-3-(1-phenylcyclopropanecarbonyl)-3-azabicyclo[3.1.0]hexane-2-carboxamido)-1-oxo-4-((S)-2-oxopyrrolidin-3-yl)butan-2-yl acetate

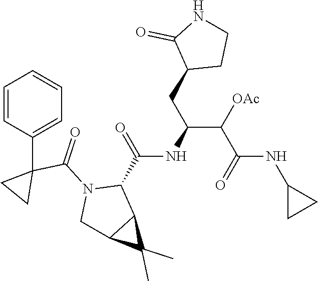 [SMILES: CC(=O)OC(C(=O)NC1CC1)[C@H](C[C@@H]1CCNC1=O)NC(=O)[C@@H]1[C@@H]2[C@H](CN1C(=O)C1(c3ccccc3)CC1)C2(C)C]

To a solution of (3S)-3-amino-1-(cyclopropylamino)-1-oxo-4-((S)-2-oxopyrrolidin-3-yl)butan-2-yl acetate (45.20 mg, 151.00 mol), (1R,2S,5S)-6,6-dimethyl-3-(1-phenylcyclopropanecarbonyl)-3-azabicyclo[3.1.0]hexane-2-carboxylic acid (60 mg, 151.00 mol, TFA) and DIEA (59 mg, 453.01 μmol, 78.90 uL) in DCM (3 mL) was added slowly HATU (115 mg, 302.00 mol) at 0° C. The resulting mixture was stirred at 25° C. for 3 hr. The reaction mixture was concentrated. Sat.aq.NH₄Cl (5 mL) was added to the residue. The resulting mixture was extracted with DCM (10 mL×3). The combined organic phase was washed with water (5 mL) dried over anhydrous Na₂SO₄, filtered and concentrated. The residue was purified by flash silica gel chromatography (Eluent of 0~25% MeOH in DCM). The title compound (75 mg, 71.50% yield) was obtained as yellow oil. ¹H NMR (400 MHz, CDCl₃) δ ppm 7.38-7.27 (m, 2H), 7.24-7.09 (m, 3H), 5.34-5.11 (m, 1H), 4.57-4.22 (m, 1H), 3.43-3.21 (m, 3H), 2.91-2.77 (m, 2H), 2.55-2.30 (m, 2H), 2.23-2.10 (m, 2H), 2.09-2.05 (m, 1H), 2.04 (s, 3H), 1.93-1.73 (m, 1H), 1.62-1.55 (m, 6H), 1.14-1.02 (m, 1H), 1.01-0.91 (m, 3H), 0.78-0.67 (m, 4H), 0.65-0.46 (m, 2H).

### Step 7: (1R,2S,5S)—N-((2S)-4-(cyclopropylamino)-3-hydroxy-4-oxo-1-((S)-2-oxopyrrolidin-3-yl)butan-2-yl)-6,6-dimethyl-3-(1-phenylcyclopropanecarbonyl)-3-azabicyclo[3.1.0]hexane-2-carboxamide

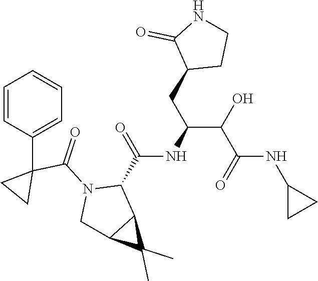 [SMILES: CC1(C)[C@@H]2[C@@H](C(=O)N[C@@H](C[C@@H]3CCNC3=O)C(O)C(=O)NC3CC3)N(C(=O)C3(c4ccccc4)CC3)C[C@@H]21]

To a solution of (3S)-1-(cyclopropylamino)-3-((1R,2S,5S)-6,6-dimethyl-3-(1-phenylcyclopropanecarbonyl)-3-azabicyclo[3.1.0]hexane-2-carboxamido)-1-oxo-4-((S)-2-oxopyrrolidin-3-yl)butan-2-yl acetate (75 mg, 132.82 mol) in MeOH (3 mL) was added K₂CO₃ (37 mg, 265.64 mol) at 0° C. And then the mixture was stirred at 25° C. for 12 hr. The reaction mixture was concentrated. Sat.aq.NH₄Cl (5 mL) was added to the residue. The resulting mixture was extracted with DCM (10 mL×3). The combined organic phase was washed with water (5 mL), dried over anhydrous Na₂SO₄, filtered and concentrated. The title compound (50 mg, crude) was obtained as a yellow solid. ¹H NMR (400 MHz, CDCl₃) δ ppm 7.37-7.27 (m, 2H), 7.25-7.09 (m, 3H), 6.50-5.78 (m, 1H), 4.53-4.33 (m, 1H), 4.29-4.13 (m, 1H), 3.52-3.22 (m, 3H), 3.01-2.78 (m, 2H), 2.65-2.34 (m, 2H), 2.27-1.76 (m, 3H), 1.75-1.56 (m, 4H), 1.39-1.04 (m, 5H), 1.02-0.86 (m, 3H), 0.81-0.66 (m, 4H), 0.61-0.49 (m, 1H)

### Step 8: (1R,2S,5S)—N—((S)-4-(cyclopropylamino)-3,4-dioxo-1-((S)-2-oxopyrrolidin-3-yl)butan-2-yl)-6,6-dimethyl-3-(1-phenylcyclopropanecarbonyl)-3-azabicyclo[3.1.0]hexane-2-carboxamide

To a solution of (1R,2S,5S)—N-((2S)-4-(cyclopropylamino)-3-hydroxy-4-oxo-1-((S)-2-oxopyrrolidin-3-yl)butan-2-yl)-6,6-dimethyl-3-(1-phenylcyclopropanecarbonyl)-3-azabicyclo[3.1.0]hexane-2-carboxamide (50 mg, 95.67 mol) in DCM (5 mL) was added Dess-Martin (122 mg, 287.01 mol). The mixture was stirred at 25° C. for 1 hr. The reaction mixture was filtered and concentrated. The residue was purified by prep-HPLC (column: YMC Triart 30×150 mm×7 μm; mobile phase: [water (NH4HCO3)-ACN]; B %: 29%-59%, 15 min). Compound 119 (3.2 mg, 6.07 μmol, 6.34% yield) was obtained as a white solid.

¹H NMR (400 MHz, CD₃OD) δ ppm 7.43-7.12 (m, 5H), 4.38-3.73 (m, 2H), 3.69-3.53 (m, 1H), 3.39-3.35 (m, 1H), 3.29-3.22 (m, 1H), 2.82-2.44 (m, 2H), 2.42-2.14 (m, 1H), 2.12-1.60 (m, 3H), 1.48-1.20 (m, 6H), 1.15-1.02 (m, 1H), 1.01-0.86 (m, 3H), 0.81-0.64 (m, 5H), 0.63-0.50 (m, 2H). ESI-MS [M+H]⁺ calc'd for C₂₉H₃₆N₄O₅, 521.27, found 521.5.

### Example 120: (1R,2S,5S)-3-(5H-[1,3]dioxolo[4,5-f]indole-6-carbonyl)-N-[(1S)-1-formyl-2-[(3S)-2-oxopyrrolidin-3-yl]ethyl]-6,6-dimethyl-3-azabicyclo[3.1.0]hexane-2-carboxamide (Compound 120)

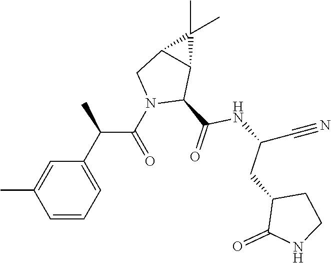 [SMILES: Cc1cccc([C@@H](C)C(=O)N2C[C@H]3[C@@H]([C@H]2C(=O)N[C@H](C#N)C[C@@H]2CCNC2=O)C3(C)C)c1]

### Step 1: tert-butyl ((S)-1-amino-1-oxo-3-((S)-2-oxopyrrolidin-3-yl)propan-2-yl)carbamate

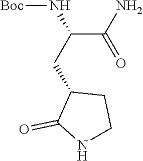 [SMILES: CC(C)(C)OC(=O)N[C@@H](C[C@@H]1CCNC1=O)C(N)=O]

A mixture of (S)-methyl 2-((tert-butoxycarbonyl)amino)-3-((S)-2-oxopyrrolidin-3-yl)propanoate (5.00 g, 17.46 mmol) in NH₃/MeOH (7 M, 50 mL) was purged with N₂ for 3 times, and then the mixture was stirred at 50° C. for 96 hours under N₂ atmosphere. The reaction mixture was concentrated directly. Crude product was used in the next step directly, no purification needed. Crude product (5.20 g, 13.42 mmol, 76.83% yield) was obtained as a yellow solid. ¹H NMR (400 MHz, CDCl₃) δ ppm 7.13 (s, 1H), 6.20-5.92 (m, 2H), 5.67 (s, 1H), 4.37 (s, 1H), 3.44-3.30 (m, 2H), 2.58-2.48 (m, 1H), 2.44-2.34 (m, 1H), 2.13-2.02 (m, 1H), 1.94-1.88 (m, 1H), 1.79-1.71 (m, 1H), 1.44 (s, 9H).

### Step 2: tert-butyl ((S)-1-cyano-2-((S)-2-oxopyrrolidin-3-yl)ethyl)carbamate

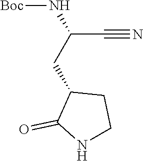 [SMILES: CC(C)(C)OC(=O)N[C@H](C#N)C[C@@H]1CCNC1=O]

To a solution of tert-butyl ((S)-1-amino-1-oxo-3-((S)-2-oxopyrrolidin-3-yl)propan-2-yl)carbamate (1.00 g, 3.69 mmol) in THE (10 mL) was added TEA (372 mg, 3.69 mmol, 513 uL) at 0° C. Then TFAA (774 mg, 3.69 mmol, 512.67 uL) was added dropwise to the mixture. The reaction was stirred at 25° C. for 2 hr. The mixture was concentrated under reduced pressure. The residue was poured into ice-water (20 mL). The aqueous phase was extracted with ethyl acetate (50 mL×3). The combined organic phase was washed with sat.aq.NaHCO₃ (30 mL) and brine (30 mL), dried with anhydrous sodium sulfate, filtered and concentrated under reduced pressure. The crude product was triturated with n-hexane (10 mL) at 25° C. for 30 minutes. The solid was filtered and dried under reduced pressure. The title compound (700 mg, 1.69 mmol, 45.74% yield) was obtained as a yellow solid which was used in the next step without further purification. ¹H NMR (400 MHz, CD₃OD) δ ppm 4.75-4.67 (m, 1H), 3.37-3.32 (m, 2H), 2.57-2.44 (m, 1H), 2.41-2.30 (m, 1H), 2.27-2.13 (m, 1H), 1.94-1.76 (m, 2H), 1.47 (s, 9H).

### Step 3: (S)-2-amino-3-((S)-2-oxopyrrolidin-3-yl)propanenitrile

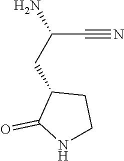 [SMILES: N#C[C@@H](N)C[C@@H]1CCNC1=O]

To a solution of tert-butyl ((S)-1-cyano-2-((S)-2-oxopyrrolidin-3-yl)ethyl)carbamate (300 mg, 1.18 mmol) in DCM (6 mL) was added TFA (3 mL) in one portion at 25° C. The mixture was stirred for 1 hr and then concentrated under reduce pressure. The title compound (300 mg, crude, TFA) was obtained as a yellow oil.

### Step 4: (1R,2S,5S)-tert-butyl 2-(((S)-1-cyano-2-((S)-2-oxopyrrolidin-3-yl)ethyl)carbamoyl)-6,6-dimethyl-3-azabicyclo[3.1.0]hexane-3-carboxylate

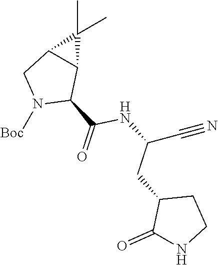 [SMILES: CC(C)(C)OC(=O)N1C[C@H]2[C@@H]([C@H]1C(=O)N[C@H](C#N)C[C@@H]1CCNC1=O)C2(C)C]

To a solution of (S)-2-amino-3-((S)-2-oxopyrrolidin-3-yl)propanenitrile (300 mg, 1.12 mmol, TFA) and (1R,2S,5S)-3-(tert-butoxycarbonyl)-6,6-dimethyl-3-azabicyclo[3.1.0]hexane-2-carboxylic acid (286 mg, 1.12 mmol) in DCM (2 mL) was added DIEA (435 mg, 3.37 mmol, 586 uL) at 0° C. Then HATU (853 mg, 2.25 mmol) was added in one portion at 0° C. The mixture was stirred at 25° C. for 2 hours. The mixture was poured into water (10 mL). The aqueous phase was extracted with DCM (30 mL×3). The combined organic phase was washed with NH₄Cl (20 mL×2), dried with anhydrous Na₂SO₄, filtered and concentrated in vacuum. The residue was purified by column chromatography (SiO₂, Petroleum ether/Ethyl acetate=0:1). The title compound (150 mg, 13.69% yield) was obtained as a white solid. ¹H NMR (400 MHz, CD₃OD) δ ppm 5.10-4.98 (m, 1H), 4.05-4.01 (m, 1H), 3.71 (dt, J=5.6, 11.2 Hz, 1H), 3.44-3.33 (m, 2H), 3.29-3.19 (m, 1H), 2.66-2.21 (m, 3H), 2.00-1.75 (m, 2H), 1.47-1.40 (m, 9H), 1.37-1.33 (m, 2H), 1.09-0.92 (m, 6H).

### Step 5: (1R,2S,5S)-tert-butyl 2-(((S)-1-cyano-2-((S)-2-oxopyrrolidin-3-yl)ethyl)carbamoyl)-6,6-dimethyl-3-azabicyclo[3.1.0]hexane-3-carboxylate

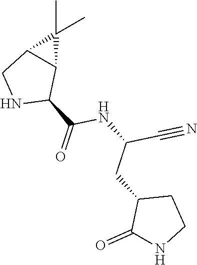 [SMILES: CC1(C)[C@@H]2[C@@H](C(=O)N[C@H](C#N)C[C@@H]3CCNC3=O)NC[C@@H]21]

To a solution of (1R,2S,5S)-tert-butyl 2-(((S)-1-cyano-2-((S)-2-oxopyrrolidin-3-yl)ethyl)carbamoyl)-6,6-dimethyl-3-azabicyclo[3.1.0]hexane-3-carboxylate (140 mg, 358.54 μmol) in DCM (5 mL) and TFA (0.5 mL) was stirred at 25° C. for 10 hr. The mixture was concentrated under reduce pressure. The title compound (145 mg, crude, TFA) was obtained as a yellow oil which was used in the next step without further purification.

### Step 6: (1R,2S,5S)-3-(5H-[1,3]dioxolo[4,5-f]indole-6-carbonyl)-N-[(1S)-1-formyl-2-[(3S)-2-oxopyrrolidin-3-yl]ethyl]-6,6-dimethyl-3-azabicyclo[3.1.0]hexane-2-carboxamide

To a solution of (R)-2-(m-tolyl)propanoic acid (21 mg, 126.12 mol), (1R,2S,5S)—N—((S)-1-cyano-2-((S)-2-oxopyrrolidin-3-yl)ethyl)-6,6-dimethyl-3-azabicyclo[3.1.0]hexane-2-carboxamide (51 mg, 126.12 mol TFA) and DIEA (49 mg, 378.35 μmol, 66.00 μL) in DCM (1 mL) was added HATU (96 mg, 252.24 mol) at 0° C. The resulting mixture was stirred at 25° C. for 10 hr. The reaction mixture was concentrated. Sat.aq.NH₄Cl (10 mL) was added to the residue. The resulting mixture was extracted with DCM (20 mL×3). The combined organic phase was washed with water (10 mL), dried over anhydrous Na₂SO₄, filtered and concentrated. The residue was purified by prep-HPLC (column: Phenomenex C18 75×30 mm×3 μm; mobile phase: [water (NH₃H₂O+NH₄HCO₃)-ACN]; B %: 26%-60%, 11 min). Compound 120 (8.1 mg, 14.71% yield, 100% purity) was obtained. ¹H NMR (400 MHz, DMSO-d₆) δ ppm 9.38-8.74 (m, 1H), 7.87-7.64 (m, 1H), 7.28-6.88 (m, 4H), 5.12-4.80 (m, 1H), 4.48-3.95 (m, 1H), 3.91-3.53 (m, 2H), 3.22-3.07 (m, 3H), 2.45-2.36 (m, 1H), 2.25 (s, 3H), 2.21-2.07 (m, 2H), 1.86-1.62 (m, 2H), 1.47-1.12 (m, 5H), 1.07-0.28 (m, 6H). ESI-MS [M+H]⁺ calc'd for C₂₅H₃₂N₄O₃, 437.55; found, 437.4.

### Example 121: (2S,4R)-1-(1H-Indole-2-carbonyl)-4-methoxy-N—((S)-1-oxo-3-((S)-2-oxopyrrolidin-3-yl)propan-2-yl)pyrrolidine-2-carboxamide (Compound 121)

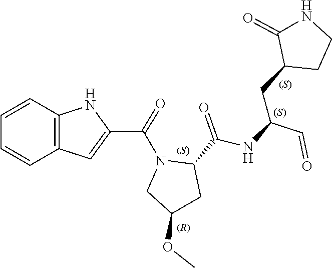

Compound 121 was synthesized by the same procedure as Compound 2. ¹H NMR (400 MHz, CD₃OD) δ ppm 7.68-7.50 (m, 1H), 7.44 (d, J=8.4 Hz, 1H), 7.27-7.17 (m, 1H), 7.12-6.83 (m, 2H), 4.77-4.63 (m, 1H), 4.50-4.28 (m, 1H), 4.21-4.07 (m, 2H), 4.04-3.89 (m, 1H), 3.85-3.56 (m, 1H), 3.46-3.31 (m, 3H), 3.30-3.23 (m, 1H), 2.84-2.62 (m, 1H), 2.49-2.34 (m, 1H), 2.24-1.98 (m, 2H), 1.91-1.50 (m, 2H), 1.43-1.15 (m, 1H). ESI-MS [M+H]⁺ calc'd for C₂₂H₂₆N₄O₅, 427.19; found, 427.4.

### Example 122: (1R,2S,5S)-6,6-Dimethyl-N—((S)-1-oxo-3-((S)-2-oxopyrrolidin-3-yl)propan-2-yl)-3-(3-phenyl-1H-indole-2-carbonyl)-3-azabicyclo[3.1.0]hexane-2-carboxamide (Compound 122)

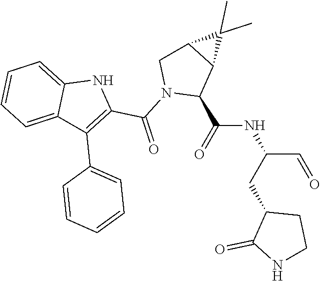 [SMILES: CC1(C)[C@@H]2[C@@H](C(=O)N[C@H](C=O)C[C@@H]3CCNC3=O)N(C(=O)c3[nH]c4ccccc4c3-c3ccccc3)C[C@@H]21]

Compound 122 was synthesized by the same procedure as Compound 1. ¹H NMR (400 MHz, CD₃OD) δ ppm 7.72-7.41 (m, 6H), 7.39-7.29 (m, 1H), 7.28-7.17 (m, 1H), 7.15-6.97 (m, 1H), 4.83-4.67 (m, 1H), 4.54-4.26 (m, 1H), 4.10-3.95 (m, 1H), 3.93-3.82 (m, 1H), 3.53-3.37 (m, 2H), 3.25-2.79 (m, 2H), 2.71-2.35 (m, 1H), 2.12-1.64 (m, 1H), 1.49-1.37 (m, 2H), 1.34-1.25 (m, 1H), 0.96-0.86 (m, 3H), 0.67-0.40 (m, 3H). ESI-MS [M+H]⁺ calc'd for C₃₀H₃₂N₄O₄, 513.24; found, 513.5.

### Example 123: (1R,2S,5S)-3-(1-(3-Chlorophenyl)cyclopropanecarbonyl)-N—((S)-1-cyano-2-((S)-2-oxopyrrolidin-3-yl)ethyl)-6,6-dimethyl-3-azabicyclo[3.1.0]hexane-2-carboxamide (Compound 123)

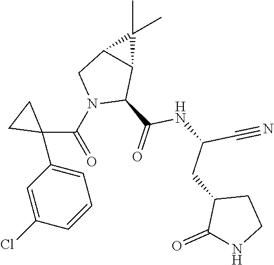 [SMILES: CC1(C)[C@@H]2[C@@H](C(=O)N[C@H](C#N)C[C@@H]3CCNC3=O)N(C(=O)C3(c4cccc(Cl)c4)CC3)C[C@@H]21]

Compound 123 was synthesized by the same procedure as Compound 120. ¹H NMR (400 MHz, DMSO-d₆) δ ppm 9.18-8.90 (m, 1H), 7.86-7.64 (m, 1H), 7.42-7.01 (m, 4H), 5.09-4.79 (m, 1H), 4.25-3.75 (m, 1H), 3.67-3.40 (m, 1H), 3.25-3.05 (m, 3H), 2.45-2.34 (m, 1H), 2.31-2.05 (m, 2H), 1.87-1.64 (m, 2H), 1.47-1.22 (m, 4H), 1.21-1.06 (m, 2H), 1.02-0.86 (m, 3H), 0.79-0.47 (m, 3H). ESI-MS [M+H]⁺ calc'd for C₂₅H₂₉ClN₄O₃, 469.98; found, 469.4.

### Example 124: (1R,2S,5S)-3-(Cyclopentanecarbonyl)-6,6-dimethyl-N—((S)-1-oxo-3-((S)-2-oxopyrrolidin-3-yl)propan-2-yl)-3-azabicyclo[3.1.0]hexane-2-carboxamide (Compound 124)

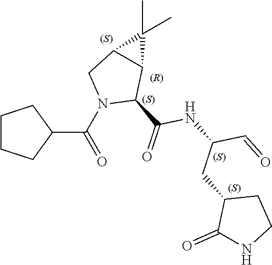

Compound 124 was synthesized by the same procedure as Compound 2. ¹H NMR (400 MHz, CD₃OD) δ ppm 4.52-4.38 (m, 1H), 4.28 (d, J=2.4 Hz, 1H), 4.03-3.57 (m, 3H), 2.95-2.80 (m, 1H), 2.65-2.48 (m, 1H), 2.38-2.25 (m, 1H), 1.95 (s, 1H), 1.92-1.65 (m, 6H), 1.65-1.22 (m, 7H), 1.09-0.95 (m, 6H). ESI-MS [M+H]⁺ calc'd for C₂₁H₃₁N₃O₄, 390.23; found, 390.4.

### Example 125: (1S,3S,5S)-2-(1H-Indole-2-carbonyl)-N—((S)-1-oxo-3-((S)-2-oxopyrrolidin-3-yl)propan-2-yl)-2-azabicyclo[3.1.0]hexane-3-carboxamide (Compound 125)

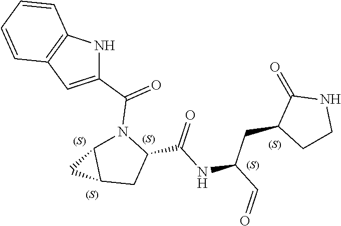

Compound 125 was synthesized by the same procedure as Compound 2. ¹H NMR (400 MHz, CDCl₃) δ ppm 9.57-9.46 (m, 1H), 9.45-9.33 (m, 1H), 8.27-8.12 (m, 1H), 7.72-7.61 (m, 1H), 7.46-7.37 (m, 1H), 7.33-7.28 (m, 1H), 7.24-7.19 (m, 1H), 7.17-7.11 (m, 1H), 5.74-5.57 (m, 1H), 5.20 (dd, J=3.2, 10.4 Hz, 1H), 4.44-4.30 (m, 1H), 3.95-3.84 (m, 1H), 3.29-3.13 (m, 2H), 2.63-2.44 (m, 2H), 2.40-2.25 (m, 2H), 2.06-1.89 (m, 2H), 1.83-1.78 (m, 1H), 1.33-1.24 (m, 1H), 1.14-1.02 (m, 1H), 0.93-0.77 (m, 1H). ESI-MS [M+H]⁺ calc'd for C₂₂H₂₄N₄O₄, 409.18; found, 409.3.

### Example 126: (S)-5-(2-Cyclohexylacetyl)-N—((S)-1-oxo-3-((S)-2-oxopyrrolidin-3-yl)propan-2-yl)-5-azaspiro[2.4]heptane-6-carboxamide (Compound 126)

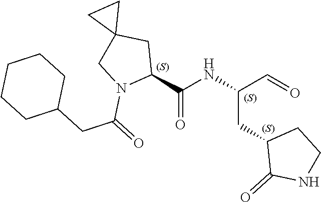

Compound 126 was synthesized by the same procedure as Compound 2. ¹H NMR (400 MHz, CDCl₃) δ ppm 9.60-9.45 (m, 1H), 9.06-8.24 (m, 1H), 6.59-5.69 (m, 1H), 4.99-4.58 (m, 1H), 4.57-4.25 (m, 1H), 3.75-3.49 (m, 1H), 3.44-3.21 (m, 3H), 2.56-2.36 (m, 2H), 2.28-2.08 (m, 3H), 2.06-1.81 (m, 8H), 1.79-1.72 (m, 2H), 1.31-1.20 (m, 2H), 1.20-1.03 (m, 1H), 1.02-0.85 (m, 2H), 0.69-0.40 (m, 4H). ESI-MS [M+H]⁺ calc'd for C₂₂H₃₃N₃O₄, 404.25; found, 404.1.

### Example 127: (S)—N—((S)-1-(benzo[d]thiazol-2-yl)-1-oxo-3-((S)-2-oxopyrrolidin-3-yl)propan-2-yl)-5-(1H-indole-2-carbonyl)-5-azaspiro[2.4]heptane-6-carboxamide (Compound 127)

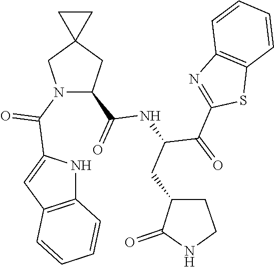 [SMILES: O=C1NCC[C@H]1C[C@H](NC(=O)[C@@H]1CC2(CC2)CN1C(=O)c1cc2ccccc2[nH]1)C(=O)c1nc2ccccc2s1]

### Example 128: (1R,2S,5S)-3-(7-Chloro-4-fluoro-1H-indole-2-carbonyl)-N—((S)-1-cyano-2-((S)-2-oxopyrrolidin-3-yl)ethyl)-6,6-dimethyl-3-azabicyclo[3.1.0]hexane-2-carboxamide (Compound 128)

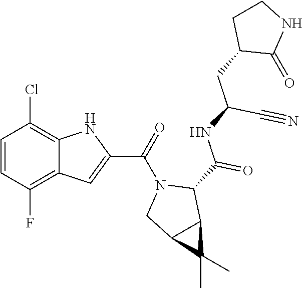 [SMILES: CC1(C)[C@@H]2[C@@H](C(=O)N[C@H](C#N)C[C@@H]3CCNC3=O)N(C(=O)c3cc4c(F)ccc(Cl)c4[nH]3)C[C@@H]21]

Compound 128 was synthesized by the same procedure as Compound 120. ¹H NMR (400 MHz, CD₃OD) δ ppm 77.30-6.72 (m, 3H), 5.09 (dd, J=5.6, 10.2 Hz, 1H), 4.86 (br d, J=6.8 Hz, 1H), 4.37-4.00 (m, 1H), 3.98-3.79 (m, 1H), 3.32-3.27 (m, 1H), 3.32-3.03 (m, 1H), 2.86-2.61 (m, 1H), 2.42-2.03 (m, 2H), 2.00-1.52 (m, 4H), 1.25-0.93 (m, 6H). ESI-MS [M+H]⁺ calc'd for C₂₄H₂₅ClFN₅O₃, 485.16; found, 486.4.

### Example 129: (2S,3aS,6aS)-1-(1H-Indole-2-carbonyl)-N—((S)-1-oxo-3-((S)-2-oxopyrrolidin-3-yl)propan-2-yl)octahydrocyclopenta[b]pyrrole-2-carboxamide (Compound 129)

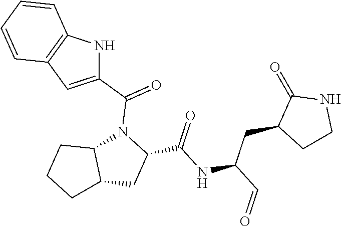 [SMILES: O=C[C@H](C[C@@H]1CCNC1=O)NC(=O)[C@@H]1C[C@@H]2CCC[C@@H]2N1C(=O)c1cc2ccccc2[nH]1]

Compound 129 was synthesized by the same procedure as Compound 2. ¹H NMR (400 MHz, CDCl₃) δ ppm 9.61-9.46 (m, 2H), 8.35-8.12 (m, 1H), 7.50-7.14 (m, 1H), 7.12-7.10 (m, 1H), 7.09-7.04 (m, 1H), 7.00-6.92 (m, 1H), 6.89-6.80 (m, 1H), 5.62-5.83 (m, 1H), 4.63-4.91 (m, 2H), 4.35-4.25 (m, 1H), 3.32-3.05 (m, 4H), 2.86-2.63 (m, 1H), 2.49-2.03 (m, 8H) 1.39-1.15 (m, 3H). ESI-MS [M+H]⁺ calc'd for C₂₄H₂₈N₄O₄, 437.21; found, 437.2.

### Example 130: (1S,2S,5R)-3-(1H-Indole-2-carbonyl)-N—((S)-1-oxo-3-((S)-2-oxopyrrolidin-3-yl)propan-2-yl)-3-azabicyclo[3.1.0]hexane-2-carboxamide (Compound 130)

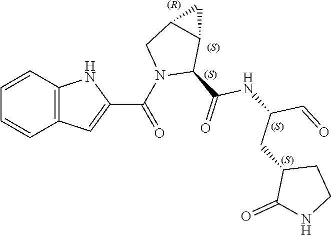

Compound 130 was synthesized by the same procedure as Compound 2. ¹H NMR (400 MHz, MeOD) δ ppm 7.65 (d, J=7.6 Hz, 1H), 7.54 (d, J=7.6 Hz, 1H), 7.42 (d, J=8.0 Hz, 1H), 7.27-7.17 (m, 1H), 7.09-6.98 (m, 1H), 6.90 (d, J=6.0 Hz, 1H), 4.25-4.12 (m, 1H), 4.02-3.73 (m, 2H), 3.35-3.32 (m, 1H), 3.26-3.15 (m, 1H), 2.95-2.87 (m, 1H), 2.57 (m, 1H), 2.36-2.26 (m, 1H), 2.10-1.29 (m, 6H), 0.90 (m, 1H), 0.46-0.16 (m, 1H) ESI-MS [M+H]⁺ calc'd for C₂₂H₂₄N₄O₄, 409.18; found, 409.1.

### Example 131: (1R,2S,5S)—N—((S)-1-Cyano-2-((S)-2-oxopyrrolidin-3-yl)ethyl)-3-(7-fluoro-4-isopropyl-1H-indole-2-carbonyl)-6,6-dimethyl-3-azabicyclo[3.1.0]hexane-2-carboxamide (Compound 131)

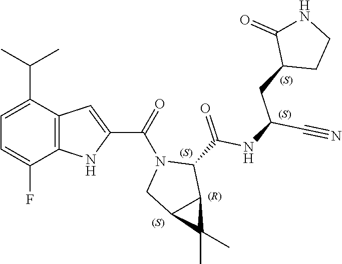

Compound 131 was synthesized by the same procedure as Compound 120. ¹H NMR (400 MHz, CD₃OD) δ ppm 7.17 (d, J=3.2 Hz, 1H), 6.95-6.83 (m, 2H), 5.11-4.91 (m, 1H), 4.34 (dd, J=5.6, 10.8 Hz, 1H), 4.04-3.80 (m, 2H), 3.43-3.34 (m, 1H), 2.76-2.61 (m, 1H), 2.45-2.24 (m, 2H), 2.09-1.66 (m, 4H), 1.64-1.46 (m, 2H), 1.40-1.28 (m, 6H), 1.17-0.95 (m, 6H). ESI-MS [M+H]⁺ calc'd for C₂₇H₃₂FN₅O₃, 494.25; found, 494.4.

### Example 132: (1R,2S,5S)-3-(7-Chloro-1H-indole-2-carbonyl)-N—((S)-1-cyano-2-((S)-2-oxopyrrolidin-3-yl)ethyl)-6,6-dimethyl-3-azabicyclo[3.1.0]hexane-2-carboxamide (Compound 132)

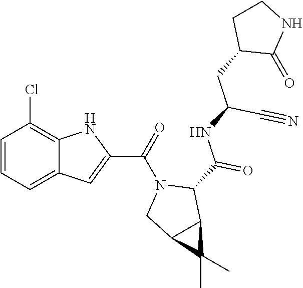 [SMILES: CC1(C)[C@@H]2[C@@H](C(=O)N[C@H](C#N)C[C@@H]3CCNC3=O)N(C(=O)c3cc4cccc(Cl)c4[nH]3)C[C@@H]21]

Compound 132 was synthesized by the same procedure as Compound 120. ¹H NMR (400 MHz, CD₃OD) δ ppm 7.58-7.10 (m, 2H), 7.09-6.74 (m, 2H), 4.97 (dd, J=5.6, 10.2 Hz, 1H), 4.20 (dd, J=5.6, 10.6 Hz, 1H), 4.03-3.60 (m, 2H), 2.64-2.47 (m, 1H), 2.31-2.19 (m, 1H), 1.99-1.69 (m, 2H), 1.68-1.45 (m, 2H), 1.43-1.28 (m, 1H), 1.27-1.17 (m, 1H), 1.08-0.88 (m, 8H). ESI-MS [M+H]⁺ calc'd for C₂₄H₂₆ClN₅O₃, 468.17; found, 468.3.

### Example 133: (2S,4R)-1-(1H-Indole-2-carbonyl)-N—((S)-1-oxo-3-((S)-2-oxopyrrolidin-3-yl)propan-2-yl)-4-phenoxypyrrolidine-2-carboxamide (Compound 133)

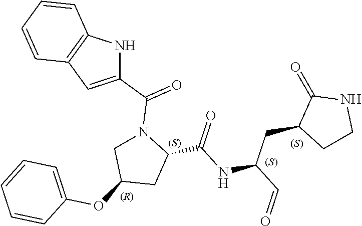

Compound 133 was synthesized by the same procedure as Compound 2. ¹H NMR (400 MHz, CDCl₃) δ ppm 9.54 (s, 1H), 8.54 (d, J=4.4 Hz, 1H), 7.65 (d, J=8.4 Hz, 1H), 7.41 (d, J=8.8 Hz, 1H), 7.33-7.26 (m, 3H), 7.12 (t, J=7.6 Hz, 1H), 6.98 (t, J=8.0 Hz, 1H), 6.93-6.85 (m, 3H), 5.94-5.70 (m, 1H), 5.17 (s, 1H), 4.96 (t, J=7.6 Hz, 1H), 4.45-4.32 (m, 2H), 4.31-4.23 (m, 1H), 3.37-3.21 (m, 2H), 2.71-2.47 (m, 3H), 2.42-2.27 (m, 1H), 2.07-1.92 (m, 2H), 1.90-1.74 (m, 2H). ESI-MS [M+H]⁺ calc'd for C₂₇H₂₈N₄O₅, 489.54; found, 489.2.

### Example 134: (2S,4S)-1-(1H-Indole-2-carbonyl)-N—((S)-1-oxo-3-((S)-2-oxopyrrolidin-3-yl)propan-2-yl)-4-phenoxypyrrolidine-2-carboxamide (Compound 134)

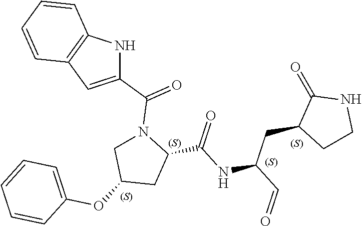

Compound 134 was synthesized by the same procedure as Compound 2. ¹H NMR (400 MHz, CDCl₃) δ ppm 9.76-9.53 (m, 1H), 9.52-9.41 (m, 1H), 7.68 (d, J=8.0 Hz, 1H), 7.43 (d, J=8.4 Hz, 1H), 7.33-7.27 (m, 3H), 7.16-7.10 (m, 1H), 7.02-6.95 (m, 2H), 6.87-6.70 (m, 3H), 5.70-5.31 (m, 1H), 5.21-5.10 (m, 1H), 5.07-4.83 (m, 1H), 4.35-4.12 (m, 3H), 3.38-2.99 (m, 2H), 2.97-2.81 (m, 1H), 2.69-2.08 (m, 4H), 1.96-1.80 (m, 2H). ESI-MS [M+H]⁺ calc'd for C₂₇H₂₈N₄O₅, 489.21; found, 489.4.

### Example 135: (1R,2S,5S)—N—((S)-1-Cyano-2-((S)-2-oxopyrrolidin-3-yl)ethyl)-6,6-dimethyl-3-(1-phenylcyclopropanecarbonyl)-3-azabicyclo[3.1.0]hexane-2-carboxamide (Compound 135)

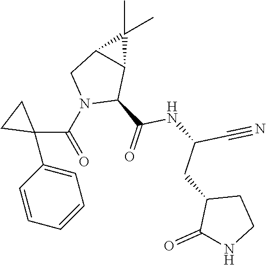 [SMILES: CC1(C)[C@@H]2[C@@H](C(=O)N[C@H](C#N)C[C@@H]3CCNC3=O)N(C(=O)C3(c4ccccc4)CC3)C[C@@H]21]

Compound 135 was synthesized by the same procedure as Compound 120. ¹H NMR (400 MHz, DMSO-d₆) δ ppm 9.21-8.82 (m, 1H), 7.87-7.63 (m, 1H), 7.39-7.07 (m, 5H), 5.11-4.77 (m, 1H), 4.29-3.77 (m, 1H), 3.27-3.03 (m, 4H), 2.45-2.35 (m, 1H), 2.31-2.06 (m, 2H), 1.89-1.63 (m, 2H), 1.44-1.05 (m, 6H), 0.99-0.83 (m, 3H), 0.76-0.37 (m, 3H). ESI-MS [M+H]⁺ calc'd for C₂₅H₃₀N₄O₃, 435.53; found, 435.4.

### Example 136: (1R,2S,5S)—N—((S)-1-Cyano-2-((S)-2-oxopyrrolidin-3-yl)ethyl)-6,6-dimethyl-3-(1-phenylcyclopentanecarbonyl)-3-azabicyclo[3.1.0]hexane-2-carboxamide (Compound 136)

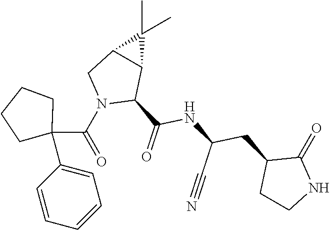 [SMILES: CC1(C)[C@@H]2[C@@H](C(=O)N[C@H](C#N)C[C@@H]3CCNC3=O)N(C(=O)C3(c4ccccc4)CCCC3)C[C@@H]21]

Compound 136 was synthesized by the same procedure as Compound 120. ¹H NMR (400 MHz, DMSO-d₆) δ ppm 7.37-7.20 (m, 5H), 5.13-4.97 (m, 1H), 4.30-4.23 (m, 1H), 3.50-3.33 (m, 3H), 3.00-2.86 (m, 1H), 2.80-2.63 (m, 1H), 2.46-2.30 (m, 3H), 2.30-2.19 (m, 2H), 1.96-1.72 (m, 5H), 1.68-1.53 (m, 2H), 1.38-1.30 (m, 1H), 1.27-1.17 (m, 1H), 0.98-0.87 (m, 3H), 0.66-0.50 (m, 3H). ESI-MS [M+H]⁺ calc'd for C₂₇H₃₄N₄O₃, 463.26; found, 463.4.

### Example 137: (2S,4R)-4-(3-Chlorobenzyl)-1-(1H-indole-2-carbonyl)-N—((S)-1-oxo-3-((S)-2-oxopyrrolidin-3-yl)propan-2-yl)pyrrolidine-2-carboxamide (Compound 137)

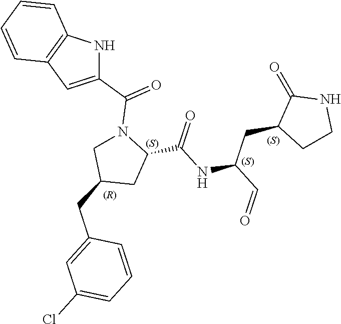

Compound 137 was synthesized by the same procedure as Compound 2. ¹H NMR (400 MHz, CD₃OD) δ ppm 7.69-7.49 (m, 1H), 7.48-7.39 (m, 1H), 7.32-7.13 (m, 5H), 7.10-6.83 (m, 2H), 4.47-4.16 (m, 1H), 4.09-3.88 (m, 1H), 3.83-3.66 (m, 1H), 3.49-3.37 (m, 0.5H), 3.00-2.90 (m, 0.5H), 2.88-2.76 (m, 2H), 2.74-2.53 (m, 2H), 2.46-2.15 (m, 2H), 2.15-1.85 (m, 3H), 1.83-1.22 (m, 4H). ESI-MS [M+H]⁺ calc'd for C₂₈H₂₉ClN₄O₄, 521.19; found, 521.4.

### Example 138: (S)-1-(3,3-Dimethylbutanoyl)-N—((S)-1-oxo-3-((S)-2-oxopyrrolidin-3-yl)propan-2-yl)pyrrolidine-2-carboxamide (Compound 138)

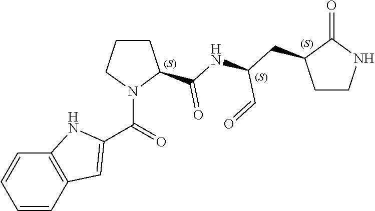

Compound 138 was synthesized by the same procedure as Compound 2. ¹H NMR (400 MHz, CD₃OD) δ ppm 7.74-7.52 (m, 1H), 7.51-7.40 (m, 1H), 7.30-7.18 (m, 1H), 7.16-6.82 (m, 2H), 5.04-5.24 (m, 1H), 4.61-3.72 (m, 1H), 4.24-3.71 (m, 4H), 3.04-2.63 (m, 1H), 2.60-2.31 (m, 2H), 2.30-2.16 (m, 2H), 2.14-1.88 (m, 2H), 1.85-1.54 (m, 1H), 1.50-1.27 (m, 1H). ESI-MS [M+H]⁺ calc'd for C₂₁H₂₄N₄O₄, 397.18; found, 397.4.

### Evaluation of the Activity of Compounds Against Sars-Cov-2 Mpro

### Materials

The hCoV Mpro are cloned, expressed in E. coli and purified by WuXi AppTec Co., Ltd. The substrate are synthesized by Genscript Biotech.

### Methods

Compounds are 3 fold serially diluted for 10 doses and added to an assay plate (384w format) using ECHO, in duplicate wells. The test concentration of test compounds is defined by sponsor. For 100% inhibition control (HPE, hundred percent effect), 2 μM of GC376 is added. For no inhibition control (ZPE, zero percent effect), DMSO is added.

25 μL of Mpro proteins are added to the assay plates containing compounds, respectively, using Multidrop. The compounds and Mpro proteins are pre-incubated at room temperature for 30 min. Then 5 L of Mpro substrates are added to the related assay plates, respectively. Each activity testing point has a relevant background control to normalize the fluorescence interference of compound. The test concentrations of the Mpro protein and substrate are shown in Table 4.

TABLE 4
The final test concentrations of Mpro protein in enzymatic assay

| | Final concentrations | | Final concentrations of | |
|---|---|---|---|---|
| Protein | of proteins | | substrates | |
| SARS-CoV-2_Mpro | 25 | nM | 25 | μM |
| HCoV-229E_Mpro | 50 | nM | 12.5 | μM |
| HCoV-NL63_Mpro | 50 | nM | 12.5 | μM |
| SARS_Mpro | 25 | nM | 25 | μM |
| HCoV-OC43_Mpro | 25 | nM | 12.5 | μM |
| HCoV-HKU1_Mpro | 12.5 | nM | 12.5 | μM |
| MERS_Mpro | 100 | nM | 25 | μM |

After 60 min incubation at 30° C., the fluorescence signal (RFU) is detected using a microplate reader M2e (SpectraMax) at Ex/Em=340 nm/490 nm.

The inhibition activity is calculated using the formula below, IC₅₀ values will be calculated using the Inhibition % data.

Inhibition %=[(CPD−BGCPD)−(ZPE−BGZPE)/(HPE−BGHPE)−(ZPE−BGZPE)]×1000%
CPD: Signal of test compounds wells, containing compound+enzyme+substrate.
ZPE: Average of signals of zero percent effective control wells, containing enzyme+substrate, no compound.
HPE: Average of signals of hundred percent effect control wells, containing GC376+enzyme+substrate.
BG: Compound background control wells, containing compound+substrate, no enzyme

IC₅₀ values of compounds are calculated with the GraphPad Prism software using the nonlinear regression model of log(inhibitor) vs. response—Variable slope (four parameters).

The mean C₅₀ (nM) values of compounds are provided in Table 5.

TABLE 5

| | Mpro_SARS- | Mpro_{—} | Mpro_{—} | Mpro_{—} | Mpro_{—} | Mpro_{—} | Mpro_SARS- |
|---|---|---|---|---|---|---|---|
| No. | CoV-2 | 229E | HKU1 | MERS | NL63 | OC43 | CoV-1 |
| 1 | 9.534 | 32.39 | 11.43 | 129.3 | 73.74 | 28.37 | 17.33 |
| 2 | 9.836 | 32.56 | 10.44 | 198.5 | 48.42 | 38.2 | 42.55 |
| 3 | 10.07 | 27.45 | 9.952 | 110.0 | 17.75 | 24.96 | 19.29 |
| 4 | 10.15 | 28.51 | 26.81 | 197.7 | 34.41 | 40.77 | 23.57 |
| 5 | 10.46 | 33.94 | 21.97 | 146.5 | 53.5 | 42.12 | 22.47 |
| 6 | 10.77 | 31.06 | 25.12 | 168 | 38.29 | 38.85 | 19.53 |
| 7 | 11.03 | 21.97 | 9.203 | 81.04 | 33.64 | 26.56 | 12.91 |
| 8 | 11.30 | 21.76 | 7.738 | 88.74 | 37.57 | 18.94 | 11.94 |
| 9 | 12.13 | 30.85 | 20.46 | 313.3 | 52.32 | 65.12 | 37.80 |
| 10 | 12.32 | 32.30 | 17.74 | 193.6 | 45.01 | 35.77 | 45.34 |
| 11 | 13.20 | 37.20 | 39.97 | 229.5 | 50.63 | 34.15 | 38.39 |
| 12 | 13.25 | 33.80 | 15.01 | 175.2 | 53.89 | 37.03 | 21.20 |
| 13 | 13.99 | 48.10 | 38.25 | 371.5 | 76.30 | 69.02 | 37.74 |
| 14 | 14.49 | 39.56 | 22.25 | 322.3 | 49.30 | 39.39 | 51.99 |
| 15 | 14.53 | 39.75 | 24.18 | 174.8 | 44.48 | 32.81 | 32.67 |
| 16 | 14.58 | 24.88 | 12.54 | 157 | 28.88 | 30.51 | 23.52 |
| 17 | 15.21 | 48.37 | 23.75 | 276.2 | 180.4 | 59.82 | 27.78 |
| 18 | 15.28 | 38.81 | 33.09 | 301.5 | 76.13 | 65.92 | 33.56 |
| 19 | 15.39 | 27.36 | 18.04 | 188.2 | 37.29 | 49.73 | 40.07 |
| 20 | 15.52 | 39.55 | 16.41 | 273.7 | 37.33 | 54.95 | 40.36 |
| 21 | 15.66 | 22.21 | 37.96 | 234.3 | 57.66 | 59.41 | 27.55 |
| 22 | 15.82 | 24.44 | 10.49 | 123.2 | 37.06 | 28.35 | 27.73 |
| 23 | 15.82 | 33.78 | 22.59 | 125.6 | 49.68 | 30.54 | 23.28 |
| 24 | 16.60 | 32.31 | 31.81 | 270.9 | 71.68 | 60.49 | 73.6 |
| 25 | 16.67 | 33.83 | 30.21 | 140.5 | 53.78 | 43.00 | 24.04 |
| 26 | 16.80 | 42.75 | 20.67 | 172.9 | 37.77 | 28.56 | 93.67 |
| 27 | 17.19 | 54.67 | 49.02 | 466.1 | 82.76 | 89.22 | 52.82 |
| 28 | 18.00 | 35.61 | 19.92 | 224.4 | 50.89 | 43.16 | 26.94 |
| 29 | 18.33 | 47.42 | 53.46 | 295.75 | 93.86 | 87.01 | 35.18 |
| 30 | 18.63 | 24.46 | 26.01 | 238.8 | 49.74 | 43.75 | 51.11 |
| 31 | 18.92 | 31.66 | 14.18 | 188.2 | 49.28 | 22.18 | 34.00 |
| 32 | 19.69 | 35.43 | 21.82 | 177.1 | 59.62 | 52.39 | 36.32 |
| 33 | 19.71 | 43.49 | 37.87 | 226.9 | 78.02 | 83.20 | 33.97 |
| 34 | 19.82 | 37.68 | 20.80 | 278.0 | 65.28 | 57.95 | 35.32 |
| 35 | 20.30 | 37.41 | 11.42 | 179.4 | 48.48 | 64.38 | 65.81 |
| 36 | 20.58 | 33.17 | 35.66 | 245.3 | 51.72 | 42.66 | 31.65 |
| 37 | 20.622 | 45.595 | 36.415 | 241.9 | 188 | 36.69 | 40.43 |
| 38 | 21.04 | 41.62 | 15.97 | 516.1 | 100.2 | 46.00 | 97.03 |
| 39 | 21.58 | 26.40 | 15.95 | 208.2 | 39.96 | 35.09 | 29.97 |
| 40 | 21.68 | 40.05 | 20.39 | 236.8 | 88.61 | 45.69 | 35.27 |
| 41 | 21.77 | | | | | | |
| 42 | 22.10 | | | | | | |
| 43 | 22.13 | 39.35 | 18.03 | 236.4 | 47.47 | 45.36 | 25.76 |
| 44 | 22.16 | | | | | | |
| 45 | 22.50 | | | | | | |
| 46 | 22.52 | | | | | | |
| 47 | 22.81 | 47.80 | 16.81 | 138.8 | 48.00 | 64.02 | 43.00 |
| 48 | 22.95 | 86.9 | 73.4 | 484.2 | 376.3 | 163.9 | 53.58 |
| 49 | 22.98 | 31.52 | 23.42 | 201.8 | 57.57 | 37.03 | 29.43 |
| 50 | 24.18 | 130.2 | 34.24 | 620.6 | 255.6 | 170.7 | 46.85 |
| 51 | 24.43 | 27.33 | 13.02 | 191.7 | 22.22 | 21.95 | 38.03 |
| 52 | 24.55 | 31.44 | 25.01 | 214 | 61.27 | 51.96 | 32.56 |
| 53 | 24.965 | 29.68 | 25.55 | 326.3 | 66.48 | 26.27 | 45.75 |
| 54 | 25.05 | 36.38 | 18.8 | 285.1 | 42.65 | 56.67 | 37.82 |
| 55 | 25.53 | 39.34 | 54.77 | 317.3 | 70.20 | 80.99 | 74.58 |
| 56 | 25.71 | 42.58 | 12.76 | 291.0 | 59.95 | 59.81 | 64.70 |
| 57 | 25.78 | | | | | | |
| 58 | 25.84 | 28.47 | 19.04 | 338.4 | 40.72 | 56.32 | 62.78 |
| 59 | 26.00 | 42.56 | 22.92 | 230.3 | 64.05 | 57.48 | 83.43 |
| 60 | 26.01 | 53.68 | 28.90 | 272.7 | 54.00 | 94.90 | 52.00 |
| 61 | 26.03 | 43.25 | 12.63 | 437.7 | 200.5 | 38.73 | 147.5 |
| 62 | 26.28 | 64.39 | 55.72 | 535.3 | 90.66 | 144.7 | 77.11 |
| 63 | 26.71 | 53.83 | 39.68 | 382.4 | 105.1 | 66.76 | 32.09 |
| 64 | 26.99 | 53.24 | 46.51 | 223.9 | 105.2 | 65.83 | 46.55 |
| 65 | 27.00 | 38.02 | 21.11 | 320.0 | 24.08 | 25.2 | 33.11 |
| 66 | 27.61 | | | | | | |
| 67 | 27.84 | 27.33 | 10.79 | 169.7 | 51.8 | 33.2 | 44.11 |
| 68 | 28.00 | | | | | | |
| 69 | 29.17 | 50.46 | 32.64 | 475.7 | 80.66 | 79.84 | 48.85 |
| 70 | 30.10 | | | | | | |
| 71 | 30.36 | 53.54 | 34.90 | 1416 | 414.5 | 133.3 | 95.65 |
| 72 | 31.28 | 30.07 | 20.83 | 345.8 | 21.74 | 49.36 | 58.90 |
| 73 | 31.75 | 62.64 | 57.45 | 325.6 | 181.6 | 71.4 | 63.13 |
| 74 | 32.00 | 55.35 | 40.87 | 478.8 | 86.85 | 86.38 | 61.45 |
| 75 | 32.29 | 63.98 | 16.58 | 604.2 | 753.9 | 56.97 | 66.02 |
| 76 | 32.74 | 26.86 | 13.10 | 200.3 | 33.04 | 30.07 | 60.56 |
| 77 | 33.12 | 41.89 | 45.26 | 401.1 | 99.19 | 83.76 | 27.63 |
| 78 | 33.32 | 37.43 | 51.13 | 478.7 | 81.28 | 66.88 | 86.78 |
| 79 | 34.49 | 50.78 | 27.1 | 176.9 | 57.77 | 38.56 | 25.94 |
| 80 | 35.01 | 28.59 | 22.49 | 383.7 | 48.21 | 63.34 | 75.47 |
| 81 | 35.30 | 78.225 | 37.05 | 2985 | 1054 | 173.2 | 124.7 |
| 82 | 35.47 | 26.15 | 10.59 | 176.1 | 30.13 | 33.72 | 35.06 |
| 83 | 35.49 | 46.34 | 39.04 | 436.3 | 58.88 | 81.26 | 115.5 |
| 84 | 36.03 | 47.63 | 17.27 | 702.8 | 184.8 | 41.16 | 88.31 |
| 85 | 36.73 | 34.57 | 23.2 | 258.9 | 53.19 | 47.87 | 48.19 |
| 86 | 39.67 | 34.2 | 33.68 | 317.9 | 78.79 | 52.47 | 69.51 |
| 87 | 40.06 | | | | | | |
| 88 | 40.30 | 41.47 | 21.18 | 239.1 | 68.54 | 44.87 | 46.18 |
| 89 | 40.90 | 44.71 | 31.04 | 395.2 | 69.50 | 88.54 | 65.44 |
| 90 | 43.62 | 60.48 | 47.99 | 654.5 | 201.5 | 129.6 | 79.43 |
| 91 | 43.91 | 92.93 | 61.08 | 396.0 | 215.5 | 110.0 | 83.35 |
| 92 | 45.01 | 43.88 | 39.36 | 418.1 | 161.7 | 108.7 | 89.64 |
| 93 | 45.05 | 32.93 | 24.02 | 317.5 | 53.19 | 55.14 | 73.23 |
| 94 | 45.08 | 69.87 | 179.7 | 567.8 | 344.1 | 230.5 | 114.9 |
| 95 | 45.51 | 67.54 | 64.42 | 412.9 | 189.2 | 123.9 | 100.4 |
| 96 | 49.24 | | | | | | |
| 97 | 49.88 | 46.00 | 27.05 | 395.3 | 88.85 | 71.97 | 75.40 |
| 98 | 51.07 | 57.00 | 7.000 | 518.0 | 105.0 | 81.00 | 206.0 |
| 99 | 51.35 | | | | | | |
| 100 | 53.98 | 106.2 | 94.45 | 876.9 | 397.7 | 133.0 | 92.50 |
| 101 | 56.31 | | | | | | |
| 102 | 63.19 | 118.0 | 83.00 | 631.0 | 199.0 | 141.0 | 204.0 |
| 103 | 63.92 | | | | | | |
| 104 | 65.07 | 116.3 | 109.2 | 684.3 | 861.4 | 196.7 | 114.3 |
| 105 | 73.19 | | | | | | |
| 106 | 73.66 | 91.18 | 39.69 | 859.1 | 156.5 | 118.4 | 176.3 |
| 107 | 76.97 | 44.24 | 46.46 | 521.7 | 62.03 | 195.0 | 186.4 |
| 108 | 78.47 | 48.00 | 29.00 | 291.0 | 38.00 | 193.0 | 159.0 |
| 109 | 80.75 | 373.8 | 48.89 | 4317 | 2325 | 270.5 | 230.5 |
| 110 | 82.21 | 104.7 | 229.5 | 1162 | 1586 | 318.3 | 204.7 |
| 111 | 84.13 | 83.26 | 73.29 | 865.5 | 134.8 | 169.0 | 118.7 |
| 112 | 84.59 | 149.5 | 119.6 | 2763 | 502.5 | 389.6 | 244.1 |
| 113 | 91.56 | 33.24 | 28.38 | 581.4 | 35.74 | 79.33 | 339.5 |
| 114 | 97.265 | 56.19 | 30.64 | 429.1 | 99.31 | 77.86 | 148.6 |
| 115 | 102.5 | 53.00 | 15.00 | 303.0 | 105.0 | 87.00 | 250.0 |
| 116 | 118.2 | | | | | | |
| 117 | 126.5 | | | | | | |
| 118 | 127.3 | 325.3 | 324.4 | 3293 | 1785 | 435.7 | 349.2 |
| 119 | 143.3 | | | | | | |
| 120 | 151.4 | 298.6 | 193.1 | 1131 | 1399 | 567.3 | 196.1 |
| 121 | 159.5 | 151.7 | 546.5 | 2630 | 198.1 | 825.1 | 294.1 |
| 122 | 162.0 | 172.1 | 365.9 | 1547 | 1024 | 423.1 | 233.9 |
| 123 | 178.2 | 458.9 | 184.6 | 1178 | 2112 | 419.3 | 223.9 |
| 124 | 211.3 | 161.8 | 407.3 | 2001 | 2500 | 489.8 | 473.0 |
| 125 | 227.5 | 115.6 | 153.8 | 915.7 | 217.5 | 283.7 | 684.7 |
| 126 | 231.3 | 56.33 | 38.43 | 358.3 | 97.66 | 112.4 | 84.51 |
| 127 | 260.9 | 95.09 | 372.1 | 409.4 | 194.8 | 507.3 | 599.8 |
| 128 | 317.6 | 1228 | 1903 | 5579 | 3981 | 1993 | 787.9 |
| 129 | 351.0 | 38.00 | 76.00 | 1108 | 51.00 | 166.0 | 874.0 |
| 130 | 372.2 | 191.0 | 87.36 | 838.3 | 130.0 | 397.5 | 832.0 |
| 131 | 400.8 | 689.9 | 1135 | 9763 | 1586 | 1545 | 772.4 |
| 132 | 433.7 | 1210 | 1911 | 5442 | 3973 | 3011 | 872.6 |
| 133 | 442.7 | | | | | | |
| 134 | 450.7 | | | | | | |
| 135 | 467.7 | 3455 | 765.8 | 4540 | 11840 | 1875 | 571.1 |
| 136 | 470.2 | 3049 | 1626 | 8406 | 13930 | 3980 | 766.6 |
| 137 | 478.0 | 1259 | 261.9 | 36260 | 6563 | 1419 | 1523 |
| 138 | 499.0 | 208.0 | 226.8 | 1729 | 417.4 | 317.3 | 690.9 |

1. A compound of formula I: 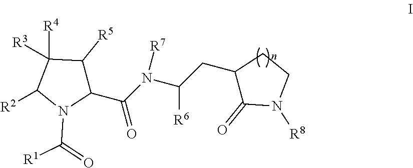
or a pharmaceutically acceptable salt thereof, wherein:
R¹ is
(a) —C₁₋₆alkyl,
(b) —C₁₋₆haloalkyl,
(c) —C₁₋₆alkylene-NHCO—C₁₋₆haloalkyl,
(d) —C₀₋₆alkylene-(saturated cycloalkyl),
(e) —C₀₋₆alkylene-heterocycloalkyl,
(f) —C₀₋₆alkylene-(tricyclic heteroaryl),
(g) -heteroarylene-cycloalkyl,
(h) -heteroarylene-aryl,
(i) 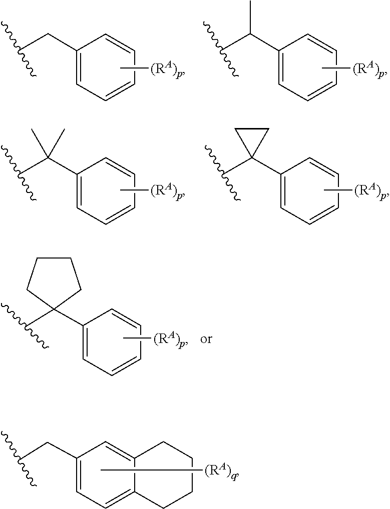
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl;
p is 0, 1, 2, 3, 4, or 5; and
q is 0, 1, 2, 3, 4, 5, 6, 7, 8, 9, 10, or 11;
(j) 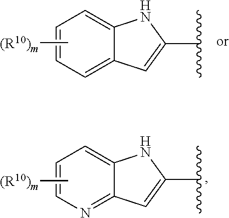
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; or
(k) 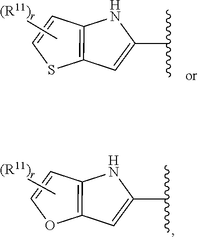
or wherein R¹¹ is halo, CN, —C₁₋₆alkyl, —C₁₋₆haloalkyl, —C₁₋₆alkoxy, or —SO₂(C₁₋₆alkyl); and r is 0 or 1;
R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl;
R² is H; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
R⁶ is 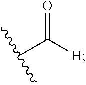
R⁷ and R are each independently H or C₁₋₆alkyl; and
n is 1, 2, or 3;
wherein at each occurrence, cycloalkyl, saturated cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl);
provided that where n is 1; R², R⁷, and R⁸ are H; R³ and R⁵ or R⁴ and R⁵ together with the carbon atom to which they are attached form a 3 membered or 5 membered-cycloalkyl; then R¹ is not

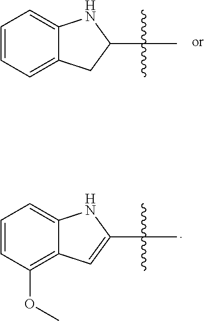 [SMILES: *C1Cc2ccccc2N1 ; *c1cc2c(OC)cccc2[nH]1]

2. The compound of claim 1, or a pharmaceutically acceptable salt thereof, wherein R¹ is
(a) —C₁₋₆alkyl,
(b) —C₁₋₆alkylene-NHCO—C₁₋₆haloalkyl,
(c) —C₀₋₆alkylene-(saturated cycloalkyl),
(d) —C₀₋₆alkylene-heterocycloalkyl,
(e) —C₀₋₆alkylene-(tricyclic heteroaryl),
(f) -heteroarylene-cycloalkyl,
(g) 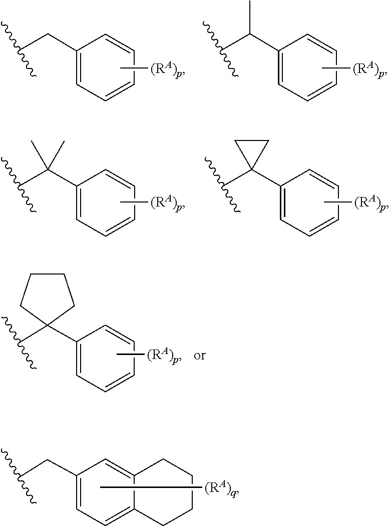
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl;
p is 0, 1, 2, 3, 4, or 5, and
q is 0, 1, 2, 3, 4, 5, 6, 7, 8, 9, 10, or 11;
(h) 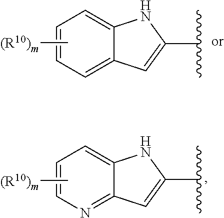
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(i) 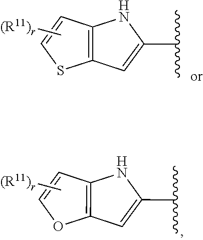
wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, cycloalkyl, heterocycloalkyl, aryl, phenyl, and heteroaryl are each independently substituted with 0, 1, or 2 substituents independently selected from the group consisting of halo, —OH, —CN, —C₁₋₆alkyl, —C₁₋₆haloalkyl, —C₁₋₆alkoxy, —SO₂(C₁₋₆alkyl), and —CO(C₁₋₆alkyl).

3. The compound of claim 1, or a pharmaceutically acceptable salt thereof, wherein R¹ is
(a) —C₁₋₆alkyl,
(b) —C₁₋₆alkylene-NHCO—C₁₋₆haloalkyl,
(c) —C₀₋₆alkylene-(saturated cycloalkyl), wherein cycloalkyl is substituted with 0 or 1 phenyl,
(d) —C₀₋₆alkylene-(3-7 membered heterocycloalkyl),
(e) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl,
(f) -(5-12 membered heteroarylene)-C₃₋₆cycloalkyl,
(g) -(5-12 membered heteroarylene)-phenyl,
(h) 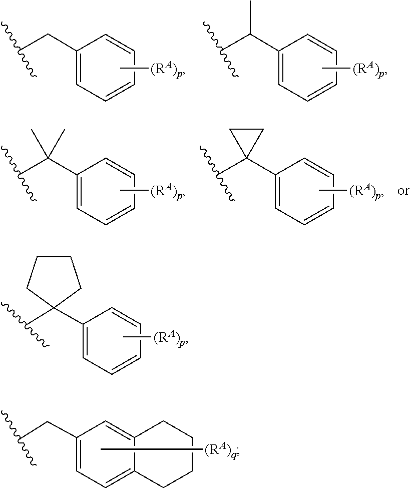
(i) 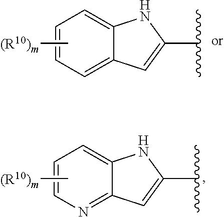
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(j) 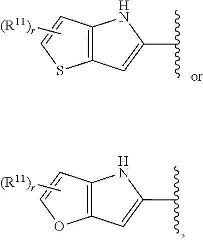
wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, heterocycloalkyl, aryl, phenyl, and heteroaryl are each independently substituted with 0, 1, or 2 substituents independently selected from the group consisting of halo, —OH, —CN, —C₁₋₆alkyl, —C₁₋₆haloalkyl, —C₁₋₆alkoxy, —SO₂(C₁₋₆alkyl), and —CO(C₁₋₆alkyl).

4. The compound of claim 3, or a pharmaceutically acceptable salt thereof, wherein R¹ is —C₀₋₆alkylene-(C₃₋₆ saturated cycloalkyl), wherein the cycloalkyl is substituted with 0, 1, or 2 substituents independently selected from the group consisting of halo, —OH, —CN, —C₁₋₆alkyl, —C₁₋₆haloalkyl, and —C₁₋₆alkoxy.

5. The compound of claim 4, or a pharmaceutically acceptable salt thereof, wherein R¹ is selected from the group consisting of

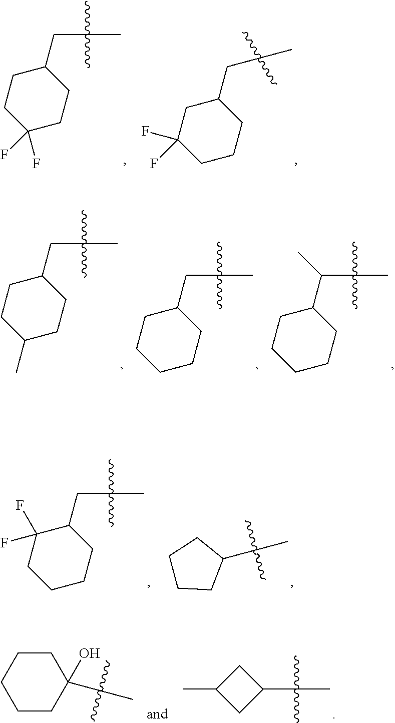

6. The compound of claim 3, or a pharmaceutically acceptable salt thereof, wherein R¹ is 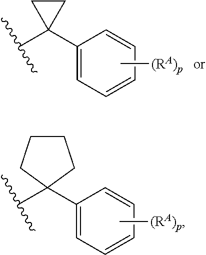
wherein R¹ is substituted with 0, 1, or 2 R^{A} substituents independently selected from the group consisting of halo, —OH, —CN, —C₁₋₆alkyl, —C₁₋₆haloalkyl, and —C₁₋₆alkoxy; and p is 0, 1, or 2.

7. The compound of claim 6, or a pharmaceutically acceptable salt thereof, wherein R¹ is halo or —C₁₋₆alkyl; and p is 0 or 1.

8. The compound of claim 1 or 7, or a pharmaceutically acceptable salt thereof, wherein R¹ is selected from the group consisting of

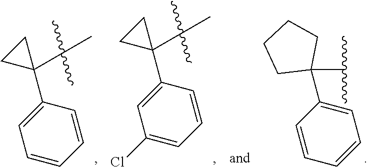

9. The compound of claim 3, or a pharmaceutically acceptable salt thereof, wherein R¹ is —C₀₋₆alkylene-(3-7 membered heterocycloalkyl), wherein the heterocycloalkyl is substituted with 0, 1, or 2 substituents selected from the group consisting of halo, —OH, —CN, —C₁₋₆alkyl, —C₁₋₆haloalkyl, and —C₁₋₆alkoxy.

10. The compound of claim 9, or a pharmaceutically acceptable salt thereof, wherein R¹ is —C₀₋₃alkylene-(5-7 membered heterocycloalkyl).

11. The compound of claim 10, or a pharmaceutically acceptable salt thereof, wherein R¹ is selected from the group consisting of

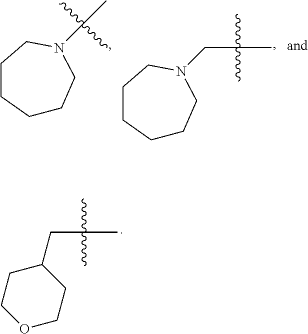 [SMILES: *N1CCCCCC1 ; *CN1CCCCCC1 ; *CC1CCOCC1]

12. The compound of claim 3, or a pharmaceutically acceptable salt thereof, wherein R¹ is 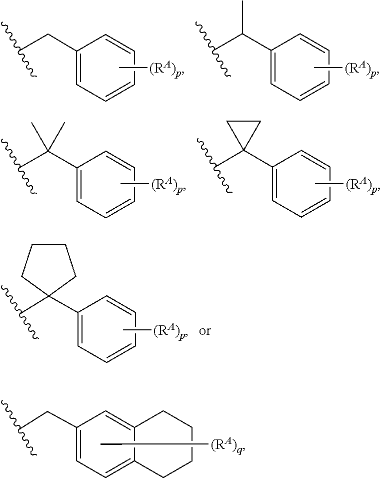
wherein R^{A} is halo, —OH, —CN, —C₁₋₆alkyl, —C₁₋₆haloalkyl, or —C₁₋₆alkoxy.

13. The compound of claim 12, or a pharmaceutically acceptable salt thereof, wherein R¹ is selected from the group consisting of

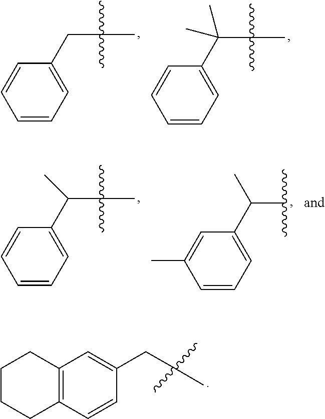

14. The compound of claim 3, or a pharmaceutically acceptable salt thereof, wherein R¹ is
(a) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl,
(b) —C₀₋₆alkylene-(8-12 membered tricyclic heteroaryl), wherein the tricyclic heteroaryl is substituted with 0 or 1 substituents selected from the group consisting of C₃₋₆cycloalkyl and phenyl, or
(c) -(5-12 membered heteroarylene)-phenyl.

15. The compound of claim 1, or a pharmaceutically acceptable salt thereof, wherein R¹ is a 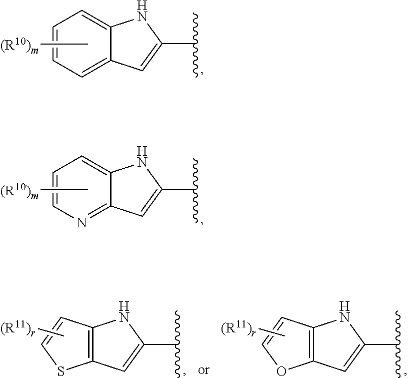
wherein R¹⁰ or R¹¹ is independently halo, —CN, —C₁₋₆alkyl, —C₁₋₆haloalkyl, —C₁₋₆alkoxy, or —SO₂(C₁₋₆alkyl).

16. The compound of claim 15, or a pharmaceutically acceptable salt thereof, wherein R¹ is 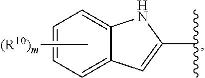
wherein R¹⁰ is halo, CN, —C₁₋₆alkyl, —C₁₋₆haloalkyl, —C₁₋₆alkoxy, or —SO₂(C₁₋₆alkyl); and m is 0, 1, or 2.

17. The compound of any one of claim 1 or 14-16, or a pharmaceutically acceptable salt thereof, wherein R¹ is selected from the group consisting of

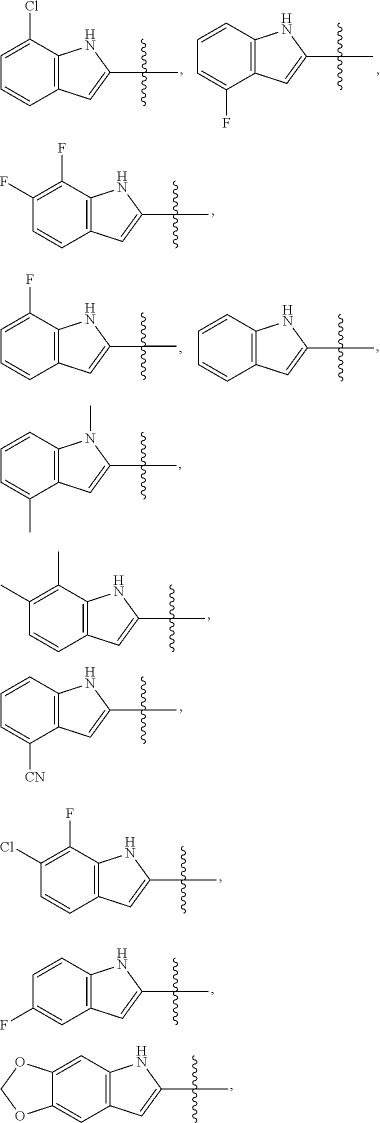

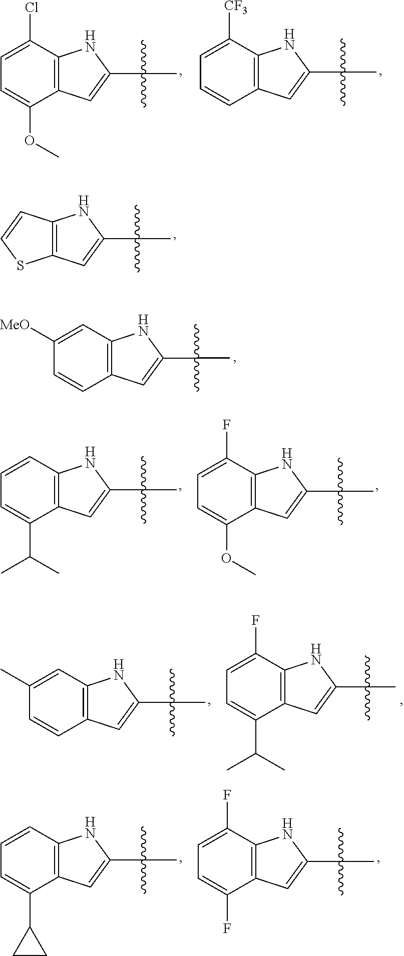

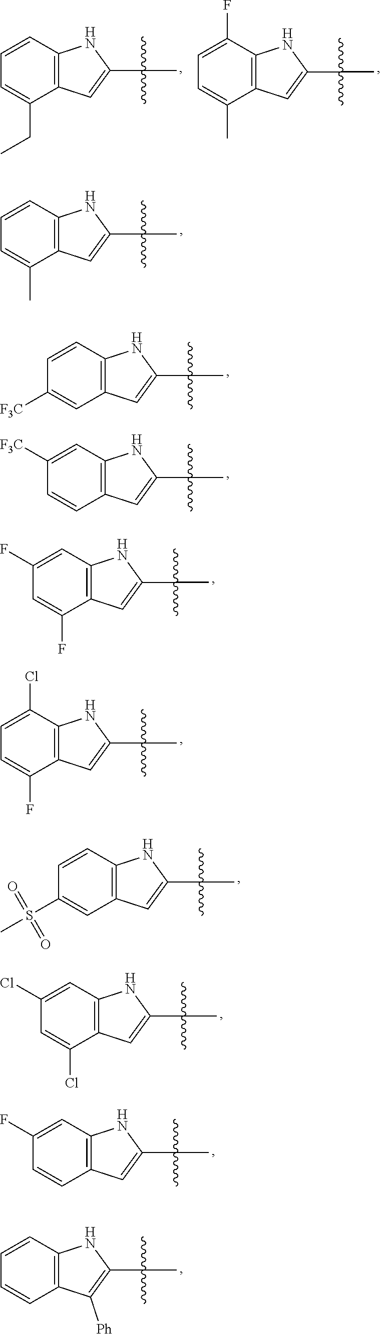

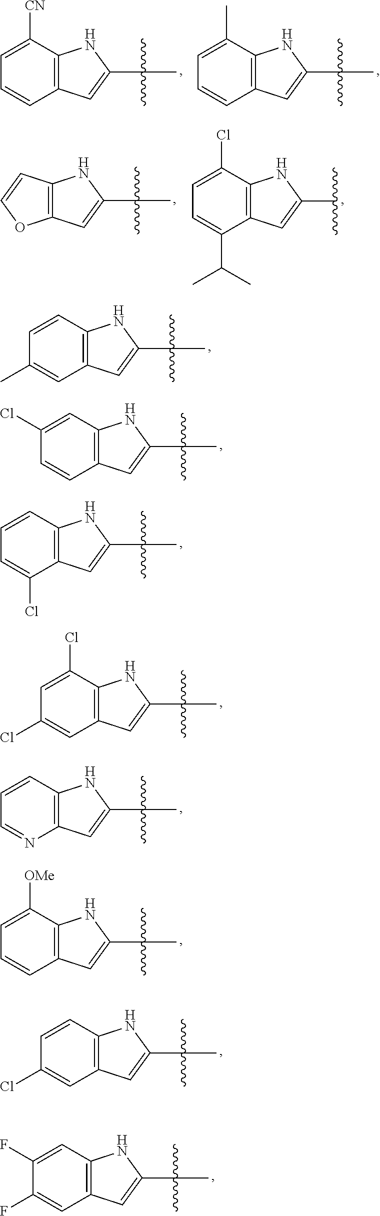

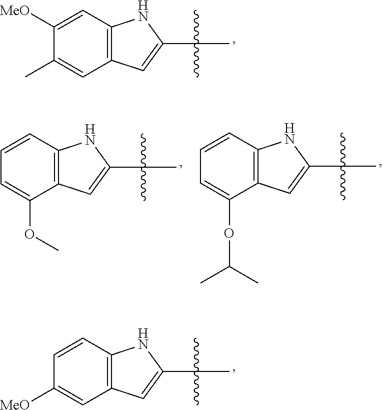 [SMILES: *c1cc2cc(C)c(OC)cc2[nH]1 ; *c1cc2c(OC)cccc2[nH]1 ; *c1cc2c(OC(C)C)cccc2[nH]1 ; *c1cc2cc(OC)ccc2[nH]1]

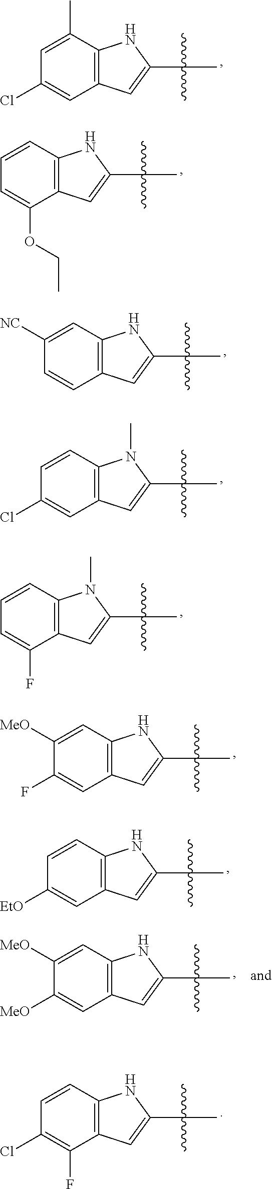

18. The compound of claim 1, or a pharmaceutically acceptable salt thereof, wherein R¹ is selected from any of the following moieties

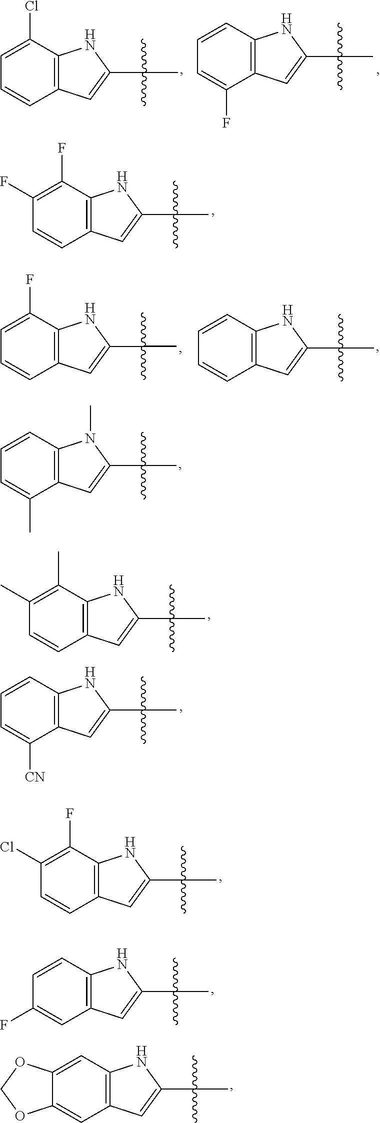

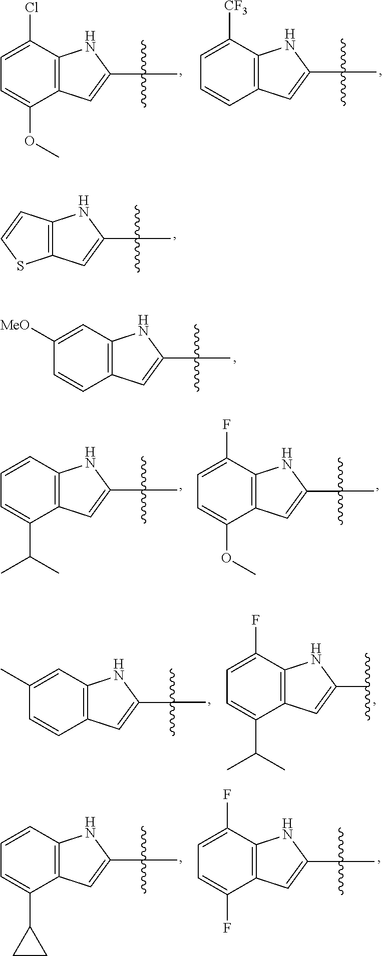

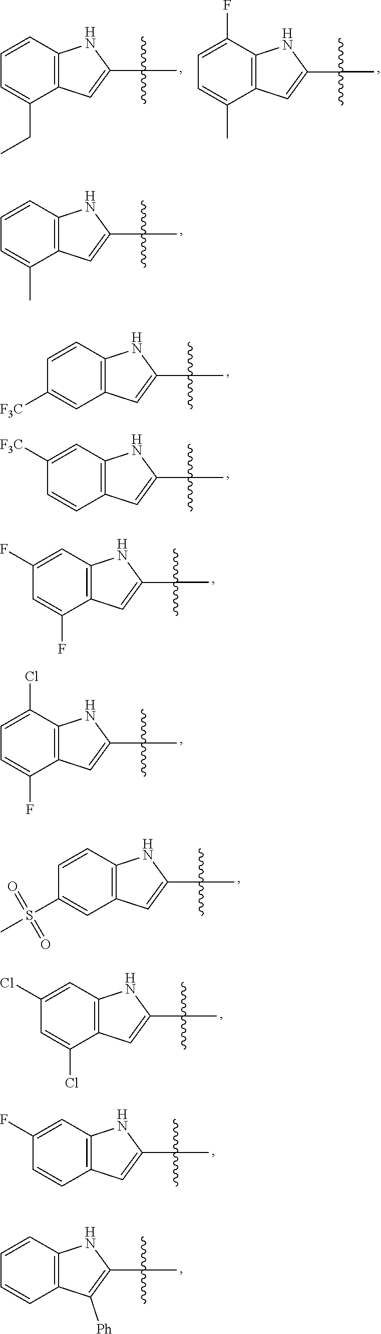

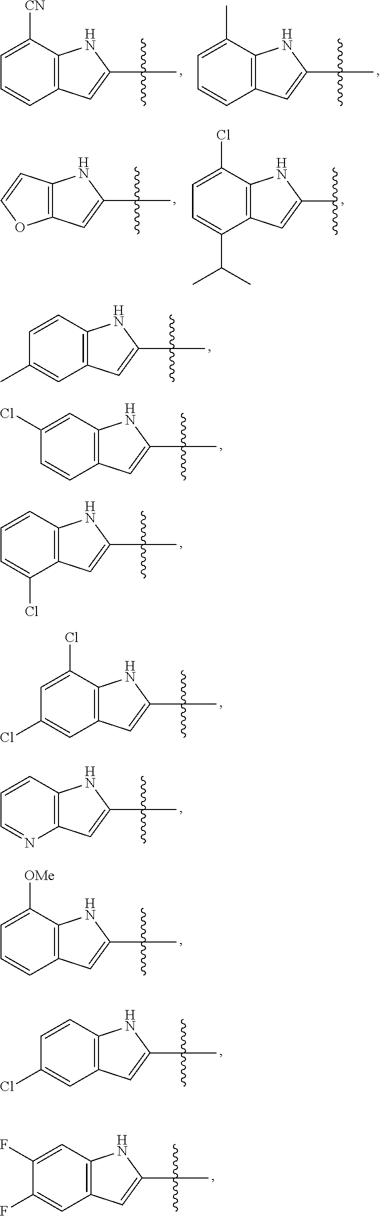

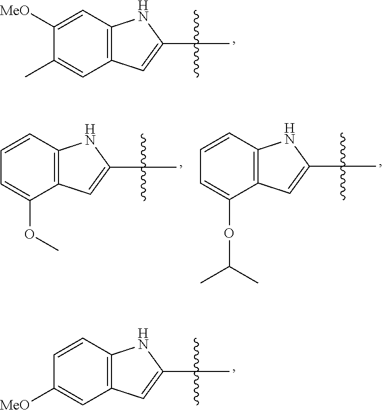 [SMILES: *c1cc2cc(C)c(OC)cc2[nH]1 ; *c1cc2c(OC)cccc2[nH]1 ; *c1cc2c(OC(C)C)cccc2[nH]1 ; *c1cc2cc(OC)ccc2[nH]1]

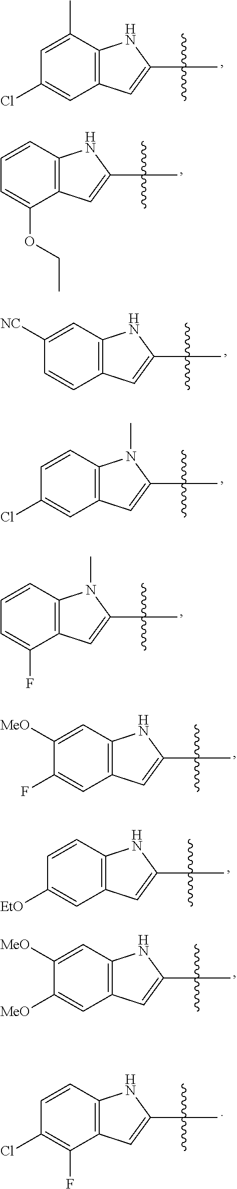

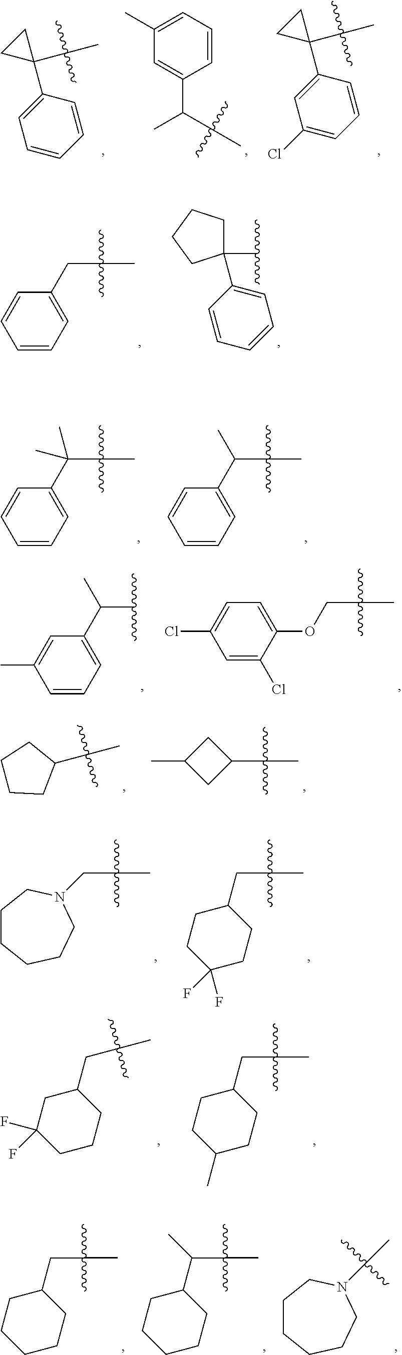

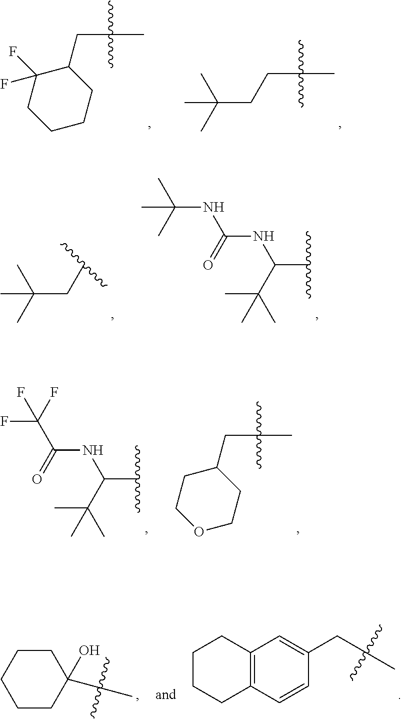

19. The compound of any one of the proceeding claims, or a pharmaceutically acceptable salt thereof, wherein
R² is H;
R³ and R⁴ are each independently —H, halo, —OH, —CN, —NH₂, —NO₂, methylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl;
R⁵ is —H, halo, —C₁₋₆alkyl, or —C₁₋₆haloalkyl; or
R³ and R⁴ together with the carbon atom to which they are attached form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
wherein at each occurrence, the cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

20. The compound of any one of the proceeding claims, or a pharmaceutically acceptable salt thereof, wherein
R² is H;
R³ and R⁴ are each independently —H, halo, —OH, —CN, methylenyl, —C₁₋₆alkyl, —C₁₋₆alkoxy, phenyl, phenoxy, benzyl, C₃-C₆cycloalkyl, 5-7 membered heterocycloalkyl, or 5-9 membered heteroaryl;
R⁵ is —H, halo, —C₁₋₆alkyl, or —C₁₋₆haloalkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
wherein at each occurrence, the cycloalkyl, heterocycloalkyl, phenyl, phenoxy, benzyl, and heteroaryl are each independently substituted with 0, 1, or 2 substituents selected from the group consisting of halo, —OH, —CN, —C₁₋₆alkyl, —C₁₋₆haloalkyl, and —C₁₋₆alkoxy.

21. The compound of any one of the proceeding claims, or a pharmaceutically acceptable salt thereof, wherein
R² is H;
R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl;
R⁵ is —H, halo, or —C₁₋₆alkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R² and R³ together with the carbon atoms to which they are attached, form a C₃-C₆cycloalkyl; or
R² and R⁵ together with the pyrrolidine ring to which they are attached form a bridged 6 to 8 membered heterocycloalkyl;
wherein at each occurrence, the cycloalkyl, heterocycloalkyl, phenyl, phenoxy, and benzyl are each independently substituted with 0, 1, or 2 substituents selected from the group consisting of halo and —C₁₋₆alkyl.

22. The compound of any one of claims 19-21, or a pharmaceutically acceptable salt thereof, wherein
R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0, 1, or 2 substituents selected from the group consisting of halo and —C₁₋₆alkyl.

23. The compound of claim 22, or a pharmaceutically acceptable salt thereof, wherein
R³ is —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl; and
R⁴ is —H, halo, or —C₁₋₆alkyl.

24. The compound of claim 23, or a pharmaceutically acceptable salt thereof, wherein
R³ is —H, —F, -Me, -Et, —OH, —CN, methylenyl, —OMe, phenyl, phenoxy, 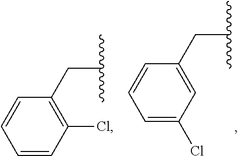
or cyclohexyl; and R⁴ is —H, —F, -Me, or -Et.

25. The compound of any one of the proceeding claims, or a pharmaceutically acceptable salt thereof, wherein R² is H and R⁵ is —H or —C₁₋₆alkyl.

26. The compound of any one of the proceeding claims, or a pharmaceutically acceptable salt thereof, wherein R² and R⁵ are H.

27. The compound of claim 19-22, or a pharmaceutically acceptable salt thereof, wherein
R³ is —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl;
R⁴ is —H, halo, or —C₁₋₆alkyl;
R² is —H; and
R⁵ is —H or —C₁₋₆alkyl.

28. The compound of claim 27, or a pharmaceutically acceptable salt thereof, wherein
R³ is —H, —F, -Me, -Et, —OH, —CN, methylenyl, —OMe, phenyl, phenoxy, 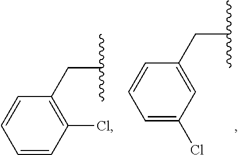
or cyclohexyl;
R⁴ is —H, —F, -Me, or -Et; and R² and R⁵ are —H.

29. The compound of claim 19-21, or a pharmaceutically acceptable salt thereof, wherein R³ and R⁴ together with the carbon atom to which they are attached, form a C₃-C₆cycloalkyl, wherein the cycloalkyl is substituted with 0, 1, or 2 substituents selected from the group consisting of halo and —C₁₋₆alkyl.

30. The compound of claim 29, or a pharmaceutically acceptable salt thereof, wherein
R³ and R⁴ together with the carbon atom to which they are attached, form a C₃-C₆cycloalkyl;
R² is H; and R⁵ is —H or —C₁₋₆alkyl.

31. The compound of claim 29, or a pharmaceutically acceptable salt thereof, wherein
R³ and R⁴ together with the carbon atom to which they are attached, form a C₃-C₄cycloalkyl; and R² and R⁵ are —H.

32. The compound of claim 19-21, or a pharmaceutically acceptable salt thereof, wherein R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl.

33. The compound of claim 32, or a pharmaceutically acceptable salt thereof, wherein
R³ and R⁵ together with the carbon atoms to which they are attached, form a C₃-C₆cycloalkyl, wherein the cycloalkyl is substituted with 0, 1, or 2 substituents which is —C₁₋₆alkyl;
R² is H; and R⁴ is —H or —C₁₋₆alkyl.

34. The compound of claim 33, or a pharmaceutically acceptable salt thereof, wherein
R³ and R⁵ together with the carbon atoms to which they are attached, form a C₃-C₅cycloalkyl, wherein the cycloalkyl is substituted with 0, 1, or 2 substituents which is -Me; and R² and R⁴ are —H.

35. The compound of claim 19-21, or a pharmaceutically acceptable salt thereof, wherein R⁴ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl.

36. The compound of claim 35, or a pharmaceutically acceptable salt thereof, wherein
R⁴ and R⁵ together with the carbon atoms to which they are attached, form a C₃-C₆cycloalkyl, wherein the cycloalkyl is substituted with 0, 1, or 2 substituents which is —C₁₋₆alkyl;
R² is H; and R³ is —H or —C₁₋₆alkyl.

37. The compound of claim 36, or a pharmaceutically acceptable salt thereof, wherein
R⁴ and R⁵ together with the carbon atoms to which they are attached, form a C₃-C₅cycloalkyl, wherein the cycloalkyl is substituted with 0, 1, or 2 substituents which is -Me; and R² and R³ are —H.

38. The compound of any one of claims 19-21, or a pharmaceutically acceptable salt thereof, wherein R² and R³ together with the carbon atoms to which they are attached, form a C₃-C₆cycloalkyl, wherein the cycloalkyl is substituted with 0, 1, or 2 substituents selected from the group consisting of halo and —C₁₋₆alkyl.

39. The compound of claim 38, or a pharmaceutically acceptable salt thereof, wherein
R² and R³ together with the carbon atoms to which they are attached, form a C₃-C₆cycloalkyl; and
R⁴ and R⁵ are each independently —H or —C₁₋₆alkyl.

40. The compound of claim 39, or a pharmaceutically acceptable salt thereof, wherein
R² and R³ together with the carbon atoms to which they are attached, form a C₃-C₆cycloalkyl; and R⁴ and R⁵ are —H.

41. The compound of any one of claims 19-21, or a pharmaceutically acceptable salt thereof, wherein R² and R⁵ together with the pyrrolidine ring to which they are attached form a bridged 6 to 8 membered heterocycloalkyl, wherein the heterocycloalkyl is substituted with 0, 1, or 2 substituents selected from the group consisting of halo and —C₁₋₆alkyl.

42. The compound of claim 41, or a pharmaceutically acceptable salt thereof, wherein
R² and R⁵ together with the pyrrolidine ring to which they are attached form a bridged 6 to 7 membered heterocycloalkyl, wherein the heterocycloalkyl is substituted with 0 or 1 substituent selected from the group consisting of halo and —C₁₋₆alkyl; and
R³ and R⁴ are each independently —H or —C₁₋₆alkyl.

43. The compound of claim 42, or a pharmaceutically acceptable salt thereof, wherein
R² and R⁵ together with the pyrrolidine ring to which they are attached form a bridged 7 membered heterocycloalkyl, wherein the heterocycloalkyl is substituted with —F; and
R³ and R⁴ are —H.

44. The compound of claim 43, or a pharmaceutically acceptable salt thereof, wherein
R² and R⁵ together with the pyrrolidine ring to which they are attached form a bridged 8 membered heterocycloalkyl, wherein the heterocycloalkyl is optionally substituted; and
R³ and R⁴ are —H.

45. The compound of any one of the the proceeding claims, or a pharmaceutically acceptable salt thereof, wherein R⁷ and R⁸ are H.

46. The compound of any one of the proceeding claims, or a pharmaceutically acceptable salt thereof, wherein n is 1 or 2.

47. The compound of any one of the proceeding claims, or a pharmaceutically acceptable salt thereof, wherein n is 1.

48. The compound of any one of the proceeding claims, or a pharmaceutically acceptable salt thereof, wherein the compound of formula I is a compound of formula A

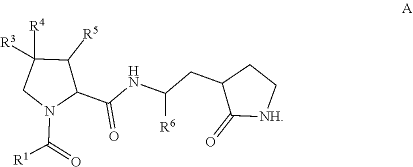

49. The compound of claim 48, or a pharmaceutically acceptable salt thereof, wherein
R¹ is
(a) —C₁₋₆alkyl,
(b) —C₁₋₆alkylene-NHCO—C₁₋₆haloalkyl,
(c) —C₀₋₆alkylene-(saturated cycloalkyl),
(d) —C₀₋₆alkylene-heterocycloalkyl,
(e) -heteroarylene-cycloalkyl, or
(f) -heteroarylene-aryl;
(g) 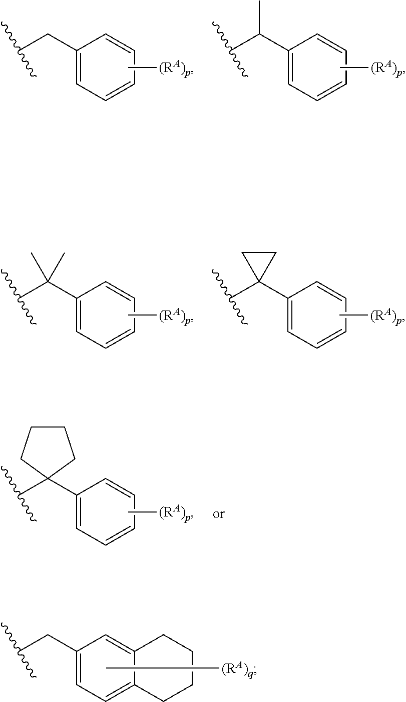
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; and q is 0, 1, 2, 3, 4, 5, 6, 7, 8, 9, 10, or 11;
(h) 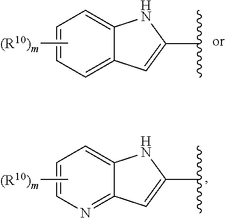
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2;
(i) 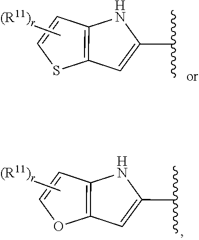
wherein R¹¹ is halo, CN, —C₁₋₆alkyl, —C₁₋₆haloalkyl, —C₁₋₆alkoxy, or —SO₂(C₁₋₆alkyl); and r is 0 or 1;
R³ and R⁴ are each independently —H, halo, —OH, —CN, —NH₂, —NO₂, methylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl;
R⁵ is —H, halo, —C₁₋₆alkyl, or —C₁₋₆haloalkyl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl; and
R⁶ is 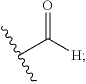
wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

50. The compound of claim 49, or a pharmaceutically acceptable salt thereof, wherein R¹ is
(a) —C₁₋₆alkylene-NHCO—C₁₋₆haloalkyl,
(b) —C₀₋₆alkylene-(saturated cycloalkyl),
(c) —C₀₋₆alkylene-(tricyclic heteroaryl),
(d) —C₀₋₆alkylene-heterocycloalkyl,
(e) 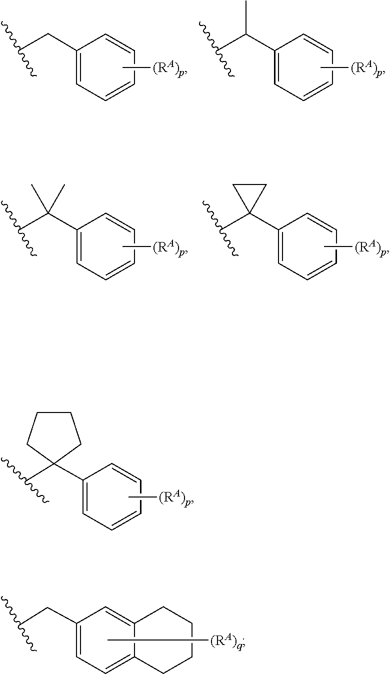
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(f) 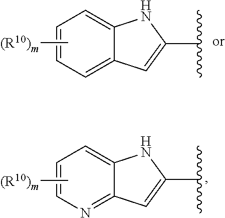
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(g) 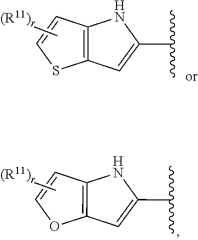
wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

51. The compound of claim 50, or a pharmaceutically acceptable salt thereof, wherein R¹ is selected from the group consisting of

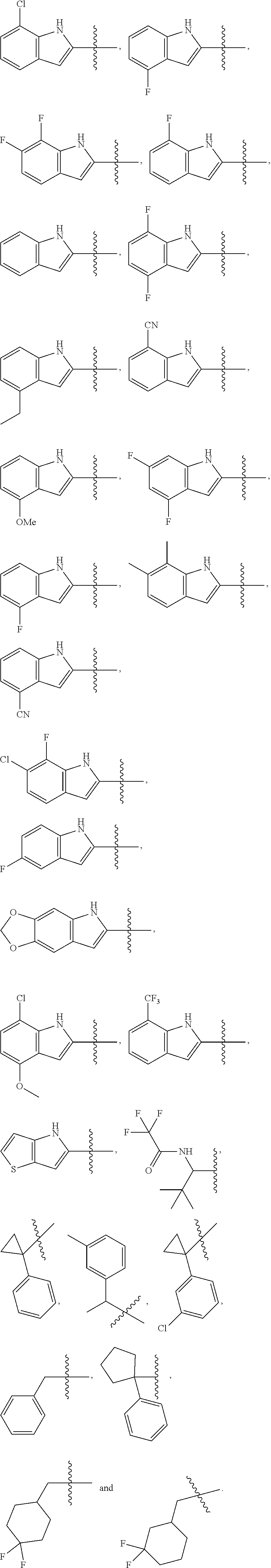

52. The compound of claim 49, or a pharmaceutically acceptable salt thereof, wherein
R³ and R⁴ are each independently —H, halo, —OH, —CN, methylenyl, —C₁₋₆alkyl, —C₁₋₆alkoxy, phenyl, phenoxy, benzyl, or C₃-C₆cycloalkyl, wherein the phenyl, phenoxy, and benzyl are each independently substituted with 0 or 1 substituent selected from the group consisting of halo and —C₁₋₆alkyl; and
R⁵ is —H or —C₁₋₆alkyl.

53. The compound of claim 52, or a pharmaceutically acceptable salt thereof, wherein
R³ is —H, —F, -Me, -Et, —OH, —CN, methylenyl, —OMe, phenyl, phenoxy, 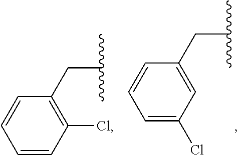
or cyclohexyl;
R⁴ is —H, —F, -Me, or -Et; and R² and R⁵ are —H.

54. The compound of claim 49, or a pharmaceutically acceptable salt thereof, wherein R³ and R⁴ together with the carbon atom to which they are attached, form a C₃-C₆cycloalkyl; and R⁵ is H.

55. The compound of claim 49, or a pharmaceutically acceptable salt thereof, wherein R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl; and R⁴ is H.

56. The compound of any one of claims 48-51, or a pharmaceutically acceptable salt thereof, wherein the compound of formula A is a compound of formula B

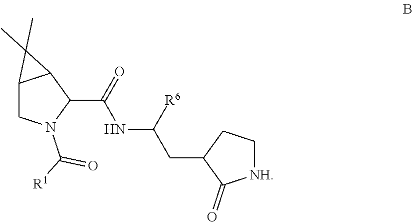

57. The compound of claim 56, or a pharmaceutically acceptable salt thereof, wherein the compound of formula B is a compound of formula B-1

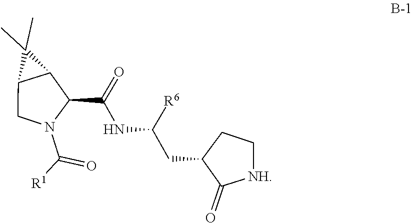

58. The compound of any one of claims 48-51, or a pharmaceutically acceptable salt thereof, wherein the compound of formula A is a compound of formula C

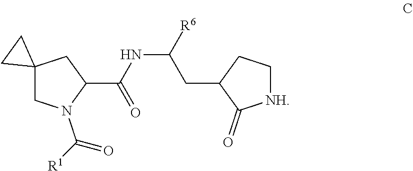

59. The compound of claim 58, or a pharmaceutically acceptable salt thereof, wherein the compound of formula C is a compound of formula C-1

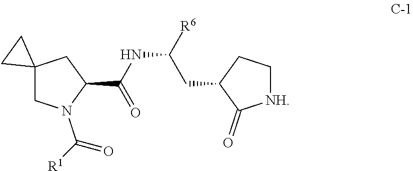

60. The compound of any one of claims 56-59, or a pharmaceutically acceptable salt thereof, wherein
R¹ is
(a) —C₁₋₆alkylene-NHCO—C₁₋₆haloalkyl,
(b) —C₀₋₆alkylene-(saturated cycloalkyl),
(c) —C₀₋₆alkylene-heterocycloalkyl,
(d) —C₀₋₆alkylene-(tricyclic heteroaryl),
(e) 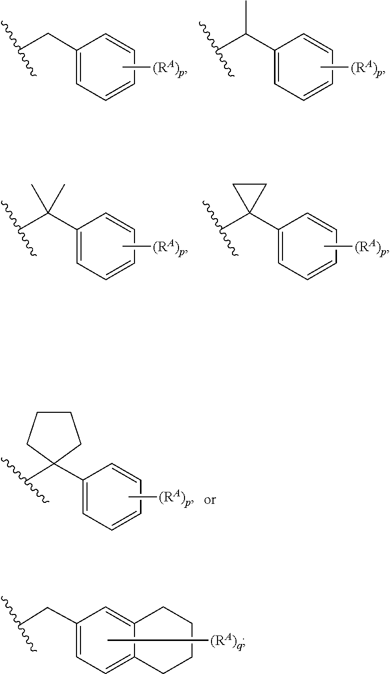
R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(f) 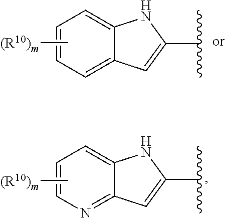
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(g) 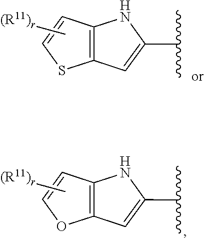
wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, and heteroaryl are each independently substituted with 0, 1, 2, or 3 substituents selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl); and
R⁶ is

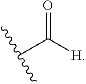

61. The compound of claim 60, or a pharmaceutically acceptable salt thereof, wherein R¹ is
(a) —C₁₋₆alkylene-NHCO—C₁₋₆haloalkyl,
(b) —C₀₋₆alkylene-(saturated cycloalkyl),
(c) —C₀₋₆alkylene-(tricyclic heteroaryl),
(d) 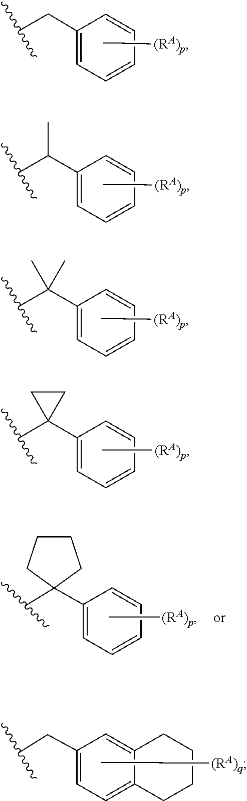
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; q is 0, 1, 2, 3, 4, 5, 6, 7, 8, 9, 10, or 11;
(e) 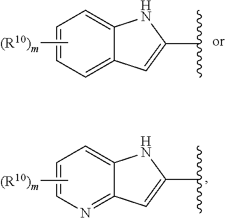
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2; and
(f) 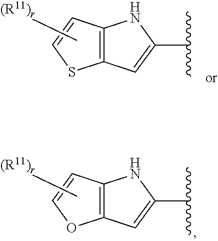
wherein R¹¹ is halo, CN, —C₁₋₆alkyl, —C₁₋₆haloalkyl, —C₁₋₆alkoxy, or —SO₂(C₁₋₆alkyl); and r is 0 or 1;
wherein at each occurrence, the cycloalkyl, saturated cycloalkyl, heterocycloalkyl, aryl, phenyl, and heteroaryl are each independently substituted with 0, 1, or 2 substituents selected from the group consisting of halo, —OH, —CN, —NH₂, —NH(C₁₋₆alkyl), —N(C₁₋₆alkyl)₂, —C₁₋₆alkyl, —C₁₋₆haloalkyl, —C₁₋₆alkoxy, —SO₂(C₁₋₆alkyl), and —CO(C₁₋₆alkyl); and
R⁶ is

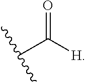

62. The compound of any one of claims 48-61, or a pharmaceutically acceptable salt thereof, wherein
R¹ is selected from the group consisting of 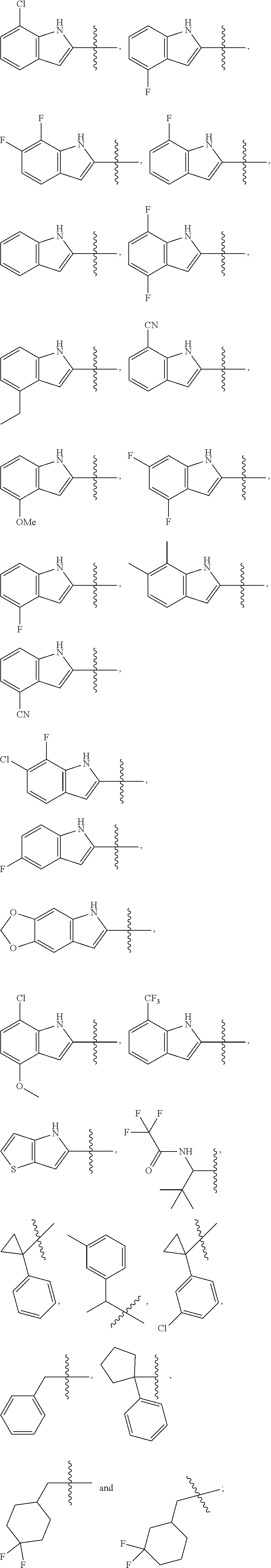
and
R⁶ is

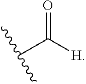

63. A compound of formula II: 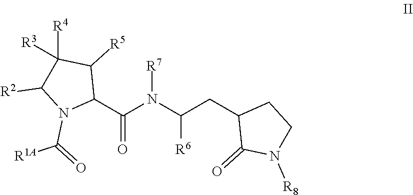
or a pharmaceutically acceptable salt thereof, wherein:
R^{1A} is H,
—C₁₋₆alkylene-NHCONH—C₁₋₆alkyl,
—C₁₋₆alkylene-NHCO(heteroaryl),
—C₁₋₆alkylene-cycloalkyl, 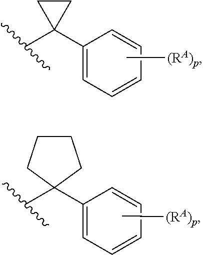
wherein R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; p is 0, 1, 2, 3, 4, or 5; 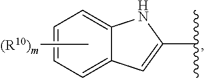
wherein R¹⁰ is halo, CN, —C₁₋₆alkyl, —C₁₋₆haloalkyl, —O(C₁₋₆ branched alkyl), or —SO₂(C₁₋₆alkyl); and m is 0, 1, or 2;
R³, R⁴, and R⁵ are each independently —H, halo, —OH, —CN, —NH₂, —NO₂, alkylenyl, —C₁₋₆alkyl, —C₁₋₆alkoxy, —C₁₋₆haloalkyl, —O—C₀₋₆alkylene-aryl, aryl, cycloalkyl, heterocycloalkyl, or heteroaryl; or
R³ and R⁴ together with the carbon atom to which they are attached, form a C₃-C₆cycloalkyl; or
R³ and R⁵ together with the carbon atoms to which they are attached, form a C₃-C₆cycloalkyl; or
R⁴ and R⁵ together with the carbon atoms to which they are attached, form a C₃-C₆cycloalkyl;
R² is H; or
R² and R³ together with the carbon atoms to which they are attached, form a C₃-C₆cycloalkyl;
R⁶ is 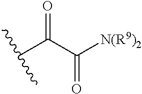
R⁷ and R⁸ are each independently H or C₁₋₆alkyl;
each R⁹ is independently H, C₁₋₆alkyl, cycloalkyl, or heteroaryl; and
R¹² is heterocyclyl or heteroaryl;
wherein at each occurrence, cycloalkyl, heterocycloalkyl, aryl, and heteroaryl are each independently substituted with 0, 1, 2, 3, or 4 substituents independently selected from the group consisting of halo, —OH, —CN, —NH₂, —NH(C₁₋₆alkyl), —N(C₁₋₆alkyl)₂, —NO₂, —C₁₋₆alkyl, —C₁₋₆haloalkyl, —C₁₋₆alkoxy, —SO₂(C₁₋₆alkyl), and —CO(C₁₋₆alkyl).

64. The compound of claim 63, or a pharmaceutically acceptable salt thereof, wherein R^{1A} is —C₁₋₆alkylene-NHCONH—C₁₋₆alkyl.

65. The compound of claim 63 or 64, or a pharmaceutically acceptable salt thereof, wherein R^{1A} is of the formula:

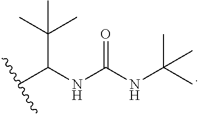 [SMILES: *C(NC(=O)NC(C)(C)C)C(C)(C)C]

66. The compound of claim 63, or a pharmaceutically acceptable salt thereof, wherein R^{1A} is —C₁₋₆alkylene-NHCO(heteroaryl).

67. The compound of claim 66, or a pharmaceutically acceptable salt thereof, wherein R^{1A} is of the formula:

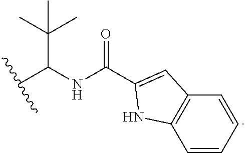 [SMILES: *C(NC(=O)c1cc2ccccc2[nH]1)C(C)(C)C]

68. The compound of claim 63, or a pharmaceutically acceptable salt thereof, wherein R^{1A} is —C₁₋₆alkylene-cycloalkyl, wherein the cycloalkyl is optionally substituted by halo, —C₁₋₆alkyl, —OH, —CN, —NH₂, or —NO₂.

69. The compound of claim 68, or a pharmaceutically acceptable salt thereof, wherein R^{1A} is of the formula:

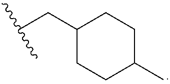 [SMILES: *CC1CCC(C)CC1]

70. The compound of claim 63, or a pharmaceutically acceptable salt thereof, wherein R^{1A} is of the formula: 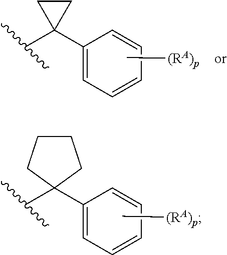
R^{A} is halo, —OH, —CN, —NH₂, —NO₂, —C₁₋₆alkyl, alkenyl, —C₁₋₆alkoxy, —C₁₋₆haloalkyl, —O—C₀₋₆alkylene-aryl, aryl, cycloalkyl, heterocycloalkyl, or heteroaryl; and
p is0, 1, 2, or 3.

71. The compound of claim 63, or a pharmaceutically acceptable salt thereof, wherein R^{1A} is of the formula:

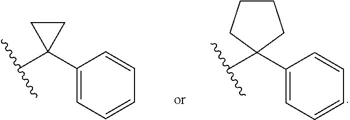 [SMILES: *C1(c2ccccc2)CC1 ; *C1(c2ccccc2)CCCC1]

72. The compound of claim 63, or a pharmaceutically acceptable salt thereof, wherein R^{1A} is of the formula: 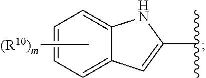
wherein R¹⁰ is halo, CN, —C₁₋₆alkyl, —C₁₋₆haloalkyl, —O(C₁₋₆ branched alkyl), or —SO₂(C₁₋₆alkyl); and
m is 0, 1, or 2.

73. The compound of claim 72, or a pharmaceutically acceptable salt thereof, wherein R^{1A} is of the formula

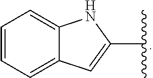 [SMILES: *c1cc2ccccc2[nH]1]

74. The compound of any one of claims 63-73, or a pharmaceutically acceptable salt thereof, wherein R² is H.

75. The compound of any one of claims 63-73, or a pharmaceutically acceptable salt thereof, wherein R³ and R⁴ together with the carbon atom to which they are attached, form a C₃-C₆cycloalkyl; and R⁵ is H.

76. The compound of any one of claims 63-73, or a pharmaceutically acceptable salt thereof, wherein R³ and R⁵ together with the carbon atoms to which they are attached, form a C₃-C₆cycloalkyl, wherein the cycloalkyl is substituted with 0, 1, or 2 substituents selected from the group consisting of halo and —C₁₋₆alkyl; and R⁴ is H.

77. The compound of any one of claims 63-76, or a pharmaceutically acceptable salt thereof, wherein R⁶ is of the formula: 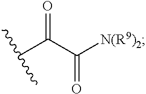
and
each R⁹ is independently H, C₁₋₆alkyl, or cycloalkyl.

78. The compound of any one of claims 63-77, or a pharmaceutically acceptable salt thereof, wherein R⁶ is of the formula:

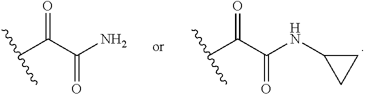 [SMILES: *C(=O)C(N)=O ; *C(=O)C(=O)NC1CC1]

79. The compound of any one of claims 63-78, or a pharmaceutically acceptable salt thereof, wherein R⁶ is of the formula:

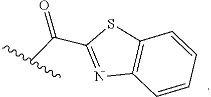 [SMILES: *C(=O)c1nc2ccccc2s1]

80. The compound of any one of claims 63-79, or a pharmaceutically acceptable salt thereof, wherein the compound of formula II is a compound of formula II-A or II-B:

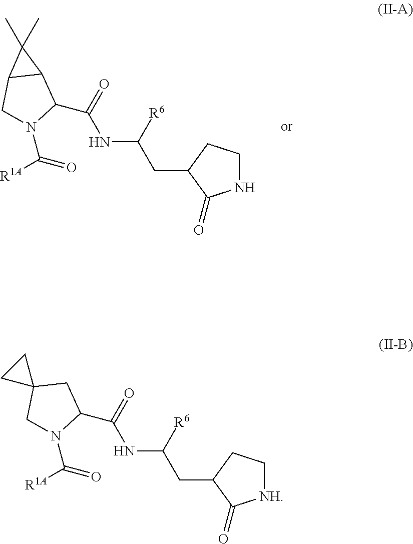

81. The compound of any one of claims 63-80, or a pharmaceutically acceptable salt thereof, wherein the compound is of the formula: 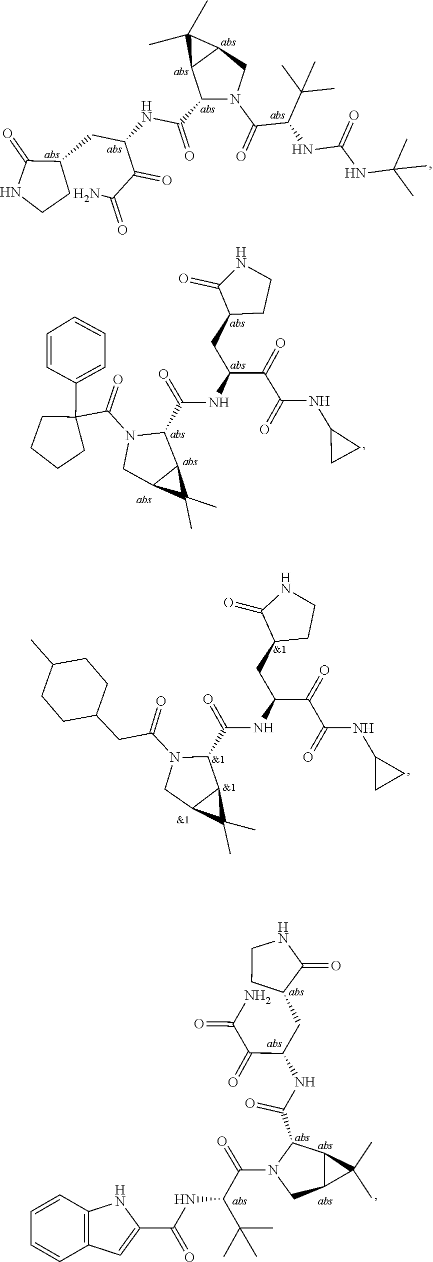 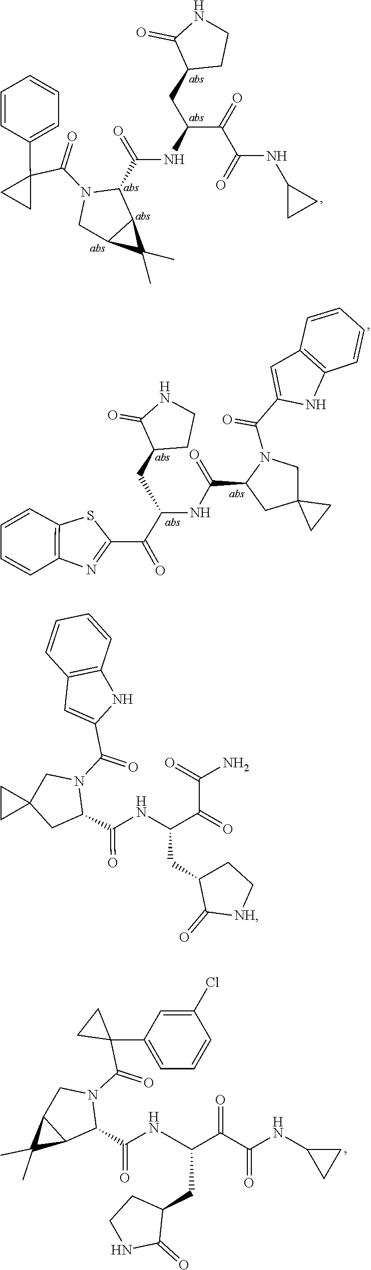 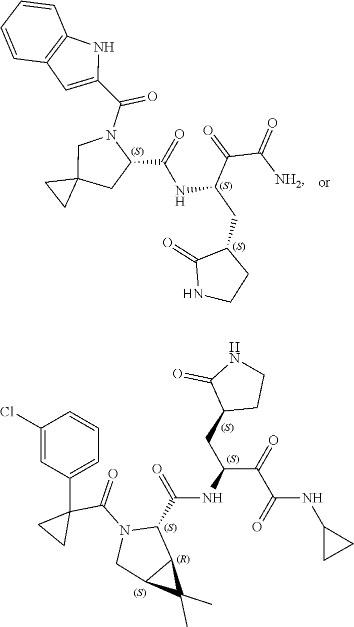
or a pharmaceutically acceptable salt thereof.

82. The compound of any one of claims 1-81 selected from:

| Compound | |
|---|---|
| No. | Chemical Name |
| 1 | (1R,2S,5S)-6,6-dimethyl-N-((S)-1-oxo-3-((S)-2-oxopyrrolidin-3-yl)propan-2-yl)-3- |
| | (1-phenylcyclopropane-1-carbonyl)-3-azabicyclo[3.1.0]hexane-2-carboxamide, |
| 2 | (S)-5-(7-chloro-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 3 | (S)-5-(4-fluoro-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 4 | (1R,2S,5S)-3-(6,7-difluoro-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 5 | (1R,2S,5S)-3-(7-chloro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 6 | (1R,2S,5S)-3-(7-fluoro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 7 | (1R,2S,5S)-6,6-dimethyl-N-((S)-1-oxo-3-((S)-2-oxopyrrolidin-3-yl)propan-2-yl)-3- |
| | ((R)-2-(m-tolyl)propanoyl)-3-azabicyclo[3.1.0]hexane-2-carboxamide, |
| 8 | (1R,2S,5S)-3-(1-(3-chlorophenyl)cyclopropane-1-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 9 | (S)-1-(7-chloro-1H-indole-2-carbonyl)-4,4-dimethyl-N-((S)-1-oxo-3-((S)-2- |
| | oxopyrrolidin-3-yl)propan-2-yl)pyrrolidine-2-carboxamide, |
| 10 | (S)-6-(1H-indole-2-carbonyl)-N-((S)-1-oxo-3-((S)-2-oxopyrrolidin-3-yl)propan-2- |
| | yl)-6-azaspiro[3.4]octane-7-carboxamide, |
| 11 | (1R,2S,5S)-3-(1H-indole-2-carbonyl)-6,6-dimethyl-N-((S)-1-oxo-3-((S)-2- |
| | oxopiperidin-3-yl)propan-2-yl)-3-azabicyclo[3.1.0]hexane-2-carboxamide, |
| 12 | (1R,2S,5S)-3-((S)-3,3-dimethyl-2-(2,2,2-trifluoroacetamido)butanoyl)-6,6-dimethyl- |
| | N-((S)-1-oxo-3-((S)-2-oxopyrrolidin-3-yl)propan-2-yl)-3-azabicyclo[3.1.0]hexane-2- |
| | carboxamide, |
| 13 | (1R,2S,5S)-3-(4,7-difluoro-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 14 | (1R,2S,5S)-3-(4-ethyl-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 15 | (1R,2S,5S)-3-(4-fluoro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 16 | (1S,2S,5R,6R)-3-(2-(4,4-difluorocyclohexyl)acetyl)-6-methyl-N-((S)-1-oxo-3-((S)-2- |
| | oxopyrrolidin-3-yl)propan-2-yl)-3-azabicyclo[3.1.0]hexane-2-carboxamide, |
| 17 | (1R,2S,5S)-6,6-dimethyl-N-((S)-1-oxo-3-((S)-2-oxopyrrolidin-3-yl)propan-2-yl)-3- |
| | (1-phenylcyclopentane-1-carbonyl)-3-azabicyclo[3.1.0]hexane-2-carboxamide, |
| 18 | (1R,2S,5S)-3-(6,7-dimethyl-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 19 | (1R,2S,5S)-3-(6-fluoro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 20 | (1R,2S,5S)-3-(4,6-difluoro-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 21 | (1R,2S,5S)-3-(6-chloro-7-fluoro-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 22 | (1R,2S,5S)-3-(2-(3,3-difluorocyclohexyl)acetyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 23 | (1R,2S,5S)-6,6-dimethyl-N-((S)-1-oxo-3-((S)-2-oxopyrrolidin-3-yl)propan-2-yl)-3- |
| | (2-phenylacetyl)-3-azabicyclo[3.1.0]hexane-2-carboxamide, |
| 24 | (1R,2S,5S)-3-(5-fluoro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 25 | (1R,2S,5S)-3-(7-chloro-1H-indole-2-carbonyl)-6,6-dimethyl-N-((S)-1-oxo-3-((S)-2- |
| | oxopiperidin-3-yl)propan-2-yl)-3-azabicyclo[3.1.0]hexane-2-carboxamide, |
| 26 | (1S,3aR,6aS)-2-(1H-indole-2-carbonyl)-N-((S)-1-oxo-3-((S)-2-oxopyrrolidin-3- |
| | yl)propan-2-yl)octahydrocyclopenta[c]pyrrole-1-carboxamide, |
| 27 | (1R,2S,5S)-3-(5H-[1,3]dioxolo[4,5-f]indole-6-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 28 | (1R,2S,5S)-3-(7-chloro-4-methoxy-1H-indole-2-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 29 | (1R,2S,5S)-6,6-dimethyl-N-((S)-1-oxo-3-((S)-2-oxopyrrolidin-3-yl)propan-2-yl)-3- |
| | (7-(trifluoromethyl)-1H-indole-2-carbonyl)-3-azabicyclo[3.1.0]hexane-2- |
| | carboxamide, |
| 30 | (1R,2S,5S)-6,6-dimethyl-N-((S)-1-oxo-3-((S)-2-oxopyrrolidin-3-yl)propan-2-yl)-3- |
| | (4H-thieno[3,2-b]pyrrole-5-carbonyl)-3-azabicyclo[3.1.0]hexane-2-carboxamide, |
| 31 | (S)-5-(4-methoxy-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 32 | (1R,2S,5S)-3-(7-cyano-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 33 | (1R,2S,5S)-6,6-dimethyl-3-(7-methyl-1H-indole-2-carbonyl)-N-((S)-1-oxo-3-((S)-2- |
| | oxopyrrolidin-3-yl)propan-2-yl)-3-azabicyclo[3.1.0]hexane-2-carboxamide, |
| 34 | (1R,2S,5S)-3-(4-isopropyl-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 36 | (1R,2S,5S)-3-(7-fluoro-4-methoxy-1H-indole-2-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 37 | (1R,2S,5S)-6,6-dimethyl-N-((S)-1-oxo-3-((S)-2-oxopyrrolidin-3-yl)propan-2-yl)-3- |
| | ((R)-2-phenylpropanoyl)-3-azabicyclo[3.1.0]hexane-2-carboxamide, |
| 38 | (2S,4S)-4-cyclohexyl-1-(4-fluoro-1H-indole-2-carbonyl)-N-((S)-1-oxo-3-((S)-2- |
| | oxopyrrolidin-3-yl)propan-2-yl)pyrrolidine-2-carboxamide, |
| 39 | (1R,2S,5S)-3-(7-fluoro-4-isopropyl-1H-indole-2-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 40 | (1R,2S,5S)-6,6-dimethyl-N-((S)-1-oxo-3-((S)-2-oxopyrrolidin-3-yl)propan-2-yl)-3- |
| | (2-(5,6,7,8-tetrahydronaphthalen-2-yl)acetyl)-3-azabicyclo[3.1.0]hexane-2- |
| | carboxamide, |
| 41 | (1R,2S,5S)-3-(4-cyclopropyl-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 42 | (1R,2S,5S)-3-(6-chloro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 43 | (1R,2S,5S)-3-(4-chloro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 44 | (1R,2S,5S)-3-(5,7-dichloro-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 46 | (S)-5-(7-cyano-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 47 | (S)-5-(1H-indole-2-carbonyl)-N-((S)-1-oxo-3-((S)-2-oxopyrrolidin-3-yl)propan-2- |
| | yl)-5-azaspiro[2.4]heptane-6-carboxamide, |
| 48 | (1R,2S,5S)-6,6-dimethyl-3-(2-methyl-2-phenylpropanoyl)-N-((S)-1-oxo-3-((S)-2- |
| | oxopyrrolidin-3-yl)propan-2-yl)-3-azabicyclo[3.1.0]hexane-2-carboxamide, |
| 49 | (1R,2S,5S)-3-(7-chloro-4-fluoro-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 51 | (S)-1-(4-fluoro-1H-indole-2-carbonyl)-4,4-dimethyl-N-((S)-1-oxo-3-((S)-2- |
| | oxopyrrolidin-3-yl)propan-2-yl)pyrrolidine-2-carboxamide, |
| 52 | (1R,2S,5S)-6,6-dimethyl-3-(2-(4-methylcyclohexyl)acetyl)-N-((S)-1-oxo-3-((S)-2- |
| | oxopyrrolidin-3-yl)propan-2-yl)-3-azabicyclo[3.1.0]hexane-2-carboxamide, |
| 53 | (1R,2S,5S)-3-(2-cyclohexylacetyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 54 | (1R,2S,5S)-3-(5,6-difluoro-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 55 | (1R,2S,5S)-3-(2-(azepan-1-yl)acetyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 56 | (1R,2S,5S)-3-(6-methoxy-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide,, |
| 57 | (S)-1-(1H-indole-2-carbonyl)-4,4-dimethyl-N-((S)-1-oxo-3-((S)-2-oxopyrrolidin-3- |
| | yl)propan-2-yl)pyrrolidine-2-carboxamide, |
| 58 | (S)-1-(7-cyano-1H-indole-2-carbonyl)-4,4-dimethyl-N-((S)-1-oxo-3-((S)-2- |
| | oxopyrrolidin-3-yl)propan-2-yl)pyrrolidine-2-carboxamide, |
| 59 | (1R,2S,5S)-3-(4-cyano-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 60 | (1R,2S,5S)-3-(1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 61 | (2S,4S)-4-cyclohexyl-1-(1H-indole-2-carbonyl)-N-((S)-1-oxo-3-((S)-2- |
| | oxopyrrolidin-3-yl)propan-2-yl)pyrrolidine-2-carboxamide, |
| 62 | (S)-1-(7-fluoro-1H-indole-2-carbonyl)-4,4-dimethyl-N-((S)-1-oxo-3-((S)-2- |
| | oxopyrrolidin-3-yl)propan-2-yl)pyrrolidine-2-carboxamide, |
| 63 | (1S,3aR,6aS)-2-(2-(2,4-dichlorophenoxy)acetyl)-N-((S)-1-oxo-3-((S)-2- |
| | oxopyrrolidin-3-yl)propan-2-yl)octahydrocyclopenta[c]pyrrole-1-carboxamide, |
| 64 | (1R,2S,5S)-3-(2-(2,2-difluorocyclohexyl)acetyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 65 | (1R,2S,5S)-3-(4-methoxy-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 66 | (1R,2S,5S)-3-(4-isopropoxy-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 67 | (1R,2S,5S)-3-((R)-2-cyclohexylpropanoyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 68 | (1R,2S,5S)-3-(5-methoxy-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 69 | (1R,2S,5S)-3-(5-chloro-7-methyl-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 70 | (1R,2S,5S)-3-(4-ethoxy-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 71 | (2S,4S)-4-cyclohexyl-1-(7-fluoro-1H-indole-2-carbonyl)-N-((S)-1-oxo-3-((S)-2- |
| | oxopyrrolidin-3-yl)propan-2-yl)pyrrolidine-2-carboxamide, |
| 72 | (1R,2S,5S)-6,6-dimethyl-3-(6-methyl-1H-indole-2-carbonyl)-N-((S)-1-oxo-3-((S)-2- |
| | oxopyrrolidin-3-yl)propan-2-yl)-3-azabicyclo[3.1.0]hexane-2-carboxamide, |
| 73 | (1R,2S,5S)-3-(4,4-dimethylpentanoyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 74 | (1R,2S,5S)-3-(6-cyano-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 75 | (2S,4S)-1-(7-cyano-1H-indole-2-carbonyl)-4-cyclohexyl-N-((S)-1-oxo-3-((S)-2- |
| | oxopyrrolidin-3-yl)propan-2-yl)pyrrolidine-2-carboxamide, |
| 76 | (S)-5-(4-cyano-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 77 | (1R,2S,5S)-3-(7-fluoro-4-methyl-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 78 | (1R,2S,5S)-3-(4H-furo[3,2-b]pyrrole-5-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 79 | (1R,2S,5S)-3-(2-cyclohexylacetyl)-6,6-dimethyl-N-((S)-1-oxo-3-((S)-2-oxopiperidin- |
| | 3-yl)propan-2-yl)-3-azabicyclo[3.1.0]hexane-2-carboxamide, |
| 80 | (1R,2S,5S)-6,6-dimethyl-3-(5-(methylsulfonyl)-1H-indole-2-carbonyl)-N-((S)-1-oxo- |
| | 3-((S)-2-oxopyrrolidin-3-yl)propan-2-yl)-3-azabicyclo[3.1.0]hexane-2-carboxamide, |
| 81 | (2S,4S)-1-(7-chloro-1H-indole-2-carbonyl)-4-cyclohexyl-N-((S)-1-oxo-3-((S)-2- |
| | oxopyrrolidin-3-yl)propan-2-yl)pyrrolidine-2-carboxamide, |
| 82 | (S)-5-(6-cyano-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 83 | (1R,2S,5S)-6,6-dimethyl-N-((S)-1-oxo-3-((S)-2-oxopyrrolidin-3-yl)propan-2-yl)-3- |
| | (1H-pyrrolo[3,2-b]pyridine-2-carbonyl)-3-azabicyclo[3.1.0]hexane-2-carboxamide, |
| 84 | (S)-N-((S)-1-oxo-3-((S)-2-oxopyrrolidin-3-yl)propan-2-yl)-6-(1-phenylcyclopropane- |
| | 1-carbonyl)-6-azaspiro[3.4]octane-7-carboxamide, |
| 85 | (1R,2S,5S)-6,6-dimethyl-3-(4-methyl-1H-indole-2-carbonyl)-N-((S)-1-oxo-3-((S)-2- |
| | oxopyrrolidin-3-yl)propan-2-yl)-3-azabicyclo[3.1.0]hexane-2-carboxamide, |
| 86 | (1R,2S,5S)-6,6-dimethyl-N-((S)-1-oxo-3-((S)-2-oxopyrrolidin-3-yl)propan-2-yl)-3- |
| | (2-(tetrahydro-2H-pyran-4-yl)acetyl)-3-azabicyclo[3.1.0]hexane-2-carboxamide, |
| 87 | (S)-5-(7-fluoro-1H-indole-2-carbonyl)-N-((S)-1-oxo-3-((S)-2-oxopyrrolidin-3- |
| | yl)propan-2-yl)-5-azaspiro[2.4]heptane-6-carboxamide, |
| 88 | (1R,2S,5S)-3-(7-chloro-4-isopropyl-1H-indole-2-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 89 | (1R,2S,5S)-3-(5-chloro-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 90 | (1R,2S,5S)-3-(5-chloro-1-methyl-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 91 | (1R,2S,5S)-3-(7-methoxy-1H-indole-2-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 92 | (1R,2S,5S)-3-((S)-2-cyclohexylpropanoyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 93 | (1R,2S,5S)-6,6-dimethyl-3-(5-methyl-1H-indole-2-carbonyl)-N-((S)-1-oxo-3-((S)-2- |
| | oxopyrrolidin-3-yl)propan-2-yl)-3-azabicyclo[3.1.0]hexane-2-carboxamide, |
| 94 | (1R,2S,5S)-3-(1-hydroxycyclohexane-1-carbonyl)-6,6-dimethyl-N-((S)-1-oxo-3-((S)- |
| | 2-oxopyrrolidin-3-yl)propan-2-yl)-3-azabicyclo[3.1.0]hexane-2-carboxamide, |
| 95 | (1R,2S,5S)-3-(3,3-dimethylbutanoyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 96 | (1R,2S,5S)-6,6-dimethyl-N-((S)-1-oxo-3-((S)-2-oxopyrrolidin-3-yl)propan-2-yl)-3- |
| | (6-(trifluoromethyl)-1H-indole-2-carbonyl)-3-azabicyclo[3.1.0]hexane-2- |
| | carboxamide, |
| 97 | (1R,2S,5S)-3-(4,6-dichloro-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 98 | (2S,4R)-4-fluoro-1-(1H-indole-2-carbonyl)-N-((S)-1-oxo-3-((S)-2-oxopyrrolidin-3- |
| | yl)propan-2-yl)pyrrolidine-2-carboxamide, |
| 100 | (1R,2S,5S)-3-(4-fluoro-1-methyl-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 101 | (1R,2S,5S)-3-(5-fluoro-6-methoxy-1H-indole-2-carbonyl)-6,6-dimethyl-N-((S)-1- |
| | oxo-3-((S)-2-oxopyrrolidin-3-yl)propan-2-yl)-3-azabicyclo[3.1.0]hexane-2- |
| | carboxamide, |
| 102 | (2S,4S)-1-(1H-indole-2-carbonyl)-4-methoxy-N-((S)-1-oxo-3-((S)-2-oxopyrrolidin- |
| | 3-yl)propan-2-yl)pyrrolidine-2-carboxamide, |
| 103 | (2S,4R)-4-cyclohexyl-1-(1H-indole-2-carbonyl)-N-((S)-1-oxo-3-((S)-2- |
| | oxopyrrolidin-3-yl)propan-2-yl)pyrrolidine-2-carboxamide, |
| 104 | (1R,2S,5S)-6,6-dimethyl-N-((S)-1-oxo-3-((S)-2-oxopyrrolidin-3-yl)propan-2-yl)-3- |
| | ((S)-2-phenylpropanoyl)-3-azabicyclo[3.1.0]hexane-2-carboxamide, |
| 106 | (1S,3S,4R,6S)-6-fluoro-2-(1H-indole-2-carbonyl)-N-((S)-1-oxo-3-((S)-2- |
| | oxopyrrolidin-3-yl)propan-2-yl)-2-azabicyclo[2.2.1 ]heptane-3-carboxamide, |
| 107 | (2S,4R)-1-(1H-indole-2-carbonyl)-4-methyl-N-((S)-1-oxo-3-((S)-2-oxopyrrolidin-3- |
| | yl)propan-2-yl)pyrrolidine-2-carboxamide, |
| 108 | (S)-1-(1H-indole-2-carbonyl)-4-methylene-N-((S)-1-oxo-3-((S)-2-oxopyrrolidin-3- |
| | yl)propan-2-yl)pyrrolidine-2-carboxamide, |
| 109 | (2S,4R)-4-(2-chlorobenzyl)-1-(1H-indole-2-carbonyl)-N-((S)-1-oxo-3-((S)-2- |
| | oxopyrrolidin-3-yl)propan-2-yl)pyrrolidine-2-carboxamide, |
| 110 | (1R,2S,5S)-6,6-dimethyl-3-(3-methylcyclobutane-1-carbonyl)-N-((S)-1-oxo-3-((S)- |
| | 2-oxopyrrolidin-3-yl)propan-2-yl)-3-azabicyclo[3.1.0]hexane-2-carboxamide, |
| 111 | (1R,2S,5S)-3-(5-ethoxy-1H-indole-2-carbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 112 | (2S,4R)-4-cyano-1-(1H-indole-2-carbonyl)-N-((S)-1-oxo-3-((S)-2-oxopyrrolidin-3- |
| | yl)propan-2-yl)pyrrolidine-2-carboxamide, |
| 113 | (2S,3aS, 7aS)-1-(1H-indole-2-carbonyl)-N-((S)-1-oxo-3-((S)-2-oxopyrrolidin-3- |
| | yl)propan-2-yl)octahydro-1H-indole-2-carboxamide, |
| 114 | (1R,2S,5S)-3-(5,6-dimethoxy-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 115 | (S)-4,4-difluoro-1-(1H-indole-2-carbonyl)-N-((S)-1-oxo-3-((S)-2-oxopyrrolidin-3- |
| | yl)propan-2-yl)pyrrolidine-2-carboxamide, |
| 117 | (1R,2S,5S)-3-(5-chloro-4-fluoro-1H-indole-2-carbonyl)-6,6-dimethyl-N-((S)-1-oxo- |
| | 3-((S)-2-oxopyrrolidin-3-yl)propan-2-yl)-3-azabicyclo[3.1.0]hexane-2-carboxamide, |
| 118 | (1R,2S,5S)-3-(1,4-dimethyl-1H-indole-2-carbonyl)-6,6-dimethyl-N-((S)-1-oxo-3- |
| | ((S)-2-oxopyrrolidin-3-yl)propan-2-yl)-3-azabicyclo[3.1.0]hexane-2-carboxamide, |
| 121 | (2S,4R)-1-(1H-indole-2-carbonyl)-4-methoxy-N-((S)-1-oxo-3-((S)-2-oxopyrrolidin- |
| | 3-yl)propan-2-yl)pyrrolidine-2-carboxamide, |
| 122 | (1R,2S,5S)-6,6-dimethyl-N-((S)-1-oxo-3-((S)-2-oxopyrrolidin-3-yl)propan-2-yl)-3- |
| | (3-phenyl-1H-indole-2-carbonyl)-3-azabicyclo[3.1.0]hexane-2-carboxamide, |
| 124 | (1R,2S,5S)-3-(cyclopentanecarbonyl)-6,6-dimethyl-N-((S)-1-oxo-3-((S)-2- |
| | oxopyrrolidin-3-yl)propan-2-yl)-3-azabicyclo[3.1.0]hexane-2-carboxamide, |
| 125 | (1S,3S,5S)-2-(1H-indole-2-carbonyl)-N-((S)-1-oxo-3-((S)-2-oxopyrrolidin-3- |
| | yl)propan-2-yl)-2-azabicyclo[3.1.0]hexane-3-carboxamide, |
| 126 | (S)-5-(2-cyclohexylacetyl)-N-((S)-1-oxo-3-((S)-2-oxopyrrolidin-3-yl)propan-2-yl)-5- |
| | azaspiro[2.4]heptane-6-carboxamide, |
| 127 | (S)-N-((S)-1-(benzo[d]thiazol-2-yl)-1-oxo-3-((S)-2-oxopyrrolidin-3-yl)propan-2-yl)- |
| | 5-(1H-indole-2-carbonyl)-5-azaspiro[2.4]heptane-6-carboxamide, |
| 129 | (2S,3aS,6aS)-1-(1H-indole-2-carbonyl)-N-((S)-1-oxo-3-((S)-2-oxopyrrolidin-3- |
| | yl)propan-2-yl)octahydrocyclopenta[b]pyrrole-2-carboxamide, |
| 130 | (1S,2S,5R)-3-(1H-indole-2-carbonyl)-N-((S)-1-oxo-3-((S)-2-oxopyrrolidin-3- |
| | yl)propan-2-yl)-3-azabicyclo[3.1.0]hexane-2-carboxamide, |
| 133 | (2S,4R)-1-(1H-indole-2-carbonyl)-N-((S)-1-oxo-3-((S)-2-oxopyrrolidin-3-yl)propan- |
| | 2-yl)-4-phenoxypyrrolidine-2-carboxamide, |
| 134 | (2S,4S)-1-(1H-indole-2-carbonyl)-N-((S)-1-oxo-3-((S)-2-oxopyrrolidin-3-yl)propan- |
| | 2-yl)-4-phenoxypyrrolidine-2-carboxamide, |
| 137 | (2S,4R)-4-(3-chlorobenzyl)-1-(1H-indole-2-carbonyl)-N-((S)-1-oxo-3-((S)-2- |
| | oxopyrrolidin-3-yl)propan-2-yl)pyrrolidine-2-carboxamide, and |
| 138 | (S)-1-(1H-indole-2-carbonyl)-N-((S)-1-oxo-3-((S)-2-oxopyrrolidin-3-yl)propan-2- |
| | yl)pyrrolidine-2-carboxamide; |

or a pharmaceutically acceptable salt thereof.

83. A pharmaceutical composition for treating a coronavirus infection in a human in need thereof, comprising a compound of any one of the proceeding claims, or a pharmaceutically acceptable salt thereof.

84. A method of treating a viral infection in a patient, comprising administering to the patient a therapeutically effective amount of a compound of any one of claims 1-82, or a pharmaceutically acceptable salt thereof, or a pharmaceutical composition of claim 88.

85. The method of claim 84, wherein the viral infection is caused by a coronavirus.

86. The method of claim 85, wherein the viral infection is a coronavirus infection caused by SARS-CoV-2, SARS-CoV, MERS-CoV, 229E, NL63, OC43, or HKUl.

87. The method of claim 86, wherein the coronavirus infection is caused by SARS-CoV-2.

88. The method of any one of claims 84-87, wherein the patient is suffering from COVID-19.

89. The method of claim 86, wherein the coronavirus infection is caused by SARS-CoV.

90. The method of claim 86, wherein the coronavirus infection is caused by MERS-CoV.

91. The method of any one of claims 84-90, wherein the method inhibits Mpro.

92. The method of any one of the preceding claims 84-91, wherein the compound or a pharmaceutically acceptable salt thereof is administered orally.

93. The method of claim 92, wherein the compound or a pharmaceutically acceptable salt thereof is administered once a day, twice a day, or three times a day.

94. The method of any one of claims 84-93, wherein the therapeutically effective amount is from 0.5 mg to 100 mg, 1 mg to 80 mg, or from 1 mg to 60 mg.

95. The method of any one of claims 84-94, wherein the compound is in a solid dosage form.

96. A compound of any one of claims 1-82 or a pharmaceutically acceptable salt or ester thereof, for use in treating a coronavirus infection in a human.

97. A compound of any one of claims 1-82 or a pharmaceutically acceptable salt or ester thereof, for use in treating SARS-CoV-2 infection in a human.

98. Use of a compound of any one of claims 1-82 or a pharmaceutically acceptable salt thereof in the manufacture of a medicament for treating a coronavirus infection.
